(19) United States
(12) Patent Application Publication
(10) Pub. No.: US 2025/0122529 A1
(43) Pub. Date: Apr. 17, 2025

(54) POLYCISTRONIC VECTORS FOR CELL-BASED THERAPIES

(71) Applicant: Sana Biotechnology, Inc., Seattle, WA (US)

(72) Inventors: Albert Ruzo MATIAS, Seattle, WA (US); William DOWDLE, Seattle, WA (US); Eleonore THAM, Seattle, WA (US); Jessica S. ELMAN, Seattle, WA (US); Adam JOHNSON, Seattle, WA (US)

(21) Appl. No.: 18/579,300

(22) PCT Filed: Jul. 18, 2022
(86) PCT No.: PCT/US2022/073862

§ 371 Date: Jan. 12, 2024

Related U.S. Application Data
(60) Provisional application No. 63/222,954, filed on Jul. 16, 2021.
(60) Provisional application No. 63/282,961, filed on Nov. 24, 2021.

(51) Int. Cl. C12N 15/86; A61K 40/11; A61K 40/31; A61K 40/42; A61P 35/00; C07K 14/705; C07K 14/725; C07K 16/28; C12N 5/0783; C12N 9/22; C12N 15/11; C12N 15/90

(52) U.S. Cl. CPC C12N 15/86; A61K 40/11; A61K 40/31; A61K 40/4211; A61K 40/4212; A61P 35/00; C07K 14/70503; C07K 14/7051; C07K 14/70517; C07K 14/70578; C07K 16/2803; C12N 5/0636; C12N 9/22; C12N 15/11; C12N 15/907; C07K 2317/53; C07K 2317/622; C07K 2319/02; C07K 2319/03; C12N 2310/20; C12N 2510/00; C12N 2740/15043

(57) ABSTRACT

Provided are polycistronic vectors for co-expression of one or more tolerogenic factors, one or more CARs, and optionally one or more safety switches, as well as compositions comprising the same, cells comprising the same and methods of generating those cells, and methods of using the disclosed vectors, compositions, and cells to treat diseases such as cancer, diabetes, and neurological diseases.

### CROSS-REFERENCE TO RELATED APPLICATIONS

This application is a national stage application under 35 U.S.C. § 371 of International Application No. PCT/US2022/073862, filed internationally on Jul. 18, 2022, which claims the benefit of and the priority to U.S. Provisional Application No. 63/222,954, filed on Jul. 16, 2021, and U.S. Provisional Application No. 63/282,961, filed on Nov. 24, 2021, the disclosure of which are hereby incorporated by reference in their entirety.

### SUBMISSION OF SEQUENCE LISTING ON ASCII TEXT FILE

The contents of the electronic sequence listing (186152008300SEQLIST.xml; Size: 194,641 bytes; and Date of Creation: Jan. 4, 2024) is herein incorporated by reference in its entirety.

### SUMMARY

An emerging cell therapy approach called adoptive cell transfer (ACT) is rapidly changing the scene of human disease treatment. ACT involves collecting cells from a patient (autologous) or healthy donors (allogeneic), adapting these cells, and transferring the cells into the patient to fight diseases. The ACT that has gained the most clinical success is chimeric antigen receptor (CAR)-T cell therapy, wherein CAR-modified T cells specific to a tumor-associated antigen are generated and infused into the patient's body to recognize and kill cancer cells that harbor the antigen on their surfaces. The CAR-T cell therapy furthest along in clinical development targets an antigen found on B cells called Cluster of Differentiation 19 (CD19) and has been approved for treatment of large B cell lymphoma (see, e.g., tisagenlecleucel, lisocabtagene maraleucel, brexucabtagene autoleucel, axicabtagene ciloleucel).

Compared with autologous cell-based strategies, off-the-shelf allogeneic cells offer several advantages, including improved access for patients, quality control, as well as avoidance of delay of treatment, malignant contamination, and cell dysfunction. However, allogeneic cells may cause serious graft-versus-host effects and be rapidly eliminated by the host immune system. Immune incompatibility thus is a significant barrier to clinical applications of ACT, and the ability of transplanted cells to evade a patient's immune system is a factor in the success of allogeneic cell therapies. Accordingly, there is a need for novel approaches, compositions, and methods for producing cell-based therapies.

The present technology provides polycistronic vectors for co-expression of one or more tolerogenic factors, one or more CARs, and optionally one or more safety switches, as well as compositions and methods of using the same to treat diseases, such as cancer, diabetes, and neurological diseases. For example, in one embodiment, the present technology provides bicistronic vectors for co-expression of a tolerogenic factor (e.g., CD47, HLA-E, HLA-G, PD-L1, CTLA-4, etc.) and a CAR (e.g., CD19 CAR, CD22 CAR, BCMA CAR, etc.), as well as compositions and methods of using the same to treat diseases. In another embodiment, the present technology provides tricistronic vectors for co-expression of a tolerogenic factor (e.g., CD47, HLA-E, HLA-G, PD-L1, CTLA-4, etc.), a CAR (e.g., CD19 CAR, CD22 CAR, BCMA CAR, etc.), and a safety switch, as well as compositions and methods of using the same to treat diseases.

In some aspects, provided is a polycistronic vector comprising (a) a first expression cassette comprising a nucleotide sequence encoding a tolerogenic factor, (b) a second expression cassette comprising a nucleotide sequence encoding a CAR, and (c) one or more cleavage sites separating the first expression cassette and the second expression cassette, wherein the first expression cassette precedes the second expression cassette in the 5′ to 3′ order.

In some embodiments, the tolerogenic factor is selected from the group consisting of A20/TNFAIP3, CD16, CD16 Fc receptor, CD24, CD35, CD39, CD46, CD47, CD52, CD55, CD59, CD200, CCL22, CTLA4-Ig, C1 inhibitor, CR1, DUX4, FASL, H2-M3, IDO1, IL15-RF, HLA-C, HLA-E, HLA-E heavy chain, HLA-G, IL-10, IL-35, MANF, PD-1, PD-L1, Serpinb9, CCl21, and Mfge8. In some embodiments, the tolerogenic factor comprises CD47, for example, human CD47. In some embodiments, the human CD47 comprises an amino acid sequence that is at least 80% identical to the amino acid sequence set forth in any one of SEQ ID NOs: 1-5. In some embodiments, the nucleotide sequence encoding CD47 is at least 80% identical to the nucleotide sequence set forth in any one of SEQ ID NOs: 129-134. In some embodiments, the CD47 is codon-optimized. In some embodiments, the nucleotide sequence encoding CD47 is at least 80% identical to the nucleotide sequence set forth in SEQ ID NO: 135.

In some embodiments, the CAR comprises CD19 CAR. In some embodiments, the CD19 CAR comprises a signal peptide, an extracellular binding domain specific to CD19, a hinge domain, a transmembrane domain, an intracellular costimulatory domain, and/or an intracellular signaling domain. In some embodiments, the signal peptide comprises a CD8α signal peptide, an IgK signal peptide, or a GMCSFR-a signal peptide. In some embodiments, the extracellular binding domain specific to CD19 comprises an scFv, for example, an scFv comprising the heavy chain variable region (V_{H}) and the light chain variable region (V_{L}) of FMC63. In some embodiments, the scFv comprises one or more complementarity determining regions (CDRs) having amino acid sequences set forth in SEQ ID NOs: 21-23 and 26-28. In some embodiments, the scFv comprises a light chain with one or more CDRs having amino acid sequences set forth in SEQ ID NOs: 21-23. In some embodiments, the scFv comprises a heavy chain with one or more CDRs having amino acid sequences set forth in SEQ ID NOs: 26-28. In some embodiments, the hinge domain comprises a CD8a hinge domain, a CD28 hinge domain, an IgG4 hinge domain, or an IgG4 hinge-CH2-CH3 domain. In some embodiments, the transmembrane comprises a CD8α transmembrane domain or a CD28 transmembrane domain. In some embodiments, the intracellular costimulatory domain comprises a 4-1 BB costimulatory domain or a CD28 costimulatory domain. In some embodiments, the intracellular signaling domain comprises a CD3 zeta (ζ) signaling domain. In some embodiments, the CD19 CAR comprises an amino acid sequence set forth in SEQ ID NO:117 or is at least 80% identical to the amino acid sequence set forth in SEQ ID NO:117. In some embodiments, the nucleotide sequence encoding the CD19 CAR comprises a nucleotide sequence set forth in SEQ ID NO:116 or is at least 80% identical to the nucleotide sequence set forth in SEQ ID NO:116. In some embodiments, the CD19 CAR comprises an amino acid sequence set forth in SEQ ID NO: 32, 34, or 36, or is at least 80% identical to the amino acid sequence set forth in SEQ ID NO: 32, 34, or 36. In some embodiments, the nucleotide sequence encoding the CD19 CAR comprises a nucleotide sequence set forth in SEQ ID NO: 31, 33, or 35, or is at least 80% identical to the nucleotide sequence set forth in SEQ ID NO: 31, 33, or 35.

In some embodiments, the CAR comprises CD20 CAR. In some embodiments, the CD20 CAR comprises a signal peptide, an extracellular binding domain specific to CD20, a hinge domain, a transmembrane domain, an intracellular costimulatory domain, and/or an intracellular signaling domain. In some embodiments, the signal peptide comprises a CD8α signal peptide, an IgK signal peptide, or a GMCSFR-a signal peptide. In some embodiments, the extracellular binding domain specific to CD20 comprises an scFv, for example, an scFv comprising the V_{L} and the V_{H} of Leu16. In some embodiments, the scFv comprises one or more CDRs having amino acid sequences set forth in SEQ ID NOs: 39-41 and 43-44. In some embodiments, the scFv comprises a light chain with one or more CDRs having amino acid sequences set forth in SEQ ID NOs: 39-41. In some embodiments, the scFv comprises a heavy chain with one or more CDRs having amino acid sequences set forth in SEQ ID NOs: 43-44. In some embodiments, the hinge domain comprises a CD8α hinge domain, a CD28 hinge domain, an IgG4 hinge domain, or an IgG4 hinge-CH2-CH3 domain. In some embodiments, the transmembrane comprises a CD8a transmembrane domain or a CD28 transmembrane domain. In some embodiments, the intracellular costimulatory domain comprises a 4-1 BB costimulatory domain or a CD28 costimulatory domain. In some embodiments, the intracellular signaling domain comprises a CD3 zeta (ζ) signaling domain.

In some embodiments, the CAR comprises CD22 CAR. In some embodiments, the CD22 CAR comprises a signal peptide, an extracellular binding domain specific to CD22, a hinge domain, a transmembrane domain, an intracellular costimulatory domain, and/or an intracellular signaling domain. In some embodiments, the signal peptide comprises a CD8α signal peptide, an IgK signal peptide, or a GMCSFR-a signal peptide. In some embodiments, the extracellular binding domain specific to CD22 comprises an scFv, for example, an scFv comprising the V_{H} and the V_{L} of m971. In some embodiments, the scFv comprises one or more CDRs having amino acid sequences set forth in SEQ ID NOs: 47-49 and 51-53. In some embodiments, the scFv comprises a heavy chain with one or more CDRs having amino acid sequences set forth in SEQ ID NOs: 47-49. In some embodiments, the scFv comprises a light chain with one or more CDRs having amino acid sequences set forth in SEQ ID NOs: 51-53. In some embodiments, the extracellular binding domain specific to CD22 comprises an scFv, for example, an scFv comprising the V_{H} and the V_{L} of m971-L7. In some embodiments, the scFv comprises one or more CDRs having amino acid sequences set forth in SEQ ID NOs: 56-58 and 60-62. In some embodiments, the scFv comprises a heavy chain with one or more CDRs having amino acid sequences set forth in SEQ ID NOs: 56-58. In some embodiments, the scFv comprises a light chain with one or more CDRs having amino acid sequences set forth in SEQ ID NOs: 60-62. In some embodiments, the hinge domain comprises a CD8a hinge domain, a CD28 hinge domain, an IgG4 hinge domain, or an IgG4 hinge-CH2-CH3 domain. In some embodiments, the transmembrane comprises a CD8a transmembrane domain or a CD28 transmembrane domain. In some embodiments, the intracellular costimulatory domain comprises a 4-1 BB costimulatory domain or a CD28 costimulatory domain. In some embodiments, the intracellular signaling domain comprises a CD3 zeta (ζ) signaling domain.

In some embodiments, the CAR comprises BCMA CAR. In some embodiments, the BCMA CAR comprises a signal peptide, an extracellular binding domain specific to BCMA, a hinge domain, a transmembrane domain, an intracellular costimulatory domain, and/or an intracellular signaling domain. In some embodiments, the signal peptide comprises a CD8a signal peptide, an IgK signal peptide, or a GMCSFR-a signal peptide. In some embodiments, the extracellular binding domain specific to BCMA comprises an scFv, for example, an scFv comprises the V_{L} and the V_{H} of C11 D5.3. In some embodiments, the scFv comprises one or more CDRs having amino acid sequences set forth in SEQ ID NOs: 65-67 and 69-71. In some embodiments, the scFv comprises a light chain with one or more CDRs having amino acid sequences set forth in SEQ ID NOs: 65-67. In some embodiments, the scFv comprises a heavy chain with one or more CDRs having amino acid sequences set forth in SEQ ID NOs: 69-71. In some embodiments, the extracellular binding domain specific to BCMA comprises an scFv, for example, an scFv comprising the V_{L} and the V_{H} of C12A3.2. In some embodiments, the scFv comprises one or more CDRs having amino acid sequences set forth in SEQ ID NOs: 74-76 and 78-80. In some embodiments, the scFv comprises a light chain with one or more CDRs having amino acid sequences set forth in SEQ ID NOs: 74-76. In some embodiments, the scFv comprises a heavy chain with one or more CDRs having amino acid sequences set forth in SEQ ID NOs: 78-80. In some embodiments, the scFv comprises the V_{L} and the V_{H} of CT103A scFv. In some embodiments, the scFv comprises one or more CDRs having amino acid sequences set forth in SEQ ID NOs: 120-122 and 124-126. In some embodiments, the scFv comprises a light chain with one or more CDRs having amino acid sequences set forth in SEQ ID NOs: 120-122. In some embodiments, the scFv comprises a heavy chain with one or more CDRs having amino acid sequences set forth in SEQ ID NOs: 124-126. In some embodiments, the extracellular binding domain specific to BCMA comprises a fully human heavy-chain variable domain (FHVH), for example, FHVH33. In some embodiments, the FHVH comprises one or more CDRs having amino acid sequences set forth in SEQ ID NOs: 82-84. In some embodiments, the hinge domain comprises a CD8a hinge domain, a CD28 hinge domain, an IgG4 hinge domain, or an IgG4 hinge-CH2-CH3 domain. In some embodiments, the transmembrane comprises a CD8a transmembrane domain or a CD28 transmembrane domain. In some embodiments, the intracellular costimulatory domain comprises a 4-1BB costimulatory domain or a CD28 costimulatory domain. In some embodiments, the intracellular signaling domain comprises a CD3 zeta (ζ) signaling domain.

In some embodiments, the one or more cleavage sites comprise a self-cleaving site, for example, a 2A site. In some embodiments, the 2A site comprises a T2A, P2A, E2A, or F2A site.

In some embodiments, the one or more cleavage sites further comprise a protease site, for example, a furin site. In some embodiments, the furin site comprises an FC1, FC2, or FC3 site. In some embodiments, the protease site precedes the 2A site in the 5′ to 3′ order.

In some embodiments, the polycistronic vector further comprises (d) a third expression cassette comprising a nucleotide sequence encoding a safety switch, wherein the third expression cassette is separated from the first expression cassette and/or the second expression cassette by one or more cleavage sites. In some embodiments, the safety switch is selected from the group consisting of herpes simplex virus thymidine kinase (HSVtk), cytosine deaminase (CyD), nitroreductase (NTR), purine nucleoside phosphorylase (PNP), horseradish peroxidase, inducible caspase 9 (iCasp9), rapamycin-activated caspase 9 (rapaCasp9), CCR4, CD16, CD19, CD20, CD30, EGFR, GD2, HER1, HER2, MUC1, PSMA, RQR8, and a CD47-SIRPa blockade agent.

In some embodiments, the polycistronic vector further comprises a promoter. In some embodiments, the promoter is a constitutive promoter, for example, an EF1α, CMV, SV40, PGK, UBC or CAG promoter. In some embodiments, the promoter is an inducible promoter, for example, a Tet-On, Tet-Off, AlcA, LexA, or Cre promoter.

In some embodiments, the polycistronic vector further comprises homology arms flanking the expression cassettes for homology directed repair (HDR)-mediated insertion into a genomic locus, for example, by use of a site-directed nuclease selected from the group consisting Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, Mad7, a zinc finger nuclease (ZFN), a transcription activator-like effector nuclease (TALEN), a meganuclease, and a CRISPR-associated transposase.

In some aspects, provided is a polycistronic vector comprising (a) a first expression cassette comprising a nucleotide sequence encoding CD47, (b) a second expression cassette comprising a nucleotide sequence encoding CD19 CAR, and (c) a 2A site separating the first expression cassette and the second expression cassette. In some aspects, provided is a polycistronic vector comprising (a) a first expression cassette comprising a nucleotide sequence encoding CD47, (b) a second expression cassette comprising a nucleotide sequence encoding CD19 CAR, and (c) a furin site and a 2A site separating the first expression cassette and the second expression cassette, wherein the furin site precedes the 2A site in the 5′ to 3′ order. In some embodiments, the polycistronic vector further comprises (d) a third expression cassette comprising a nucleotide sequence encoding a safety switch, wherein the third expression cassette is separated from the first expression cassette and/or the second expression cassette by a 2A site.

In some aspects, provided is a polycistronic vector comprising (a) a first expression cassette comprising a nucleotide sequence encoding a tolerogenic factor, (b) a second expression cassette comprising a nucleotide sequence encoding CD19 CAR, and (c) one or more cleavage sites separating the first expression cassette and the second expression cassette, wherein the CD19 CAR comprises a CD8a signal peptide, an FMC63 scFv, a CD8a hinge domain, a CD8a transmembrane domain, a 4-1 BB costimulatory domain, and a CD3ζ signaling domain. In some embodiments, the tolerogenic factor comprises CD47.

In some aspects, provided is a polycistronic vector comprising (a) a first expression cassette comprising a nucleotide sequence encoding a tolerogenic factor, (b) a second expression cassette comprising a nucleotide sequence encoding CD19 CAR, and (c) one or more cleavage sites separating the first expression cassette and the second expression cassette, wherein the CD19 CAR comprises a GMCSFR-a signal peptide, an FMC63 scFv, an IgG4 hinge domain, a CD28 transmembrane domain, a 4-1 BB costimulatory domain, and a CD3ζ signaling domain. In some embodiments, the tolerogenic factor comprises CD47.

In some aspects, provided is a polycistronic vector comprising (a) a first expression cassette comprising a nucleotide sequence encoding a tolerogenic factor, (b) a second expression cassette comprising a nucleotide sequence encoding CD19 CAR, and (c) one or more cleavage sites separating the first expression cassette and the second expression cassette, wherein the CD19 CAR comprises a GMCSFR-a signal peptide, an FMC63 scFv, a CD28 hinge domain, a CD28 transmembrane domain, a CD28 costimulatory domain, and a CD3ζ signaling domain. In some embodiments, the tolerogenic factor comprises CD47.

In some aspects, provided is a polycistronic vector comprising (a) a first expression cassette comprising a nucleotide sequence encoding CD47, (b) a second expression cassette comprising a nucleotide sequence encoding CD20 CAR, and (c) a 2A site separating the first expression cassette and the second expression cassette. In some aspects, provided is a polycistronic vector comprising (a) a first expression cassette comprising a nucleotide sequence encoding CD47, (b) a second expression cassette comprising a nucleotide sequence encoding CD20 CAR, and (c) a furin site and a 2A site separating the first expression cassette and the second expression cassette, wherein the furin site precedes the 2A site in the 5′ to 3′ order. In some embodiments, the polycistronic vector further comprises (d) a third expression cassette comprising a nucleotide sequence encoding a safety switch, wherein the third expression cassette is separated from the first expression cassette and/or the second expression cassette by a 2A site.

In some aspects, provided is a polycistronic vector comprising (a) a first expression cassette comprising a nucleotide sequence encoding CD47, (b) a second expression cassette comprising a nucleotide sequence encoding CD22 CAR, and (c) a 2A site separating the first expression cassette and the second expression cassette. In some aspects, provided is a polycistronic vector comprising (a) a first expression cassette comprising a nucleotide sequence encoding CD47, (b) a second expression cassette comprising a nucleotide sequence encoding CD22 CAR, and (c) a furin site and a 2A site separating the first expression cassette and the second expression cassette, wherein the furin site precedes the 2A site in the 5′ to 3′ order. In some embodiments, the polycistronic vector further comprises (d) a third expression cassette comprising a nucleotide sequence encoding a safety switch, wherein the third expression cassette is separated from the first expression cassette and/or the second expression cassette by a 2A site.

In some aspects, provided is a polycistronic vector comprising (a) a first expression cassette comprising a nucleotide sequence encoding CD47, (b) a second expression cassette comprising a nucleotide sequence encoding CD19 CAR, (c) a third expression cassette comprising a nucleotide sequence encoding CD22 CAR, and (d) a 2A site separating any two neighboring expression cassettes. In some aspects, provided is a polycistronic vector comprising (a) a first expression cassette comprising a nucleotide sequence encoding CD47, (b) a second expression cassette comprising a nucleotide sequence encoding CD19 CAR, (c) a third expression cassette comprising a nucleotide sequence encoding CD22 CAR, and (d) a furin site and a 2A site separating any two neighboring expression cassettes, wherein the furin site precedes the 2A site in the 5′ to 3′ order. In some embodiments, the polycistronic vector further comprises (e) a fourth expression cassette comprising a nucleotide sequence encoding a safety switch, wherein the fourth expression cassette is separated from the first expression cassette, the second expression cassette, and/or the third expression cassette by a 2A site.

In some aspects, provided is a polycistronic vector comprising (a) a first expression cassette comprising a nucleotide sequence encoding CD47, (b) a second expression cassette comprising a nucleotide sequence encoding BCMA CAR, and (c) a 2A site separating the first expression cassette and the second expression cassette. In some aspects, provided is a polycistronic vector comprising (a) a first expression cassette comprising a nucleotide sequence encoding CD47, (b) a second expression cassette comprising a nucleotide sequence encoding BCMA CAR, and (c) a furin site and a 2A site separating the first expression cassette and the second expression cassette, wherein the furin site precedes the 2A site in the 5′ to 3′ order. In some embodiments, the polycistronic vector further comprises (d) a third expression cassette comprising a nucleotide sequence encoding a safety switch, wherein the third expression cassette is separated from the first expression cassette and/or the second expression cassette by a 2A site.

In some aspects, provided is a polycistronic vector comprising (a) a first expression cassette comprising a nucleotide sequence encoding a tolerogenic factor, (b) a second expression cassette comprising a nucleotide sequence encoding BCMA CAR, and (c) one or more cleavage sites separating the first expression cassette and the second expression cassette, wherein the BCMA CAR comprises a BB2121 binder, a CD8a hinge domain, a CD8a transmembrane domain, a 4-1 BB costimulatory domain, and a CD3ζ signaling domain. In some embodiments, the tolerogenic factor comprises CD47.

In some aspects, provided is a polycistronic vector comprising (a) a first expression cassette comprising a nucleotide sequence encoding a tolerogenic factor, (b) a second expression cassette comprising a nucleotide sequence encoding BCMA CAR, and (c) one or more cleavage sites separating the first expression cassette and the second expression cassette, wherein the BCMA CAR comprises a CD8a signal peptide, an CT103A scFv, a CD8a hinge domain, a CD8a transmembrane domain, a 4-1 BB costimulatory domain, and a CD3ζ signaling domain. In some embodiments, the BCMA comprises an amino acid sequence set forth in SEQ ID NO:128. In some embodiments, the tolerogenic factor comprises CD47.

In some aspects, provided is a polycistronic vector comprising (a) a first expression cassette comprising a nucleotide sequence encoding CD47, (b) a second expression cassette comprising a nucleotide sequence encoding a safety switch, and (c) a 2A site separating the first expression cassette and the second expression cassette. In some aspects, provided is a polycistronic vector comprising (a) a first expression cassette comprising a nucleotide sequence encoding CD47, (b) a second expression cassette comprising a nucleotide sequence encoding a safety switch, and (c) a furin site and a 2A site separating the first expression cassette and the second expression cassette, wherein the furin site precedes the 2A site in the 5′ to 3′ order.

In some aspects, provided are viruses containing the polycistronic vector or a fragment thereof according to various embodiments of the present technology. In some embodiments, the virus is an adenovirus, adeno-associated virus, retrovirus, lentivirus, or phage.

In some aspects, provided are host cells containing the polycistronic vector or a fragment thereof according to various embodiments of the present technology. In some embodiments, the host cell is an autologous cell. In some embodiments, the host cell is an allogeneic cell. In some embodiments, the host cell is an embryonic stem cell (ESC) or an induced pluripotent stem cell (iPSC). In some embodiments, the host cell is differentiated from an ESC or an iPSC. In some embodiments, the host cell is a primary cell. In some embodiments, the host cell is a T cell, a natural killer (NK) cell, or a natural killer T (NKT) cell. In some embodiments, the host cell is a β islet cell. In some embodiments, the host cell is a glial progenitor cell (GPC).

In some embodiments, the polycistronic vector or a fragment thereof is inserted into a specific genomic locus of the host cell selected from the group consisting of a B2M locus, a TAP1 locus, a CIITA locus, a TRAC locus, a TRBC locus, a MIC-A locus, a MIC-B locus, and a safe harbor locus. In some embodiments, the safe harbor locus is selected from the group consisting of an AAVS1, ABO, CCR5, CLYBL, CXCR4, F3, FUT1, HMGB1, KDM5D, LRP1, MICA, MICB, RHD, ROSA26, and SHS231 locus. In some embodiments, the insertion is by homology directed repair (HDR), for example, using a site-directed nuclease selected from the group consisting of Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, Mad7, a zinc finger nuclease (ZFN), a transcription activator-like effector nuclease (TALEN), a meganuclease, and a CRISPR-associated transposase.

In some embodiments the host cell is modified to have reduced expression of one or more MHC I molecules and/or one or more MHC II molecules, optionally, wherein the one or more MHC I molecules are selected from the group consisting of HLA-A, HLA-B, HLA-C, and optionally, wherein the one or more MHC II molecules are selected from the group consisting of HLA-DR, HLA-DQ, HLA-DP, HLA-DM, and HLA-DO. In some embodiments, the host cell has reduced expression of B2M, TAP1, and/or CIITA. In some embodiments, the host cell has B2M, TAP1, and/or CIITA knockout. In some embodiments, the B2M, TAP1, and/or CIITA knockout occur in both alleles. In some embodiments, the host cell has reduced expression of MIC-A and/or MIC-B. In some embodiments, the host cell has MIC-A and/or MIC-B knockout. In some embodiments, the MIC-A and/or MIC-B knockout occur in both alleles.

In some aspects, provided are T cells containing a polycistronic vector or fragments thereof, wherein the polycistronic vector comprising a first expression cassette comprising a nucleotide sequence encoding CD47 a second expression cassette comprising a nucleotide sequence encoding a CAR. In some aspects, provided are T cells containing a polycistronic vector or a fragment thereof, wherein the polycistronic vector comprising a first expression cassette comprising a nucleotide sequence encoding CD47 a second expression cassette comprising a nucleotide sequence encoding a CAR, and wherein the T cell has B2M, TAP1, and/or CIITA knockout. In some embodiments, the B2M, TAP1, and/or CIITA knockout occur in both alleles. In some embodiments, the polycistronic vector or fragment thereof is inserted into a specific genomic locus of the T cell selected from the group consisting of a B2M locus, a TAP1 locus, a CIITA locus, a TRAC locus, a TRBC locus, a MIC-A locus, a MIC-B locus, and a safe harbor locus. In some embodiments, the safe harbor locus is selected from the group consisting of an AAVS1, ABO, CCR5, CLYBL, CXCR4, F3, FUT1, HMGB1, KDM5D, LRP1, MICA, MICB, RHD, ROSA26, and SHS231 locus. In some embodiments, the CAR comprises CD19 CAR, CD20 CAR, CD22 CAR, or BCMA CAR. In some embodiments, the CAR is CD19 CAR comprising a CD8a signal peptide, an FMC63 scFv, a CD8a hinge domain, a CD8a transmembrane domain, a 4-1 BB costimulatory domain, and a CD3ζ signaling domain. In some embodiments, the CAR is CD19 CAR comprising a GMCSFR-a signal peptide, an FMC63 scFv, an IgG4 hinge domain, a CD28 transmembrane domain, a 4-11BB costimulatory domain, and a CD3ζ signaling domain. In some embodiments, the CAR is CD19 CAR comprising a GMCSFR-a signal peptide, an FMC63 scFv, a CD28 hinge domain, a CD28 transmembrane domain, a CD28 costimulatory domain, and a CD3ζ signaling domain. In some embodiments, the CAR is CD19 CAR comprising an amino acid sequence set forth in SEQ ID NO:117 or is at least 80% identical to the amino acid sequence set forth in SEQ ID NO:117. In some embodiments, the CAR is CD19 CAR comprising an amino acid sequence set forth in SEQ ID NO: 32, 34, or 36, or an amino acid sequence that is at least 80% identical to the amino acid sequence set forth in SEQ ID NO: 32, 34, or 36. In some embodiments, the CAR is BCMA CAR comprising a CD8a signal peptide, a CT103A scFv, a CD8a hinge domain, a CD8a transmembrane domain, a 4-1 BB costimulatory domain, and a CD3ζ signaling domain. In some embodiments, the CAR is BCMA CAR comprising an amino acid sequence set forth in SEQ ID NO:128 or is at least 80% identical to the amino acid sequence set forth in SEQ ID NO:128.

In some aspects, provided are cells having a genomic locus modified by HDR, wherein the genomic locus is selected from the group consisting of a B2M locus, a TAP1 locus, a CIITA locus, a TRAC locus, a TRBC locus, a MIC-A locus, a MIC-B locus, and a safe harbor locus. In some embodiments, the safe harbor locus is selected from the group consisting of an AAVS1, ABO, CCR5, CLYBL, CXCR4, F3, FUT1, HMGB1, KDM5D, LRP1, MICA, MICB, RHD, ROSA26, and SHS231 locus. In some embodiments, the HDR uses a site-directed nuclease selected from the group consisting of Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, Mad7, a zinc finger nuclease (ZFN), a transcription activator-like effector nuclease (TALEN), a meganuclease, and a CRISPR-associated transposase.

In some aspects, provided are iPSC-derived β islet cells having (1) reduced expression of MHC I and/or MHC II; and (2) a transgene comprising CD47 and a safety switch inserted at a safe harbor locus, wherein the safe harbor locus is selected from the group consisting of an AAVS1, ABO, CCR5, CLYBL, CXCR4, F3, FUT1, HMGB1, KDM5D, LRP1, MICA, MICB, RHD, ROSA26, and SHS231 locus. In some aspects, provided are iPSC-derived β islet cells having (1) reduced expression of MHC I and/or MHC II; and (2) a transgene comprising CD47 and HSVtk flanked by CLYBL homology arms, wherein the transgene is inserted at the CLYBL locus. In some embodiments, the iPSC-derived β islet cell has B2M, TAP1, and/or CIITA knockout. In some embodiments, the B2M, TAP1, and/or CIITA knockout occur in both alleles.

In some aspects, provided are ESC-derived GPCs having (1) reduced expression of MHC I and/or MHC II; and (2) a transgene comprising CD47 and a safety switch inserted at a safe harbor locus, wherein the safe harbor locus is selected from the group consisting of an AAVS1, ABO, CCR5, CLYBL, CXCR4, F3, FUT1, HMGB1, KDM5D, LRP1, MICA, MICB, RHD, ROSA26, and SHS231 locus. In some aspects, provided are ESC-derived GPCs having (1) reduced expression of MHC I and/or MHC II; and (2) a transgene comprising CD47 and HSVtk flanked by CLYBL homology arms, wherein the transgene is inserted at the CLYBL locus. In some embodiments, the ESC-derived GPC has B2M, TAP1, and/or CIITA knockout. In some embodiments, the B2M, TAP1, and/or CIITA knockout occur in both alleles.

In some aspects, provided are compositions comprising the polycistronic vector according to various embodiments of the present technology. In some aspects, provided are compositions comprising the virus according to various embodiments of the present technology. In some aspects, provided are pharmaceutical compositions comprising the host cell or cell according to various embodiments of the present technology.

In some aspects, provided are guide RNAs (gRNAs) for use in HDR-mediated insertion of a transgene into a genomic locus selected from the group consisting of a B2M locus, a TAP1 locus, a CIITA locus, a TRAC locus, a TRBC locus, a MIC-A locus, a MIC-B locus, and a safe harbor locus. In some embodiments, the safe harbor locus is selected from the group consisting of an AAVS1, ABO, CCR5, CLYBL, CXCR4, F3, FUT1, HMGB1, KDM5D, LRP1, MICA, MICB, RHD, ROSA26, and SHS231 locus.

In some embodiments, the gRNA comprises a crRNA and optionally a tracrRNA. In some embodiments, the gRNA comprises a crRNA and a tracrRNA as two separate molecules. In some embodiments, the gRNA comprises a crRNA and a tracrRNA as a single guide RNA (sgRNA). In some embodiments, the sgRNA comprises a complementary region, a crRNA repeat region, a tetraloop, and a tracrRNA.

In some embodiments, the crRNA repeat region comprises, consists of, or consists essentially of a nucleotide sequence set forth in in SEQ ID NO:95, SEQ ID NO:99, SEQ ID NO:103, or SEQ ID NO:108. In some embodiments, the tetraloop comprises, consists of, or consists essentially of a nucleotide sequence set forth in SEQ ID NO:96 or SEQ ID NO:107. In some embodiments, the tracrRNA comprises, consists of, or consists essentially of a nucleotide sequence set forth in SEQ ID NO:97, SEQ ID NO:101, SEQ ID NO:105, or SEQ ID NO:106.

In some embodiments, the crRNA comprises a complementary region specific to a region of the AAVS1, CLYBL, or CCR5 locus. In some embodiments, the region is a coding sequence (CDS), an exon, an intron, a sequence spanning a portion of an exon and a portion of an adjacent intron, or a regulatory region. In some embodiments, the complementary region comprises, consists of, or consists essentially of a nucleotide sequence set forth in SEQ ID NO:110, SEQ ID NO:111, or SEQ ID NO:112.

In some aspects, provided are guide RNAs (gRNAs) for use in HDR-mediated insertion of a transgene into a genomic locus, wherein the genomic locus is located within 4000 bp of a locus at Chromosome 19: 55,117,222-55,112,796, Chromosome 13: 99,773,011-99,858,860, or Chromosome 3: 46,372,892-46,376,206. In certain of these embodiments, the locus is within 4000 bp, within 3500 bp, within 3000 bp, within 2500 bp, within 2000 bp, within 1500 bp, within 1000 bp, or within 500 bp of Chromosome 19: 55,115,674, Chromosome 13: 99,822,980, or Chromosome 3: 46,373,180. In certain embodiments, the gRNA is configured to produce a cut site at Chromosome 19: 55,115,674, Chromosome 13: 99,822,980, or Chromosome 3: 46,373,180, or within 5, 10, 15, 20, 30, 40, or 50 nucleotides of Chromosome 19: 55,115,674, Chromosome 13: 99,822,980, or Chromosome 3: 46,373,180.

In some embodiments, provided are compositions comprising the gRNA according to various embodiments of the present technology. In some embodiments, the compositions further comprise a site-directed nuclease or a nucleotide sequence encoding a site-directed nuclease protein as described herein.

In some embodiments, the compositions comprising the gRNA according to various embodiments of the present technology are formulated for delivery into a cell. In some embodiments, the cell further comprises a site-directed nuclease or a nucleotide sequence encoding a site-directed nuclease protein as described herein.

In some aspects, provided are methods of HDR-mediated insertion of a transgene into a genomic locus, comprising introducing a gRNA, a site-directed nuclease or a nucleotide sequence encoding a site-directed nuclease, and a transgene flanked by homology arms into a host cell, wherein the sited-directed nuclease is selected from the group consisting of Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, Mad7, a zinc finger nuclease (ZFN), a transcription activator-like effector nuclease (TALEN), a meganuclease, and a CRISPR-associated transposase. In some embodiments, the genomic locus is selected from the group consisting of a B2M locus, a TAP1 locus, a CIITA locus, a TRAC locus, a TRBC locus, a MIC-A locus, a MIC-B locus, and a safe harbor locus. In some embodiments, the safe harbor locus is selected from the group consisting of an AAVS1, ABO, CCR5, CLYBL, CXCR4, F3, FUT1, HMGB1, KDM5D, LRP1, MICA, MICB, RHD, ROSA26, and SHS231 locus.

In some embodiments, the method comprises introducing a gRNA accordingly to various embodiments of the present technology. In some embodiments, the genomic locus is located within 4000 bp of a locus at Chromosome 19: 55,117,222-55,112,796, Chromosome 13: 99,773,011-99,858,860, or Chromosome 3: 46,372,892-46,376,206. In certain of these embodiments, the locus is within 4000 bp, within 3500 bp, within 3000 bp, within 2500 bp, within 2000 bp, within 1500 bp, within 1000 bp, or within 500 bp of Chromosome 19: 55,115,674, Chromosome 13: 99,822,980, or Chromosome 3: 46,373,180. In certain embodiments, the gRNA is configured to produce a cut site at Chromosome 19: 55,115,674, Chromosome 13: 99,822,980, or Chromosome 3: 46,373,180, or within 5, 10, 15, 20, 30, 40, or 50 nucleotides of Chromosome 19: 55,115,674, Chromosome 13: 99,822,980, or Chromosome 3: 46,373,180.

In some embodiments, the transgene comprises (a) a first expression cassette comprising a nucleotide sequence encoding a tolerogenic factor, (b) a second expression cassette comprising a nucleotide sequence encoding a chimeric antigen receptor (CAR), (c) a third expression cassette comprising a nucleotide sequence encoding a safety switch, and/or (d) one or more cleavage sites separating the first expression cassette, the second expression cassette, and/or the third expression cassette.

In some aspects, provided are methods of identifying new genomic loci for HDR-mediated insertion of a transgene, comprising (a) locating a genomic locus based on a known gRNA; and (b) scanning a region of about 500 to 4000 bp on either side of the genomic locus for a PAM sequence. In some embodiments, the genomic locus is located within 4000 bp of a locus at Chromosome 19: 55,117,222-55,112,796, Chromosome 13: 99,773,011-99,858,860, or Chromosome 3:46,372,892-46,376,206. In some embodiments, the genomic locus is located at a position selected from the group consisting of Chromosome 19: 55,115,674 or a position within 5, 10, 15, 20, 30, 40, or 50 nucleotides of Chromosome 19: 55,115,674; Chromosome 13: 99,822,980, or a position within 5, 10, 15, 20, 30, 40, or 50 nucleotides of Chromosome 13: 99,822,980; and Chromosome 3: 46,373,180, or a position within 5, 10, 15, 20, 30, 40, or 50 nucleotides of Chromosome 3: 46,373,180.

In some aspects, provided are methods of treating a disease in a subject in need thereof, comprising administering the subject the host cell or cell according to various embodiments of the present technology, or compositions comprising the same.

In some embodiments, the disease is cancer, for example, a cancer associated with CD19, CD20, CD22, and/or BCMA expression. In some embodiments, the cancer is a hematologic malignancy. In some embodiments, the hematologic malignancy is selected from the group consisting of myeloid neoplasm, myelodysplastic syndromes (MDS), myeloproliferative/myelodysplastic syndromes, acute lymphoid leukemia (ALL), chronic lymphocytic leukemia (CLL), acute myeloid leukemia (AML), chronic myelogenous leukemia (CML), B cell acute lymphoid leukemia (B-ALL), T cell acute lymphoid leukemia (T-ALL), T cell lymphoma, and B cell lymphoma. In some embodiments, the hematologic malignancy is B lymphocyte-derived malignancy.

In some embodiments, the disease is an autoimmune disease, including, for example, lupus, systemic lupus erythematosus, rheumatoid arthritis, psoriasis, psoriatic arthritis, multiple sclerosis, Crohn's disease, ulcerative colitis, Addison's disease, Graves' disease, Sjögren's syndrome, Hashimoto's thyroiditis, and celiac disease.

In some embodiments, the disease is diabetes mellitus, including, for example, Type I diabetes, Type II diabetes, prediabetes, and gestational diabetes.

In some embodiments, the disease is a neurological disease, including, for example, catalepsy, epilepsy, encephalitis, meningitis, migraine, Huntington's, Alzheimer's, Parkinson's, Pelizaeus-Merzbacher disease, and multiple sclerosis.

In some aspects, provided are compositions comprising a first polycistronic vector and a second polycistronic vector, wherein the first polycistronic vector comprises a nucleotide sequence encoding a first tolerogenic factor and a nucleotide sequence encoding a first CAR separated by one or more cleavage sites; and the second polycistronic vector comprises a nucleotide sequence encoding a second tolerogenic factor and a nucleotide sequence encoding a second CAR separated by one or more cleavage sites. In some embodiments, the first tolerogenic factor and the second tolerogenic factor are independently selected from the group consisting of A20/TNFAIP3, CD16, CD16 Fc receptor, CD24, CD35, CD39, CD46, CD47, CD52, CD55, CD59, CD200, CCL22, CTLA4-Ig, C1 inhibitor, CR1, DUX4, FASL, H2-M3, IDO1, IL15-RF, HLA-C, HLA-E, HLA-E heavy chain, HLA-G, IL-10, IL-35, MANF, PD-1, PD-L1, Serpinb9, CCl21, and Mfge8. In some embodiments, the first tolerogenic factor and the second tolerogenic factor comprise CD47. In some embodiments, the CD47 comprises an amino acid sequence that is at least 80% identical to the amino acid sequence set forth in any one of SEQ ID NOs: 1-5. In some embodiments, the first CAR and the second CAR and different and independently selected from the group consisting of CD19 CAR, CD20 CAR, CD22 CAR, and BCMA CAR. In some embodiments, the first CAR and/or the second CAR comprises an amino acid sequence set forth in any one of SEQ ID NOs: 32, 34, 36, 117, 128, and 136, or is at least 80% identical to the amino acid sequence set forth in any one of SEQ ID NOs: 32, 34, 36, 117, 128, and 136. In some embodiments, the first CAR is CD19 CAR and the second CAR is CD22 CAR.

In some aspects, provided are compositions comprising a first virus comprising a first polycistronic vector and a second virus comprising a second polycistronic vector, wherein the first polycistronic vector comprises a nucleotide sequence encoding a first tolerogenic factor and a nucleotide sequence encoding a first CAR separated by one or more cleavage sites; and the second polycistronic vector comprises a nucleotide sequence encoding a second tolerogenic factor and a nucleotide sequence encoding a second CAR separated by one or more cleavage sites. In some embodiments, the first virus and/or the second virus is an adenovirus, adeno-associated virus, retrovirus, lentivirus, or phage. In some embodiments, the first tolerogenic factor and the second tolerogenic factor are independently selected from the group consisting of A20/TNFAIP3, CD16, CD16 Fc receptor, CD24, CD35, CD39, CD46, CD47, CD52, CD55, CD59, CD200, CCL22, CTLA4-Ig, C1 inhibitor, CR1, DUX4, FASL, H2-M3, IDO1, IL15-RF, HLA-C, HLA-E, HLA-E heavy chain, HLA-G, IL-10, IL-35, MANF, PD-1, PD-L1, Serpinb9, CCl21, and Mfge8. In some embodiments, the first tolerogenic factor and the second tolerogenic factor comprise CD47. In some embodiments, the CD47 comprises an amino acid sequence that is at least 80% identical to the amino acid sequence set forth in any one of SEQ ID NOs: 1-5. In some embodiments, the first CAR and the second CAR and different and independently selected from the group consisting of CD19 CAR, CD20 CAR, CD22 CAR, and BCMA CAR. In some embodiments, the first CAR and/or the second CAR comprises an amino acid sequence set forth in any one of SEQ ID NOs: 32, 34, 36, 117, 128, and 136, or is at least 80% identical to the amino acid sequence set forth in any one of SEQ ID NOs: 32, 34, 36, 117, 128, and 136. In some embodiments, the first CAR is CD19 CAR and the second CAR is CD22 CAR.

In some aspects, provided are methods of generating a heterogenous population of host cells, comprising introducing to the population of host cells the composition according to various embodiments of the present technology. In some aspects, provided are heterogenous populations of host cells generated by the method as described. In some aspects, provided are pharmaceutical compositions comprising the population of host cells as described. In some aspects, provided are methods of treating a disease in a subject in need thereof, comprising administering the subject the population of host cells as described or a pharmaceutical composition containing the same.

In some aspects, provided is a host cell having reduced expression of one or more MHC class I molecules, one or more MHC class II molecules, MIC-A, and/or MIC-B and increased expression of one or more tolerogenic factors selected from the group consisting of HLA-E, CD24, CD47, PD-L1, CD46, CD55, CD59, and C1 inhibitor, wherein the one or more tolerogenic factors are carried by the polycistronic vector of any one of claims 1 to 118 or a fragment thereof.

In some embodiments, the reduced expression of MHC class I molecules is by reduced expression of B2M. In some embodiments, the reduced expression of MHC class II molecules is by reduced expression of CIITA. In some embodiments, the reduced expression of MHC class I molecules and/or MHC class II molecules is by reduced expression of MIC-A and/or MIC-B. In some embodiments, the host cell has knockout of one or more MHC class I molecules, one or more MHC class II molecules, MIC-A, and/or MIC-B. In some embodiments, the host cell has knockout of one or more MHC class I molecules, one or more MHC class II molecules, MIC-A, and MIC-B. In some embodiments, the host cell has knockout of MHC class I molecules, MHC class II molecules, MIC-A, and MIC-B. In some embodiments, the knockout is by use of a site-directed nuclease selected from the group consisting of Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, Mad7, a zinc finger nuclease (ZFN), a transcription activator-like effector nuclease (TALEN), a meganuclease, and a CRISPR-associated transposase.

In some embodiments, the one or more tolerogenic factors comprise HLA-E. In some embodiments, the one or more tolerogenic factors is one or more tolerogenic factors comprise CD24. In some embodiments, the one or more tolerogenic factors comprise CD47. In some embodiments, the one or more tolerogenic factors comprise PD-L1. In some embodiments, the one or more tolerogenic factors comprise CD24, CD47, and PD-L1. In some embodiments, the one or more tolerogenic factors comprise CD46. In some embodiments, the one or more tolerogenic factors comprise CD55. In some embodiments, the one or more tolerogenic factors comprise CD59. In some embodiments, the one or more tolerogenic factors comprise C1 inhibitor. In some embodiments, the one or more tolerogenic factors comprise CD46, CD55, CD59, and C1 inhibitor. In some embodiments, the one or more tolerogenic factors comprise HLA-E, CD24, CD47, PD-L1, CD46, CD55, CD59, and C1 inhibitor.

In some embodiments, the host cell is a pluripotent stem cell (PSC). In some embodiments, the PSC is an ESC or an iPSC. In some embodiments, the host cell is differentiated from an ESC or an iPSC. In some embodiments, the host cell is a primary cell. In some embodiments, the host cell is a T cell, a NK cell, a NKT cell, a β islet cell, or a GPC.

In some embodiments, the polycistronic vector further comprises a nucleotide sequence encoding an additional exogenous component. In some embodiments, the additional exogenous component is a safety switch selected from the group consisting of herpes simplex virus thymidine kinase (HSVtk), cytosine deaminase (CyD), nitroreductase (NTR), purine nucleoside phosphorylase (PNP), horseradish peroxidase, inducible caspase 9 (iCasp9), and rapamycin-activated caspase 9 (rapaCasp9).

In some aspects, provided is a T cell having reduced expression of one or more MHC class I molecules, one or more MHC class II molecules, MIC-A, and/or MIC-B and increased expression of one or more tolerogenic factors selected from the group consisting of HLA-E, CD24, CD47, PD-L1, CD46, CD55, CD59, and C1 inhibitor, wherein the one or more tolerogenic factors are carried by the polycistronic vector of any one of claims 1 to 118 or a fragment thereof.

In some aspects, provided is an NK cell having reduced expression of one or more MHC class I molecules, one or more MHC class II molecules, MIC-A, and/or MIC-B and increased expression of one or more tolerogenic factors selected from the group consisting of HLA-E, CD24, CD47, PD-L1, CD46, CD55, CD59, and C1 inhibitor, wherein the one or more tolerogenic factors are carried by the polycistronic vector of any one of claims 1 to 118 or a fragment thereof.

In some aspects, provided is an islet cell having reduced expression of one or more MHC class I molecules, one or more MHC class II molecules, MIC-A, and/or MIC-B and increased expression of one or more tolerogenic factors selected from the group consisting of HLA-E, CD24, CD47, PD-L1, CD46, CD55, CD59, and C1 inhibitor, wherein the one or more tolerogenic factors are carried by the polycistronic vector of any one of claims 1 to 118 or a fragment thereof.

In some embodiments, the reduced expression of MHC class I molecules is by reduced expression of B2M. In some embodiments, the reduced expression of MHC class II molecules is by reduced expression of CIITA. In some embodiments, the reduced expression of MHC class I molecules and/or MHC class II molecules is by reduced expression of MIC-A and/or MIC-B. In some embodiments, the cell has knockout of one or more MHC class I molecules, one or more MHC class II molecules, MIC-A, and/or MIC-B. In some embodiments, the cell has knockout of one or more MHC class I molecules, one or more MHC class II molecules, MIC-A, and MIC-B. In some embodiments, the cell has knockout of MHC class I molecules, MHC class II molecules, MIC-A, and MIC-B. In some embodiments, the knockout is by use of a site-directed nuclease selected from the group consisting of Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, Mad7, a zinc finger nuclease (ZFN), a transcription activator-like effector nuclease (TALEN), a meganuclease, and a CRISPR-associated transposase.

In some embodiments, the one or more tolerogenic factors comprise HLA-E. In some embodiments, the one or more tolerogenic factors is one or more tolerogenic factors comprise CD24. In some embodiments, the one or more tolerogenic factors comprise CD47. In some embodiments, the one or more tolerogenic factors comprise PD-L1. In some embodiments, the one or more tolerogenic factors comprise CD24, CD47, and PD-L1. In some embodiments, the one or more tolerogenic factors comprise CD46. In some embodiments, the one or more tolerogenic factors comprise CD55. In some embodiments, the one or more tolerogenic factors comprise CD59. In some embodiments, the one or more tolerogenic factors comprise C1 inhibitor. In some embodiments, the one or more tolerogenic factors comprise CD46, CD55, CD59, and C1 inhibitor. In some embodiments, the one or more tolerogenic factors comprise HLA-E, CD24, CD47, PD-L1, CD46, CD55, CD59, and C1 inhibitor.

In some embodiments, the host cell is differentiated from an ESC or an iPSC. In some embodiments, the host cell is a primary cell.

In some embodiments, the polycistronic vector further comprises a nucleotide sequence encoding an additional exogenous component. In some embodiments, the additional exogenous component is a safety switch selected from the group consisting of herpes simplex virus thymidine kinase (HSVtk), cytosine deaminase (CyD), nitroreductase (NTR), purine nucleoside phosphorylase (PNP), horseradish peroxidase, inducible caspase 9 (iCasp9), and rapamycin-activated caspase 9 (rapaCasp9).

### BRIEF DESCRIPTION OF THE DRAWINGS

FIGS. 1A-1C are schematics showing the design of a bicistronic vector according to certain embodiments of the present technology and alternative designs. FIG. 1A shows the general design of a CD47-CD19 CAR bicistronic vector in which the vector has an EF1a promoter, a coding sequence for CD47, a cleavage site, and a coding sequence for CD19 CAR. The cleavage site can optionally have a furin site in addition to a 2A site (top), or a 2A site only (bottom). FIG. 1B shows the co-transduction design, using two lentiviral vectors respectively encoding CD19 CAR and CD47 to transduce cells. FIG. 1C shows an alternative design of the CD19 CAR-CD47 vector in which the 5′ to 3′ order of CD47 and CD19 CAR is reversed, i.e., placing CD19 CAR in the first expression cassette and CD47 in the second expression cassette of the bicistronic vector. FC, furin cleavage site.

FIG. 2 is flow cytometry plots showing the expression levels of CD47 (vertical scale) and CD19 CAR (horizontal scale) in primary T cells transfected with the vector construct(s) as indicated. LV, lentiviral virus; *co, codon-optimized; Koz, Kozak consensus sequence.

FIG. 3 shows the expression level of CD47 in cells transduced with different vector constructs as indicated.

FIG. 4 shows the expression level of CD19 CAR in cells transduced with different vector constructs as indicated.

FIG. 5 shows the total CD47 molecule quantities in HIP CAR-T cells (with HLA-I/II knockout (KO) by way of B2M and CIITA deletion) transduced with different vector constructs as indicated.

FIG. 6 shows in vitro xCELLigence assay results of natural killing (NK) and macrophage killing in CD47-CD19 CAR-transduced HIP CAR-T cells. KO, knockout; copt, codon-optimized.

FIG. 7A is a schematic showing the design of a bicistronic vector for co-expression of CD47 and a safety switch. The bicistronic vector includes a construct which has a CAG promoter, a coding sequence for CD47, and a coding sequence for a safety switch (e.g., cytosine deaminase (CyD), herpes simplex virus thymidine kinase (HSVtk)) followed by an HA tag. The coding sequence for CD47 and the coding sequence for the safety switch are separated by a 2A site. In the 5′ to 3′ order, the coding sequence for the safety switch can precede the coding sequence for CD47 (top) or vice versa (bottom). The construct is flanked by the left and right homology arms (LHA, RHA) that are complementary of the region flanking the CLYBL safe harbor locus for directed insertion through homology-directed repair (HDR). FIG. 7B shows the design of a bicistronic vector for co-expression of CD47 and CyD according to certain embodiments of the present technology. The bicistronic vector includes a construct having a CAG promoter, a coding sequence for CyD followed by an HA tag, a 2A site, and a coding sequence for CD47. The construct is flanked by CLYBL LHA and RHA for site-directed insertion into the CLYBL safe harbor locus. FIG. 7C shows the design of another bicistronic vector for co-expression of CD47 and HSVtk according to certain embodiments of the present technology. The bicistronic vector includes a construct having a CAG promoter, a coding sequence for CD47, a 2A site, and a coding sequence for HSVtk followed by an HA tag. The construct is flanked by CLYBL LHA and RHA for site-directed insertion into the CLYBL safe harbor locus.

FIG. 8 shows CD47 expression levels in wild-type (WT) versus CyD-CD47 transduced (clone 2-G11 was depicted as representative) iPSCs and post differentiation.

FIG. 9 shows CyD kill curve as measured by cell count in clones 2-G09 and 2-G11 in the presence of different concentrations (μM) of 5-fluorocytosine (5-FC). Hollow triangles represent iPSCs; filled triangles represent differentiated cells.

FIG. 10 shows CD47 expression levels in CD47-HSVtk clones 1-B10, 1-C02, 2-F09, and 1-H04 over a two-week period.

FIG. 11 shows CD47 fold overexpression levels relative to WT in CD47-HSVtk clones 1-B10, 1-C02, 2-F09, and 1-H04, with the CyD-CD47 clone 2-G09 as a control.

FIG. 12 shows HSVtk kill curve as measured by viable cell count % in CD47-HSVtk clones 1-B10, 1-C02, 2-F09, 1-G10, and 1-H04 in the presence of different concentrations (μM) of ganciclovir (GCV). Calculated IC50 and R square (R²) values for each clone are also shown.

FIG. 13 shows designs of a CD47-CD19 CAR bicistronic vector (top) and a CD47-CD22 CAR bicistronic vector (bottom).

FIG. 14 shows physical and functional assays of the CD47-CD19 CAR, CD47-CD22 CAR (PLAS2199), and CD22 CAR-CD47 (PLAS2218) lentiviruses. Left panel, genome quantitation assay (GQA); middle panel, functional titer assay; right panel, particle to infectivity assay.

FIG. 15 shows a workflow of the dual transduction approach using CD47-CD19 CAR and CD47-CD22 CAR/CD22 CAR-CD47 lentiviruses.

FIG. 16 shows an example gating strategy of flow cytometry analysis of the transduced T cells to examine CD47, CD19 CAR, and CD22 CAR expression levels.

FIG. 17 is flow cytometry plots showing the expression levels of CD19 CAR (vertical scale) and CD22 CAR (horizontal scale) in CD4+ (top panels) and CD8+ (bottom panels) T cells transduced with the lentivirus(es) as indicated.

FIG. 18 shows efficiency of single and dual transduction approaches, as indicated by percentage of CAR-expressing cells, across different donors and virus lots. Top panels show percentage of total product populations; bottom panels show percentage of total transduced cells. Each symbol color represents an independent donor, CD19 CAR virus, or CD22 CAR virus, respectively.

FIG. 19 shows median fluorescent intensity (MFI) of CD47, CD19 CAR, and CD22 CAR expression levels in donor cells transduced with the lentivirus(es) as indicated.

FIG. 20 shows CD47 expression levels as quantified by QiFi (Agilent) analysis and vector copy number (VCN) in donor cells transduced with the lentivirus(es) as indicated.

FIG. 21A shows a workflow of an assay testing the VCN of single and dual transduction approaches using the lentivirus(es) as indicated. FIG. 21B shows the VCN from dual and single transduced cells.

FIG. 22 shows cytotoxicity (IncuCyte®, top panels) and cytokine (Meso Scale Discovery, bottom panels) assay results of red fluorescent protein (RFP)-labeled control NALM, NALM CD19 knockout (KO), and NALM CD22 KO cells treated with T cells single transduced with CD47-CD19 CAR (CD19 CAR-T cells), T cells single transduced with CD47-CD22 CAR (CD22 CAR-T cells), or T cells dual transduced with CD47-CD19 CAR/CD47-CD22 CAR (CD19×CD22 CAR-T cells) as indicated. Cytotoxicity was measured by total integrated intensity of RFP over time.

FIG. 23 shows cytotoxicity as measured by luciferase assay of T cells transduced with (1) mock; (2) CD47-CD19 CAR lentivirus alone; (2) CD22 CAR-CD47 lentivirus alone; (3) CD47-CD22 CAR lentivirus alone; (4) CD47-CD19 CAR lentivirus and CD22 CAR-CD47 lentivirus mixture (CD47-CD19 CAR×CD22 CAR-CD47); and (5) CD47-CD19 CAR lentivirus and CD47-CD22 CAR lentivirus mixture (CD47-CD19 CAR×CD47-CD22 CAR) against various NALM and RAJI tumor cells at various effector (E) to target (T) (E:T) ratios as indicated. E:T ratio was defined as the ratio of T cell number to NALM or RAJI cell number.

FIG. 24 shows a workflow of producing, sorting, and testing of single or dual transduced CAR-T cells. CAR Selection 1 involves anti-idiotype-biotin and anti-biotin microbeads for CD19 CAR and dual transduced T cells and soluble CD22-biotin for CD22 CAR; CAR Selection 2 involves soluble CD22-biotin for CD22 CAR in dual transduced population.

FIG. 25 is flow cytometry plots showing the expression levels of CD19 CAR (vertical scale) and CD22 CAR (horizontal scale) in CD4+(left panels) and CD8+(right panels) T cells transduced with the lentivirus(es) as indicated before (top panels) and after (bottom panels) sorting.

FIG. 26 shows cytotoxicity curve of RFP-labeled NALM cells treated with mock, CD19 CAR-T, CD22 CAR-T, or CD19×CD22 CAR-T cells as indicated. Cytotoxicity was measured by total integrated intensity of RFP over time. E:T ratios were indicated at the bottom left corner of each graph and were based on the ratio of CAR-expressing T cell number to NALM cell number.

FIG. 27 shows cytotoxicity curve of RFP-labeled CD19 KO NALM cells treated with mock, CD19 CAR-T, CD22 CAR-T, or CD19×CD22 CAR-T cells as indicated. Cytotoxicity was measured by total integrated intensity of RFP over time. E:T ratios were indicated at the bottom left corner of each graph and were based on the ratio of CAR-expressing T cell number to NALM cell number.

FIG. 28 shows cytotoxicity curve of RFP-labeled CD22 KO NALM cells treated with mock, CD19 CAR-T, CD22 CAR-T, or CD19×CD22 CAR-T cells as indicated. Cytotoxicity was measured by total integrated intensity of RFP over time. E:T ratios were indicated at the bottom left corner of each graph and were based on the ratio of CAR-expressing T cell number to NALM cell number.

FIG. 29 shows cytokine levels (vertical scale) of control NALM, CD19 KO NALM, and CD22 KO NALM cells treated with mock, CD19 CAR-T, CD22 CAR-T, or CD19×CD22 CAR-T cells at various E:T ratios (horizontal scale) as indicated.

FIG. 30 shows a study design testing the abilities of CD22 CAR-T and dual transduced CD19×CD22 CAR-T cells to control tumor growth in CD19 KO NALM tumor engrafted mouse models.

FIGS. 31-34 show effective inhibition of tumor growth by CD22 CAR-T and CD19 CAR×CD22 CAR-T cells in CD19 KO NALM (FIGS. 31-32) and RAJI (FIGS. 33-34) mouse models as measured by bioluminescence of tumor cells (FIGS. 31, 33) and whole mouse imaging (FIGS. 32, 34).

FIG. 35 shows a study design comparing the abilities of dual transduced, or dual transduced and sorted, CD19×CD22 CAR-T cells to control tumor growth to that of a combination of CD19 CAR- and CD22 CAR-T cells in NALM tumor engrafted mouse models.

FIG. 36 shows more effective inhibition of tumor growth by of dual transduced, or dual transduced and sorted, CD19×CD22 CAR-T cells compared to mixtures of single transduced CAR-T cells in NALM mouse models as measured by bioluminescence of tumor cells.

### DETAILED DESCRIPTION

The present disclosure provides polycistronic vectors for co-expression of one or more tolerogenic factors (e.g., CD47, HLA-E, HLA-G, PD-L1, CTLA-4, etc.), one or more CARs (e.g., CD19 CAR, CD22 CAR, BCMA CAR, etc.), and optionally one or more safety switches in a host cell for various cell-based therapies. As explained herein, overexpression of the tolerogenic factor, for example, in allogeneic cells and often in addition to other genetic modifications (e.g., B2M and CIITA knockout), can improve the hypoimmunogenicity of the resulting cells so that they will not be subject to immune rejection when transplanted into a recipient, thus increasing the effectiveness of cell-based therapies. Inclusion of a safety switch in the polycistronic vector, on the other hand, allows for controlled killing of the cells in the event of cytotoxicity or other negative consequences to the recipient, thus increasing the safety of cell-based therapies, including those using tolerogenic factors.

While the present disclosure is capable of being embodied in various forms, the description below of several embodiments is made with the understanding that the present disclosure is to be considered as an exemplification of the invention and is not intended to limit the invention to the specific embodiments illustrated. Headings are provided for convenience only and are not to be construed to limit the invention in any manner. Embodiments illustrated under any heading may be combined with embodiments illustrated under any other heading.

The use of numerical values in the various quantitative values specified in this application, unless expressly indicated otherwise, are stated as approximations as though the minimum and maximum values within the stated ranges were both preceded by the word “about.” It is to be understood, although not always explicitly stated, that all numerical designations are preceded by the term “about.” It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios, such as about 2, about 3, and about 4, and sub-ranges, such as about 10 to about 50, about 20 to about 100, and so forth. It also is to be understood, although not always explicitly stated, that the reagents described herein are merely exemplary and that equivalents of such are known in the art.

To the extent any materials incorporated by reference herein conflict with the present disclosure, the present disclosure controls.

### Definitions

The term “about,” as used herein when referring to a measurable value, such as an amount or concentration and the like, is meant to encompass variations of 20%, 10%, 5%, 1%, 0.5%, or even 0.1% of the specified amount.

The term “antibody” is used to denote, in addition to natural antibodies, genetically engineered or otherwise modified forms of immunoglobulins or portions thereof, including chimeric antibodies, human antibodies, humanized antibodies, or synthetic antibodies. The antibodies may be monoclonal or polyclonal antibodies. In those embodiments wherein an antibody is an immunogenically active portion of an immunoglobulin molecule, the antibody may include, but is not limited to, a single chain variable fragment antibody (scFv), disulfide linked Fv, single domain antibody (sdAb), VHH antibody, antigen-binding fragment (Fab), Fab′, F(ab′)2 fragment, or diabody. An scFv antibody is derived from an antibody by linking the variable regions of the heavy (V_{H}) and light (V_{L}) chains of the immunoglobulin with a short linker peptide. Similarly, a disulfide linked Fv antibody can be generated by linking the V_{H} and V_{L} using an interdomain disulfide bond. On the other hand, sdAbs consist of only the variable region from either the heavy or light chain and usually are the smallest antigen-binding fragments of antibodies. A VHH antibody is the antigen binding fragment of heavy chain only. A diabody is a dimer of scFv fragment that consists of the V_{H} and V_{L} regions noncovalent connected by a small peptide linker or covalently linked to each other. The antibodies disclosed herein, including those that comprise an immunogenically active portion of an immunoglobulin molecule, retain the ability to bind a specific antigen.

The term “antigen” as used herein refers to a molecule capable of provoking an immune response. Antigens include but are not limited to cells, cell extracts, proteins, polypeptides, peptides, polysaccharides, polysaccharide conjugates, peptide and non-peptide mimics of polysaccharides and other molecules, small molecules, lipids, glycolipids, carbohydrates, viruses and viral extracts and multicellular organisms such as parasites and allergens. The term antigen broadly includes any type of molecule which is recognized by a host immune system as being foreign.

The term “autoimmune disease,” “autoimmune disorder,” “inflammatory disease,” or “inflammatory disorder” refers to any disease or disorder in which the subject mounts an immune response against its own tissues and/or cells. Autoimmune disorders can affect almost every organ system in the subject (e.g., human), including, but not limited to, diseases of the nervous, gastrointestinal, and endocrine systems, as well as skin and other connective tissues, eyes, blood and blood vessels. Examples of autoimmune diseases include, but are not limited to Hashimoto's thyroiditis, systemic lupus erythematosus, Sjögren's syndrome, Graves' disease, scleroderma, rheumatoid arthritis, multiple sclerosis, myasthenia gravis and diabetes.

A “binding domain,” also referred to as a “binding region,” refers to an antibody or portion thereof that possesses the ability to specifically and non-covalently associate, unite, or combine with a target. A binding domain includes any naturally occurring, synthetic, semi-synthetic, or recombinantly produced binding partner for a biological molecule, a molecular complex, or other target of interest. Exemplary binding domains include receptor ectodomains, ligands, scFvs, disulfide linked Fvs, sdAbs, VHH antibodies, Fab fragments, Fab′ fragments, F(ab′)₂ fragments, diabodies, or other synthetic polypeptides selected for their specific ability to bind to a biological molecule, a molecular complex, or other target of interest.

The term “chimeric antigen receptors (CARs),” also known as chimeric T cell receptors or artificial T cell receptors, refers to artificially engineered receptors that combine both antigen-binding and T cell activating functions. CARs may include an extracellular portion comprising a binding domain, such as one obtained or derived from an antibody (e.g., an scFv). The extracellular portion may be linked through a transmembrane domain to one or more intracellular signaling or effector domains. CARs can optionally contain an intracellular costimulatory domain(s). See, e.g Sadelain et al., Cancer Discov., 3(4):388-398 (2013); see also Harris & Kranz, Trends Pharmacol. Sci., 37(3):220-230 (2016); Stone et al., Cancer Immunol. Immunother., 63(11):1163-1176 (2014). CARs can be introduced to be expressed on the surface of a T cell, so that the T cell can target and kill target cells (e.g., cancer cells) that express the antigen the CAR is designed to bind.

The term “codon-optimized” or “codon optimization” when referring to a nucleotide sequence is based on the discovery that the frequency of occurrence of synonymous codons (i.e., codons that code for the same amino acid) in coding nucleotide is biased in different species. Such codon degeneracy allows an identical polypeptide to be encoded by a variety of nucleotide sequences. Codon optimization refers to the process of substituting certain codons in a coding nucleotide sequence with synonymous codons based on the host cell's preference without changing the resulting polypeptide sequence. A variety of codon optimization methods are known in the art, and include, for example, methods disclosed in at least U.S. Pat. Nos. 5,786,464 and 6,114,148.

The term “complementarity determining regions (CDRs)” is synonymous with “hypervariable region” or “HVR,” and is known in the art to refer to sequences of amino acids within antibody variable regions, which, in general, confer antigen specificity and/or binding affinity and are separated from one another in primary structure by framework sequence. In some cases, framework amino acids can also contribute to binding. In general, there are three CDRs in each variable region. Variable domain sequences can be aligned to a numbering scheme (e.g., Kabat, EU, international ImMunoGeneTics information System® (IMGT®), and Aho), which can allow equivalent residue positions to be annotated and for different molecules to be compared using the Antibody Numbering and Antigen Receptor Classification (ANARCI) software tool (2016, Bioinformatics 15:298-300).

The term “construct” refers to any polynucleotide that contains a recombinant nucleic acid molecule. A construct may be present in a vector (e.g., a bacterial vector, a viral vector) or may be integrated into a genome. A “vector” is a nucleic acid molecule that is capable of introducing a specific nucleic acid sequence into a cell or into another nucleic acid sequence, or as a means of transporting another nucleic acid molecule. Vectors may be, for example, plasmids, cosmids, viruses, an RNA vector, or a linear or circular DNA or RNA molecule that may include chromosomal, non-chromosomal, semi-synthetic, or synthetic nucleic acid molecules. Exemplary vectors are those capable of autonomous replication (episomal vector), capable of delivering a polynucleotide to a cell genome (e.g., viral vector), or capable of expressing nucleic acid molecules to which they are linked (expression vectors).

The term “epitope” includes any molecule, structure, amino acid sequence, or protein determinant that is recognized and specifically bound by a cognate binding molecule, such as an antibody or a T cell receptor, or other binding molecule, domain, or protein.

The term “expression” refers to the process by which a polypeptide is produced based on the encoding sequence of a nucleic acid molecule, such as a gene. The process may include transcription, post-transcriptional control, post-transcriptional modification, translation, post-translational control, post-translational modification, or any combination thereof. An expressed nucleic acid molecule is typically operably linked to an expression control sequence (e.g., a promoter).

The term “host cell” as used herein refers to a cell or microorganism targeted for genetic modification by introduction of a construct or vector carrying a nucleotide sequence for expression of a protein or polypeptide of interest. In certain embodiments, when the protein to be expressed includes a CAR, the host cell is usually a T cell.

The term “hypoimmunogenicity,” “hypoimmunogeneic,” “hypoimmunogenic,” “hypoimmunity,” or “hypoimmune” is used interchangeably to describe a cell being less prone to immune rejection by a subject into which such cell is transplanted. For example, relative to an unaltered or unmodified wild-type cell, such a hypoimmunogenic cell may be about 2.5%, 5%, 10%, 20%, 30%, 40%, 50%, 60%, 70%, 80%, 90%, 95%, 97.5%, 99% or more less prone to immune rejection by a subject into which such cell is transplanted. In some examples described herein, genome editing technologies are used to modulate the expression of MHC I and MHC II genes, and thus, to generate a hypoimmunogenic cell. In other examples described herein, a tolerogenic factor is introduced into a cell and when expressed can modulate or affect the ability of the cell to be recognized by host immune system and thus confer hypoimmunogenicity. Hypoimmunogenicity of a cell can be determined by evaluating the cell's ability to elicit adaptive and innate immune responses. Such immune response can be measured using assays recognized by those skilled in the art, for example, by measuring the effect of a hypoimmunogenic cell on T cell proliferation, T cell activation, T cell killing, NK cell proliferation, NK cell activation, and macrophage activity. Hypoimmunogenic cells may undergo decreased killing by T cells and/or NK cells upon administration to a subject or show decreased macrophage engulfment compared to an unmodified or wildtype cell. In some cases, a hypoimmunogenic cell elicits a reduced or diminished immune response in a recipient subject compared to a corresponding unmodified wild-type cell. In some cases, a hypoimmunogenic cell is nonimmunogenic or fails to elicit an immune response in a recipient subject. Detailed descriptions of hypoimmunogenic cells, methods of producing the same, and methods of using the same are found in WO2016183041 filed May 9, 2015; WO2018132783 filed Jan. 14, 2018; WO2018176390 filed Mar. 20, 2018; WO2020018615 filed Jul. 17, 2019; WO2020018620 filed Jul. 17, 2019; WO2021022223 filed Jul. 31, 2020; WO2021022223 filed Jul. 31, 2020; WO2021041316 filed Aug. 24, 2020; WO2021222285 filed Apr. 27, 2021, 2020; and WO2021222285 filed Apr. 27, 2021, the disclosures including the examples, sequence listings and figures are incorporated herein by reference in their entirety.

An “intracellular signaling domain” or “effector domain” is an intracellular portion or domain of a CAR or receptor that can directly or indirectly promote a biological or physiological response in a cell when receiving an appropriate signal. In certain embodiments, an effector domain is from a protein or portion thereof or protein complex that receives a signal when bound to a target or cognate molecule, or when the protein or portion thereof or protein complex binds directly to a target or cognate molecule and triggers a signal from the effector domain.

The term “nucleic acid” or “polynucleotide” refers to a polymeric compound including covalently linked nucleotides comprising natural subunits (e.g., purine or pyrimidine bases). Purine bases include adenine and guanine, and pyrimidine bases include uracil, thymine, and cytosine. Nucleic acid molecules include polyribonucleic acid (RNA) and polydeoxyribonucleic acid (DNA), which includes cDNA, genomic DNA, and synthetic DNA, either of which may be single- or double-stranded. A nucleic acid molecule encoding an amino acid sequence includes all nucleotide sequences that encode the same amino acid sequence.

The term “operably linked” refers to the association of two or more nucleic acid molecules on a single nucleic acid fragment so that the function of one is affected by the other.

The term “patient” refers to an animal, for example, a human to whom treatment, including prophylactic treatment, with the cells as described herein, is provided. For treatment of those infections, conditions or disease states, which are specific for a specific animal such as a human patient, the term patient refers to that specific animal. The term “patient” also encompasses any vertebrate including but not limited to mammals, reptiles, amphibians and fish. However, advantageously, the patient is a mammal such as a human, or other mammals such as a domesticated mammal, e.g., dog, cat, horse, and the like, or production mammal, e.g., cow, sheep, pig, and the like.

The term “safe harbor” or “safe harbor locus” refers to a gene locus that allow safe expression of a transgene or an exogenous gene. The transgene or exogenous gene can be inserted into any suitable region of a safe harbor locus that allows for safe expression of the gene, including, for example, an intron, an exon, or a coding sequence region (CDS) in a safe harbor locus.

The term “safety switch” used herein refers to a system for controlling the expression of a gene or protein of interest that, when downregulated or upregulated, leads to clearance or death of the cell, e.g., through recognition by the host's immune system. A safety switch can be designed to be triggered by an exogenous molecule in case of an adverse clinical event. A safety switch can be engineered by regulating the expression on the DNA, RNA and protein levels. A safety switch includes a protein or molecule that allows for the control of cellular activity in response to an adverse event. In some embodiments, the safety switch is a “kill switch” that is expressed in an inactive state and is fatal to a cell expressing the safety switch upon activation of the switch by a selective, externally provided agent. In some embodiments, the safety switch gene is cis-acting in relation to the gene of interest in a construct. Activation of the safety switch causes the cell to kill solely itself or itself and neighboring cells through apoptosis or necrosis.

The term “subject” refers to a mammalian subject, preferably a human. A “subject in need thereof” may refer to a subject who has been diagnosed with a disease, or is at an elevated risk of developing a disease. The phrases “subject” and “patient” are used interchangeably herein.

A “therapeutically effective amount” as used herein is an amount that produces a desired effect in a subject for treating a disease. In certain embodiments, the therapeutically effective amount is an amount that yields maximum therapeutic effect. In other embodiments, the therapeutically effective amount yields a therapeutic effect that is less than the maximum therapeutic effect. For example, a therapeutically effective amount may be an amount that produces a therapeutic effect while avoiding one or more side effects associated with a dosage that yields maximum therapeutic effect. A therapeutically effective amount for a particular composition will vary based on a variety of factors, including, but not limited, to the characteristics of the therapeutic composition (e.g., activity, pharmacokinetics, pharmacodynamics, and bioavailability), the physiological condition of the subject (e.g., age, body weight, sex, disease type and stage, medical history, general physical condition, responsiveness to a given dosage, and other present medications), the nature of any pharmaceutically acceptable carriers, excipients, and preservatives in the composition, and the route of administration. One skilled in the clinical and pharmacological arts will be able to determine a therapeutically effective amount through routine experimentation, namely by monitoring a subject's response to administration of the host cell, or a pharmaceutical composition containing the same, and adjusting the dosage accordingly. For additional guidance, see, e.g., Remington: The Science and Practice of Pharmacy, 22^{nd} Edition, Pharmaceutical Press, London, 2012, and Goodman & Gilman's The Pharmacological Basis of Therapeutics, 12^{th} Edition, McGraw-Hill, New York, NY, 2011, the entire disclosures of which are incorporated by reference herein.

The term “tolerogenic factor” as used herein includes hypoimmunity factors, complement inhibitors, and other factors that modulate or affect the ability of a cell to be recognized by the immune system of a host or recipient subject upon administration, transplantation, or engraftment.

A “transmembrane region” is a portion of a transmembrane protein that can insert into or span a cell membrane.

The terms “treat,” “treating,” and “treatment” as used herein with regard to cancer refers to alleviating the cancer partially or entirely; preventing the cancer; decreasing the likelihood of occurrence or recurrence of the cancer; slowing the progression or development of the cancer; eliminating, reducing, or slowing the development of one or more symptoms associated with the cancer; or increasing progression-free or overall survival of the cancer. For example, “treating” may refer to preventing or slowing the existing cancer from growing larger; preventing or slowing the formation or metastasis of cancer; and/or slowing the development of certain symptoms of the cancer. In some embodiments, the term “treat,” “treating,” or “treatment” means that the subject has a reduced number or size of cancer cells comparing to a subject without being administered with the treatment. In some embodiments, the term “treat,” “treating,” or “treatment” means that one or more symptoms of the cancer are alleviated in a subject receiving the treatment as disclosed and described herein comparing to a subject who does not receive such treatment.

The term “variable region” or “variable domain” refers to a portion of an antibody heavy or light chain that is involved in antigen binding. Variable domains of antibody heavy (V_{H}) and light (V_{L}) chains each generally comprise four generally conserved framework regions (FRs) and three complementarity determining regions (CDRs). Framework regions separate CDRs, such that CDRs are situated between framework regions.

A “vector” refers to a DNA construct containing a nucleic acid molecule that is operably linked to a suitable control sequence capable of effecting the expression of the nucleic acid molecule in a suitable host. Such control sequences may include a promoter to effect transcription, an optional operator sequence to control such transcription, a sequence encoding suitable mRNA ribosome binding sites, and sequences which control termination of transcription and translation. The vector may be a plasmid, a phage particle, a virus, or simply a potential genomic insert. Once transformed into a suitable host, the vector may replicate and function independently of the host genome, or may, in some instances, integrate into the genome itself.

### Polycistronic Vectors and Compositions Thereof

In some aspects, the present technology provides polycistronic vectors comprising two or more expression cassettes for co-expression of two or more proteins of interest in a host cell. In some embodiments, the polycistronic vector comprises two expression cassettes, i.e., is bicistronic. In some embodiments, the polycistronic vector comprises three expression cassettes, i.e., is tricistronic. In some embodiments, the polycistronic vector comprises four expression cassettes, i.e., is quadcistronic. In some embodiments, the polycistronic vector comprises more than four expression cassettes. In any of these embodiments, each of the expression cassettes comprises a nucleotide sequence encoding a protein of interest. In certain embodiments, the two or more genes being expressed are under the control of a single promoter and are separated from one another by one or more cleavage sites to achieve co-expression of the proteins of interest from one transcript. In other embodiments, the two or more genes may be under the control of separate promoters.

### Tolerogenic Factor

In certain embodiments, the polycistronic vector may comprise one or more expression cassettes each comprising a nucleotide sequence encoding a tolerogenic factor. The tolerogenic factors expressed by the one or more expression cassettes may be the same or different. In some embodiments, the tolerogenic factor is selected from the group consisting of A20/TNFAIP3, CD16, CD16 Fc receptor, CD24, CD35, CD39, CD46, CD47, CD52, CD55, CD59, CD200, CCL22, CTLA4-Ig, C1 inhibitor, complement receptor (CR1), DUX4, FASL, H2-M3, IDO1, IL15-RF, HLA-C, HLA-E, HLA-E heavy chain, HLA-G, IL-10, IL-35, MANF, PD-1, PD-L1, Serpinb9, CCl21, Mfge8, and truncations, modifications, or fusions of any of the above. In the embodiments where the polycistronic vector comprises two or more expression cassettes encoding two or more tolerogenic factors, the two or more tolerogenic factors can be independently selected from the group consisting of A20/TNFAIP3, CD16, CD16 Fc receptor, CD24, CD35, CD39, CD46, CD47, CD52, CD55, CD59, CD200, CCL22, CTLA4-Ig, C1 inhibitor, complement receptor (CR1), DUX4, FASL, H2-M3, IDO1, IL15-RF, HLA-C, HLA-E, HLA-E heavy chain, HLA-G, IL-10, IL-35, MANF, PD-1, PD-L1, Serpinb9, CCl21, Mfge8, truncations, modifications, or fusions of any of the above, or any combinations thereof.

In some embodiments, the tolerogenic factor is CD47. CD47 is a leukocyte surface antigen and has a role in cell adhesion and modulation of integrins. It is expressed on the surface of a cell (e.g., a T cell) and signals to circulating macrophages not to phagocytize the cell. Overexpression of CD47 thus can reduce the immunogenicity of the cell when grafted and improve immune protection in allogeneic recipients. CD47 is a transmembrane protein that, in humans, is encoded by the CD47 gene. It is a member of the immunoglobulin (Ig) superfamily. CD47 has a molecular weight of about ˜50 kDa. It is glycosylated and ubiquitously expressed by virtually all cells in the human body. It has a single IgV-like domain at its N-terminus, a highly hydrophobic stretch with five membrane-spanning segments, and an alternatively spliced cytoplasmic tail at its C-terminus. In addition, it has two extracellular regions and two intracellular regions between neighboring membrane-spanning segments. A signal peptide, when it exists on a CD47 isoform, is located at the N-terminus of the IgV-like domain.

CD47 is involved in a range of cellular processes, including apoptosis, proliferation, adhesion, and migration. CD47 interacts with multiple extracellular ligands, such as TSP-1, integrins, other CD47 proteins, and SIRPa. The CD47/SIRPa interaction regulates a multitude of intercellular interactions in many body systems, such as the immune system where it regulates lymphocyte homeostasis, dendritic cell (DC) maturation and activation, proper localization of certain DC subsets in secondary lymphoid organs, and cellular transmigration. CD47 on cells, including on donor cells in the context of transplantation or cell therapy applications, can function as a “marker of self” and regulate phagocytosis by binding to SIRPa on the surface of circulating immune cells to deliver an inhibitory “don't kill me” signal. CD47-SIRPa binding results in phosphorylation of immunoreceptor tyrosine-based inhibition motifs (ITIMs) on SIRPa, which triggers recruitment of the SHP1 and SHP2 Src homology phosphatases. These phosphatases, in turn, inhibit accumulation of myosin II at the phagocytic synapse, preventing phagocytosis (Fujioka et al., Mol. Cell. Biol., 16:6887-6899 (1996)). Phagocytosis of target cells by macrophages is ultimately regulated by a balance of activating signals (e.g., FcγR, CRT, LRP-1) and inhibitory signals (e.g., SIRPa-CD47). Elevated expression of CD47 can help the cell evade immune surveillance, subsequent destruction, and innate immune cell killing. Thus, CD47 can be used as a tolerogenic factor to induce immune tolerance, for example, when there is pathological or undesirable activation of an otherwise normal immune response. This can occur, for example, when a patient develops an immune reaction to donor antigens after receiving an allogeneic transplantation or an allogeneic cell therapy, or when the body responds inappropriately to self-antigens implicated in autoimmune diseases.

The human CD47 gene has six naturally occurring transcripts, five of which each encode a protein isoform of CD47 (Ensembl, Gene: CD47, ENSG00000196776). The six transcripts are named CD47-201, CD47-202, CD47-203, CD47-204, CD47-205, and CD47-206. The coding DNA sequence (CDS) of the six transcripts are as set forth in SEQ ID NOs: 129-134, respectively. The amino acid sequences of the five protein isoforms are as set forth in SEQ ID NOs:1-5 respectively (see Table 1).

Transcript CD47-201 (SEQ ID NO:129) encodes isoform CD47-201 (SEQ ID NO:1), which has 305 amino acids. Isoform CD47-201 has a C-terminal truncation of 18 amino acids from isoform CD47-202. All splice junctions of the CD47-201 transcript are supported by at least one non-suspect mRNA.

Transcript CD47-202 (SEQ ID NO:130) encodes isoform CD47-202 (SEQ ID NO:2), which has 323 amino acids. CD47-202 is the longest transcript of the human CD47 gene. It is designated as the representative transcript in the Ensembl database. In identifying the representative transcript, Ensembl aims to identity the transcript that, on balance, has the highest coverage of conserved exons, highest expression, longest coding sequence and is represented in other key resources, such as NCBI and UniProt. All splice junctions of the CD47-202 transcript are supported by at least one non-suspect mRNA. Amino acids 1-18 are the signal peptide.

Transcript CD47-203 (SEQ ID NO:131) encodes isoform CD47-203 (SEQ ID NO:3), which has 86 amino acids. The only support for the transcript model is from a single expressed sequence tag (EST).

Transcript CD47-204 (SEQ ID NO:132) does not encode protein. All splice junctions of this transcript are supported by at least one non-suspect mRNA

Transcript CD47-205 (SEQ ID NO:133) encodes isoform CD47-205 (SEQ ID NO:4), which has 109 amino acids. Isoform 205 comprises 3 transmembrane domains and a truncated intracellular domain from isoform CD47-202. The best supporting mRNA for the transcript model is flagged as suspect or the support is from multiple ESTs.

Transcript CD47-206 (SEQ ID NO:134) encodes isoform CD47-206 (SEQ ID NO:5), which has 183 amino acids. Isoform 206 comprises a truncated extracellular domain and 5 transmembrane domains from isoform CD47-202.

In some embodiments, the polycistronic vector comprises an expression cassette that contains a nucleotide sequence encoding CD47. In some embodiments, the CD47 is human CD47, and in some of these embodiments, the human CD47 comprises or consists of an amino acid sequence set forth in any one of SEQ ID NOs: 1-5, or is at least 80% identical (e.g., at least 80%, at least 85%, at least 90%, at least 95%, at least 96%, at least 97%, at least 98%, at least 99%, or 100% identical) to the amino acid sequence set forth in any one of SEQ ID NOs: 1-5. In some embodiments, the human CD47 comprises or consists of an amino acid sequence set forth in SEQ ID NO:2 or is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:2. In some embodiments, the nucleotide sequence encoding CD47 corresponds to an mRNA sequence of human CD47. In some embodiments, the nucleotide sequence encoding CD47 is at least 80% identical (e.g., at least 80%, at least 85%, at least 90%, at least 95%, at least 96%, at least 97%, at least 98%, at least 99%, or 100% identical) to the nucleotide sequence set forth in any one of SEQ ID NOs: 129-134. In some embodiments, the nucleotide sequence encoding CD47 is at least 80% identical (e.g., at least 80%, at least 85%, at least 90%, at least 95%, at least 96%, at least 97%, at least 98%, at least 99%, or 100% identical) to the nucleotide sequence set forth in SEQ ID NO:130.

In some embodiments, the nucleotide sequence encoding CD47 is codon-optimized for expression in a mammalian cell, for example, a human cell. In some embodiments, the codon-optimized nucleotide sequence encoding CD47 is at least 80% identical (e.g., at least 80%, at least 85%, at least 90%, at least 95%, at least 96%, at least 97%, at least 98%, at least 99%, or 100% identical) to the nucleotide sequence set forth in SEQ ID NO:135.

TABLE 1
Exemplary sequences of CD47

| SEQ ID NO: | Sequence | Description |
|---|---|---|
| 1 | MWPLVAALLLGSACCGSAQLLFNKTKSVEFTFCNDTVVIPCFVTNMEAQNTTEVY | CD47-201 |
| | VKWKFKGRDIYTFDGALNKSTVPTDFSSAKIEVSQLLKGDASLKMDKSDAVSHTG | amino acid |
| | NYTCEVTELTREGETIIELKYRVVSWFSPNENILIVIFPIFAILLFWGQFGIKTL | sequence |
| | KYRSGGMDEKTIALLVAGLVITVIVIVGAILFVPGEYSLKNATGLGLIVTSTGIL | |
| | ILLHYYVFSTAIGLTSFVIAILVIQVIAYILAVVGLSLCIAACIPMHGPLLISGL | |
| | SILALAQLLGLVYMKFVASNQKTIQPPRNN | |
| 2 | MWPLVAALLLGSACCGSAQLLFNKTKSVEFTFCNDTVVIPCFVTNMEAQNTTEVY | CD47-202 |
| | VKWKFKGRDIYTFDGALNKSTVPTDFSSAKIEVSQLLKGDASLKMDKSDAVSHTG | amino acid |
| | NYTCEVTELTREGETIIELKYRVVSWFSPNENILIVIFPIFAILLFWGQFGIKTL | sequence |
| | KYRSGGMDEKTIALLVAGLVITVIVIVGAILFVPGEYSLKNATGLGLIVTSTGIL | |
| | ILLHYYVFSTAIGLTSFVIAILVIQVIAYILAVVGLSLCIAACIPMHGPLLISGL | |
| | SILALAQLLGLVYMKFVASNQKTIQPPRKAVEEPLNAFKESKGMMNDE | |
| 3 | XWTESLYCGVYTNAWPSSDFRFEYLSSSTITWTSLYEICGVQRDTYSQLKGKKRQ | CD47-203 |
| | ASNQKTIQPPRKAVEEPLNAFKESKGMMNDE | amino acid |
| | | sequence |
| 4 | XKNATGLGLIVTSTGILILLHYYVFSTAIGLTSFVIAILVIQVIAYILAVVGLSL | CD47-205 |
| | CIAACIPMHGPLLISGLSILALAQLLGLVYMKFVASNQKTIQPPRKAVEEPLNE | amino acid |
| | | sequence |
| 5 | MEAQNTTEVYVKWKFKGRDIYTFDGALNKSTVPTDFSSAKIEVSQLLKGDASLKM | CD47-206 |
| | DKSDAVSHTGNYTCEVTELTREGETIIELKYRVVSWFSPNENILIVIFPIFAILL | amino acid |
| | FWGQFGIKTLKYRSGGMDEKTIALLVAGLVITVIVIVGAILFVPGEYSLKNATGL | sequence |
| | GLIVTSTGILILLHYYVF | |
| 129 | atgtggcccctggtagcggcgctgttgctgggctcggcgtgctgcggatcagctc | CD47-201 |
| | agctactatttaataaaacaaaatctgtagaattcacgttttgtaatgacactgt | CDS |
| | cgtcattccatgctttgttactaatatggaggcacaaaacactactgaagtatac | nucleotide |
| | gtaaagtggaaatttaaaggaagagatatttacacctttgatggagctctaaaca | sequence |
| | agtccactgtccccactgactttagtagtgcaaaaattgaagtctcacaattact | |
| | aaaaggagatgcctctttgaagatggataagagtgatgctgtctcacacacagga | |
| | aactacacttgtgaagtaacagaattaaccagagaaggtgaaacgatcatcgagc | |
| | taaaatatcgtgttgtttcatggttttctccaaatgaaaatattcttattgttat | |
| | tttcccaatttttgctatactcctgttctggggacagtttggtattaaaacactt | |
| | aaatatagatccggtggtatggatgagaaaacaattgctttacttgttgctggac | |
| | tagtgatcactgtcattgtcattgttggagccattcttttcgtcccaggtgaata | |
| | ttcattaaagaatgctactggccttggtttaattgtgacttctacagggatatta | |
| | atattacttcactactatgtgtttagtacagcgattggattaacctccttcgtca | |
| | ttgccatattggttattcaggtgatagcctatatcctcgctgtggttggactgag | |
| | tctctgtattgcggcgtgtataccaatgcatggccctcttctgatttcaggtttg | |
| | agtatcttagctctagcacaattacttggactagtttatatgaaatttgtggctt | |
| | ccaatcagaagactatacaacctcctaggaataactga | |
| 130 | atgtggcccctggtagcggcgctgttgctgggctcggcgtgctgcggatcagctc | CD47-202 |
| | agctactatttaataaaacaaaatctgtagaattcacgttttgtaatgacactgt | CDS |
| | cgtcattccatgctttgttactaatatggaggcacaaaacactactgaagtatac | nucleotide |
| | gtaaagtggaaatttaaaggaagagatatttacacctttgatggagctctaaaca | sequence |
| | agtccactgtccccactgactttagtagtgcaaaaattgaagtctcacaattact | |
| | aaaaggagatgcctctttgaagatggataagagtgatgctgtctcacacacagga | |
| | aactacacttgtgaagtaacagaattaaccagagaaggtgaaacgatcatcgagc | |
| | taaaatatcgtgttgtttcatggttttctccaaatgaaaatattcttattgttat | |
| | tttcccaatttttgctatactcctgttctggggacagtttggtattaaaacactt | |
| | aaatatagatccggtggtatggatgagaaaacaattgctttacttgttgctggac | |
| | tagtgatcactgtcattgtcattgttggagccattcttttcgtcccaggtgaata | |
| | ttcattaaagaatgctactggccttggtttaattgtgacttctacagggatatta | |
| | atattacttcactactatgtgtttagtacagcgattggattaacctccttcgtca | |
| | ttgccatattggttattcaggtgatagcctatatcctcgctgtggttggactgag | |
| | tctctgtattgcggcgtgtataccaatgcatggccctcttctgatttcaggtttg | |
| | agtatcttagctctagcacaattacttggactagtttatatgaaatttgtggctt | |
| | ccaatcagaagactatacaacctcctaggaaagctgtagaggaaccccttaatgc | |
| | attcaaagaatcaaaaggaatgatgaatgatgaataa | |
| 131 | tggactgagtctctgtattgcggcgtgtataccaatgcatggccctcttctgatt | CD47-203 |
| | tcaggtttgagtatcttagctctagcacaattacttggactagtttatatgaaat | CDS |
| | ttgtggggttcagagggacacctacagtcagttgaaaggcaagaagagacaagct | nucleotide |
| | tccaatcagaagactatacaacctcctaggaaagctgtagaggaaccccttaatg | sequence |
| | cattcaaagaatcaaaaggaatgatgaatgatgaataa | |
| 132 | agatattttcttgttcaatttaaggagaggtaaatttggtatcaatagaaaaaat | CD47-204 |
| | gtttctgaaaaatttaaaccctggaaatgtatttatggcatggagtcagatgttt | CDS |
| | cagggagagaagaacaaatcaagaagcattgcaagtatgctcatatggaatgctt | nucleotide |
| | aaggcttgtggttaaaaaatatatatatatggctgtcaatgtcttaggctcatgg | sequence |
| | tagcagcagaaatcgtaataattcttttgtcacatgggttatatccatattggag | |
| | agaattaactcaggtgaaattaacttgtacactgtttggttttataatatttaga | |
| | gggatcacaactgactgatgtccctttgaagtaccattcttcataaatctttttt | |
| | tttcagaatgggccagccaactgtgacatcccttggatcggagatttagaactag | |
| | aaagtattctttctacattattagggaagaaaaggagttacttggcggttagcaa | |
| | tattctattttgttttgttttgtttttagagacagggtctcattatgttgaccag | |
| | gctggcctcgagctcctgggctcaagcaatgctcccacctcagcctoccaagtag | |
| | ctgggactacaggcatgtgccactacacctggcagtgtttattctgataaataca | |
| | tttatgagctcaaaaatgtaactctaaaaccttatctctgaacttccatattacc | |
| | atcagaaatttagatagttgtttagttctctttttctttgtagaacatagatata | |
| | aggcatggtttcattgaagtcagttgtatatacatgtaactatcctgatgttccc | |
| | aaataaagctctgtatttctgcttagtttattggggaggctgctaaatgtagtgc | |
| | atcccaacccattttaccctgttctactttaaaaagaggttggcttcttgtttgg | |
| | atacaaggaccaagtcactcccccaggttcctccacagtaagggaggcctattta | |
| | aagccgcccatggcactaacagaaactggactcctatgagctcagatacataact | |
| | gggcctcacaggggtgggacagtatgtagtctaggaattggaaggatccattcca | |
| | tatcaaagaactgaagcatcgtgttgccctctcagcagcaagagtaaggtgatgc | |
| | ccctgtcagttatagttcctgagttcctctgtctttgattctttgcctattagcc | |
| | agctagctcaccctcttgtttatgccactgttttttatcctattcatgccttctc | |
| | acagacaacttttcttacctacagctttggactcatccttgtctcctttctgttt | |
| | tttttcactttcccttcccatcaccaactttctgggtttttttctgtttcttctt | |
| | agagtccagtggcagggagaaacttgtcagtccagtctgttgccatttttcctgt | |
| | ttgagaaagactcaccagcttttggctggctcacagattggctttccttgggtca | |
| | ggacccacccttttccctgccagctttggaagcttgacagaattcgagtgtgcag | |
| | tggtggtaaataaatagtaaggaacacagagcagtcctggaggcgtgcctccatc | |
| | tgctgatgagaaaatccagtgctgtcatccagcccaggtcccagcggaatgggcc | |
| | tctctgttcagtaggatccccctcctgctgagtggttcatggcatgtttctgttc | |
| | aacgcttttccatctgtaggattcttattctgtatttatttgtttttttgggttt | |
| | ttttattttttgagatggagtctcgctctgtcgcccaggctggagtgcagtggca | |
| | cgaccccagctcgctgcagcctctgcctcccaggacgagggagatcctcccacct | |
| | cagccttccacgtagctgggactacaggcatgcaccacaggcatgcaccaccacg | |
| | ccagctaatttttgtatttttggtagagacagggttgcatcatgttgcccaggct | |
| | ggtcttgaatgcctgagctcaagcaatctatttgccttggcctcccaaagtgctg | |
| | ggattacaggcatgagccaccacggccagccttctcatttgttttttttataagg | |
| | aagctatctcttcttccctccccaactagggtattctttttccctttcgtcactt | |
| | tgctcatgtactgtattccttcaacttcattaatgaatccatttggaagcagtga | |
| | aaaaggcaactcagaaagctaagaagaaatagatagaggaatactcagagctatc | |
| | tgagtattttctttagtttgttagctctttggagctttgaaactggaaagaccca | |
| | gggagtgatgtggagaaagagactgagcttgtaagacacaggagcagtgagctaa | |
| | gggagatggagtagtggggacaaattctggcacattctgtctacactctgggtag | |
| | atagaggagggaggatggagcacccatggtgggggtatgttggtgacagcatttt | |
| | cccaccagccagtgtaacaagtggctgatttgggggaaagatggcataaacaaat | |
| | gagagaatgtgtttactatttgatgtagatgggttatttgcttcatttttcaaat | |
| | cagtgtatataatcaagaatattcagcatgtttgaatagactgtcagagctggaa | |
| | ctctttcattaacatctctggcacctttagttttagccctgaacattttatotta | |
| | aaattaaacattaccaaatgccttagtttatttcatttattaaatttatattctt | |
| | atttgttatttatatcagcttccaatcagaagactatacaacctcctaggaataa | |
| | ctgaagtgaagtgatggactccgatttggagagtagtaagacgtgaaaggaatac | |
| | acttgtgtttaagcaccatggccttgatgattcactgttggggagaagaaacaag | |
| | aaaagtaactggttgtcacctatgagacccttacgtgattgttagttaagttttt | |
| | attcaaagcagctgtaatttagttaataaaataattatgatctatgttgtttgcc | |
| | caattgagatccagttttttgttgttatttttaatcaattaggggcaatagtaga | |
| | atggacaatttccaagaatgatgcctttcaggtcctagggcctctggcctctagg | |
| | taaccagtttaaattggttcagggtgataactacttagcactgccctggtgatta | |
| | cccagagatatctatgaaaaccagtggcttccatcaaacctttgccaactcaggt | |
| | tcacagcagctttgggcagttatggcagtatggcattagctgagaggtgtctgcc | |
| | acttctgggtcaatggaataataaattaagtacaggcaggaatttggttgggagc | |
| | atcttgtatgatctccgtatgatgtgatattgatggagatagtggtcctcattct | |
| | tgggggttgccattcccacattcccccttcaacaaacagtgtaacaggtccttcc | |
| | cagatttagggtacttttattgatggatatgttttccttttattcacataacccc | |
| | ttgaaaccctgtcttgtcctcctgttacttgcttctgctgtacaagatgtagcac | |
| | cttttctcctctttgaacatggtctagtgacacggtagcaccagttgcaggaagg | |
| | agccagacttgttctcagagcactgtgttcacacttttcagcaaaaatagctatg | |
| | gttgtaacatatgtattcccttcctctgatttgaaggcaaaaatctacagtgttt | |
| | cttcacttcttttctgatctggggcatgaaaaaagcaagattgaaatttgaacta | |
| | tgagtctcctgcatggcaacaaaatgtgtgtcaccatcaggccaacaggccagcc | |
| | cttgaatggggatttattactgttgtatctatgttgcatgataaacattcatcac | |
| | cttcctcctgtagtcctgcctcgtactccccttcccctatgattgaaaagtaaac | |
| | aaaacccacatttcctatcctggttagaagaaaattaatgttctgacagttgtga | |
| | tcgcctggagtacttttagacttttagcattcgttttttacctgtttgtggatgt | |
| | gtgtttgtatgtgcatacgtatgagataggcacatgcatcttctgtatggacaaa | |
| | ggtggggtacctacaggagagcaaaggttaattttgtgcttttagtaaaaacatt | |
| | taaatacaaagttctttattgggtggaattatatttgatgcaaatatttgatcac | |
| | ttaaaacttttaaaacttctaggtaatttgccacgctttttgactgctcaccaat | |
| | accctgtaaaaatacgtaattcttcctgtttgtgtaataagatattcatatttgt | |
| | agttgcattaataatagttatttcttagtccatcagatgttcccgtgtgcctctt | |
| | ttatgccaaattgattgtcatatttcatgttgggaccaagtagtttgcccatggc | |
| | aaacctaaatttatgacctgctgaggcctctcagaaaactgagcatactagcaag | |
| | acagctcttcttgaaaaaaaaaatatgtatacacaaatatatacgtatatctata | |
| | tatacgtatgtatatacacacatgtatattcttccttgattgtgtagctgtccaa | |
| | aataataacatatatagagggagctgtattcctttatacaaatctgatggctcct | |
| | gcagcactttttccttctgaaaatatttacattttgctaacctagtttgttactt | |
| | taaaaatcagttttgatgaaaggagggaaaagcagatggacttgaaaaagatcca | |
| | agctcctattagaaaaggtatgaaaatctttatagtaaaattttttataaactaa | |
| | agttgtaccttttaatatgtagtaaactctcatttatttggggttcgctcttgga | |
| | tctcatccatccattgtgttctctttaatgctgcctgccttttgaggcattcact | |
| | gccctagacaatgccaccagagatagtgggggaaatgccagatgaaaccaactct | |
| | tgctctcactagttgtcagottctctggataagtgaccacagaagcaggagtcct | |
| | cctgcttgggcatcattgggccagttccttctctttaaatcagatttgtaatggc | |
| | tcccaaattccatcacatcacatttaaattgcagacagtgttttgcacatcatgt | |
| | atctgttttgtcccataatatgctttttactccctgatcccagtttctgctgttg | |
| | actcttccattcagttttatttattgtgtgttctcacagtgacaccatttgtcct | |
| | tttctgcaacaacctttccagctacttttgccaaattctatttgtcttctccttc | |
| | aaaacattctcctttgcagttcctcttcatctgtgtagctgctcttttgtctctt | |
| | aacttaccattcctatagtactttatgcatctctgcttagttctattagtttttt | |
| | ggccttgctcttctccttgattttaaaattccttctatagctagagcttttcttt | |
| | ctttcattctctcttcctgcagtgttttgcatacatcagaagctaggtacataag | |
| | ttaaatgattgagagttggctgtatttagatttatcactttttaatagggtgagc | |
| | ttgagagttttctttctttctgttttttttttttgtttttttttttttttttttt | |
| | ttttttttttttgactaatttcacatgctctaaaaaccttcaaaggtgattattt | |
| | ttctcctggaaactccaggtccattctgtttaaatccctaagaatgtcagaatta | |
| | aaataacagggctatcccgtaattggaaatatttcttttttcaggatgctatagt | |
| | caatttagtaagtgaccaccaaattgttatttgcactaacaaagctcaaaacacg | |
| | ataagtttactcctccatctcagtaataaaaattaagctgtaatcaaccttctag | |
| | gtttctcttgtcttaaaatgggtattcaaaaatggggatctgtggtgtatgtatg | |
| | gaaacacatactccttaatttacctgttgttggaaactggagaaatgattgtcgg | |
| | gcaaccgtttattttttattgtattttatttggttgagggatttttttataaaca | |
| | gttttacttgtgtcatattttaaaattactaactgccatcacctgctggggtcct | |
| | ttgttaggtcattttcagtgactaatagggataatccaggtaactttgaagagat | |
| | gagcagtgagtgaccaggcagtttttctgcctttagctttgacagttcttaatta | |
| | agatcattgaagaccagctttctcataaatttctctttttgaaaaaaagaaagca | |
| | tttgtactaagctcctctgtaagacaacatcttaaatcttaaaagtgttgttatc | |
| | atgactggtgagagaagaaaacattttgtttttattaaatggagcattatttaca | |
| | aaaagccattgttgagaattagatcccacatcgtataaatatctattaaccattc | |
| | taaataaagagaactccagtgttgctatgtgcaagatcctctcttggagcttttt | |
| | tgcatagcaattaaaggtgtgctatttgtcagtagccatttttttgcagtgattt | |
| | gaagaccaaagttgttttacagctgtgttaccgttaaaggtttttttttttatat | |
| | gtattaaatcaatttatcactgtttaaagctttgaatatctgcaatctttgccaa | |
| | ggtacttttttatttaaaaaaaaacataactttgtaaatattaccctgtaatatt | |
| | atatatacttaataaaacattttaagctat | |
| 133 | aagaatgctactggccttggtttaattgtgacttctacagggatattaatattac | CD47-205 |
| | ttcactactatgtgtttagtacagcgattggattaacctccttcgtcattgccat | CDS |
| | attggttattcaggtgatagcctatatcctcgctgtggttggactgagtctctgt | nucleotide |
| | attgcggcgtgtataccaatgcatggccctcttctgatttcaggtttgagtatct | sequence |
| | tagctctagcacaattacttggactagtttatatgaaatttgtggcttccaatca | |
| | gaagactatacaacctcctaggaaagctgtagaggaaccccttaatgaataa | |
| 134 | atggaggcacaaaacactactgaagtatacgtaaagtggaaatttaaaggaagag | CD47-206 |
| | atatttacacctttgatggagctctaaacaagtccactgtccccactgactttag | CDS |
| | tagtgcaaaaattgaagtctcacaattactaaaaggagatgcctctttgaagatg | nucleotide |
| | gataagagtgatgctgtctcacacacaggaaactacacttgtgaagtaacagaat | sequence |
| | taaccagagaaggtgaaacgatcatcgagctaaaatatcgtgttgtttcatggtt | |
| | ttctccaaatgaaaatattcttattgttattttcccaatttttgctatactcctg | |
| | ttctggggacagtttggtattaaaacacttaaatatagatccggtggtatggatg | |
| | agaaaacaattgctttacttgttgctggactagtgatcactgtcattgtcattgt | |
| | tggagccattcttttcgtcccaggtgaatattcattaaagaatgctactggcctt | |
| | ggtttaattgtgacttctacagggatattaatattacttcactactatgtgttt | |
| 135 | atgtggcccctggtcgccgccctgttgctgggctcggcatgctgcggatcagctc | Codon- |
| | agctactgtttaataaaacaaaatctgtagaattcacgttttgtaacgacactgt | optimized |
| | cgtgatcccatgctttgttactaatatggaggcacaaaacaccactgaagtgtac | nucleotide |
| | gtgaagtggaaattcaaaggcagagacatttacacctttgacggcgccctcaaca | sequence |
| | agtccaccgtgcccactgactttagtagcgcaaaaattgaggtcagccaattact | encoding |
| | aaaaggagatgcctctttgaagatggacaagagcgatgctgtcagccacacaggg | CD47 |
| | aactacacttgtgaagtaacagagttaacccgcgaaggtgaaacgatcatcgagc | |
| | tgaagtatcgagtggtgtcctggttttctccgaacgagaatatccttatcgtaat | |
| | tttcccaattttcgctatcctcctgttctggggccagtttggtatcaagacactc | |
| | aaatatcggtccggtgggatggatgagaagacaattgccctgcttgttgctggac | |
| | tcgtgatcaccgtcatcgtgattgttggggccatccttttcgtcccaggggagta | |
| | cagcctgaagaatgctacgggcctgggattaattgtgacctctacagggatactc | |
| | atcctgcttcactactatgtgttcagtaccgcgattggactgacctccttcgtca | |
| | ttgccatattggtgattcaggtgatagcctacatcctcgccgtggttggcctgag | |
| | tctctgtatcgcggcgtgcatacccatgcatggccctcttctgatttcagggttg | |
| | agtatcctcgcactagcacagttgctgggactggtttatatgaaatttgtggcct | |
| | ccaaccagaagactatacagcctcctaggaaggctgtagaggagcccctgaatgc | |
| | attcaaggaatcaaaaggcatgatgaatgatgaa | |

### CAR

In certain embodiments, the polycistronic vector may comprise one or more expression cassettes each comprising a nucleotide sequence encoding a CAR. CARs (also known as chimeric immunoreceptors, chimeric T cell receptors, or artificial T cell receptors) are receptor proteins that have been engineered to give host cells (e.g., T cells) the new ability to target a specific protein. The receptors are chimeric because they combine both antigen-binding and T cell activating functions into a single receptor. The polycistronic vector of the present technology may be used to express one or more CARs in a host cell (e.g., a T cell) for use in cell-based therapies against various target antigens. The CARs expressed by the one or more expression cassettes may be the same or different. In any of these embodiments, the CAR may comprise an extracellular binding domain (also referred to as a binder) that specifically binds a target antigen, a transmembrane domain, and an intracellular signaling domain. In certain embodiments, the CAR may further comprise one or more additional elements, including one or more signal peptides, one or more extracellular hinge domains, and/or one or more intracellular costimulatory domains. Domains may be directly adjacent to one another, or there may be one or more amino acids linking the domains. The nucleotide sequence encoding a CAR may be derived from a mammalian sequence, for example, a mouse sequence, a primate sequence, a human sequence, or combinations thereof. In the cases where the nucleotide sequence encoding a CAR is non-human, the sequence of the CAR may be humanized. The nucleotide sequence encoding a CAR may also be codon-optimized for expression in a mammalian cell, for example, a human cell. In any of these embodiments, the nucleotide sequence encoding a CAR may be at least 80% identical (e.g., at least 80%, at least 85%, at least 90%, at least 95%, at least 96%, at least 97%, at least 98%, at least 99%, or 100% identical) to any of the nucleotide sequences disclosed herein. The sequence variations may be due to codon-optimalization, humanization, restriction enzyme-based cloning scars, and/or additional amino acid residues linking the functional domains, etc.

In certain embodiments, the CAR may comprise a signal peptide at the N-terminus. Non-limiting examples of signal peptides include CD8a signal peptide, IgK signal peptide, and granulocyte-macrophage colony-stimulating factor receptor subunit alpha (GMCSFR-α, also known as colony stimulating factor 2 receptor subunit alpha (CSF2RA)) signal peptide, and variants thereof, the amino acid sequences of which are provided in Table 2 below.

TABLE 2
Exemplary sequences of signal peptides

| SEQ ID | | |
|---|---|---|
| NO: | Sequence | Description |
| 6 | MALPVTALLLPLALLLHAARP | CD8α signal peptide |
| 7 | METDTLLLWVLLLWVPGSTG | IgK signal peptide |
| 8 | MLLLVTSLLLCELPHPAFLLI | GMCSFR-α (CSF2RA) |
| | P | signal peptide |

In certain embodiments, the CAR may comprise an extracellular binding domain, also referred to as a binder. In certain embodiments, the extracellular binding domain may comprise one or more antibodies specific to one target antigen or multiple target antigens. The antibody may be an antibody fragment, for example, an scFv, or a single-domain antibody fragment, for example, a VHH. In certain embodiments, the scFv may comprise a heavy chain variable region (V_{H}) and a light chain variable region (V_{L}) of an antibody connected by a linker. The V_{H} and the V_{L} may be connected in either order, i.e., V_{H}-linker-V_{L} or V_{L}-linker-V_{H}. Non-limiting examples of linkers include Whitlow linker, (G₄S)ₙ (n can be a positive integer, e.g., 1, 2, 3, 4, 5, 6, etc.) linker, and variants thereof. In certain embodiments, the antigen may be an antigen that is exclusively or preferentially expressed on tumor cells, or an antigen that is characteristic of an autoimmune or inflammatory disease. Exemplary target antigens include, but are not limited to, B cell maturation agent (BCMA), CA9, CD5, CD19, CD20, CD22, CD23, CD30, CD33, CD38, CD44, CD70, CD133, CD174, CD274, CD276, CEACAM5, CSPG4, EGFR, EGFRvIII, EPCAM, EPHA2, ERBB2, FAP, FOLH1, FOLR1, GD2, GPC3, GPNMB, IL1RAP, IL3BA, IL13RA2, Kappa, KDR, L1CAM, Lambda, MET, MS4A1, MSLN, MUC1, NCAM1, PDCD1, PSCA, ROR1, SDC1, SLAMF7, TEM1, TNFRSF8, TNFRSF17, ULBP1 ULBP2, G-protein coupled receptor family C group 5 member D (GPRC5D) (associated with leukemias); CS1/SLAMF7, CD38, CD138, GPRC5D, TACI, and BCMA (associated with myelomas); GD2, HER2, EGFR, EGFRvIII, B7H3, PSMA, PSCA, CAIX, CD171, CEA, CSPG4, EPHA2, FAP, FRα, IL-13Ra, Mesothelin, MUC1, MUC16, and ROR1 (associated with solid tumors). In any of these embodiments, the extracellular binding domain of the CAR can be codon-optimized for expression in a host cell or have variant sequences to increase functions of the extracellular binding domain.

In certain embodiments, the CAR may comprise a hinge domain, also referred to as a spacer. The terms “hinge” and “spacer” may be used interchangeably in the present disclosure. Non-limiting examples of hinge domains include CD8a hinge domain, CD28 hinge domain, IgG4 hinge domain, IgG4 hinge-CH2-CH3 domain, and variants thereof, the amino acid sequences of which are provided in Table 3 below.

TABLE 3
Exemplary sequences of hinge domains

| SEQ ID | | |
|---|---|---|
| NO: | Sequence | Description |
| 9 | TTTPAPRPPTPAPTIASQPLSLRPEACRP | CD8α hinge |
| | AAGGAVHTRGLDFACD | domain |
| 10 | IEVMYPPPYLDNEKSNGTIIHVKGKHLCP | CD28 hinge |
| | SPLFPGPSKP | domain |
| 113 | AAAIEVMYPPPYLDNEKSNGTIIHVKGKH | CD28 hinge |
| | LCPSPLFPGPSKP | domain |
| 11 | ESKYGPPCPPCP | IgG4 hinge |
| | | domain |
| 12 | ESKYGPPCPSCP | IgG4 hinge |
| | | domain |
| 13 | ESKYGPPCPPCPAPEFLGGPSVFLFPPKP | IgG4 hinge- |
| | KDTLMISRTPEVTCVVVDVSQEDPEVQFN | CH2-CH3 |
| | WYVDGVEVHNAKTKPREEQFNSTYRVVSV | domain |
| | LTVLHQDWLNGKEYKCKVSNKGLPSSIEK | |
| | TISKAKGQPREPQVYTLPPSQEEMTKNQV | |
| | SLTCLVKGFYPSDIAVEWESNGQPENNYK | |
| | TTPPVLDSDGSFFLYSRLTVDKSRWQEGN | |
| | VFSCSVMHEALHNHYTQKSLSLSLGK | |

In certain embodiments, the CAR may comprise a transmembrane domain. In certain embodiments, the transmembrane domain may comprise a transmembrane region of the alpha, beta, or zeta chain of a T cell receptor, CD28, CMEs, CD45, CD4, CD5, CD8, CD9, CD16, CD22, CD33, CD37, CD64, CD80, CD86, CD134, CD137, CD154, or a functional variant thereof, including the human versions of each of these sequences. In other embodiments, the transmembrane domain may comprise a transmembrane region of CD8α, CD8β, 4-1 BB/CD137, CD28, CD34, CD4, FcεRIγ, CD16, OX40/CD134, CD3ζ, CMEε, CD3γ, CD3δ, TCRα, TCRβ, TCRζ, CD32, CD64, CD64, CD45, CD5, CD9, CD22, CD37, CD80, CD86, CD40, CD40L/CD154, VEGFR2, FAS, and FGFR2B3, or a functional variant thereof, including the human versions of each of these sequences. Table 4 provides the amino acid sequences of a few exemplary transmembrane domains.

TABLE 4
Exemplary sequences of transmembrane domains

| SEQ ID | | |
|---|---|---|
| NO: | Sequence | Description |
| 14 | IYIWAPLAGTCGVLLLSLVITLYC | CD8α |
| | | transmembrane |
| | | domain |
| 15 | FWVLVVVGGVLACYSLLVTVAFII | CD28 |
| | FWV | transmembrane |
| | | domain |
| 114 | MFWVLVVVGGVLACYSLLVTVAFI | CD28 |
| | IFWV | transmembrane |
| | | domain |

In certain embodiments, the CAR may comprise an intracellular costimulatory domain and/or an intracellular signaling domain. In certain embodiments, the intracellular costimulatory domain and/or intracellular signaling domain may comprise one or more signaling domains selected from B7-1/CD80, B7-2/CD86, B7-H1/PD-L1, B7-H2, B7-H3, B7-H4, B7-H6, B7-H7, BTLA/CD272, CD28, CTLA-4, Gi24/VISTA/B7-H5, ICOS/CD278, PD-1, PD-L2/B7-DC, PDCD6, 4-1 BB/TNFSF9/CD137, 4-1 BB Ligand/TNFSF9, BAFF/BLyS/TNFSF13B, BAFF R/TNFRSF13C, CD27/TNFRSF7, CD27 Ligand/TNFSF7, CD30/TNFRSF8, CD30 Ligand/TNFSF8, CD40/TNFRSF5, CD40/TNFSF5, CD40 Ligand/TNFSF5, DR3/TNFRSF25, GITR/TNFRSF18, GITR Ligand/TNFSF18, HVEM/TNFRSF14, LIGHT/TNFSF14, Lymphotoxin-alpha/TNFp, OX40/TNFRSF4, OX40 Ligand/TNFSF4, RELT/TNFRSF19L, TACI/TNFRSF13B, TL1A/TNFSF15, TNFa, TNF RII/TNFRSF1B, 2B4/CD244/SLAMF4, BLAME/SLAMF8, CD2, CD2F-10/SLAMF9, CD48/SLAMF2, CD58/LFA-3, CD84/SLAMF5, CD229/SLAMF3, CRACC/SLAMF7, NTB-A/SLAMF6, SLAM/CD150, CD2, CD7, CD53, CD82/Kai-1, CD90/Thy1, CD96, CD160, CD200, CD300a/LMIR1, HLA Class I, HLA-DR, Ikaros, Integrin alpha 4/CD49d, Integrin alpha 4 beta 1, Integrin alpha 4 beta 7/LPAM-1, LAG-3, TCL1A, TCL1B, CRTAM, DAP12, Dectin-1/CLEC7A, DPPIV/CD26, EphB6, TIM-1/KIM-1/HAVCR, TIM-4, TSLP, TSLP R, lymphocyte function associated antigen-1 (LFA-1), NKG2C, CD3ζ, an immunoreceptor tyrosine-based activation motif (ITAM), CD27, CD28, 4-1BB, CD134/OX40, CD30, CD40, PD-1, ICOS, lymphocyte function-associated antigen-1 (LFA-1), CD2, CD7, LIGHT, NKG2C, B7-H3, a ligand that specifically binds with CD83, and a functional variant thereof including the human versions of each of these sequences. In some embodiments, the intracellular costimulatory domain and/or intracellular signaling domain comprises one or more signaling domains selected from a CD3ζ domain, an ITAM, a CD28 domain, 4-1 BB domain, or a functional variant thereof. Table 5 provides the amino acid sequences of a few exemplary intracellular costimulatory and/or signaling domains. In certain embodiments, as in the case of tisagenlecleucel as described below, the CD3ζ signaling domain of SEQ ID NO:18 may have a mutation, e.g., a glutamine (Q) to lysine (K) mutation, at amino acid position 14 (see SEQ ID NO:115).

TABLE 5
Exemplary sequences of intracellular
costimulatory and/or signaling domains

| SEQ ID | | |
|---|---|---|
| NO: | Sequence | Description |
| 16 | KRGRKKLLYIFKQPFMRPVQTTQEED | 4-1BB |
| | GCSCRFPEEEEGGCEL | costimulatory |
| | | domain |
| 17 | RSKRSRLLHSDYMNMTPRRPGPTRKH | CD28 |
| | YQPYAPPRDFAAYRS | costimulatory |
| | | domain |
| 18 | RVKFSRSADAPAYQQGQNQLYNELNL | CD3ζ |
| | GRREEYDVLDKRRGRDPEMGGKPRRK | signaling |
| | NPQEGLYNELQKDKMAEAYSEIGMKG | domain |
| | ERRRGKGHDGLYQGLSTATKDTYDAL | |
| | HMQALPPR | |
| 115 | RVKFSRSADAPAYKQGQNQLYNELNL | CD3ζ signaling |
| | GRREEYDVLDKRRGRDPEMGGKPRRK | domain (with |
| | NPQEGLYNELQKDKMAEAYSEIGMKG | Q to K |
| | ERRRGKGHDGLYQGLSTATKDTYDAL | mutation at |
| | HMQALPPR | position 14) |

In certain embodiments where the polycistronic vector encodes two or more CARs, the two or more CARs may comprise the same functional domains, or one or more different functional domains, as described. For example, the two or more CARs may comprise different signal peptides, extracellular binding domains, hinge domains, transmembrane domains, costimulatory domains, and/or intracellular signaling domains, in order to minimize the risk of recombination due to sequence similarities. Or, alternatively, the two or more CARs may comprise the same functional domains. In the cases where the same domain(s) and/or backbone are used, it is optional to introduce codon divergence at the nucleotide sequence level to minimize the risk of recombination.

### CD19 CAR

In some embodiments, the CAR is a CD19 CAR, and in these embodiments, the polycistronic vector comprises an expression cassette that contains a nucleotide sequence encoding a CD19 CAR. In some embodiments, the CD19 CAR may comprise a signal peptide, an extracellular binding domain that specifically binds CD19, a hinge domain, a transmembrane domain, an intracellular costimulatory domain, and/or an intracellular signaling domain in tandem.

In some embodiments, the signal peptide of the CD19 CAR comprises a CD8α signal peptide. In some embodiments, the CD8a signal peptide comprises or consists of an amino acid sequence set forth in SEQ ID NO:6 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:6. In some embodiments, the signal peptide comprises an IgK signal peptide. In some embodiments, the IgK signal peptide comprises or consists of an amino acid sequence set forth in SEQ ID NO:7 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:7. In some embodiments, the signal peptide comprises a GMCSFR-a or CSF2RA signal peptide. In some embodiments, the GMCSFR-a or CSF2RA signal peptide comprises or consists of an amino acid sequence set forth in SEQ ID NO:8 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:8.

In some embodiments, the extracellular binding domain of the CD19 CAR is specific to CD19, for example, human CD19. The extracellular binding domain of the CD19 CAR can be codon-optimized for expression in a host cell or to have variant sequences to increase functions of the extracellular binding domain. In some embodiments, the extracellular binding domain comprises an immunogenically active portion of an immunoglobulin molecule, for example, an scFv.

In some embodiments, the extracellular binding domain of the CD19 CAR comprises an scFv derived from the FMC63 monoclonal antibody (FMC63), which comprises the heavy chain variable region (V_{H}) and the light chain variable region (V_{L}) of FMC63 connected by a linker. FMC63 and the derived scFv have been described in Nicholson et al., Mol. Immun. 34(16-17):1157-1165 (1997) and PCT Application Publication No. WO2018213337, the entire contents of each of which are incorporated by reference herein. In some embodiments, the amino acid sequences of the entire FMC63-derived scFv (also referred to as FMC63 scFv) and its different portions are provided in Table 6 below. In some embodiments, the CD19-specific scFv comprises or consists of an amino acid sequence set forth in SEQ ID NO:19, 20, or 25, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:19, 20, or 25. In some embodiments, the CD19-specific scFv may comprise one or more CDRs having amino acid sequences set forth in SEQ ID NOs: 21-23 and 26-28. In some embodiments, the CD19-specific scFv may comprise a light chain with one or more CDRs having amino acid sequences set forth in SEQ ID NOs: 21-23. In some embodiments, the CD19-specific scFv may comprise a heavy chain with one or more CDRs having amino acid sequences set forth in SEQ ID NOs: 26-28. In any of these embodiments, the CD19-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the CD19 CAR comprises or consists of the one or more CDRs as described herein.

In some embodiments, the linker linking the V_{H} and the V_{L} portions of the scFv is a Whitlow linker having an amino acid sequence set forth in SEQ ID NO:24. In some embodiments, the Whitlow linker may be replaced by a different linker, for example, a (G₄S)ₙ (n can be a positive integer, e.g., 1, 2, 3, 4, 5, or 6) linker. In some embodiments, the linker is a 3×G₄S linker having an amino acid sequence set forth in SEQ ID NO:30, which gives rise to a different FMC63-derived scFv having an amino acid sequence set forth in SEQ ID NO:29. In certain of these embodiments, the CD19-specific scFv comprises or consists of an amino acid sequence set forth in SEQ ID NO:29 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:29.

TABLE 6
Exemplary sequences of anti-CD19
scFv and components

| SEQ | | |
|---|---|---|
| ID | | |
| NO: | Amino Acid Sequence | Description |
| 19 | DIQMTQTTSSLSASLGDRVTISCRA | Anti-CD19 FMC63 |
| | SQDISKYLNWYQQKPDGTVKLLIYH | scFv entire |
| | TSRLHSGVPSRFSGSGSGTDYSLTI | sequence, with |
| | SNLEQEDIATYFCQQGNTLPYTFGG | Whitlow linker |
| | GTKLEITGSTSGSGKPGSGEGSTKG | |
| | EVKLQESGPGLVAPSQSLSVTCTVS | |
| | GVSLPDYGVSWIRQPPRKGLEWLGV | |
| | IWGSETTYYNSALKSRLTIIKDNSK | |
| | SQVFLKMNSLQTDDTAIYYCAKHYY | |
| | YGGSYAMDYWGQGTSVTVSS | |
| 20 | DIQMTQTTSSLSASLGDRVTISCRA | Anti-CD19 FMC63 |
| | SQDISKYLNWYQQKPDGTVKLLIYH | scFv light chain |
| | TSRLHSGVPSRFSGSGSGTDYSLTI | variable region |
| | SNLEQEDIATYFCQQGNTLPYTFGG | |
| | GTKLEIT | |
| 21 | QDISKY | Anti-CD19 FMC63 |
| | | scFv light chain |
| | | CDR1 |
| 22 | HTS | Anti-CD19 FMC63 |
| | | scFv light chain |
| | | CDR2 |
| 23 | QQGNTLPYT | Anti-CD19 FMC63 |
| | | scFv light chain |
| | | CDR3 |
| 24 | GSTSGSGKPGSGEGSTKG | Whitlow linker |
| 25 | EVKLQESGPGLVAPSQSLSVTCTVS | Anti-CD19 FMC63 |
| | GVSLPDYGVSWIRQPPRKGLEWLGV | scFv heavy chain |
| | IWGSETTYYNSALKSRLTIIKDNSK | variable region |
| | SQVFLKMNSLQTDDTAIYYCAKHYY | |
| | YGGSYAMDYWGQGTSVTVSS | |
| 26 | GVSLPDYG | Anti-CD19 FMC63 |
| | | scFv heavy chain |
| | | CDR1 |
| 27 | IWGSETT | Anti-CD19 FMC63 |
| | | scFv heavy chain |
| | | CDR2 |
| 28 | AKHYYYGGSYAMDY | Anti-CD19 FMC63 |
| | | scFv heavy chain |
| | | CDR3 |
| 29 | DIQMTQTTSSLSASLGDRVTISCRA | Anti-CD19 FMC63 |
| | SQDISKYLNWYQQKPDGTVKLLIYH | scFv entire |
| | TSRLHSGVPSRFSGSGSGTDYSLTI | sequence, with |
| | SNLEQEDIATYFCQQGNTLPYTFGG | 3xG4S linker |
| | GTKLEITGGGGSGGGGSGGGGSEVK | |
| | LQESGPGLVAPSQSLSVTCTVSGVS | |
| | LPDYGVSWIRQPPRKGLEWLGVIWG | |
| | SETTYYNSALKSRLTIIKDNSKSQV | |
| | FLKMNSLQTDDTAIYYCAKHYYYGG | |
| | SYAMDYWGQGTSVTVSS | |
| 30 | GGGGSGGGGSGGGGS | 3xG4S linker |

In some embodiments, the extracellular binding domain of the CD19 CAR is derived from an antibody specific to CD19, including, for example, SJ25C1 (Bejcek et al., Cancer Res. 55:2346-2351 (1995)), HD37 (Pezutto et al., J. Immunol. 138(9):2793-2799 (1987)), 4G₇ (Meeker et al., Hybridoma 3:305-320 (1984)), B43 (Bejcek (1995)), BLY3 (Bejcek (1995)), B4 (Freedman et al., 70:418-427 (1987)), B4 HB12b (Kansas & Tedder, J. Immunol. 147:4094-4102 (1991); Yazawa et al., Proc. Natl. Acad. Sci. USA 102:15178-15183 (2005); Herbst et al., J. Pharmacol. Exp. Ther. 335:213-222 (2010)), BU12 (Callard et al., J. Immunology, 148(10): 2983-2987 (1992)), and CLB-CD19 (De Rie Cell. Immunol. 118:368-381(1989)). In any of these embodiments, the extracellular binding domain of the CD19 CAR can comprise or consist of the V_{H}, the V_{L}, and/or one or more CDRs of any of the antibodies.

In some embodiments, the hinge domain of the CD19 CAR comprises a CD8α hinge domain, for example, a human CD8a hinge domain. In some embodiments, the CD8α hinge domain comprises or consists of an amino acid sequence set forth in SEQ ID NO:9 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:9. In some embodiments, the hinge domain comprises a CD28 hinge domain, for example, a human CD28 hinge domain. In some embodiments, the CD28 hinge domain comprises or consists of an amino acid sequence set forth in SEQ ID NO:10 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:10. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO:11 or SEQ ID NO:12, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:11 or SEQ ID NO:12. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO:13 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:13.

In some embodiments, the transmembrane domain of the CD19 CAR comprises a CD8α transmembrane domain, for example, a human CD8α transmembrane domain. In some embodiments, the CD8α transmembrane domain comprises or consists of an amino acid sequence set forth in SEQ ID NO:14 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:14. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO:15 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:15.

In some embodiments, the intracellular costimulatory domain of the CD19 CAR comprises a 4-1 BB costimulatory domain. 4-1 BB, also known as CD137, transmits a potent costimulatory signal to T cells, promoting differentiation and enhancing long-term survival of T lymphocytes. In some embodiments, the 4-1 BB costimulatory domain is human. In some embodiments, the 4-1BB costimulatory domain comprises or consists of an amino acid sequence set forth in SEQ ID NO:16 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:16. In some embodiments, the intracellular costimulatory domain comprises a CD28 costimulatory domain. CD28 is another co-stimulatory molecule on T cells. In some embodiments, the CD28 costimulatory domain is human. In some embodiments, the CD28 costimulatory domain comprises or consists of an amino acid sequence set forth in SEQ ID NO:17 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:17. In some embodiments, the intracellular costimulatory domain of the CD19 CAR comprises a 4-1 BB costimulatory domain and a CD28 costimulatory domain as described.

In some embodiments, the intracellular signaling domain of the CD19 CAR comprises a CD3 zeta (ζ) signaling domain. CD3ζ associates with T cell receptors (TCRs) to produce a signal and contains immunoreceptor tyrosine-based activation motifs (ITAMs). The CD3ζ signaling domain refers to amino acid residues from the cytoplasmic domain of the zeta chain that are sufficient to functionally transmit an initial signal necessary for T cell activation. In some embodiments, the CD3ζ signaling domain is human. In some embodiments, the CD3ζ signaling domain comprises or consists of an amino acid sequence set forth in SEQ ID NO:18 or SEQ ID NO:115, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:18 or SEQ ID NO:115.

In some embodiments, the polycistronic vector comprises an expression cassette that contains a nucleotide sequence encoding a CD19 CAR, including, for example, a CD19 CAR comprising the CD19-specific scFv having sequences set forth in SEQ ID NO:19 or SEQ ID NO:29, the CD8α hinge domain of SEQ ID NO:9, the CD8α transmembrane domain of SEQ ID NO:14, the 4-1BB costimulatory domain of SEQ ID NO:16, the CD3ζ signaling domain of SEQ ID NO:18 or SEQ ID NO:115, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the CD19 CAR may additionally comprise a signal peptide (e.g., a CD8α signal peptide) as described.

In some embodiments, the polycistronic vector comprises an expression cassette that contains a nucleotide sequence encoding a CD19 CAR, including, for example, a CD19 CAR comprising the CD19-specific scFv having sequences set forth in SEQ ID NO:19 or SEQ ID NO:29, the IgG4 hinge domain of SEQ ID NO:11 or SEQ ID NO:12, the CD28 transmembrane domain of SEQ ID NO:15, the 4-1 BB costimulatory domain of SEQ ID NO:16, the CD3ζ signaling domain of SEQ ID NO:18 or SEQ ID NO:115, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the CD19 CAR may additionally comprise a signal peptide (e.g., a CD8α signal peptide) as described.

In some embodiments, the polycistronic vector comprises an expression cassette that contains a nucleotide sequence encoding a CD19 CAR, including, for example, a CD19 CAR comprising the CD19-specific scFv having sequences set forth in SEQ ID NO:19 or SEQ ID NO:29, the CD28 hinge domain of SEQ ID NO:10, the CD28 transmembrane domain of SEQ ID NO:15, the CD28 costimulatory domain of SEQ ID NO:17, the CD3ζ signaling domain of SEQ ID NO:18 or SEQ ID NO:115, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the CD19 CAR may additionally comprise a signal peptide (e.g., a CD8α signal peptide) as described.

In some embodiments, the polycistronic vector comprises an expression cassette that contains a nucleotide sequence encoding a CD19 CAR as set forth in SEQ ID NO:116 or is at least 80% identical (e.g., at least 80%, at least 85%, at least 90%, at least 95%, at least 96%, at least 97%, at least 98%, at least 99%, or 100% identical) to the nucleotide sequence set forth in SEQ ID NO:116 (see Table 7). The encoded CD19 CAR has a corresponding amino acid sequence set forth in SEQ ID NO:117 or is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:117, with the following components: CD8α signal peptide, FMC63 scFv (V_{L}-Whitlow linker-V_{H}), CD8α hinge domain, CD8α transmembrane domain, 4-1 BB costimulatory domain, and CD3ζ signaling domain.

In some embodiments, the polycistronic vector comprises an expression cassette that contains a nucleotide sequence encoding a commercially available embodiment of CD19 CAR. Non-limiting examples of commercially available embodiments of CD19 CARs expressed and/or encoded by T cells include tisagenlecleucel, lisocabtagene maraleucel, axicabtagene ciloleucel, and brexucabtagene autoleucel.

In some embodiments, the polycistronic vector comprises an expression cassette that contains a nucleotide sequence encoding tisagenlecleucel or portions thereof. Tisagenlecleucel comprises a CD19 CAR with the following components: CD8α signal peptide, FMC63 scFv (V_{L}-3×G₄S linker-V_{H}), CD8α hinge domain, CD8α transmembrane domain, 4-1 BB costimulatory domain, and CD3ζ signaling domain. The nucleotide and amino acid sequence of the CD19 CAR in tisagenlecleucel are provided in Table 7, with annotations of the sequences provided in Table 8.

In some embodiments, the polycistronic vector comprises an expression cassette that contains a nucleotide sequence encoding lisocabtagene maraleucel or portions thereof. Lisocabtagene maraleucel comprises a CD19 CAR with the following components: GMCSFR-α or CSF2RA signal peptide, FMC63 scFv (V_{L}-Whitlow linker-V_{H}), IgG4 hinge domain, CD28 transmembrane domain, 4-1 BB costimulatory domain, and CD3ζ signaling domain. The nucleotide and amino acid sequence of the CD19 CAR in lisocabtagene maraleucel are provided in Table 7, with annotations of the sequences provided in Table 9.

In some embodiments, the polycistronic vector comprises an expression cassette that contains a nucleotide sequence encoding axicabtagene ciloleucel or portions thereof. Axicabtagene ciloleucel comprises a CD19 CAR with the following components: GMCSFR-α or CSF2RA signal peptide, FMC63 scFv (V_{L}-Whitlow linker-V_{H}), CD28 hinge domain, CD28 transmembrane domain, CD28 costimulatory domain, and CD3ζ signaling domain. The nucleotide and amino acid sequence of the CD19 CAR in axicabtagene ciloleucel are provided in Table 7, with annotations of the sequences provided in Table 10.

In some embodiments, the polycistronic vector comprises an expression cassette that contains a nucleotide sequence encoding brexucabtagene autoleucel or portions thereof. Brexucabtagene autoleucel comprises a CD19 CAR with the following components: GMCSFR-α signal peptide, FMC63 scFv, CD28 hinge domain, CD28 transmembrane domain, CD28 costimulatory domain, and CD3ζ signaling domain.

In some embodiments, the polycistronic vector comprises an expression cassette that contains a nucleotide sequence encoding a CD19 CAR as set forth in SEQ ID NO: 31, 33, or 35, or is at least 80% identical (e.g., at least 80%, at least 85%, at least 90%, at least 95%, at least 96%, at least 97%, at least 98%, at least 99%, or 100% identical) to the nucleotide sequence set forth in SEQ ID NO: 31, 33, or 35. The encoded CD19 CAR has a corresponding amino acid sequence set forth in SEQ ID NO: 32, 34, or 36, respectively, or is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 32, 34, or 36, respectively.

TABLE 7
Exemplary sequences of CD19 CARs

| SEQ ID NO: | Sequence | Description |
|---|---|---|
| 116 | atggccttaccagtgaccgccttgctcctgccgctggccttg | Exemplary CD19 |
| | ctgctccacgccgccaggccggacatccagatgacacag | CAR nucleotide |
| | actacatcctccctgtctgcctctctgggagacagagtcacc | sequence |
| | atcagttgcagggcaagtcaggacattagtaaatatttaaa | |
| | ttggtatcagcagaaaccagatggaactgttaaactcctga | |
| | tctaccatacatcaagattacactcaggagtcccatcaagg | |
| | ttcagtggcagtgggtctggaacagattattctctcaccatta | |
| | gcaacctggagcaagaagatattgccacttacttttgccaa | |
| | cagggtaatacgcttccgtacacgttcggaggggggacc | |
| | aagctggagatcacaggctccacctctggatccggcaag | |
| | cccggatctggcgagggatccaccaagggcgaggtgaa | |
| | actgcaggagtcaggacctggcctggtggcgccctcaca | |
| | gagcctgtccgtcacatgcactgtctcaggggtctcattacc | |
| | cgactatggtgtaagctggattcgccagcctccacgaaag | |
| | ggtctggagtggctgggagtaatatggggtagtgaaacca | |
| | catactataattcagctctcaaatccagactgaccatcatca | |
| | aggacaactccaagagccaagttttcttaaaaatgaacag | |
| | tctgcaaactgatgacacagccatttactactgtgccaaac | |
| | attattactacggtggtagctatgctatggactactggggcc | |
| | aaggaacctcagtcaccgtctcctcaaccacgacgccag | |
| | cgccgcgaccaccaacaccggcgcccaccatcgcgtcg | |
| | cagcccctgtccctgcgcccagaggcgtgccggccagcg | |
| | gcggggggcgcagtgcacacgagggggctggacttcgc | |
| | ctgtgatatctacatctgggcgcccttggccgggacttgtgg | |
| | ggtccttctcctgtcactggttatcaccctttactgcaaacgg | |
| | ggcagaaagaaactcctgtatatattcaaacaaccatttat | |
| | gagaccagtacaaactactcaagaggaagatggctgtag | |
| | ctgccgatttccagaagaagaagaaggaggatgtgaact | |
| | gagagtgaagttcagcaggagcgcagacgcccccgcgt | |
| | accagcagggccagaaccagctctataacgagctcaatc | |
| | taggacgaagagaggagtacgatgttttggacaagagac | |
| | gtggccgggaccctgagatggggggaaagccgagaag | |
| | gaagaaccctcaggaaggcctgtacaatgaactgcaga | |
| | aagataagatggcggaggcctacagtgagattgggatga | |
| | aaggcgagcgccggaggggcaaggggcacgatggcct | |
| | ttaccagggtctcagtacagccaccaaggacacctacga | |
| | cgcccttcacatgcaggccctgccccctcgc | |
| 117 | MALPVTALLLPLALLLHAARPDIQMTQTTS | Exemplary CD19 |
| | SLSASLGDRVTISCRASQDISKYLNWYQQ | CAR amino acid |
| | KPDGTVKLLIYHTSRLHSGVPSRFSGSGS | sequence |
| | GTDYSLTISNLEQEDIATYFCQQGNTLPYT | |
| | FGGGTKLEITGSTSGSGKPGSGEGSTKGE | |
| | VKLQESGPGLVAPSQSLSVTCTVSGVSLP | |
| | DYGVSWIRQPPRKGLEWLGVIWGSETTY | |
| | YNSALKSRLTIIKDNSKSQVFLKMNSLQTD | |
| | DTAIYYCAKHYYYGGSYAMDYWGQGTSV | |
| | TVSSTTTPAPRPPTPAPTIASQPLSLRPEA | |
| | CRPAAGGAVHTRGLDFACDIYIWAPLAGT | |
| | CGVLLLSLVITLYCKRGRKKLLYIFKQPFM | |
| | RPVQTTQEEDGCSCRFPEEEEGGCELRV | |
| | KFSRSADAPAYQQGQNQLYNELNLGRRE | |
| | EYDVLDKRRGRDPEMGGKPRRKNPQEGL | |
| | YNELQKDKMAEAYSEIGMKGERRRGKGH | |
| | DGLYQGLSTATKDTYDALHMQALPPR | |
| 31 | atggccttaccagtgaccgccttgctcctgccgctggccttg | Tisagenlecleucel |
| | ctgctccacgccgccaggccggacatccagatgacacag | CD19 CAR |
| | actacatcctccctgtctgcctctctgggagacagagtcacc | nucleotide |
| | atcagttgcagggcaagtcaggacattagtaaatatttaaa | sequence |
| | ttggtatcagcagaaaccagatggaactgttaaactcctga | |
| | tctaccatacatcaagattacactcaggagtcccatcaagg | |
| | ttcagtggcagtgggtctggaacagattattctctcaccatta | |
| | gcaacctggagcaagaagatattgccacttacttttgccaa | |
| | cagggtaatacgcttccgtacacgttcggaggggggacc | |
| | aagctggagatcacaggtggcggtggctcgggcggtggt | |
| | gggtcgggtggcggcggatctgaggtgaaactgcaggag | |
| | tcaggacctggcctggtggcgccctcacagagcctgtccg | |
| | tcacatgcactgtctcaggggtctcattacccgactatggtgt | |
| | aagctggattcgccagcctccacgaaagggtctggagtg | |
| | gctgggagtaatatggggtagtgaaaccacatactataatt | |
| | cagctctcaaatccagactgaccatcatcaaggacaactc | |
| | caagagccaagttttcttaaaaatgaacagtctgcaaactg | |
| | atgacacagccatttactactgtgccaaacattattactacg | |
| | gtggtagctatgctatggactactggggccaaggaacctc | |
| | agtcaccgtctcctcaaccacgacgccagcgccgcgacc | |
| | accaacaccggcgcccaccatcgcgtcgcagcccctgtc | |
| | cctgcgcccagaggcgtgccggccagcggcggggggc | |
| | gcagtgcacacgagggggctggacttcgcctgtgatatct | |
| | acatctgggcgcccttggccgggacttgtggggtccttctcc | |
| | tgtcactggttatcaccctttactgcaaacggggcagaaag | |
| | aaactcctgtatatattcaaacaaccatttatgagaccagta | |
| | caaactactcaagaggaagatggctgtagctgccgatttc | |
| | cagaagaagaagaaggaggatgtgaactgagagtgaa | |
| | gttcagcaggagcgcagacgcccccgcgtacaagcagg | |
| | gccagaaccagctctataacgagctcaatctaggacgaa | |
| | gagaggagtacgatgttttggacaagagacgtggccggg | |
| | accctgagatggggggaaagccgagaaggaagaaccc | |
| | tcaggaaggcctgtacaatgaactgcagaaagataagat | |
| | ggcggaggcctacagtgagattgggatgaaaggcgagc | |
| | gccggaggggcaaggggcacgatggcctttaccagggt | |
| | ctcagtacagccaccaaggacacctacgacgcccttcac | |
| | atgcaggccctgccccctcgc | |
| 32 | MALPVTALLLPLALLLHAARPDIQMTQTTS | Tisagenlecleucel |
| | SLSASLGDRVTISCRASQDISKYLNWYQQ | CD19 CAR |
| | KPDGTVKLLIYHTSRLHSGVPSRFSGSGS | amino acid |
| | GTDYSLTISNLEQEDIATYFCQQGNTLPYT | sequence |
| | FGGGTKLEITGGGGSGGGGSGGGGSEVK | |
| | LQESGPGLVAPSQSLSVTCTVSGVSLPDY | |
| | GVSWIRQPPRKGLEWLGVIWGSETTYYN | |
| | SALKSRLTIIKDNSKSQVFLKMNSLQTDDT | |
| | AIYYCAKHYYYGGSYAMDYWGQGTSVTV | |
| | SSTTTPAPRPPTPAPTIASQPLSLRPEACR | |
| | PAAGGAVHTRGLDFACDIYIWAPLAGTCG | |
| | VLLLSLVITLYCKRGRKKLLYIFKQPFMRPV | |
| | QTTQEEDGCSCRFPEEEEGGCELRVKFS | |
| | RSADAPAYKQGQNQLYNELNLGRREEYD | |
| | VLDKRRGRDPEMGGKPRRKNPQEGLYNE | |
| | LQKDKMAEAYSEIGMKGERRRGKGHDGL | |
| | YQGLSTATKDTYDALHMQALPPR | |
| 33 | atgctgctgctggtgaccagcctgctgctgtgcgagctgcc | Lisocabtagene |
| | ccaccccgcctttctgctgatccccgacatccagatgaccc | maraleucel |
| | agaccacctccagcctgagcgccagcctgggcgaccgg | CD19 CAR |
| | gtgaccatcagctgccgggccagccaggacatcagcaa | nucleotide |
| | gtacctgaactggtatcagcagaagcccgacggcaccgt | sequence |
| | caagctgctgatctaccacaccagccggctgcacagcgg | |
| | cgtgcccagccggtttagcggcagcggctccggcaccga | |
| | ctacagcctgaccatctccaacctggaacaggaagatatc | |
| | gccacctacttttgccagcagggcaacacactgccctaca | |
| | cctttggcggcggaacaaagctggaaatcaccggcagca | |
| | cctccggcagcggcaagcctggcagcggcgagggcag | |
| | caccaagggcgaggtgaagctgcaggaaagcggccct | |
| | ggcctggtggcccccagccagagcctgagcgtgacctgc | |
| | accgtgagcggcgtgagcctgcccgactacggcgtgagc | |
| | tggatccggcagccccccaggaagggcctggaatggctg | |
| | ggcgtgatctggggcagcgagaccacctactacaacagc | |
| | gccctgaagagccggctgaccatcatcaaggacaacag | |
| | caagagccaggtgttcctgaagatgaacagcctgcagac | |
| | cgacgacaccgccatctactactgcgccaagcactactac | |
| | tacggcggcagctacgccatggactactggggccagggc | |
| | accagcgtgaccgtgagcagcgaatctaagtacggaccg | |
| | ccctgccccccttgccctatgttctgggtgctggtggtggtcg | |
| | gaggcgtgctggcctgctacagcctgctggtcaccgtggc | |
| | cttcatcatcttttgggtgaaacggggcagaaagaaactcc | |
| | tgtatatattcaaacaaccatttatgagaccagtacaaacta | |
| | ctcaagaggaagatggctgtagctgccgatttccagaaga | |
| | agaagaaggaggatgtgaactgcgggtgaagttcagca | |
| | gaagcgccgacgcccctgcctaccagcagggccagaat | |
| | cagctgtacaacgagctgaacctgggcagaagggaaga | |
| | gtacgacgtcctggataagcggagaggccgggaccctg | |
| | agatgggcggcaagcctcggcggaagaacccccagga | |
| | aggcctgtataacgaactgcagaaagacaagatggccg | |
| | aggcctacagcgagatcggcatgaagggcgagcggag | |
| | gcggggcaagggccacgacggcctgtatcagggcctgtc | |
| | caccgccaccaaggatacctacgacgccctgcacatgca | |
| | ggccctgcccccaagg | |
| 34 | MLLLVTSLLLCELPHPAFLLIPDIQMTQTTS | Lisocabtagene |
| | SLSASLGDRVTISCRASQDISKYLNWYQQ | maraleucel |
| | KPDGTVKLLIYHTSRLHSGVPSRFSGSGS | CD19 CAR |
| | GTDYSLTISNLEQEDIATYFCQQGNTLPYT | amino acid |
| | FGGGTKLEITGSTSGSGKPGSGEGSTKGE | sequence |
| | VKLQESGPGLVAPSQSLSVTCTVSGVSLP | |
| | DYGVSWIRQPPRKGLEWLGVIWGSETTY | |
| | YNSALKSRLTIIKDNSKSQVFLKMNSLQTD | |
| | DTAIYYCAKHYYYGGSYAMDYWGQGTSV | |
| | TVSSESKYGPPCPPCPMFWVLVVVGGVL | |
| | ACYSLLVTVAFIIFWVKRGRKKLLYIFKQPF | |
| | MRPVQTTQEEDGCSCRFPEEEEGGCELR | |
| | VKFSRSADAPAYQQGQNQLYNELNLGRR | |
| | EEYDVLDKRRGRDPEMGGKPRRKNPQE | |
| | GLYNELQKDKMAEAYSEIGMKGERRRGK | |
| | GHDGLYQGLSTATKDTYDALHMQALPPR | |
| 35 | atgcttctcctggtgacaagccttctgctctgtgagttaccac | Axicabtagene |
| | acccagcattcctcctgatcccagacatccagatgacaca | ciloleucel CD19 |
| | gactacatcctccctgtctgcctctctgggagacagagtca | CAR nucleotide |
| | ccatcagttgcagggcaagtcaggacattagtaaatattta | sequence |
| | aattggtatcagcagaaaccagatggaactgttaaactcct | |
| | gatctaccatacatcaagattacactcaggagtcccatcaa | |
| | ggttcagtggcagtgggtctggaacagattattctctcacca | |
| | ttagcaacctggagcaagaagatattgccacttacttttgcc | |
| | aacagggtaatacgcttccgtacacgttcggagggggga | |
| | ctaagttggaaataacaggctccacctctggatccggcaa | |
| | gcccggatctggcgagggatccaccaagggcgaggtga | |
| | aactgcaggagtcaggacctggcctggtggcgccctcac | |
| | agagcctgtccgtcacatgcactgtctcaggggtctcattac | |
| | ccgactatggtgtaagctggattcgccagcctccacgaaa | |
| | gggtctggagtggctgggagtaatatggggtagtgaaacc | |
| | acatactataattcagctctcaaatccagactgaccatcatc | |
| | aaggacaactccaagagccaagttttcttaaaaatgaaca | |
| | gtctgcaaactgatgacacagccatttactactgtgccaaa | |
| | cattattactacggtggtagctatgctatggactactggggtc | |
| | aaggaacctcagtcaccgtctcctcagcggccgcaattga | |
| | agttatgtatcctcctccttacctagacaatgagaagagca | |
| | atggaaccattatccatgtgaaagggaaacacctttgtcca | |
| | agtcccctatttcccggaccttctaagcccttttgggtgctggt | |
| | ggtggttgggggagtcctggcttgctatagcttgctagtaac | |
| | agtggcctttattattttctgggtgaggagtaagaggagcag | |
| | gctcctgcacagtgactacatgaacatgactccccgccgc | |
| | cccgggcccacccgcaagcattaccagccctatgcccca | |
| | ccacgcgacttcgcagcctatcgctccagagtgaagttca | |
| | gcaggagcgcagacgcccccgcgtaccagcagggcca | |
| | gaaccagctctataacgagctcaatctaggacgaagaga | |
| | ggagtacgatgttttggacaagagacgtggccgggaccct | |
| | gagatggggggaaagccgagaaggaagaaccctcagg | |
| | aaggcctgtacaatgaactgcagaaagataagatggcgg | |
| | aggcctacagtgagattgggatgaaaggcgagcgccgg | |
| | aggggcaaggggcacgatggcctttaccagggtctcagt | |
| | acagccaccaaggacacctacgacgcccttcacatgcag | |
| | gccctgccccctcgc | |
| 36 | MLLLVTSLLLCELPHPAFLLIPDIQMTQTTS | Axicabtagene |
| | SLSASLGDRVTISCRASQDISKYLNWYQQ | ciloleucel CD19 |
| | KPDGTVKLLIYHTSRLHSGVPSRFSGSGS | CAR amino acid |
| | GTDYSLTISNLEQEDIATYFCQQGNTLPYT | sequence |
| | FGGGTKLEITGSTSGSGKPGSGEGSTKGE | |
| | VKLQESGPGLVAPSQSLSVTCTVSGVSLP | |
| | DYGVSWIRQPPRKGLEWLGVIWGSETTY | |
| | YNSALKSRLTIIKDNSKSQVFLKMNSLQTD | |
| | DTAIYYCAKHYYYGGSYAMDYWGQGTSV | |
| | TVSSAAAIEVMYPPPYLDNEKSNGTIIHVK | |
| | GKHLCPSPLFPGPSKPFWVLVVVGGVLAC | |
| | YSLLVTVAFIIFWVRSKRSRLLHSDYMNMT | |
| | PRRPGPTRKHYQPYAPPRDFAAYRSRVK | |
| | FSRSADAPAYQQGQNQLYNELNLGRREE | |
| | YDVLDKRRGRDPEMGGKPRRKNPQEGLY | |
| | NELQKDKMAEAYSEIGMKGERRRGKGHD | |
| | GLYQGLSTATKDTYDALHMQALPPR | |

TABLE 8
Annotation of tisagenlecleucel CD19 CAR sequences

| | Nucleotide | Amino Acid |
|---|---|---|
| Feature | Sequence Position | Sequence Position |
| CD8a signal peptide | 1-63 | 1-21 |
| FMC63 scFv | 64-789 | 22-263 |
| (V_{L}-3xG₄S linker-V_{H}) | | |
| CD8a hinge domain | 790-924 | 264-308 |
| CD8a transmembrane domain | 925-996 | 309-332 |
| 4-1BB costimulatory domain | 997-1122 | 333-374 |
| CD3ζ signaling domain | 1123-1458 | 375-486 |

TABLE 9
Annotation of lisocabtagene maraleucel CD19 CAR sequences

| | Nucleotide | Amino Acid |
|---|---|---|
| Feature | Sequence Position | Sequence Position |
| GMCSFR-a signal peptide | 1-66 | 1-22 |
| FMC63 scFv | 67-801 | 23-267 |
| (V_{L}-Whitlow linker-V_{H}) | | |
| IgG4 hinge domain | 802-837 | 268-279 |
| CD28 transmembrane domain | 838-921 | 280-307 |
| 4-1BB costimulatory domain | 922-1047 | 308-349 |
| CD3ζ signaling domain | 1048-1383 | 350-461 |

TABLE 10
Annotation of axicabtagene ciloleucel CD19 CAR sequences

| | Nucleotide | Amino Acid |
|---|---|---|
| Feature | Sequence Position | Sequence Position |
| CSF2RA signal peptide | 1-66 | 1-22 |
| FMC63 scFv | 67-801 | 23-267 |
| (V_{L}-Whitlow linker-V_{H}) | | |
| CD28 hinge domain | 802-927 | 268-309 |
| CD28 transmembrane domain | 928-1008 | 310-336 |
| CD28 costimulatory domain | 1009-1131 | 337-377 |
| CD3ζ signaling domain | 1132-1467 | 378-489 |

In some embodiments, the polycistronic vector comprises an expression cassette that contains a nucleotide sequence encoding CD19 CAR as set forth in SEQ ID NO: 31, 33, or 35, or at least 80% identical (e.g., at least 80%, at least 85%, at least 90%, at least 95%, at least 96%, at least 97%, at least 98%, at least 99%, or 100% identical) to the nucleotide sequence set forth in SEQ ID NO: 31, 33, or 35. In certain embodiments, the nucleotide sequence may be codon optimized for expression in a mammalian cell, for example, a human cell. The encoded CD19 CAR has a corresponding amino acid sequence set forth in SEQ ID NO: 32, 34, or 36, respectively, is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 32, 34, or 36, respectively.

### CD20 CAR

In some embodiments, the CAR is a CD20 CAR, and in these embodiments, the polycistronic vector comprises an expression cassette that contains a nucleotide sequence encoding a CD20 CAR. CD20 is an antigen found on the surface of B cells as early at the pro-B phase and progressively at increasing levels until B cell maturity, as well as on the cells of most B-cell neoplasms. CD20 positive cells are also sometimes found in cases of Hodgkins disease, myeloma, and thymoma. In some embodiments, the CD20 CAR may comprise a signal peptide, an extracellular binding domain that specifically binds CD20, a hinge domain, a transmembrane domain, an intracellular costimulatory domain, and/or an intracellular signaling domain in tandem.

In some embodiments, the signal peptide of the CD20 CAR comprises a CD8α signal peptide. In some embodiments, the CD8α signal peptide comprises or consists of an amino acid sequence set forth in SEQ ID NO:6 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:6. In some embodiments, the signal peptide comprises an IgK signal peptide. In some embodiments, the IgK signal peptide comprises or consists of an amino acid sequence set forth in SEQ ID NO:7 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:7. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:8 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:8.

In some embodiments, the extracellular binding domain of the CD20 CAR is specific to CD20, for example, human CD20. The extracellular binding domain of the CD20 CAR can be codon-optimized for expression in a host cell or to have variant sequences to increase functions of the extracellular binding domain. In some embodiments, the extracellular binding domain comprises an immunogenically active portion of an immunoglobulin molecule, for example, an scFv.

In some embodiments, the extracellular binding domain of the CD20 CAR is derived from an antibody specific to CD20, including, for example, Leu16, IF5, 1.5.3, rituximab, obinutuzumab, ibritumomab, ofatumumab, tositumumab, odronextamab, veltuzumab, ublituximab, and ocrelizumab. In some embodiments, the CD20 CAR is derived from a CAR specific to CD20, including, for example, MB-106 (Fred Hutchinson Cancer Research Center, see Shadman et al., Blood 134(Suppl.1):3235 (2019)) UCART20 (Cellectis, www.cellbiomedgroup.com), or C-CAR066 (Cellular Biomedicine Group, see Liang et al., J. Clin. Oncol. 39(15) suppl:2508 (2021)). In any of these embodiments, the extracellular binding domain of the CD20 CAR can comprise or consist of the V_{H}, the V_{L}, and/or one or more CDRs of any of the antibodies.

In some embodiments, the extracellular binding domain of the CD20 CAR comprises an scFv derived from the Leu16 monoclonal antibody, which comprises the heavy chain variable region (V_{H}) and the light chain variable region (V_{L}) of Leu16 connected by a linker. See Wu et al., Protein Engineering. 14(12):1025-1033 (2001). In some embodiments, the linker is a (G₄S)ₙ (n can be a positive integer, e.g., 1, 2, 3, 4, 5, or 6) linker, for example, a 3×G₄S linker. In other embodiments, the linker is a Whitlow linker as described herein. In some embodiments, the amino acid sequences of different portions of the entire Leu16-derived scFv (also referred to as Leu16 scFv) and its different portions are provided in Table 11 below. In some embodiments, the CD20-specific scFv comprises or consists of an amino acid sequence set forth in SEQ ID NO:37, 38, or 42, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:37, 38, or 42. In some embodiments, the CD20-specific scFv may comprise one or more CDRs having amino acid sequences set forth in SEQ ID NOs: 39-41, 43 and 44. In some embodiments, the CD20-specific scFv may comprise a light chain with one or more CDRs having amino acid sequences set forth in SEQ ID NOs: 39-41. In some embodiments, the CD20-specific scFv may comprise a heavy chain with one or more CDRs having amino acid sequences set forth in SEQ ID NOs: 43-44. In any of these embodiments, the CD20-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the CD20 CAR comprises or consists of the one or more CDRs as described herein.

TABLE 11
Exemplary sequences of anti-CD20 scFv and components

| SEQ ID NO: | Amino Acid Sequence | Description |
|---|---|---|
| 37 | DIVLTQSPAILSASPGEKVTMTCRA | Anti-CD20 Leu16 scFv |
| | SSSVNYMDWYQKKPGSSPKPWIYA | entire sequence, with |
| | TSNLASGVPARFSGSGSGTSYSLTI | Whitlow linker |
| | SRVEAEDAATYYCQQWSFNPPTFG | |
| | GGTKLEIKGSTSGSGKPGSGEGST | |
| | KGEVQLQQSGAELVKPGASVKMSC | |
| | KASGYTFTSYNMHWVKQTPGQGL | |
| | EWIGAIYPGNGDTSYNQKFKGKATL | |
| | TADKSSSTAYMQLSSLTSEDSADY | |
| | YCARSNYYGSSYWFFDVWGAGTT | |
| | VTVSS | |
| 38 | DIVLTQSPAILSASPGEKVTMTCRA | Anti-CD20 Leu16 scFv |
| | SSSVNYMDWYQKKPGSSPKPWIYA | light chain variable |
| | TSNLASGVPARFSGSGSGTSYSLTI | region |
| | SRVEAEDAATYYCQQWSFNPPTFG | |
| | GGTKLEIK | |
| 39 | RASSSVNYMD | Anti-CD20 Leu16 scFv |
| | | light chain CDR1 |
| 40 | ATSNLAS | Anti-CD20 Leu16 scFv |
| | | light chain CDR2 |
| 41 | QQWSFNPPT | Anti-CD20 Leu16 scFv |
| | | light chain CDR3 |
| 42 | EVQLQQSGAELVKPGASVKMSCKA | Anti-CD20 Leu16 scFv |
| | SGYTFTSYNMHWVKQTPGQGLEWI | heavy chain |
| | GAIYPGNGDTSYNQKFKGKATLTA | |
| | DKSSSTAYMQLSSLTSEDSADYYC | |
| | ARSNYYGSSYWFFDVWGAGTTVT | |
| | VSS | |
| 43 | SYNMH | Anti-CD20 Leu16 scFv |
| | | heavy chain CDR1 |
| 44 | AIYPGNGDTSYNQKFKG | Anti-CD20 Leu16 scFv |
| | | heavy chain CDR2 |

In some embodiments, the hinge domain of the CD20 CAR comprises a CD8α hinge domain, for example, a human CD8α hinge domain. In some embodiments, the CD8α hinge domain comprises or consists of an amino acid sequence set forth in SEQ ID NO:9 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:9. In some embodiments, the hinge domain comprises a CD28 hinge domain, for example, a human CD28 hinge domain. In some embodiments, the CD28 hinge domain comprises or consists of an amino acid sequence set forth in SEQ ID NO:10 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:10. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO:11 or SEQ ID NO:12, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:11 or SEQ ID NO:12. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO:13 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:13.

In some embodiments, the transmembrane domain of the CD20 CAR comprises a CD8α transmembrane domain, for example, a human CD8α transmembrane domain. In some embodiments, the CD8α transmembrane domain comprises or consists of an amino acid sequence set forth in SEQ ID NO:14 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:14. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO:15 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:15.

In some embodiments, the intracellular costimulatory domain of the CD20 CAR comprises a 4-1 BB costimulatory domain, for example, a human 4-1 BB costimulatory domain. In some embodiments, the 4-1 BB costimulatory domain comprises or consists of an amino acid sequence set forth in SEQ ID NO:16 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:16. In some embodiments, the intracellular costimulatory domain comprises a CD28 costimulatory domain, for example, a human CD28 costimulatory domain. In some embodiments, the CD28 costimulatory domain comprises or consists of an amino acid sequence set forth in SEQ ID NO:17 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:17.

In some embodiments, the intracellular signaling domain of the CD20 CAR comprises a CD3 zeta (ζ) signaling domain, for example, a human CD3ζ signaling domain. In some embodiments, the CD3ζ signaling domain comprises or consists of an amino acid sequence set forth in SEQ ID NO:18 or SEQ ID NO:115, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:18 or SEQ ID NO:115.

In some embodiments, the polycistronic vector comprises an expression cassette that contains a nucleotide sequence encoding a CD20 CAR, including, for example, a CD20 CAR comprising the CD20-specific scFv having sequences set forth in SEQ ID NO:37, the CD8α hinge domain of SEQ ID NO:9, the CD8α transmembrane domain of SEQ ID NO:14, the 4-1 BB costimulatory domain of SEQ ID NO:16, the CD3ζ signaling domain of SEQ ID NO:18 or SEQ ID NO:115, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD20 CAR, including, for example, a CD20 CAR comprising the CD20-specific scFv having sequences set forth in SEQ ID NO:37, the CD28 hinge domain of SEQ ID NO:10, the CD8α transmembrane domain of SEQ ID NO:14, the 4-1BB costimulatory domain of SEQ ID NO:16, the CD3ζ signaling domain of SEQ ID NO:18 or SEQ ID NO:115, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD20 CAR, including, for example, a CD20 CAR comprising the CD20-specific scFv having sequences set forth in SEQ ID NO:37, the IgG4 hinge domain of SEQ ID NO:11 or SEQ ID NO:12, the CD8α transmembrane domain of SEQ ID NO:14, the 4-1BB costimulatory domain of SEQ ID NO:16, the CD3ζ signaling domain of SEQ ID NO:18 or SEQ ID NO:115, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD20 CAR, including, for example, a CD20 CAR comprising the CD20-specific scFv having sequences set forth in SEQ ID NO:37, the CD8α hinge domain of SEQ ID NO:9, the CD28 transmembrane domain of SEQ ID NO:15, the 4-1 BB costimulatory domain of SEQ ID NO:16, the CD3ζ signaling domain of SEQ ID NO:18 or SEQ ID NO:115, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD20 CAR, including, for example, a CD20 CAR comprising the CD20-specific scFv having sequences set forth in SEQ ID NO:37, the CD28 hinge domain of SEQ ID NO:10, the CD28 transmembrane domain of SEQ ID NO:15, the 4-1 BB costimulatory domain of SEQ ID NO:16, the CD3ζ signaling domain of SEQ ID NO:18 or SEQ ID NO:115, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD20 CAR, including, for example, a CD20 CAR comprising the CD20-specific scFv having sequences set forth in SEQ ID NO:37, the IgG4 hinge domain of SEQ ID NO:11 or SEQ ID NO:12, the CD28 transmembrane domain of SEQ ID NO:15, the 4-1BB costimulatory domain of SEQ ID NO:16, the CD3ζ signaling domain of SEQ ID NO:18 or SEQ ID NO:115, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

### CD22 CAR

In some embodiments, the CAR is a CD22 CAR, and in these embodiments, the polycistronic vector comprises an expression cassette that contains a nucleotide sequence encoding a CD22 CAR. CD22, which is a transmembrane protein found mostly on the surface of mature B cells that functions as an inhibitory receptor for B cell receptor (BCR) signaling. CD22 is expressed in 60-70% of B cell lymphomas and leukemias (e.g., B-chronic lymphocytic leukemia, hairy cell leukemia, acute lymphocytic leukemia (ALL), and Burkitt's lymphoma) and is not present on the cell surface in early stages of B cell development or on stem cells. In some embodiments, the CD22 CAR may comprise a signal peptide, an extracellular binding domain that specifically binds CD22, a hinge domain, a transmembrane domain, an intracellular costimulatory domain, and/or an intracellular signaling domain in tandem.

In some embodiments, the signal peptide of the CD22 CAR comprises a CD8α signal peptide. In some embodiments, the CD8α signal peptide comprises or consists of an amino acid sequence set forth in SEQ ID NO:6 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:6. In some embodiments, the signal peptide comprises an IgK signal peptide. In some embodiments, the IgK signal peptide comprises or consists of an amino acid sequence set forth in SEQ ID NO:7 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:7. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:8 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:8.

In some embodiments, the extracellular binding domain of the CD22 CAR is specific to CD22, for example, human CD22. The extracellular binding domain of the CD22 CAR can be codon-optimized for expression in a host cell or to have variant sequences to increase functions of the extracellular binding domain. In some embodiments, the extracellular binding domain comprises an immunogenically active portion of an immunoglobulin molecule, for example, an scFv.

In some embodiments, the extracellular binding domain of the CD22 CAR is derived from an antibody specific to CD22, including, for example, SM03, inotuzumab, epratuzumab, moxetumomab, and pinatuzumab. In any of these embodiments, the extracellular binding domain of the CD22 CAR can comprise or consist of the V_{H}, the V_{L}, and/or one or more CDRs of any of the antibodies.

In some embodiments, the extracellular binding domain of the CD22 CAR comprises an scFv derived from the m971 monoclonal antibody (m971), which comprises the heavy chain variable region (V_{H}) and the light chain variable region (V_{L}) of m971 connected by a linker. In some embodiments, the linker is a (G₄S)ₙ (n can be a positive integer, e.g., 1, 2, 3, 4, 5, or 6) linker, for example, a 3×G₄S linker. In other embodiments, the Whitlow linker may be used instead. In some embodiments, the amino acid sequences of the entire m971-derived scFv (also referred to as m971 scFv) and its different portions are provided in Table 12 below. In some embodiments, the CD22-specific scFv comprises or consists of an amino acid sequence set forth in SEQ ID NO:45, 46, or 50, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:45, 46, or 50. In some embodiments, the CD22-specific scFv may comprise one or more CDRs having amino acid sequences set forth in SEQ ID NOs: 47-49 and 51-53. In some embodiments, the CD22-specific scFv may comprise a heavy chain with one or more CDRs having amino acid sequences set forth in SEQ ID NOs: 47-49. In some embodiments, the CD22-specific scFv may comprise a light chain with one or more CDRs having amino acid sequences set forth in SEQ ID NOs: 51-53. In any of these embodiments, the CD22-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the CD22 CAR comprises or consists of the one or more CDRs as described herein.

In some embodiments, the extracellular binding domain of the CD22 CAR comprises an scFv derived from m971-L7, which is an affinity matured variant of m971 with significantly improved CD22 binding affinity compared to the parental antibody m971 (improved from about 2 nM to less than 50 pM). In some embodiments, the scFv derived from m971-L7 comprises the V_{H} and the V_{L} of m971-L7 connected by a (G₄S)ₙ (n can be a positive integer, e.g., 1, 2, 3, 4, 5, or 6) linker, for example, a 3×G₄S linker. In other embodiments, the Whitlow linker may be used instead. In some embodiments, the amino acid sequences of the entire m971-L7-derived scFv (also referred to as m971-L7 scFv) and its different portions are provided in Table 12 below. In some embodiments, the CD22-specific scFv comprises or consists of an amino acid sequence set forth in SEQ ID NO:54, 55, or 59, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:54, 55, or 59. In some embodiments, the CD22-specific scFv may comprise one or more CDRs having amino acid sequences set forth in SEQ ID NOs: 56-58 and 60-62. In some embodiments, the CD22-specific scFv may comprise a heavy chain with one or more CDRs having amino acid sequences set forth in SEQ ID NOs: 56-58. In some embodiments, the CD22-specific scFv may comprise a light chain with one or more CDRs having amino acid sequences set forth in SEQ ID NOs: 60-62. In any of these embodiments, the CD22-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the CD22 CAR comprises or consists of the one or more CDRs as described herein.

TABLE 12
Exemplary sequences of anti-CD22 CAR, scFv, and components

| SEQ ID NO: | Amino Acid Sequence | Description |
|---|---|---|
| 45 | QVQLQQSGPGLVKPSQTLSLTCAIS | Anti-CD22 m971 scFv |
| | GDSVSSNSAAWNWIRQSPSRGLE | entire sequence, with |
| | WLGRTYYRSKWYNDYAVSVKSRITI | 3xG₄S linker |
| | NPDTSKNQFSLQLNSVTPEDTAVY | |
| | YCAREVTGDLEDAFDIWGQGTMVT | |
| | VSSGGGGSGGGGSGGGGSDIQMT | |
| | QSPSSLSASVGDRVTITCRASQTIW | |
| | SYLNWYQQRPGKAPNLLIYAASSL | |
| | QSGVPSRFSGRGSGTDFTLTISSLQ | |
| | AEDFATYYCQQSYSIPQTFGQGTKL | |
| | EIK | |
| 46 | QVQLQQSGPGLVKPSQTLSLTCAIS | Anti-CD22 m971 scFv |
| | GDSVSSNSAAWNWIRQSPSRGLE | heavy chain variable |
| | WLGRTYYRSKWYNDYAVSVKSRITI | region |
| | NPDTSKNQFSLQLNSVTPEDTAVY | |
| | YCAREVTGDLEDAFDIWGQGTMVT | |
| | VSS | |
| 47 | GDSVSSNSAA | Anti-CD22 m971 scFv |
| | | heavy chain CDR1 |
| 48 | TYYRSKWYN | Anti-CD22 m971 scFv |
| | | heavy chain CDR2 |
| 49 | AREVTGDLEDAFDI | Anti-CD22 m971 scFv |
| | | heavy chain CDR3 |
| 50 | DIQMTQSPSSLSASVGDRVTITCRA | Anti-CD22 m971 scFv |
| | SQTIWSYLNWYQQRPGKAPNLLIYA | light chain |
| | ASSLQSGVPSRFSGRGSGTDFTLTI | |
| | SSLQAEDFATYYCQQSYSIPQTFG | |
| | QGTKLEIK | |
| 51 | QTIWSY | Anti-CD22 m971 scFv |
| | | light chain CDR1 |
| 52 | AAS | Anti-CD22 m971 scFv |
| | | light chain CDR2 |
| 53 | QQSYSIPQT | Anti-CD22 m971 scFv |
| | | light chain CDR3 |
| 54 | QVQLQQSGPGMVKPSQTLSLTCAI | Anti-CD22 m971-L7 |
| | SGDSVSSNSVAWNWIRQSPSRGLE | scFv entire sequence, |
| | WLGRTYYRSTWYNDYAVSMKSRIT | with 3xG4S linker |
| | INPDTNKNQFSLQLNSVTPEDTAVY | |
| | YCAREVTGDLEDAFDIWGQGTMVT | |
| | VSSGGGGSGGGGSGGGGSDIQMI | |
| | QSPSSLSASVGDRVTITCRASQTIW | |
| | SYLNWYRQRPGEAPNLLIYAASSLQ | |
| | SGVPSRFSGRGSGTDFTLTISSLQA | |
| | EDFATYYCQQSYSIPQTFGQGTKLE | |
| | IK | |
| 55 | QVQLQQSGPGMVKPSQTLSLTCAI | Anti-CD22 m971-L7 |
| | SGDSVSSNSVAWNWIRQSPSRGLE | scFv heavy chain |
| | WLGRTYYRSTWYNDYAVSMKSRIT | variable region |
| | INPDTNKNQFSLQLNSVTPEDTAVY | |
| | YCAREVTGDLEDAFDIWGQGTMVT | |
| | VSS | |
| 56 | GDSVSSNSVA | Anti-CD22 m971-L7 |
| | | scFv heavy chain |
| | | CDR1 |
| 57 | TYYRSTWYN | Anti-CD22 m971-L7 |
| | | scFv heavy chain |
| | | CDR2 |
| 58 | AREVTGDLEDAFDI | Anti-CD22 m971-L7 |
| | | scFv heavy chain |
| | | CDR3 |
| 59 | DIQMIQSPSSLSASVGDRVTITCRA | Anti-CD22 m971-L7 |
| | SQTIWSYLNWYRQRPGEAPNLLIYA | scFv light chain |
| | ASSLQSGVPSRFSGRGSGTDFTLTI | variable region |
| | SSLQAEDFATYYCQQSYSIPQTFG | |
| | QGTKLEIK | |
| 60 | QTIWSY | Anti-CD22 m971-L7 |
| | | scFv light chain CDR1 |
| 61 | AAS | Anti-CD22 m971-L7 |
| | | scFv light chain CDR2 |
| 62 | QQSYSIPQT | Anti-CD22 m971-L7 |
| | | scFv light chain CDR3 |
| 136 | MLLLVTSLLLCELPHPAFLLIPQVQL | Exemplary CD22 CAR |
| | QQSGPGLVKPSQTLSLTCAISGDSV | amino acid sequence |
| | SSNSAAWNWIRQSPSRGLEWLGR | |
| | TYYRSKWYNDYAVSVKSRITINPDT | |
| | SKNQFSLQLNSVTPEDTAVYYCAR | |
| | EVTGDLEDAFDIWGQGTMVTVSSG | |
| | GGGSDIQMTQSPSSLSASVGDRVTI | |
| | TCRASQTIWSYLNWYQQRPGKAPN | |
| | LLIYAASSLQSGVPSRFSGRGSGTD | |
| | FTLTISSLQAEDFATYYCQQSYSIPQ | |
| | TFGQGTKLEIKAAATTTPAPRPPTP | |
| | APTIASQPLSLRPEACRPAAGGAVH | |
| | TRGLDFACDIYIWAPLAGTCGVLLL | |
| | SLVITLYCKRGRKKLLYIFKQPFMRP | |
| | VQTTQEEDGCSCRFPEEEEGGCEL | |
| | RVKFSRSADAPAYKQGQNQLYNEL | |
| | NLGRREEYDVLDKRRGRDPEMGG | |
| | KPRRKNPQEGLYNELQKDKMAEAY | |
| | SEIGMKGERRRGKGHDGLYQGLST | |
| | ATKDTYDALHMQALPPR | |

In some embodiments, the extracellular binding domain of the CD22 CAR comprises immunotoxins HA22 or BL122. Immunotoxins BL22 and HA22 are therapeutic agents that comprise an scFv specific for CD22 fused to a bacterial toxin, and thus can bind to the surface of the cancer cells that express CD22 and kill the cancer cells. BL22 comprises a dsFv of an anti-CD22 antibody, RFB4, fused to a 38-kDa truncated form of Pseudomonas exotoxin A (Bang et al., Clin. Cancer Res., 11:1545-50 (2005)). HA22 (CAT8015, moxetumomab pasudotox) is a mutated, higher affinity version of BL22 (Ho et al., J. Biol. Chem., 280(1): 607-17 (2005)). Suitable sequences of antigen binding domains of HA22 and BL22 specific to CD22 are disclosed in, for example, U.S. Pat. Nos. 7,541,034; 7,355,012; and 7,982,011, which are hereby incorporated by reference in their entirety.

In some embodiments, the hinge domain of the CD22 CAR comprises a CD8α hinge domain, for example, a human CD8α hinge domain. In some embodiments, the CD8α hinge domain comprises or consists of an amino acid sequence set forth in SEQ ID NO:9 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:9. In some embodiments, the hinge domain comprises a CD28 hinge domain, for example, a human CD28 hinge domain. In some embodiments, the CD28 hinge domain comprises or consists of an amino acid sequence set forth in SEQ ID NO:10 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:10. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO:11 or SEQ ID NO:12, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:11 or SEQ ID NO:12. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO:13 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:13.

In some embodiments, the transmembrane domain of the CD22 CAR comprises a CD8α transmembrane domain, for example, a human CD8α transmembrane domain. In some embodiments, the CD8α transmembrane domain comprises or consists of an amino acid sequence set forth in SEQ ID NO:14 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:14. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO:15 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:15.

In some embodiments, the intracellular costimulatory domain of the CD22 CAR comprises a 4-1 BB costimulatory domain, for example, a human 4-1 BB costimulatory domain. In some embodiments, the 4-1 BB costimulatory domain comprises or consists of an amino acid sequence set forth in SEQ ID NO:16 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:16. In some embodiments, the intracellular costimulatory domain comprises a CD28 costimulatory domain, for example, a human CD28 costimulatory domain. In some embodiments, the CD28 costimulatory domain comprises or consists of an amino acid sequence set forth in SEQ ID NO:17 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:17.

In some embodiments, the intracellular signaling domain of the CD22 CAR comprises a CD3 zeta (ζ) signaling domain, for example, a human CD3ζ signaling domain. In some embodiments, the CD3ζ signaling domain comprises or consists of an amino acid sequence set forth in SEQ ID NO:18 or SEQ ID NO:115, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:18 or SEQ ID NO:115.

In some embodiments, the polycistronic vector comprises an expression cassette that contains a nucleotide sequence encoding a CD22 CAR, including, for example, a CD22 CAR comprising the CD22-specific scFv having sequences set forth in SEQ ID NO:45 or SEQ ID NO:54, the CD8α hinge domain of SEQ ID NO:9, the CD8α transmembrane domain of SEQ ID NO:14, the 4-1BB costimulatory domain of SEQ ID NO:16, the CD3ζ signaling domain of SEQ ID NO:18 or SEQ ID NO:115, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD22 CAR, including, for example, a CD22 CAR comprising the CD22-specific scFv having sequences set forth in SEQ ID NO:45 or SEQ ID NO:54, the CD28 hinge domain of SEQ ID NO:10, the CD8α transmembrane domain of SEQ ID NO:14, the 4-1BB costimulatory domain of SEQ ID NO:16, the CD3ζ signaling domain of SEQ ID NO:18 or SEQ ID NO:115, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD22 CAR, including, for example, a CD22 CAR comprising the CD22-specific scFv having sequences set forth in SEQ ID NO:45 or SEQ ID NO:54, the IgG4 hinge domain of SEQ ID NO:11 or SEQ ID NO:12, the CD8α transmembrane domain of SEQ ID NO:14, the 4-1 BB costimulatory domain of SEQ ID NO:16, the CD3ζ signaling domain of SEQ ID NO:18 or SEQ ID NO:115, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD22 CAR, including, for example, a CD22 CAR comprising the CD22-specific scFv having sequences set forth in SEQ ID NO:45 or SEQ ID NO:54, the CD8α hinge domain of SEQ ID NO:9, the CD28 transmembrane domain of SEQ ID NO:15, the 4-1BB costimulatory domain of SEQ ID NO:16, the CD3ζ signaling domain of SEQ ID NO:18 or SEQ ID NO:115, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD22 CAR, including, for example, a CD22 CAR comprising the CD22-specific scFv having sequences set forth in SEQ ID NO:45 or SEQ ID NO:54, the CD28 hinge domain of SEQ ID NO:10, the CD28 transmembrane domain of SEQ ID NO:15, the 4-1BB costimulatory domain of SEQ ID NO:16, the CD3ζ signaling domain of SEQ ID NO:18 or SEQ ID NO:115, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD22 CAR, including, for example, a CD22 CAR comprising the CD22-specific scFv having sequences set forth in SEQ ID NO:45 or SEQ ID NO:54, the IgG4 hinge domain of SEQ ID NO:11 or SEQ ID NO:12, the CD28 transmembrane domain of SEQ ID NO:15, the 4-1 BB costimulatory domain of SEQ ID NO:16, the CD3ζ signaling domain of SEQ ID NO:18 or SEQ ID NO:115, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD22 CAR having an amino acid sequence set forth in SEQ ID NO:136 or that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:136, with the following components: GMCSFR-α signal peptide, m971 scFv (V_{H}-G₄S linker-V_{L}), CD8α hinge domain, CD8α transmembrane domain, 4-1 BB costimulatory domain, and CD3ζ signaling domain (see Table 12).

### BCMA CAR

In some embodiments, the CAR is a BCMA CAR, and in these embodiments, the polycistronic vector comprises an expression cassette that contains a nucleotide sequence encoding a BCMA CAR. BCMA is a tumor necrosis family receptor (TNFR) member expressed on cells of the B cell lineage, with the highest expression on terminally differentiated B cells or mature B lymphocytes. BCMA is involved in mediating the survival of plasma cells for maintaining long-term humoral immunity. The expression of BCMA has been recently linked to a number of cancers, such as multiple myeloma, Hodgkin's and non-Hodgkin's lymphoma, various leukemias, and glioblastoma. In some embodiments, the BCMA CAR may comprise a signal peptide, an extracellular binding domain that specifically binds BCMA, a hinge domain, a transmembrane domain, an intracellular costimulatory domain, and/or an intracellular signaling domain in tandem.

In some embodiments, the signal peptide of the BCMA CAR comprises a CD8α signal peptide. In some embodiments, the CD8α signal peptide comprises or consists of an amino acid sequence set forth in SEQ ID NO:6 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:6. In some embodiments, the signal peptide comprises an IgK signal peptide. In some embodiments, the IgK signal peptide comprises or consists of an amino acid sequence set forth in SEQ ID NO:7 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:7. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:8 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:8.

In some embodiments, the extracellular binding domain of the BCMA CAR is specific to BCMA, for example, human BCMA. The extracellular binding domain of the BCMA CAR can be codon-optimized for expression in a host cell or to have variant sequences to increase functions of the extracellular binding domain.

In some embodiments, the extracellular binding domain comprises an immunogenically active portion of an immunoglobulin molecule, for example, an scFv. In some embodiments, the extracellular binding domain of the BCMA CAR is derived from an antibody specific to BCMA, including, for example, belantamab, erlanatamab, teclistamab, LCAR-B38M, and ciltacabtagene. In any of these embodiments, the extracellular binding domain of the BCMA CAR can comprise or consist of the V_{H}, the V_{L}, and/or one or more CDRs of any of the antibodies.

In some embodiments, the extracellular binding domain of the BCMA CAR comprises an scFv derived from C11 D5.3, a murine monoclonal antibody as described in Carpenter et al., Clin. Cancer Res. 19(8):2048-2060 (2013). See also PCT Application Publication No. WO2010104949. The C11 D5.3-derived scFv may comprise the heavy chain variable region (V_{H}) and the light chain variable region (V_{L}) of C11 D5.3 connected by the Whitlow linker, the amino acid sequences of which is provided in Table 13 below. In some embodiments, the BCMA-specific extracellular binding domain comprises or consists of an amino acid sequence set forth in SEQ ID NO:63, 64, or 68, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:63, 64, or 68. In some embodiments, the BCMA-specific extracellular binding domain may comprise one or more CDRs having amino acid sequences set forth in SEQ ID NOs: 65-67 and 69-71. In some embodiments, the BCMA-specific extracellular binding domain may comprise a light chain with one or more CDRs having amino acid sequences set forth in SEQ ID NOs: 65-67. In some embodiments, the BCMA-specific extracellular binding domain may comprise a heavy chain with one or more CDRs having amino acid sequences set forth in SEQ ID NOs: 69-71. In any of these embodiments, the BCMA-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the BCMA CAR comprises or consists of the one or more CDRs as described herein.

In some embodiments, the extracellular binding domain of the BCMA CAR comprises an scFv derived from another murine monoclonal antibody, C12A3.2, as described in Carpenter et al., Clin. Cancer Res. 19(8):2048-2060 (2013) and PCT Application Publication No. WO2010104949, the amino acid sequence of which is also provided in Table 13 below. In some embodiments, the BCMA-specific extracellular binding domain comprises or consists of an amino acid sequence set forth in SEQ ID NO:72, 73, or 77, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:72, 73, or 77. In some embodiments, the BCMA-specific extracellular binding domain may comprise one or more CDRs having amino acid sequences set forth in SEQ ID NOs: 74-76 and 78-80. In some embodiments, the BCMA-specific extracellular binding domain may comprise a light chain with one or more CDRs having amino acid sequences set forth in SEQ ID NOs: 74-76. In some embodiments, the BCMA-specific extracellular binding domain may comprise a heavy chain with one or more CDRs having amino acid sequences set forth in SEQ ID NOs: 78-80. In any of these embodiments, the BCMA-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the BCMA CAR comprises or consists of the one or more CDRs as described herein.

In some embodiments, the extracellular binding domain of the BCMA CAR comprises a murine monoclonal antibody with high specificity to human BCMA, referred to as BB2121 in Friedman et al., Hum. Gene Ther. 29(5):585-601 (2018)). See also, PCT Application Publication No. WO2012163805.

In some embodiments, the extracellular binding domain of the BCMA CAR comprises single variable fragments of two heavy chains (VHH) that can bind to two epitopes of BCMA as described in Zhao et al., J. Hematol. Oncol. 11(1):141 (2018), also referred to as LCAR-B38M. See also, PCT Application Publication No. WO2018028647.

In some embodiments, the extracellular binding domain of the BCMA CAR comprises a fully human heavy-chain variable domain (FHVH) as described in Lam et al., Nat. Commun. 11(1):283 (2020), also referred to as FHVH33. See also, PCT Application Publication No. WO2019006072. The amino acid sequences of FHVH33 and its CDRs are provided in Table 13 below. In some embodiments, the BCMA-specific extracellular binding domain comprises or consists of an amino acid sequence set forth in SEQ ID NO:81 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:81. In some embodiments, the BCMA-specific extracellular binding domain may comprise one or more CDRs having amino acid sequences set forth in SEQ ID NOs: 82-84. In any of these embodiments, the BCMA-specific extracellular binding domain may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the BCMA CAR comprises or consists of the one or more CDRs as described herein.

In some embodiments, the extracellular binding domain of the BCMA CAR comprises an scFv derived from CT103A (or CAR0085) as described in U.S. Pat. No. 11,026,975 B2, the amino acid sequence of which is provided in Table 13 below. In some embodiments, the BCMA-specific extracellular binding domain comprises or consists of an amino acid sequence set forth in SEQ ID NO:118, 119, or 123, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 118, 119, or 123. In some embodiments, the BCMA-specific extracellular binding domain may comprise one or more CDRs having amino acid sequences set forth in SEQ ID NOs: 120-122 and 124-126. In some embodiments, the BCMA-specific extracellular binding domain may comprise a light chain with one or more CDRs having amino acid sequences set forth in SEQ ID NOs: 120-122. In some embodiments, the BCMA-specific extracellular binding domain may comprise a heavy chain with one or more CDRs having amino acid sequences set forth in SEQ ID NOs: 124-126. In any of these embodiments, the BCMA-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the BCMA CAR comprises or consists of the one or more CDRs as described herein.

Additionally, CARs and binders directed to BCMA have been described in U.S. Application Publication Nos. 2020/0246381 A1 and 2020/0339699 A1, the entire contents of each of which are incorporated by reference herein.

TABLE 13
Exemplary sequences of anti-BCMA binder and components

| SEQ ID NO: | Amino Acid Sequence | Description |
|---|---|---|
| 63 | DIVLTQSPASLAMSLGKRATISCRA | Anti-BCMA C11D5.3 |
| | SESVSVIGAHLIHWYQQKPGQPPKL | scFv entire sequence, |
| | LIYLASNLETGVPARFSGSGSGTDF | with Whitlow linker |
| | TLTIDPVEEDDVAIYSCLQSRIFPRT | |
| | FGGGTKLEIKGSTSGSGKPGSGEG | |
| | STKGQIQLVQSGPELKKPGETVKIS | |
| | CKASGYTFTDYSINWVKRAPGKGL | |
| | KWMGWINTETREPAYAYDFRGRFA | |
| | FSLETSASTAYLQINNLKYEDTATYF | |
| | CALDYSYAMDYWGQGTSVTVSS | |
| 64 | DIVLTQSPASLAMSLGKRATISCRA | Anti-BCMA C11D5.3 |
| | SESVSVIGAHLIHWYQQKPGQPPKL | scFv light chain |
| | LIYLASNLETGVPARFSGSGSGTDF | variable region |
| | TLTIDPVEEDDVAIYSCLQSRIFPRT | |
| | FGGGTKLEIK | |
| 65 | RASESVSVIGAHLIH | Anti-BCMA C11D5.3 |
| | | scFv light chain CDR1 |
| 66 | LASNLET | Anti-BCMA C11D5.3 |
| | | scFv light chain CDR2 |
| 67 | LOSRIFPRT | Anti-BCMA C11D5.3 |
| | | scFv light chain CDR3 |
| 68 | QIQLVQSGPELKKPGETVKISCKAS | Anti-BCMA C11D5.3 |
| | GYTFTDYSINWVKRAPGKGLKWMG | scFv heavy chain |
| | WINTETREPAYAYDFRGRFAFSLET | variable region |
| | SASTAYLQINNLKYEDTATYFCALD | |
| | YSYAMDYWGQGTSVTVSS | |
| 69 | DYSIN | Anti-BCMA C11D5.3 |
| | | scFv heavy chain |
| | | CDR1 |
| 70 | WINTETREPAYAYDFRG | Anti-BCMA C11D5.3 |
| | | scFv heavy chain |
| | | CDR2 |
| 71 | DYSYAMDY | Anti-BCMA C11D5.3 |
| | | scFv heavy chain |
| | | CDR3 |
| 72 | DIVLTQSPPSLAMSLGKRATISCRA | Anti-BCMA C12A3.2 |
| | SESVTILGSHLIYWYQQKPGQPPTL | scFv entire sequence, |
| | LIQLASNVQTGVPARFSGSGSRTDF | with Whitlow linker |
| | TLTIDPVEEDDVAVYYCLQSRTIPRT | |
| | FGGGTKLEIKGSTSGSGKPGSGEG | |
| | STKGQIQLVQSGPELKKPGETVKIS | |
| | CKASGYTFRHYSMNWVKQAPGKG | |
| | LKWMGRINTESGVPIYADDFKGRFA | |
| | FSVETSASTAYLVINNLKDEDTASYF | |
| | CSNDYLYSLDFWGQGTALTVSS | |
| 73 | DIVLTQSPPSLAMSLGKRATISCRA | Anti-BCMA C12A3.2 |
| | SESVTILGSHLIYWYQQKPGQPPTL | scFv light chain |
| | LIQLASNVQTGVPARFSGSGSRTDF | variable region |
| | TLTIDPVEEDDVAVYYCLQSRTIPRT | |
| | FGGGTKLEIK | |
| 74 | RASESVTILGSHLIY | Anti-BCMA C12A3.2 |
| | | scFv light chain CDR1 |
| 75 | LASNVQT | Anti-BCMA C12A3.2 |
| | | scFv light chain CDR2 |
| 76 | LQSRTIPRT | Anti-BCMA C12A3.2 |
| | | scFv light chain CDR3 |
| 77 | QIQLVQSGPELKKPGETVKISCKAS | Anti-BCMA C12A3.2 |
| | GYTFRHYSMNWVKQAPGKGLKWM | scFv heavy chain |
| | GRINTESGVPIYADDFKGRFAFSVE | variable region |
| | TSASTAYLVINNLKDEDTASYFCSN | |
| | DYLYSLDFWGQGTALTVSS | |
| 78 | HYSMN | Anti-BCMA C12A3.2 |
| | | scFv heavy chain |
| | | CDR1 |
| 79 | RINTESGVPIYADDFKG | Anti-BCMA C12A3.2 |
| | | scFv heavy chain |
| | | CDR2 |
| 80 | DYLYSLDF | Anti-BCMA C12A3.2 |
| | | scFv heavy chain |
| | | CDR3 |
| 81 | EVQLLESGGGLVQPGGSLRLSCAA | Anti-BCMA FHVH33 |
| | SGFTFSSYAMSWVRQAPGKGLEW | entire sequence |
| | VSSISGSGDYIYYADSVKGRFTISRD | |
| | ISKNTLYLQMNSLRAEDTAVYYCAK | |
| | EGTGANSSLADYRGQGTLVTVSS | |
| 82 | GFTFSSYA | Anti-BCMA FHVH33 |
| | | CDR1 |
| 83 | ISGSGDYI | Anti-BCMA FHVH33 |
| | | CDR2 |
| 84 | AKEGTGANSSLADY | Anti-BCMA FHVH33 |
| | | CDR3 |
| 118 | DIQMTQSPSSLSASVGDRVTITCRA | Anti-BCMA CT103A |
| | SQSISSYLNWYQQKPGKAPKLLIYA | scFv entire sequence, |
| | ASSLQSGVPSRFSGSGSGTDFTLTI | with Whitlow linker |
| | SSLQPEDFATYYCQQKYDLLTFGG | |
| | GTKVEIKGSTSGSGKPGSGEGSTK | |
| | GQLQLQESGPGLVKPSETLSLTCTV | |
| | SGGSISSSSYYWGWIRQPPGKGLE | |
| | WIGSISYSGSTYYNPSLKSRVTISVD | |
| | TSKNQFSLKLSSVTAADTAVYYCAR | |
| | DRGDTILDVWGQGTMVTVSS | |
| 119 | DIQMTQSPSSLSASVGDRVTITCRA | Anti-BCMA CT103A |
| | SQSISSYLNWYQQKPGKAPKLLIYA | scFv light chain |
| | ASSLQSGVPSRFSGSGSGTDFTLTI | variable region |
| | SSLQPEDFATYYCQQKYDLLTFGG | |
| | GTKVEIK | |
| 120 | QSISSY | Anti-BCMA CT103A |
| | | scFv light chain CDR1 |
| 121 | AAS | Anti-BCMA CT103A |
| | | scFv light chain CDR2 |
| 122 | QQKYDLLT | Anti-BCMA CT103A |
| | | scFv light chain CDR3 |
| 123 | QLQLQESGPGLVKPSETLSLTCTVS | Anti-BCMA CT103A |
| | GGSISSSSYYWGWIRQPPGKGLEW | scFv heavy chain |
| | IGSISYSGSTYYNPSLKSRVTISVDT | variable region |
| | SKNQFSLKLSSVTAADTAVYYCARD | |
| | RGDTILDVWGQGTMVTVSS | |
| 124 | GGSISSSSYY | Anti-BCMA CT103A |
| | | scFv heavy chain |
| | | CDR1 |
| 125 | ISYSGST | Anti-BCMA CT103A |
| | | scFv heavy chain |
| | | CDR2 |
| 126 | ARDRGDTILDV | Anti-BCMA CT103A |
| | | scFv heavy chain |
| | | CDR3 |

In some embodiments, the hinge domain of the BCMA CAR comprises a CD8α hinge domain, for example, a human CD8α hinge domain. In some embodiments, the CD8α hinge domain comprises or consists of an amino acid sequence set forth in SEQ ID NO:9 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:9. In some embodiments, the hinge domain comprises a CD28 hinge domain, for example, a human CD28 hinge domain. In some embodiments, the CD28 hinge domain comprises or consists of an amino acid sequence set forth in SEQ ID NO:10 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:10. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO:11 or SEQ ID NO:12, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:11 or SEQ ID NO:12. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO:13 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:13.

In some embodiments, the transmembrane domain of the BCMA CAR comprises a CD8α transmembrane domain, for example, a human CD8α transmembrane domain. In some embodiments, the CD8α transmembrane domain comprises or consists of an amino acid sequence set forth in SEQ ID NO:14 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:14. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO:15 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:15.

In some embodiments, the intracellular costimulatory domain of the BCMA CAR comprises a 4-1 BB costimulatory domain, for example, a human 4-1 BB costimulatory domain. In some embodiments, the 4-1 BB costimulatory domain comprises or consists of an amino acid sequence set forth in SEQ ID NO:16 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:16. In some embodiments, the intracellular costimulatory domain comprises a CD28 costimulatory domain, for example, a human CD28 costimulatory domain. In some embodiments, the CD28 costimulatory domain comprises or consists of an amino acid sequence set forth in SEQ ID NO:17 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:17.

In some embodiments, the intracellular signaling domain of the BCMA CAR comprises a CD3 zeta (ζ) signaling domain, for example, a human CD3ζ signaling domain. In some embodiments, the CD3ζ signaling domain comprises or consists of an amino acid sequence set forth in SEQ ID NO:18 or SEQ ID NO:115, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:18 or SEQ ID NO:115.

In some embodiments, the polycistronic vector comprises an expression cassette that contains a nucleotide sequence encoding a BCMA CAR, including, for example, a BCMA CAR comprising any of the BCMA-specific extracellular binding domains as described, the CD8α hinge domain of SEQ ID NO:9, the CD8α transmembrane domain of SEQ ID NO:14, the 4-1BB costimulatory domain of SEQ ID NO:16, the CD3ζ signaling domain of SEQ ID NO:18 or SEQ ID NO:115, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the BCMA CAR may additionally comprise a signal peptide (e.g., a CD8α signal peptide) as described.

In some embodiments, the polycistronic vector comprises an expression cassette that contains a nucleotide sequence encoding a BCMA CAR, including, for example, a BCMA CAR comprising any of the BCMA-specific extracellular binding domains as described, the CD8α hinge domain of SEQ ID NO:9, the CD8α transmembrane domain of SEQ ID NO:14, the CD28 costimulatory domain of SEQ ID NO:17, the CD3ζ signaling domain of SEQ ID NO:18 or SEQ ID NO:115, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the BCMA CAR may additionally comprise a signal peptide as described.

In some embodiments, the polycistronic vector comprises an expression cassette that contains a nucleotide sequence encoding a BCMA CAR as set forth in SEQ ID NO:127 or is at least 80% identical (e.g., at least 80%, at least 85%, at least 90%, at least 95%, at least 96%, at least 97%, at least 98%, at least 99%, or 100% identical) to the nucleotide sequence set forth in SEQ ID NO:127 (see Table 14). The encoded BCMA CAR has a corresponding amino acid sequence set forth in SEQ ID NO:128 or is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:128, with the following components: CD8α signal peptide, CT103A scFv (V_{L}-Whitlow linker-V_{H}), CD8α hinge domain, CD8α transmembrane domain, 4-1 BB costimulatory domain, and CD3ζ signaling domain.

In some embodiments, the polycistronic vector comprises an expression cassette that contains a nucleotide sequence encoding a commercially available embodiment of BCMA CAR, including, for example, idecabtagene vicleucel (ide-cel, also called bb2121). In some embodiments, the polycistronic vector comprises an expression cassette that contains a nucleotide sequence encoding idecabtagene vicleucel or portions thereof. Idecabtagene vicleucel comprises a BCMA CAR with the following components: the BB2121 binder, CD8α hinge domain, CD8α transmembrane domain, 4-1 BB costimulatory domain, and CD3 signaling domain.

TABLE 14
Exemplary sequences of BCMA CARs

| SEQ ID NO: | Sequence | Description |
|---|---|---|
| 127 | atggccttaccagtgaccgccttgctcctgccgctggcctt | Exemplary BCMA |
| | gctgctccacgccgccaggccggacatccagatgaccc | CAR nucleotide |
| | agtctccatcctccctgtctgcatctgtaggagacagagtc | sequence |
| | accatcacttgccgggcaagtcagagcattagcagctatt | |
| | taaattggtatcagcagaaaccagggaaagcccctaag | |
| | ctcctgatctatgctgcatccagtttgcaaagtggggtccc | |
| | atcaaggttcagtggcagtggatctgggacagatttcactc | |
| | tcaccatcagcagtctgcaacctgaagattttgcaacttac | |
| | tactgtcagcaaaaatacgacctcctcacttttggcggag | |
| | ggaccaaggttgagatcaaaggcagcaccagcggctc | |
| | cggcaagcctggctctggcgagggcagcacaaaggga | |
| | cagctgcagctgcaggagtcgggcccaggactggtgaa | |
| | gccttcggagaccctgtccctcacctgcactgtctctggtg | |
| | gctccatcagcagtagtagttactactggggctggatccg | |
| | ccagcccccagggaaggggctggagtggattgggagta | |
| | tctcctatagtgggagcacctactacaacccgtccctcaa | |
| | gagtcgagtcaccatatccgtagacacgtccaagaacc | |
| | agttctccctgaagctgagttctgtgaccgccgcagacac | |
| | ggcggtgtactactgcgccagagatcgtggagacaccat | |
| | actagacgtatggggtcagggtacaatggtcaccgtcag | |
| | ctcattcgtgcccgtgttcctgcccgccaaacctaccacca | |
| | cccctgcccctagacctcccaccccagccccaacaatcg | |
| | ccagccagcctctgtctctgcggcccgaagcctgtagac | |
| | ctgctgccggcggagccgtgcacaccagaggcctggac | |
| | ttcgcctgcgacatctacatctgggcccctctggccggca | |
| | cctgtggcgtgctgctgctgagcctggtgatcaccctgtact | |
| | gcaaccaccggaacaaacggggcagaaagaaactcc | |
| | tgtatatattcaaacaaccatttatgagaccagtacaaact | |
| | actcaagaggaagatggctgtagctgccgatttccagaa | |
| | gaagaagaaggaggatgtgaactgagagtgaagttca | |
| | gcagatccgccgacgcccctgcctaccagcagggaca | |
| | gaaccagctgtacaacgagctgaacctgggcagacgg | |
| | gaagagtacgacgtgctggacaagcggagaggccgg | |
| | gaccccgagatgggcggaaagcccagacggaagaac | |
| | ccccaggaaggcctgtataacgaactgcagaaagaca | |
| | agatggccgaggcctacagcgagatcggcatgaaggg | |
| | cgagcggaggcgcggcaagggccacgatggcctgtac | |
| | cagggcctgagcaccgccaccaaggacacctacgacg | |
| | ccctgcacatgcaggccctgccccccaga | |
| 128 | MALPVTALLLPLALLLHAARPDIQMTQSP | Exemplary BCMA |
| | SSLSASVGDRVTITCRASQSISSYLNWYQ | CAR amino acid |
| | QKPGKAPKLLIYAASSLQSGVPSRFSGSG | sequence |
| | SGTDFTLTISSLQPEDFATYYCQQKYDLL | |
| | TFGGGTKVEIKGSTSGSGKPGSGEGSTK | |
| | GQLQLQESGPGLVKPSETLSLTCTVSGG | |
| | SISSSSYYWGWIRQPPGKGLEWIGSISYS | |
| | GSTYYNPSLKSRVTISVDTSKNQFSLKLS | |
| | SVTAADTAVYYCARDRGDTILDVWGQGT | |
| | MVTVSSFVPVFLPAKPTTTPAPRPPTPAP | |
| | TIASQPLSLRPEACRPAAGGAVHTRGLDF | |
| | ACDIYIWAPLAGTCGVLLLSLVITLYCNHR | |
| | NKRGRKKLLYIFKQPFMRPVQTTQEEDG | |
| | CSCRFPEEEEGGCELRVKFSRSADAPAY | |
| | QQGQNQLYNELNLGRREEYDVLDKRRG | |
| | RDPEMGGKPRRKNPQEGLYNELQKDKM | |
| | AEAYSEIGMKGERRRGKGHDGLYQGLST | |
| | ATKDTYDALHMQALPPR | |

### Safety Switch

In certain embodiments, the polycistronic vector may comprise one or more expression cassettes each comprising a nucleotide sequence encoding a safety switch. A safety switch can be used in the polycistronic vector of the present technology to induce death or apoptosis of host cells containing the polycistronic vector, for example if the cells grow and divide in an undesired manner or cause excessive toxicity to the host. Thus, the use of safety switches enables one to conditionally eliminate aberrant cells in vivo and can be a critical step for the application of cell therapies in the clinic. Safety switches and their uses thereof are described in, for example, Düzgüne§, Origins of Suicide Gene Therapy (2019); Düzgüne§ (eds), Suicide Gene Therapy. Methods in Molecular Biology, vol. 1895 (Humana Press, New York, NY) (for HSVtk, cytosine deaminase, nitroreductase, purine nucleoside phosphorylase, and horseradish peroxidase); Zhou and Brenner, Exp Hematol 44(11):1013-1019 (2016) (for iCaspase9); Wang et al., Blood 18(5):1255-1263 (2001) (for huEGFR); U.S. Patent Application Publication No. 20180002397 (for HER1); and Philip et al., Blood124(8):1277-1287 (2014) (for RQR8).

In some embodiments, the safety switch can cause cell death in a controlled manner, for example, in the presence of a drug or prodrug or upon activation by a selective exogenous compound. In some embodiments, expression of the safety switch is regulated either by a promoter of the polycistronic vector, in the case of genomic location-independent transcriptional regulation, or by an endogenous promoter, in the case of site-specific integration of the construct into target gene locus.

In some embodiments, the safety switch comprises a “suicide gene” or “suicide switch”. The suicide gene can cause the death of the cells should they grow and divide in an undesired manner. The suicide gene may encode a protein that results in cell killing only when activated by a specific compound, for example, an enzyme that selectively converts a nontoxic compound into highly toxic metabolites.

In some embodiments, the safety switch of the polycistronic vector is selected from the group consisting of herpes simplex virus thymidine kinase (HSVtk), cytosine deaminase (CyD), nitroreductase (NTR), purine nucleoside phosphorylase (PNP), horseradish peroxidase, inducible caspase 9 (iCasp9), rapamycin-activated caspase 9 (rapaCasp9), CCR4, CD16, CD19, CD20, CD30, EGFR, GD2, HER1, HER2, MUC1, PSMA, and RQR8.

In some embodiments, the safety switch of the polycistronic vector may be a transgene encoding a product with cell killing capabilities when activated by a drug or prodrug, for example, by turning a non-toxic prodrug to a toxic metabolite inside the cell. In these embodiments, cell killing is activated by contacting a cell comprising the vector with the drug or prodrug. In some cases, the safety switch is HSVtk, which converts ganciclovir (GCV) to GCV-triphosphate, thereby interfering with DNA synthesis and killing dividing cells. In some cases, the safety switch is CyD or a variant thereof, which converts the antifungal drug 5-fluorocytosine (5-FC) to cytotoxic 5-fluorouracil (5-FU) by catalyzing the hydrolytic deamination of cytosine into uracil. 5-FU is further converted to potent anti-metabolites (5-FdUMP, 5-FdUTP, 5-FUTP) by cellular enzymes. These compounds inhibit thymidylate synthase and the production of RNA and DNA, resulting in cell death. In some cases, the safety switch is NTR or a variant thereof, which can act on the prodrug CB1954 via reduction of the nitro groups to reactive N-hydroxylamine intermediates that are toxic in proliferating and nonproliferating cells. In some cases, the safety switch is PNP or a variant thereof, which can turn prodrug 6-methylpurine deoxyriboside or fludarabine into toxic metabolites to both proliferating and nonproliferating cells. In some cases, the safety switch is horseradish peroxidase or a variant thereof, which can catalyze indole-3-acetic acid (IAA) to a potent cytotoxin and thus achieve cell killing.

In some embodiments, the safety switch of the polycistronic vector may be an iCasp9. Caspase 9 is a component of the intrinsic mitochondrial apoptotic pathway which, under physiological conditions, is activated by the release of cytochrome C from damaged mitochondria. Activated caspase 9 then activates caspase 3, which triggers terminal effector molecules leading to apoptosis. The iCasp9 may be generated by fusing a truncated caspase 9 (without its physiological dimerization domain or caspase activation domain) to a FK506 binding protein (FKBP), FKBP12-F36V, via a peptide linker. The iCasp9 has low dimer-independent basal activity and can be stably expressed in host cells (e.g., human T cells) without impairing their phenotype, function, or antigen specificity. However, in the presence of chemical inducer of dimerization (CID), such as rimiducid (AP1903), AP20187, and rapamycin, iCasp9 can undergo inducible dimerization and activate the downstream caspase molecules, resulting in apoptosis of cells expressing the iCasp9. See, e.g., PCT Application Publication No. WO2011/146862; Stasi et al., N. Engl. J. Med. 365; 18 (2011); Tey et al., Biol. Blood Marrow Transplant 13:913-924 (2007). In particular, the rapamycin-inducible caspase 9 variant is called rapaCasp9. See Stavrou et al., Mol. Ther. 26(5):1266-1276 (2018). Thus, iCasp9 can be used as a safety switch in the present polycistronic vector to achieve controlled killing of the host cells.

In some embodiments, the safety switch of the polycistronic vector may be a membrane-expressed protein which allows for cell depletion after administration of a specific antibody to that protein. Safety switches of this category may include, for example, CCR4, CD16, CD19, CD20, CD30, EGFR, GD2, HER1, HER2, MUC1, PSMA, or RQR8. These proteins may have surface epitopes that can be targeted by specific antibodies.

In some embodiments, the safety switch comprises CCR4, which can be recognized by an anti-CCR4 antibody. Non-limiting examples of suitable anti-CCR4 antibodies include mogamulizumab and biosimilars thereof.

In some embodiments, the safety switch comprises CD16 or CD30, which can be recognized by an anti-CD16 or anti-CD30 antibody. Non-limiting examples of such anti-CD16 or anti-CD30 antibody include AFM13 and biosimilars thereof.

In some embodiments, the safety switch comprises CD19, which can be recognized by an anti-CD19 antibody. Non-limiting examples of such anti-CD19 antibody include MOR208 and biosimilars thereof.

In some embodiments, the safety switch comprises CD20, which can be recognized by an anti-CD20 antibody. Non-limiting examples of such anti-CD20 antibody include obinutuzumab, ublituximab, ocaratuzumab, rituximab, rituximab-RLIb, and biosimilars thereof. Cells that express the safety switch are thus CD20-positive and can be targeted for killing through administration of an anti-CD20 antibody as described.

In some embodiments, the safety switch comprises EGFR, which can be recognized by an anti-EGFR antibody. Non-limiting examples of such anti-EGFR antibody include tomuzotuximab, RO5083945 (GA201), cetuximab, and biosimilars thereof.

In some embodiments, the safety switch comprises GD2, which can be recognized by an anti-GD2 antibody. Non-limiting examples of such anti-GD2 antibody include Hul4.18K322A, Hul4.18-IL2, Hu3F8, dinituximab, c.60C3-RLIc, and biosimilars thereof.

In some embodiments, the safety switch comprises HER1, which can be recognized by an anti-HER1 antibody. Non-limiting examples of such anti-HER1 antibody include cetuximab and biosimilars thereof.

In some embodiments, the safety switch comprises HER2, which can be recognized by an anti-HER2 antibody. Non-limiting examples of such anti-HER2 antibody include margetuximab, trastuzumab, TrasGEX, and biosimilars thereof.

In some embodiments, the safety switch comprises MUC1, which can be recognized by an anti-MUC1 antibody. Non-limiting examples of such anti-MUC1 antibody include gatipotuzumab and biosimilars thereof.

In some embodiments, the safety switch comprises PSMA, which can be recognized by an anti-PSMA antibody. Non-limiting examples of such anti-PSMA antibody include KM2812 and biosimilars thereof.

In some embodiments, the safety switch comprises RQR8, which can be recognized by an anti-RQR8 antibody. Non-limiting examples of such anti-RQR8 antibody include rituximab and biosimilars thereof.

In some embodiments, the safety switch comprises HSVtk and a membrane-expressed protein, for example, CCR4, CD16, CD19, CD20, CD30, EGFR, GD2, HER1, HER2, MUC1, PSMA, and RQR8.

In some embodiments, the safety switch comprises a therapeutic agent that recognizes one or more tolerogenic factor expressed on the surface of a modified cell. In some embodiments, the safety switch comprises a therapeutic agent that inhibits or blocks the interaction of CD47 and signal regulatory protein alpha (SIRPa), e.g., CD47-SIRPa blockade agent. SIRPa is a transmembrane receptor protein on circulating immune cells. When CD47 expressed by a foreign cell binds to SIRPa, it delivers an inhibitory “don't eat me” signal to a recipient's immune system and thus evades rejection by the recipient's immune system. Interruption of the CD47-SIRPα interaction would thus abolish the immuno-protective masking, resulting in elimination of the foreign cell by the recipient's immune system. In some embodiments, the CD47-SIRPα blockade agent is an agent that neutralizes, blocks, antagonizes, or interferes with the cell surface expression of CD47, SIRPa, or both. In some embodiments, the CD47-SIRPα blockade agent inhibits or blocks the interaction of CD47, SIRPa, or both. In some embodiments, a CD47-SIRPα blockade agent (e.g., a CD47-SIRPα blocking, inhibiting, reducing, antagonizing, neutralizing, or interfering agent) comprises an agent selected from a group that includes an antibody or fragment thereof that binds CD47, a bispecific antibody that binds CD47, an immunocytokine fusion protein that bind CD47, a CD47 containing fusion protein, an antibody or fragment thereof that binds SIRPα, a bispecific antibody that binds SIRPα, an immunocytokine fusion protein that bind SIRPα, an SIRPα containing fusion protein, or any combination thereof.

In some embodiments, the CD47-SIRPα blockade agent comprises a CD47-binding domain. In some embodiments, the CD47-binding domain comprises SIRPα or a fragment thereof. In some embodiments, the CD47-SIRPα blockade agent comprises an immunoglobulin G (IgG) Fc domain. In some embodiments, the IgG Fc domain comprises an IgG1 Fc domain. In some embodiments, the IgG1 Fc domain comprises a fragment of a human antibody. In some embodiments, the CD47-SIRPα blockade agent is selected from the group consisting of TTI-621, TTI-622, and ALX148. In some embodiments, the CD47-SIRPα blockade agent is TTI-621. In some embodiments, the CD47-SIRPα blockade agent is TTI-622. In some embodiments, the CD47-SIRPα blockade agent is ALX148. In some embodiments, the IgG Fc domain comprises an IgG4 Fc domain. In some embodiments, the CD47-SIRPα blockade agent is an antibody. In some embodiments, the antibody is selected from the group consisting of MIAP410, B6H12, and Magrolimab. In some embodiments, the antibody is MIAP410. In some embodiments, the antibody is B6H12. In some embodiments, the antibody is Magrolimab. In some embodiments, the antibody is selected from the group consisting of AO-176, IB1188 (letaplimab), STI-6643, and ZL-1201. In some embodiments, the antibody is AO-176 (Arch). In some embodiments, the antibody is IBI188 (letaplimab) (Innovent). In some embodiments, the antibody is STI-6643 (Sorrento). In some embodiments, the antibody is ZL-1201 (Zai).

In some embodiments, useful antibodies or fragments thereof that bind CD47 can be selected from a group that includes magrolimab ((Hu5F9-G4)) (Forty Seven, Inc.; Gilead Sciences, Inc.), urabrelimab, CC-90002 (Celgene; Bristol-Myers Squibb), IBI-188 (Innovent Biologics), IBI-322 (Innovent Biologics), TG-1801 (TG Therapeutics; also known as NI-1701, Novimmune SA), ALX148 (ALX Oncology), TJ011133 (also known as TJC4, I-Mab Biopharma), FA3M3, ZL-1201 (Zai Lab Co., Ltd), AK117 (Akesbio Australia Pty, Ltd.), AO-176 (Arch Oncology), SRF231 (Surface Oncology), GenSci-059 (GeneScience), C47B1157 (Janssen Research and Development), C47B1161 (Janssen Research and Development), C47B1167 (Janssen Research and Development), C47B222 (Janssen Research and Development), C47B227 (Janssen Research and Development), Vx-1004 (Corvus Pharmaceuticals), HMBD004 (Hummingbird Bioscience Pte Ltd), SHR-1603 (Hengrui), AMMS4-G4 (Beijing Institute of Biotechnology), RTX-CD47 (University of Groningen), and IMC-002. (Samsung Biologics; ImmuneOncia Therapeutics). In some embodiments, the antibody or fragment thereof does not compete for CD47 binding with an antibody selected from a group that includes magrolimab, urabrelimab, CC-90002, IBI-188, IBI-322, TG-1801 (NI-1701), ALX148, TJ011133, FA3M3, ZL1201, AK117, AO-176, SRF231, GenSci-059, C47B157, C47B161, C47B167, C47B222, C47B227, Vx-1004, HMBD004, SHR-1603, AMMS4-G4, RTX-CD47, and IMC-002. In some embodiments, the antibody or fragment thereof competes for CD47 binding with an antibody selected from magrolimab, urabrelimab, CC-90002, IBI-188, IBI-322, TG-1801 (NI-1701), ALX148, TJ011133, FA3M3, ZL1201, AK117, AO-176, SRF231, GenSci-059, C47B157, C47B161, C47B1167, C47B222, C47B227, Vx-1004, HMBD004, SHR-1603, AMMS4-G4, RTX-CD47, and IMC-002. In some embodiments, the antibody or fragment thereof that binds CD47 is selected from a group that includes a single-chain Fv fragment (scFv) against CD47, a Fab against CD47, a VHH nanobody against CD47, a DARPin against CD47, and variants thereof. In some embodiments, the scFv against CD47, a Fab against CD47, and variants thereof are based on the antigen binding domains of any of the antibodies selected from a group that includes magrolimab, urabrelimab, CC-90002, IBI-188, IBI-322, TG-1801 (NI-1701), ALX148, TJ011133, FA3M3, ZL1201, AK117, AO-176, SRF231, GenSci-059, C47B157, C47B161, C47B167, C47B222, C47B227, Vx-1004, HMBD004, SHR-1603, AMMS4-G4, RTX-CD47, and IMC-002. Additional information regarding CD47-SIRPα blockade agents can be found in PCT Application Publication No. WO2022076928, the entire contents of which are incorporated herein by reference.

In certain embodiments, the polycistronic vector may comprise one or more expression cassettes each comprising a nucleotide sequence encoding a transgene of interest for use in cell therapy, for example, cancer therapy. In any of these embodiments, the polycistronic vector may be designed to enable co-expression of one or more tolerogenic factors, one or more CARs, one or more safety switches, and/or one or more other transgenes of interest for cell therapy, with the expression cassettes separated by one or more cleavage sites as described.

### Specific Exemplary Embodiments

In some aspects, provided is a polycistronic vector comprising (a) a first expression cassette comprising a nucleotide sequence encoding CD47, (b) a second expression cassette comprising a nucleotide sequence encoding CD19 CAR, and (c) a 2A site separating the first expression cassette and the second expression cassette. In some aspects, provided is a polycistronic vector comprising (a) a first expression cassette comprising a nucleotide sequence encoding CD47, (b) a second expression cassette comprising a nucleotide sequence encoding CD19 CAR, and (c) a furin site and a 2A site separating the first expression cassette and the second expression cassette, wherein the furin site precedes the 2A site in the 5′ to 3′ order. In some embodiments, the polycistronic vector further comprises (d) a third expression cassette comprising a nucleotide sequence encoding a safety switch, wherein the third expression cassette is separated from the first expression cassette and/or the second expression cassette by a 2A site, or a 2A and a furin site.

In some aspects, provided is a polycistronic vector comprising (a) a first expression cassette comprising a nucleotide sequence encoding CD47, (b) a second expression cassette comprising a nucleotide sequence encoding CD20 CAR, and (c) a 2A site separating the first expression cassette and the second expression cassette. In some aspects, provided is a polycistronic vector comprising (a) a first expression cassette comprising a nucleotide sequence encoding CD47, (b) a second expression cassette comprising a nucleotide sequence encoding CD20 CAR, and (c) a furin site and a 2A site separating the first expression cassette and the second expression cassette, wherein the furin site precedes the 2A site in the 5′ to 3′ order. In some embodiments, the polycistronic vector further comprises (d) a third expression cassette comprising a nucleotide sequence encoding a safety switch, wherein the third expression cassette is separated from the first expression cassette and/or the second expression cassette by a 2A site, or a 2A and a furin site.

In some aspects, provided is a polycistronic vector comprising (a) a first expression cassette comprising a nucleotide sequence encoding CD47, (b) a second expression cassette comprising a nucleotide sequence encoding CD22 CAR, and (c) a 2A site separating the first expression cassette and the second expression cassette. In some aspects, provided is a polycistronic vector comprising (a) a first expression cassette comprising a nucleotide sequence encoding CD47, (b) a second expression cassette comprising a nucleotide sequence encoding CD22 CAR, and (c) a furin site and a 2A site separating the first expression cassette and the second expression cassette, wherein the furin site precedes the 2A site in the 5′ to 3′ order. In some embodiments, the polycistronic vector further comprises (d) a third expression cassette comprising a nucleotide sequence encoding a safety switch, wherein the third expression cassette is separated from the first expression cassette and/or the second expression cassette by a 2A site, or a 2A and a furin site.

In some aspects, provided is a polycistronic vector comprising (a) a first expression cassette comprising a nucleotide sequence encoding CD47, (b) a second expression cassette comprising a nucleotide sequence encoding CD19 CAR, (c) a third expression cassette comprising a nucleotide sequence encoding CD22 CAR, and (d) a 2A site, or a 2A and a furin site, separating any two neighboring expression cassettes. In some embodiments, the polycistronic vector further comprises (e) a fourth expression cassette comprising a nucleotide sequence encoding a safety switch, wherein the fourth expression cassette is separated from the first expression cassette, the second expression cassette, and/or the third expression cassette by a 2A site, or a 2A and a furin site.

In some aspects, provided is a polycistronic vector comprising (a) a first expression cassette comprising a nucleotide sequence encoding CD47, (b) a second expression cassette comprising a nucleotide sequence encoding BCMA CAR, and (c) a 2A site separating the first expression cassette and the second expression cassette. In some aspects, provided is a polycistronic vector comprising (a) a first expression cassette comprising a nucleotide sequence encoding CD47, (b) a second expression cassette comprising a nucleotide sequence encoding BCMA CAR, and (c) a furin site and a 2A site separating the first expression cassette and the second expression cassette, wherein the furin site precedes the 2A site in the 5′ to 3′ order. In some embodiments, the polycistronic vector further comprises (d) a third expression cassette comprising a nucleotide sequence encoding a safety switch, wherein the third expression cassette is separated from the first expression cassette and/or the second expression cassette by a 2A site, or a 2A and a furin site.

In some aspects, provided is a polycistronic vector comprising (a) a first expression cassette comprising a nucleotide sequence encoding CD47, (b) a second expression cassette comprising a nucleotide sequence encoding a safety switch, and (c) a 2A site, or a 2A and a furin site, separating the first expression cassette and the second expression cassette.

In some embodiments, the polycistronic vector includes a first expression cassette comprising a nucleotide sequence encoding CD47 and a second expression cassette comprising a nucleotide sequence encoding CD19 CAR separated by one or more cleavage sites as described (e.g., a 2A site, a 2A and a furin site). The 5′ to 3′ order of the first and second expression cassettes may be reversed, i.e., the CD47 gene may either precede or follow the CD19 CAR gene. However, as shown in the examples, placing the CD47 gene before the CD19 CAR gene in the 5′ to 3′ order may increase expression of CD47 to improve immune protection of the host cell from innate immune cell killing. In certain of these embodiments, the polycistronic vector may additionally include a third expression cassette comprising a nucleotide sequence encoding a safety switch separated from the first and/or the second expression cassette by one or more cleavage sites as described (e.g., a 2A site, a 2A and a furin site). The placement of the safety switch can be varied, for example, before the CD47 and the CD19 CAR cassettes, in between the CD47 and the CD19 CAR cassettes, or after the CD47 and the CD219 CAR cassettes in the 5′ to 3′ order.

In some embodiments, the polycistronic vector includes a first expression cassette comprising a nucleotide sequence encoding CD47 and a second expression cassette comprising a nucleotide sequence encoding CD20 CAR separated by one or more cleavage sites as described (e.g., a 2A site, a 2A and a furin site). In certain of these embodiments, the polycistronic vector may additionally include a third expression cassette comprising a nucleotide sequence encoding a safety switch separated from the first and/or the second expression cassette by one or more cleavage sites as described (e.g., a 2A site, a 2A and a furin site). The placement of the safety switch can be varied, for example, before the CD47 and the CD20 CAR cassettes, in between the CD47 and the CD20 CAR cassettes, or after the CD47 and the CD20 CAR cassettes in the 5′ to 3′ order.

In some embodiments, the polycistronic vector includes a first expression cassette comprising a nucleotide sequence encoding CD47 and a second expression cassette comprising a nucleotide sequence encoding CD22 CAR separated by one or more cleavage sites as described (e.g., a 2A site, a 2A and a furin site). In certain of these embodiments, the polycistronic vector may additionally include a third expression cassette comprising a nucleotide sequence encoding a safety switch separated from the first and/or the second expression cassette by one or more cleavage sites as described (e.g., a 2A site, a 2A and a furin site). The placement of the safety switch can be varied, for example, before the CD47 and the CD22 CAR cassettes, in between the CD47 and the CD22 CAR cassettes, or after the CD47 and the CD22 CAR cassettes in the 5′ to 3′ order.

In some embodiments, the polycistronic vector includes a first expression cassette comprising a nucleotide sequence encoding CD47, a second expression cassette comprising a nucleotide sequence encoding CD22 CAR, and a third expression cassette comprising a nucleotide sequence encoding CD19 CAR, all of which are separated from one another by one or more cleavage sites as described (e.g., a 2A site, a 2A and a furin site). Such a polycistronic vector allows for simultaneous expression of two CARs (i.e., CD22 CAR and CD19 CAR) in a host cell. To minimize the risk of recombination due to sequence similarities, the CD22 CAR and the CD19 CAR may comprise one or more different non-antigen binding domains, for example, the CARs may incorporate different hinge domains, transmembrane domains, costimulatory domains, and/or intracellular signaling domains. As one non-limiting example, one CAR may comprise one transmembrane domain (e.g., CD28 transmembrane domain) while the other CAR comprises a different transmembrane domain (e.g., CD8α transmembrane domain). As another non-limiting example, one CAR may comprise one costimulatory domain (e.g., 4-1 BB costimulatory domain) while the other CAR comprises a different costimulatory domain (e.g., CD28 costimulatory domain). Or, alternatively, the CD22 CAR and the CD19 CARs may comprise the same non-antigen binding domains but have codon divergence introduced at the nucleotide sequence level to minimize the risk of recombination. In certain of these embodiments, the polycistronic vector may additionally include a fourth expression cassette comprising a nucleotide sequence encoding a safety switch separated from the first, the second, and/or the third expression cassette by one or more cleavage sites as described (e.g., a 2A site, a 2A and a furin site). The placement of the safety switch can be varied, for example, before all other expression cassettes, in between two expression cassettes, or after all other expression cassettes in the 5′ to 3′ order.

In some embodiments, the polycistronic vector includes a first expression cassette comprising a nucleotide sequence encoding CD47 and a second expression cassette comprising a nucleotide sequence encoding BCMA CAR separated by one or more cleavage sites as described (e.g., a 2A site, a 2A and a furin site). In certain of these embodiments, the polycistronic vector may additionally include a third expression cassette comprising a nucleotide sequence encoding a safety switch separated from the first and/or the second expression cassette by one or more cleavage sites as described (e.g., a 2A site, a 2A and a furin site). The placement of the safety switch can be varied, for example, before the CD47 and the BCMA CAR cassettes, in between the CD47 and the BCMA CAR cassettes, or after the CD47 and the BCMA CAR cassettes in the 5′ to 3′ order.

In some embodiments, the polycistronic vector includes a first expression cassette comprising a nucleotide sequence encoding CD47 and a second expression cassette comprises a nucleotide sequence encoding a safety switch separated by one or more cleavage sites as described (e.g., a 2A site, a 2A and a furin site). The safety switch can either precede or follow the CD47 in the 5′ to 3′ order.

In some embodiments, the polycistronic vector includes a first expression cassette comprising a nucleotide sequence encoding CD47 and a second expression cassette comprises a nucleotide sequence encoding a safety switch separated by one or more cleavage sites as described (e.g., a 2A site, a 2A and a furin site), wherein the safety switch comprises HSVtk and a membrane-expressed protein (e.g., CCR4, CD16, CD19, CD20, CD30, EGFR, GD2, HER1, HER2, MUC1, PSMA, RQR8, and a CD47-SIRPα blockade agent). In certain of these embodiments, the CD47 and safety switch transgenes are flanked by homology arms for use in site-directed insertion (knock-in) into specified loci in a host cell, for example, by homology directed repair (HDR)-based approaches as described. For example, the transgenes can be flanked by CLYBL left homology arm (LHA) and right homology arm (RHA)) to be inserted into the CLYBL locus of a host cell, for example, a β islet cell or glial progenitor cell (GPC).

### Cleavage Site

In certain embodiments, the two or more expression cassettes of the polycistronic vector of the present technology may be separated by one or more cleavage sites. As the name suggests, a polycistronic vector allows simultaneous expression of two or more separate proteins from one mRNA transcript in a host cell. Cleavage sites can be used in the design of a polycistronic vector to achieve such co-expression of multiple genes.

In some embodiments, the one or more cleavage sites comprise one or more self-cleaving sites. In some embodiments, the self-cleaving site comprises a 2A site. 2A peptides are a class of 18-22 amino acid-long peptides first discovered in picornaviruses and can induce ribosomal skipping during translation of a protein, thus producing equal amounts of multiple genes from the same mRNA transcript. 2A peptides function to “cleave” an mRNA transcript by making the ribosome skip the synthesis of a peptide bond at the C-terminus, between the glycine (G) and proline (P) residues, leading to separation between the end of the 2A sequence and the next peptide downstream. There are four 2A peptides commonly employed in molecular biology, T2A, P2A, E2A, and F2A, the sequences of which are summarized in Table 15. A glycine-serine-glycine (GSG) linker is optionally added to the N-terminal of a 2A peptide to increase cleavage efficiency. The use of “( )” around a sequence in the present disclosure means that the enclosed sequence is optional.

TABLE 15
Sequences of 2A peptides

| SEQ | | 2A |
|---|---|---|
| ID NO: | Amino Acid Sequence | Peptide |
| 85 | (GSG)EGRGSLLTCGDVEENPGP | T2A |
| 86 | (GSG)ATNFSLLKQAGDVEENPGP | P2A |
| 87 | (GSG)QCTNYALLKLAGDVESNPGP | E2A |
| 88 | (GSG)VKQTLNFDLLKLAGDVESNPGP | F2A |

In some embodiments, the one or more cleavage sites additionally comprise one or more protease sites. The one or more protease sites can either precede or follow the self-cleavage sites (e.g., 2A sites) in the 5′ to 3′ order of the polycistronic vector. The protease site may be cleaved by a protease after translation of the full transcript or after translation of each expression cassette such that the first expression product is released prior to translation of the next expression cassette. In these embodiments, having a protease site in addition to the 2A site, especially preceding the 2A site in the 5′ to 3′ order, may reduce the number of extra amino acid residues attached to the expressed proteins of interest. In some embodiments, the protease site comprises a furin site, also known as a Paired basic Amino acid Cleaving Enzyme (PACE) site. There are at least three furin cleavage sequences, FC1, FC2, and FC3, the amino acid sequences of which are summarized in Table 16. Similar to the 2A sites, one or more optional glycine-serine-glycine (GSG) sequences can be included for cleavage efficiency.

TABLE 16
Sequences of furin sites

| SEQ ID NO: | Amino Acid Sequence | Furin site |
|---|---|---|
| 89 | RRRR(GSG) | FC1 |
| 90 | RKRR(GSG) | FC2 |
| 91 | RKRR(GSG)TPDPW(GSG) | FC3 |

In some embodiments, the one or more cleavage sites comprise one or more self-cleaving sites, one or more protease sites, and/or any combination thereof. For example, the cleavage site can include a 2A site alone. For another example, the cleavage site can include a FC2 or FC3 site, followed by a 2A site. In these embodiments, the one or more self-cleaving sites may be the same or different. Similarly, the one or more protease sites may be the same or different.

### Vector and Compositions Thereof

In some embodiments, the vector used for polycistronic expression of two or more proteins of the present technology can be any type of vector suitable for introduction of nucleotide sequences into a host cell, including, for example, plasmids, adenoviral vectors, adeno-associated viral vectors, retroviral vectors, lentiviral vectors, phages, and homology-directed repair (HDR)-based donor vectors.

In some embodiments, the construct comprising the expression cassettes and the cleavage site according to various embodiments of the present technology may be operatively linked to certain regulatory elements of the vector. As known to a skilled artisan, expression vectors are typically engineered to contain polynucleotide sequences that are needed to affect the expression and processing of coding sequences to which they are operatively linked. Expression control sequences may include appropriate transcription initiation, termination, promoter, and enhancer sequences; efficient RNA processing signals, such as splicing and polyadenylation signals; sequences that stabilize cytoplasmic mRNA; sequences that enhance translation efficiency; sequences that enhance protein stability; and possibly sequences that enhance protein secretion. Expression control sequences may be operatively linked if they are contiguous with the gene of interest and expression control sequences that act in trans or at a distance to control the gene of interest.

In some embodiments, the promoter is one that drives constitutive gene expression in mammalian cells. Those frequently used include, for example, elongation factor 1 alpha (EF1a) promoter, cytomegalovirus (CMV) immediate-early promoter (Greenaway et al., Gene 18: 355-360 (1982)), simian vacuolating virus 40 (SV40) early promoter (Fiers et al., Nature 273:113-120 (1978)), spleen focus-forming virus (SFFV) promoter, phosphoglycerate kinase (PGK) promoter (Adra et al., Gene 60(1):65-74 (1987)), human beta actin promoter, polyubiquitin C gene (UBC) promoter, and CAG promoter (Nitoshi et al., Gene 108:193-199 (1991)). An example of a promoter that is capable of expressing a CAR transgene in a mammalian cell (e.g., a T cell) is the EF1α promoter. The native EF1α promoter drives expression of the alpha subunit of the elongation factor-1 complex, which is responsible for the enzymatic delivery of aminoacyl tRNAs to the ribosome. The EF1α promoter has been extensively used in mammalian expression plasmids and has been shown to be effective in driving CAR expression from transgenes cloned into a lentiviral vector. See, e.g., Milone et al., Mol. Ther. 17(8):1453-1464 (2009).

In some embodiments, the promoter is an inducible promoter. Unlike constitutive promoters, inducible promoters can switch between an on and an off state in response to certain stimuli (e.g., chemical agents, temperature, light) and can be regulated in tissue- or cell-specific manners. Non-limiting examples of frequently used inducible promoters include the tetracycline On (Tet-On) system and the tetracycline Off (Tet-Off) system, which utilize tetracycline response elements (TRE) placed upstream of a minimal promoter (e.g., CMV promoter) (Gossen & Bujard, Proc. Natl. Acad. Sci. USA 89(12):5547-5551 (1992)). The TRE is made of 7 repeats of a 19-nucleotide tetracycline operator (tetO) sequence and can be recognized by the tetracycline repressor (tetR). In the Tet-Off system, a tetracycline-controlled transactivator (tTA) was developed by fusing the tetR with the activating domain of virion protein 16 of herpes simplex virus. In the absence of tetracycline or its analogs (e.g., doxycycline), the tTA will bind the tetO sequences of the TRE and drives expression; in the presence of tetracycline, the rTA will bind to tetracycline and not to the TRE, resulting in reduced gene expression. Conversely, in the Tet-On system, a reverse transactivator (rtTA) was generated by mutagenesis of amino acid residues important for tetracycline-dependent repression, and the rtTA binds at the TRE and drives gene expression in the presence of tetracycline or doxycycline (Gossen et al., Science 268(5218):1766-1769 (1995)). Other examples of inducible promoters include, for example, AlcA, LexA, and Cre.

In some embodiments, the polycistronic vector comprises a Kozak consensus sequence before the first expression cassette. A Kozak consensus sequence is a nucleic acid motif that functions as the protein translation initiation site in most eukaryotic mRNA transcripts and mediates ribosome assembly and translation initiation. In some embodiments, the Kozak consensus sequence comprises or consists of the sequence set forth in SEQ ID NO:92, wherein r is a purine (i.e., a or g): (gcc)gccrccatgg (SEQ ID NO:92).

In some embodiments, the polycistronic vector comprises a Woodchuck Hepatitis Virus (WHV) Posttranscriptional Regulatory Element (WPRE) after the second expression cassette. A WPRE is a DNA sequence that, when transcribed, creates a tertiary structure enhancing expression. The WPRE sequence is commonly used to increase expression of genes delivered by viral vectors. In some embodiments, the WPRE sequence comprises or consists of an amino acid sequence set forth in SEQ ID NO:93 or an amino acid sequence that is at least 80% identical (e.g., at least 80%, at least 85%, at least 90%, at least 95%, at least 96%, at least 97%, at least 98%, at least 99%, or 100% identical) to the sequence set forth in SEQ ID NO:93:

(SEQ ID NO: 93)
aatcaacctctggattacaaaatttgtgaaagattgactggtattctta
actatgttgctccttttacgctatgtggatacgctgctttaatgccttt
gtatcatgctattgcttcccgtatggctttcattttctcctccttgtat
aaatcctggttgctgtctctttatgaggagttgtggcccgttgtcaggc
aacgtggcgtggtgtgcactgtgtttgctgacgcaacccccactggttg
gggcattgccaccacctgtcagctcctttccgggactttcgctttcccc
ctccctattgccacggeggaactcatcgccgcctgccttgcccgctgct
ggacaggggctcggctgttgggcactgacaattccgtggtgttgtcggg
gaaatcatcgtcctttccttggctgctcgcctgtgttgccacctggatt
ctgcgcgggacgtccttctgctacgtcccttcggccctcaatccagcgg
accttccttcccgcggcctgctgccggctctgcggcctcttccgcgtct
tcgccttcgccctcagacgagtcggatctccctttgggccgcctccccg
c.

In some embodiments, the polycistronic vector comprises homology arms flanking a fragment containing the expression cassettes and/or promoter for use in site-directed insertion (knock-in) into specified loci in a host cell, for example, by homology directed repair (HDR)-based approaches as described. A fragment of the polycistronic vector to be inserted, usually containing at least the expression cassettes and optionally also containing the promoter, would be flanked by homologous sequence immediately upstream and downstream of the target insertion site (i.e., left homology arm (LHA) and right homology arm (RHA)). The homology arms are specifically designed for the target genomic locus for the fragment to serve as a template for HDR. The length of each homology arm is generally dependent on the size of the insert being introduced, with larger insertions requiring longer homology arms.

In some aspects, the present technology provides compositions comprising a polycistronic vector according to various embodiments disclosed herein.

In some embodiments, the composition may comprise one polycistronic vector according to one embodiment disclosed herein. In some embodiments, the composition may comprise a mixture of two or more polycistronic vectors according to various embodiments disclosed herein. In these embodiments, the two or more polycistronic vectors are different, e.g., contain expression cassettes encoding different proteins of interest (e.g., different CARs), and the composition may be used for transfection or transduction to generate a heterogenous population of host cells. By a non-limiting example, the composition may comprise two polycistronic vectors, one encoding CD47 and CD19 CAR, and the other encoding CD47 and CD22 CAR.

In some embodiments, the composition may further comprise one or more pharmaceutically acceptable carriers, excipients, preservatives, or a combination thereof. A “pharmaceutically acceptable carrier or excipient” refers to a pharmaceutically acceptable material, composition, or vehicle that is involved in carrying or transporting a compound of interest from one tissue, organ, or portion of the body to another tissue, organ, or portion of the body. For example, the carrier or excipient may be a liquid or solid filler, diluent, excipient, solvent, or encapsulating material, or some combination thereof. Each component of the carrier or excipient must be “pharmaceutically acceptable,” in that it must be compatible with the other ingredients of the formulation. It also must be suitable for contact with any tissue, organ, or portion of the body that it may encounter, meaning that it must not carry a risk of toxicity, irritation, allergic response, immunogenicity, or any other complication that excessively outweighs its therapeutic benefits. Suitable excipients include water, saline, dextrose, glycerol, or the like and combinations thereof. In some embodiments, compositions comprising host cells as disclosed herein further comprise a suitable infusion media.

### Viruses and Compositions Thereof

In some aspects, the present technology provides a virus comprising a polycistronic vector according to various embodiments disclosed herein for transducing a host cell. In certain embodiments, the polycistronic vector of the present technology can be packaged in the form of a virus or into a virus for host cell transduction. The virus can be any type of virus suitable for transducing a host cell and introducing nucleotide sequences into the host cell, including, for example, adenoviruses, adeno-associated viruses, retroviruses, lentiviruses, and phages.

In some aspects, the present technology provides a composition comprising a virus according to various embodiments disclosed herein. In some embodiments, the composition may comprise a polycistronic vector packaged into a virus according to one embodiment disclosed herein. In some embodiments, the composition may comprise a mixture of two or more viruses according to various embodiments disclosed herein. For example, the composition may comprise a first polycistronic vector packaged into a first virus and a second polycistronic vector packaged into a second virus. In any of these embodiments, the two or more viruses each have a polycistronic vector packaged into the first that is different from each other, e.g., contains expression cassettes encoding different proteins of interest (e.g., different CARs), and the composition may be used to transduce host cells to generate a heterogenous population. By a non-limiting example, the composition may comprise two viruses, one containing a polycistronic vector encoding CD47 and CD19 CAR, the other containing a polycistronic vector encoding CD47 and CD22 CAR. The composition may then be used to transduce host cells to generate a mixture of cells, some expressing CD47 and CD19 CAR, some expressing CD47 and CD22 CAR, and some expressing CD47, CD19 CAR, and CD22 CAR.

### Methods of Generating, Host Cells, and Compositions Thereof

### Methods of Generating Host Cells

In some aspects, the present technology provides methods for generating a population of host cells that contain the polycistronic vector according to various embodiments disclosed herein for cell therapy.

In some embodiments, the method comprises introducing a polycistronic vector according to various embodiments of the present technology, or a composition or a virus containing the same, into a population of host cells for use in adoptive cell therapy. Host cells may be transformed to incorporate the polycistronic vector by any known method in the field, including, for example, viral transduction, calcium phosphate transfection, lipid-mediated transfection, DEAE-dextran, electroporation, microinjection, nucleoporation, liposomes, nanoparticles, or other methods. For example, the polycistronic vector in the form of a viral vector or virus as described may be used to transduce a population of host cells. The transformed host cells can be collected and/or screened using known techniques, and the various subpopulations or combinations thereof can be enriched or depleted by known techniques, such as by affinity binding to antibodies, flow cytometry, fluorescence activated cell sorting (FACS), or immunomagnetic selection. After introduction into the host cell, the polycistronic vector or fragments thereof can be integrated into the genome of the host cell either through random insertion or through site-directed insertion (knock-in) as described.

In some embodiments, the population of host cells generated by the methods as described has at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% of the host cells in the population having the polycistronic vector incorporated into the cells. In some embodiments, the population of host cells generated by methods according to various embodiments of the present technology has at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% of the host cells in the population expressing one or more proteins of interest encoded by the polycistronic vector.

In certain of these embodiments, the method may further comprise introducing a second polycistronic vector according to various embodiments of the present technology, or a composition or a virus containing the same, into the population of host cells. This step of introducing a second polycistronic vector, or a composition or a virus containing the same, may occur at the same time as or after the step of introducing the first polycistronic vector, or a composition or a virus containing the same, into the host cells. In these embodiments, the first and second polycistronic vectors may be different, e.g., contain expression cassettes encoding different proteins of interest (e.g., different CARs), and the generated host cells may be a heterogenous population. By a non-limiting example, a first polycistronic vector encoding CD47 and CD19 CAR and a second polycistronic vector encoding CD47 and CD22 CAR can be used sequentially or in combination to transform host cells to generate a heterogenous population where some cells express CD47 and CD19 CAR, some cells express CD47 and CD22 CAR, and some other cells express CD47, CD19, and CD22 CAR. In some embodiments, the population of heterogenous host cells generated by the methods as described has at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% of the host cells in the population having the second polycistronic vector incorporated into the cells. In some embodiments, the population of heterogenous host cells generated by the methods as described has at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% of the host cells in the population expressing one or more proteins of interest encoded by the second polycistronic vector. In certain of these embodiments, the method may further comprise a step of sorting or isolating a sub-population of the generated heterogenous population of host cells where at least 50%, at least 55%, at least 60%, at least 65%, at least 70%, at least 75%, at least 80%, at least 85%, at least 90%, at least 95%, or at least 100% of the host cells in the sub-population have both the first and second polycistronic vectors incorporated into the cells, or least 50%, at least 55%, at least 60%, at least 65%, at least 70%, at least 75%, at least 80%, at least 85%, at least 90%, at least 95%, or at least 100% of the host cells in the sub-population express the proteins of interest encoded by both the first and second polycistronic vectors.

In some embodiments, the method comprises introducing a mixture or combination of two or more polycistronic vectors according to various embodiments of the present technology, or a composition containing the same, into the population of host cells. In these embodiments, the two or more polycistronic vectors may be different, e.g., contain expression cassettes encoding different proteins of interest (e.g., different CARs), and the generated host cells may be a heterogenous population. The mixture of polycistronic vectors may be in the form of a mixture of viral vectors or viruses as described. By a non-limiting example, a mixture of two polycistronic vectors, one encoding CD47 and CD19 CAR and the other encoding CD47 and CD22 CAR, can be used to transform host cells to generate a heterogenous population where some cells express CD47 and CD19 CAR, some cells express CD47 and CD22 CAR, and some cells express CD47, CD19, and CD22 CAR. In some embodiments, the population of heterogenous host cells generated by the methods as described has at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% of the host cells in the population having at least one of the two of more polycistronic vectors incorporated into the cells. In some embodiments, the population of heterogenous host cells generated by the methods as described has at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% of the host cells in the population expressing the proteins of interest encoded by at least one of the two or more polycistronic vectors. In certain of these embodiments, the method may further comprise a step of sorting or isolating a sub-population of the generated heterogenous population of host cells where at least 50%, at least 55%, at least 60%, at least 65%, at least 70%, at least 75%, at least 80%, at least 85%, at least 90%, at least 95%, or at least 100% of the host cells in the sub-population have the two or more polycistronic vectors incorporated into the cells, or least 50%, at least 55%, at least 60%, at least 65%, at least 70%, at least 75%, at least 80%, at least 85%, at least 90%, at least 95%, or at least 100% of the host cells in the sub-population express the proteins of interest encoded by the two or more polycistronic vectors.

In any of these embodiments, the method may further comprise performing additional modifications of the host cells to reduce the immunogenicity of these cells. In cell therapy, when the host cells are allogeneic, i.e., derived from a person other than the recipient, additional modifications are needed to reduce potential graft-versus-host risks after infusion into the recipient or risks of being eliminated by the recipient's innate immune system. In some embodiments, the additional modifications comprise reducing or eliminating the expression of major histocompatibility complex (MHC) class I and/or II (MHC I and/or MHC II) molecules in the host cells. This step of modifying MHC 1 and/or MHC II molecules may occur before, at the same time as, or after the step of introducing a first polycistronic vector or a composition containing the same, and/or the step of the introducing a second polycistronic vector or a composition containing the same into the host cells.

MHC I and/or MHC II genes encode cell surface molecules specialized to present antigenic peptides to immune cells. Reduced expression of MHC I and/or MHC II in allogeneic cells may prevent recognition of these cells by the immune cells of the recipient and thus rejection of the graft. The MHC in humans is called human leukocyte antigen (HLA). Class I HLA (HLA I) corresponding to MHC I include the HLA-A, HLA-B, and HLA-C genes, and Class II HLA (HLA I) corresponding to MHC II include the HLA-DR, HLA-DQ, HLA-DP, HLA-DM, and HLA-DO genes.

In some embodiments, the additional modifications of the host cells to reduce the immunogenicity of these cells comprise genetically modifying the cells to reduce expression of one or more immune factors, including, for example, CIITA, β₂ microglobulin (B2M), NLRC5, CTLA-4, PD-1, HLA-A, HLA-BM, HLA-C, RFX-ANK, NFY-A, RFX5, RFX-AP, NFY-B, NFY-C, IRF1, MIC-A, MIC-B, and TAP1.

In some embodiments, the allogeneic host cells may be modified to have reduced expression of MHC I genes by targeting and modulating one or more of the HLA loci individually, such as HLA-A, HLA-B, and/or HLA-C, or collectively with HLA-Razor. In some embodiments, the modulation occurs through insertion-deletion (indel) modifications of one of more of the HLA loci, including HLA-A, HLA-B, and/or HLA-C, for example, by using the CRISPR/Cas system as described. By modulating (e.g., reducing or deleting) expression of any of the HLA genes, the cell is can be rendered hypoimmunogenic and have a reduced ability to induce an immune response in a recipient subject. In some embodiments, reduced expression of any of the HLA loci reduces or eliminates expression of one or more of the HLA-A, HLA-B, and HLA-C genes. In some embodiments, the host cell has HLA-A, HLA-B, and/or HLA-C knockout. In some embodiments, the genetic modification targeting any of the HLA loci comprises inserting an exogenous nucleic acid encoding a polypeptide as disclosed herein (e.g., a polycistronic vector or a fragment thereof) at the HLA locus, according to embodiments disclosed herein. In certain of these embodiments, insertion of the polycistronic vector or fragment thereof into any of the HLA loci results in HLA-A, HLA-B, and/or HLA-C knockout.

In some embodiments, the allogeneic host cells may be modified to have reduced expression of MHC I genes by targeting and modulating the B2M locus. The B2M gene encodes a component of MHC I molecules. In some embodiments, the modulation occurs through insertion-deletion (indel) modifications of the B2M locus, for example, by using the CRISPR/Cas system as described. By modulating (e.g., reducing or deleting) expression of B2M, surface trafficking of MHC I molecules is blocked, and the cell is thus rendered hypoimmunogenic. In some embodiments, the allogeneic host cell modified to have reduced expression of MHC I genes has a reduced ability to induce an immune response in a recipient subject. In some embodiments, reduced expression of B2M reduces or eliminates expression of one or more of the HLA-A, HLA-B, and HLA-C genes. In some embodiments, the host cell has B2M knockout. In some embodiments, the genetic modification targeting the B2M locus comprises inserting an exogenous nucleic acid encoding a polypeptide as disclosed herein (e.g., a polycistronic vector or a fragment thereof) at the B2M locus, according to embodiments disclosed herein. In certain of these embodiments, insertion of the polycistronic vector or fragment thereof into the B2M locus results in B2M knockout.

In some embodiments, the allogeneic host cells may be modified to have reduced expression of MHC I genes by targeting and modulating the TAP1 locus. TAP1 encoded by the TAP1 gene assembles with TAP2 encoded by the TAP2 gene to form the transporter associated with antigen processing (TAP) complex, which is found in the endoplasmic reticulum (ER) and transports peptides of foreign origin into the ER to be attached to MHC class I proteins for presentation on the cell surface to the immune system. In some embodiments, the modulation occurs through insertion-deletion (indel) modifications of the TAP1 locus, for example, by using the CRISPR/Cas system as described. By modulating (e.g., reducing or deleting) expression of TAP1, surface trafficking of MHC I molecules is blocked, and the cell is thus rendered hypoimmunogenic. In some embodiments, reduced expression of TAP1 reduces or eliminates expression of one or more of the HLA-A, HLA-B, and HLA-C genes. In some embodiments, the host cell has TAP1 knockout. In some embodiments, the genetic modification targeting the TAP1 locus comprises inserting an exogenous nucleic acid encoding a polypeptide as disclosed herein (e.g., a polycistronic vector or a fragment thereof) at the TAP1 locus, according to embodiments disclosed herein. In certain of these embodiments, insertion of the polycistronic vector or fragment thereof into the TAP1 locus results in TAP1 knockout.

In some embodiments, the allogeneic host cells may be modified to have reduced expression of MHC II genes by overexpression of CD74.

In some embodiments, the allogeneic host cells may be modified to have reduced expression of MHC II genes by targeting and modulating the class II transactivator (CIITA) locus. CIITA is a member of the nucleotide binding domain (NBD) leucine-rich repeat (LRR) family of proteins and regulates the transcription of MHC II by associating with the MHC enhanceosome. In some embodiments, the modulation occurs through insertion-deletion (indel) modifications of the CIITA locus, for example, by using the CRISPR/Cas system as described. In some embodiments, reduced expression of CIITA reduces or eliminates expression of one or more of the HLA-DR, HLA-DQ, HLA-DP, HLA-DM, and HLA-DO genes. In some embodiments, the host cell has CIITA knockout. In some embodiments, the genetic modification targeting the CIITA locus comprises inserting an exogenous nucleic acid encoding a polypeptide as disclosed herein (e.g., a polycistronic vector or a fragment thereof) at the CIITA locus, according to embodiments disclosed herein. In certain of these embodiments, insertion of the polycistronic vector or fragment thereof into the CIITA locus results in CIITA knockout.

In some embodiments, the allogeneic host cells have genetic modifications at the B2M, TAP1, and/or CIITA loci, have B2M, TAP1, and/or CIITA knockout, or have CD74 overexpression. The B2M, TAP1, and/or CIITA knockout can occur at one allele, or both alleles, of the respective gene locus. In some embodiments, the B2M, TAP1, and/or CIITA loci are modified so that the allogeneic host cell has reduced or no expression of B2M, TAP1 and/or CIITA, respectively. In these embodiments, the allogeneic host cell has reduced expression of MHC I and/or MHC II genes (HLA I and/or HLA II in humans) as a result of B2M, TAP1, and/or CIITA deletion or knockout, or overexpression of CD74.

In certain embodiments, the allogeneic host cells have a genetic modification of MIC-A. MIC-A is a protein having known isoforms and variants (see, e.g., UniProt Q29983, accessed Jul. 18, 2022); all such forms of MIC-A are encompassed by the disclosure provided herein. In some embodiments, the genetic modification occurs using a CRISPR/Cas system. For example, in some embodiments, a gRNA with a targeting sequence GATGACCCTGGCTCATATCA (SEQ ID NO:137) can be used. In some embodiments, methods of gene editing with a CRISPR/Cas system and gRNA targeting MIC-A, such as with a targeting sequence GATGACCCTGGCTCATATCA (SEQ ID NO:137), knocks out all alleles of MIC-A in a cell, such as a host cell.

In some embodiments, the engineered cell comprises a modification, such as a genetic modification, targeting the MIC-A gene. In some embodiments, the genetic modification targeting the MIC-A gene is by using a targeted nuclease system that comprises a Cas protein or a polynucleotide encoding a Cas protein, and at least one guide ribonucleic acid sequence for specifically targeting the MICA gene.

In some embodiments, a polycistronic vector is inserted at the MIC-A gene.

In certain embodiments, the allogeneic host cells have a genetic modification of MIC-B. MIC-B is a protein having known isoforms and variants (see, e.g., UniProt Q29980, accessed Jul. 18, 2022); all such forms of MIC-B are encompassed by the disclosure provided herein. In some embodiments, the genetic modification occurs using a CRISPR/Cas system. For example, in some embodiments, a gRNA with a targeting sequence GTTTCTGCCTGTCATAGCGC (SEQ ID NO:138) can be used. In some embodiments, methods of gene editing with a CRISPR/Cas system and gRNA targeting MIC-B, such as with a targeting sequence GTTTCTGCCTGTCATAGCGC (SEQ ID NO:138) knocks out all alleles of MIC-B in a cell, such as a host cell.

In some embodiments, the engineered cell comprises a modification, such as a genetic modification, targeting the MIC-B gene. In some embodiments, the genetic modification targeting the MIC-B gene is by using a targeted nuclease system that comprises a Cas protein or a polynucleotide encoding a Cas protein, and at least one guide ribonucleic acid sequence for specifically targeting the MIC-B gene.

In some embodiments, a polycistronic vector is inserted at the MIC-B gene.

### Host Cells

In some aspects, the present technology provides a population of host cells, such as T cells, natural killer (NK) cells, natural killer T (NKT) cells, pluripotent stem cells (PSCs), or cells derived therefrom, generated by methods as described that contain the polycistronic vector according to various embodiments disclosed herein. In some embodiments, the host cells may be a heterogenous population, e.g., mixtures of cells that contain different polycistronic vectors according to various embodiments disclosed herein.

In some embodiments, the host cells are T cells, for example, naïve T cells, helper T cells (CD4+), cytotoxic T cells (CD8+), regulatory T cells (Treg), central memory T cells (TcM), effector memory T cells (TEM), stem cell memory T cells (TScM), or any combination thereof. In some embodiments, the host cell expresses a tolerogenic factor (e.g., CD47, HLA-E, HLA-G, PD-L1, CTLA-4), a CAR (e.g., CD19 CAR, CD22 CAR, BCMA CAR), and/or a safety switch encoded by a polycistronic vector according to various embodiments disclosed therein. In these embodiments, the host cell recognizes and initiates an immune response to a target cell expressing the antigen the CAR is designed to target (e.g., CD19, CD22, BCMA), and the host cell possesses hypoimmunity in an allogeneic recipient due to expression of the tolerogenic factor.

In some embodiments, the T cells are autologous, i.e., obtained from the subject who will receive the T cells after modification. In some embodiments, the T cells are allogeneic, i.e., obtained from someone other than the subject who will receive the T cells after modification. In either of these embodiments, the T cells can be primary T cells obtained from a number of sources, including peripheral blood mononuclear cells, bone marrow, lymph node tissue, cord blood, thymus tissue, tissue from a site of infection, ascites, pleural effusion, spleen tissue, and tumors. In other embodiments, for example, in the case of allogeneic T cells, the T cells can be derived or differentiated from PSCs such as embryonic stem cells (ESCs) or induced pluripotent cells (iPSCs).

In some embodiments, the host cells are NK cells. NK cells (also defined as large granular lymphocytes) represent a cell lineage differentiated from the common lymphoid progenitor (which also gives rise to B lymphocytes and T lymphocytes). Unlike T-cells, NK cells do not naturally express CD3 at the plasma membrane. Importantly, NK cells do not express a TCR and typically also lack other antigen-specific cell surface receptors. NK cells' cytotoxic activity does not require sensitization but is enhanced by activation with a variety of cytokines including IL-2. NK cells are generally thought to lack appropriate or complete signaling pathways necessary for antigen-receptor-mediated signaling, and thus are not thought to be capable of antigen receptor-dependent signaling, activation and expansion. NK cells are cytotoxic, and they balance activating and inhibitory receptor signaling to modulate their cytotoxic activity. For instance, NK cells expressing CD16 may bind to the Fc domain of antibodies bound to an infected cell, resulting in NK cell activation. By contrast, activity is reduced against cells expressing high levels of MHC class I proteins. On contact with a target cell, NK cells release proteins such as perforin, and enzymes such as proteases (granzymes). Perforin can form pores in the cell membrane of a target cell, inducing apoptosis or cell lysis. In some embodiments, the NK cells are autologous, i.e., obtained from the subject who will receive the NK cells after modification. In some embodiments, the NK cells are allogeneic, i.e., obtained from someone other than the subject who will receive the NK cells after modification. In either of these embodiments, the NK cells can be primary NK cells obtained from a number of sources, including peripheral blood mononuclear cells, bone marrow, lymph node tissue, cord blood, thymus tissue, tissue from a site of infection, ascites, pleural effusion, spleen tissue, and tumors. In some embodiments, the NK cells can be derived or differentiated from ESCs or iPSCs. There are a number of techniques that can be used to generate NK cells from pluripotent stem cells (e.g., iPSCs). See, for example, Zhu et al., Methods Mol Biol. 2019; 2048:107-119; Knorr et al., Stem Cells Transl Med. 2013 2(4):274-83. doi: 10.5966/sctm.2012-0084; Zeng et al., Stem Cell Reports. 2017 Dec. 12; 9(6):1796-1812; Ni et al., Methods Mol Biol. 2013; 1029:33-41; Bernareggi et al., Exp Hematol. 2019 71:13-23; Shankar et al., Stem Cell Res Ther. 2020; 11(1):234, all of which are incorporated herein by reference in their entirety and specifically for the methodologies and reagents for differentiation. Differentiation can be assayed as is known in the art, generally by evaluating the presence of NK cell associated and/or specific markers, including, but not limited to, CD56, KIRs, CD16, NKp44, NKp46, NKG2D, TRAIL, CD122, CD27, CD244, NK1.1, NKG2A/C, NCR1, Ly49, CD49b, CD11b, KLRG1, CD43, CD62L, and/or CD226.

In some embodiments, the host cells are NKT cells. NKT cells are a heterogeneous group of T cells that share properties of both T cells and NK cells. Many of these cells recognize the non-polymorphic CD1d molecule, an antigen-presenting molecule that binds self and foreign lipids and glycolipids. They constitute only approximately 1% of all peripheral blood T cells. In some embodiments, the NKT cells are autologous, i.e., obtained from the subject who will receive the NKT cells after modification. In some embodiments, the NKT cells are allogeneic, i.e., obtained from someone other than the subject who will receive the NKT cells after modification. In either of these embodiments, the NKT cells can be primary NKT cells obtained from a number of sources, including peripheral blood mononuclear cells, bone marrow, lymph node tissue, cord blood, thymus tissue, tissue from a site of infection, ascites, pleural effusion, spleen tissue, and tumors. In some embodiments, the NKT cells can be derived or differentiated from ESCs or iPSCs.

In some embodiments, the host cells are pancreatic islet cells, including, for example, β cells (also referred to as beta cells or β islet cells). Exemplary pancreatic islet cell types include, but are not limited to, pancreatic islet progenitor cells, immature pancreatic islet cells, mature pancreatic islet cells, and the like. In some embodiments, the p islet cells are autologous, i.e., obtained from the subject who will receive the β islet cells after modification. In some embodiments, the β islet cells are allogeneic, i.e., obtained from someone other than the subject who will receive the β islet cells after modification. In either of these embodiments, the β islet cells can be primary β islet cells. In some embodiments, the β islet cells can be derived or differentiated from ESCs or iPSCs. Useful method for differentiating pluripotent stem cells into pancreatic islet cells are disclosed, for example, in U.S. Pat. Nos. 9,683,215; 9,157,062; and 8,927,280.

In some embodiments, the β islet cells produced by the methods as disclosed herein secretes insulin. In some embodiments, a β islet cell exhibits at least two characteristics of an endogenous pancreatic islet cell, for example, but not limited to, secretion of insulin in response to an increase in glucose, and expression of beta cell markers. In some embodiments, the β islet cells disclosed herein are administered to a subject to treat diabetes. Exemplary β cell markers or β cell progenitor markers include, but are not limited to, c-peptide, Pdxl, glucose transporter 2 (Glut2), HNF6, VEGF, glucokinase (GCK), prohormone convertase (PC 1/3), Cdcpl, NeuroD, Ngn3, Nkx2.2, Nkx6.1, Nkx6.2, Pax4, Pax6, Ptfla, Isll, Sox9, Soxl7, and FoxA2. In some embodiments, the PSCs are differentiated into beta-like cells or islet organoids for transplantation to address type I diabetes mellitus (T1 DM). Cell systems are a promising way to address T1 DM, see, e.g., Ellis et al., Nat Rev Gastroenterol Hepatol. 2017 October; 14(10):612-628, incorporated herein by reference. Additionally, Pagliuca et al. (Cell, 2014, 159(2):428-39) reports on the successful differentiation of β cells from hiPSCs, the contents incorporated herein by reference in its entirety and in particular for the methods and reagents disclosed there for the large-scale production of functional human β cells from human pluripotent stem cells). Furthermore, Vegas et al. shows the production of human β cells from human pluripotent stem cells followed by encapsulation to avoid immune rejection by the recipient; Vegas et al., Nat Med, 2016, 22(3):306-11, incorporated herein by reference in its entirety and in particular for the methods and reagents disclosed there for the large-scale production of functional human β cells from human pluripotent stem cells. Additional disclosure of pancreatic islet cells including pancreatic β islet cells for use in the present technology are found in WO2020/018615, the disclosure is herein incorporated by reference in its entirety.

In some embodiments, the host cells are primary cells. In some embodiments, the host cells are PSCs, for example, ESCs or iPSCs. In some embodiments, the host cells are cells differentiated from ESCs or iPSCs. ESCs and iPSCs have the ability to differentiated into any cell type of the body, including, for example, neurons, astrocytes, oligodendrocytes, retinal epithelial cells, epidermal cells, hair cells, keratinocytes, hepatocytes, pancreatic β islet cells, intestinal epithelial cells, lung alveolar cells, hematopoietic cells, endothelial cells, cardiomyocytes, smooth muscle cells, skeletal muscle cells, renal cells, adipocytes, chondrocytes, and osteocytes. In some embodiments, the host cell is a T cell, an NK cell, or an NKT cell. In some embodiments, the host cell is a β islet cell or a glial progenitor cell (GPC).

### Host Cell Compositions

In some aspects, the present technology provides compositions comprising a population of host cells according to various embodiments disclosed herein.

In some embodiments, the composition can have various formulations, for example, injectable formulations, lyophilized formulations, liquid formulations, oral formulations, etc., depending on the suitable routes of administration.

In some embodiments, the composition can be co-formulated in the same dosage unit or can be individually formulated in separate dosage units. The terms “dose unit” and “dosage unit” herein refer to a portion of a composition that contains an amount of a therapeutic agent suitable for a single administration to provide a therapeutic effect. Such dosage units may be administered one to a plurality (i.e., 1 to 10, 1 to 8, 1 to 6, 1 to 4, or 1 to 2) of times per day, or as many times as needed to elicit a therapeutic response.

In some embodiments, a single dosage unit includes at least about 1×10², 5×10², 1×10³, 5×10³, 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, or 5×10¹⁰ cells.

### Insertion of the Polycistronic Vector in the Host Cell

In some aspects, the polycistronic vector of the present technology or a fragment thereof can be integrated into the genome of the host cell. Provided herein in certain embodiments are methods and compositions for carrying out this integration. In those embodiments wherein a fragment of the polycistronic vector is integrated into the genome, said fragment includes at least the expression cassettes containing the transgene(s) of interest (e.g., the tolerogenic factor, CAR, and/or safety switch), and may optionally include the promoter(s).

### Random Insertion

In some embodiments, the polycistronic vector of the present technology or a fragment thereof is inserted into a random genomic locus of a host cell. As known to a person skilled in the art, viral vectors, including, for example, retroviral vectors, lentiviral vectors, adenoviral vectors, and adeno-associated viral vectors, are commonly used to deliver genetic material into host cells and randomly insert the foreign or exogenous gene into the host cell genome to facilitate stable expression and replication of the gene.

### Site-Directed Insertion (Knock-In)

In some embodiments, the polycistronic vector of the present technology or a fragment thereof is inserted into a specific genomic locus of the host cell. A number of gene editing methods can be used to insert the polycistronic vector or fragments thereof into the specific genomic locus of choice. Gene editing is a type of genetic engineering in which a nucleotide sequence may be inserted, deleted, modified, or replaced in the genome of a living organism. Current gene editing techniques generally utilize the innate mechanism for cells to repair double-strand breaks (DSBs) in DNA.

Eukaryotic cells repair DSBs by two primary repair pathways: non-homologous end-joining (NHEJ) and homology-directed repair (HDR). HDR typically occurs during late S phase or G2 phase, when a sister chromatid is available to serve as a repair template. NHEJ is more common and can occur during any phase of the cell cycle, but it is more error prone. In gene editing, NHEJ is generally used to produce insertion/deletion mutations (indels), which can produce targeted loss of function in a target gene by shifting the open reading frame (ORF) and producing alterations in the coding region or an associated regulatory region. HDR, on the other hand, is a preferred pathway for producing targeted knock-ins, knockouts, or insertions of specific mutations in the presence of a repair template with homologous sequences. Several methods are known to a skilled artisan to improve HDR efficiency, including, for example, chemical modulation (e.g., treating cells with inhibitors of key enzymes in the NHEJ pathway); timed delivery of the gene editing system at S and G2 phases of the cell cycle; cell cycle arrest at S and G2 phases; and introduction of repair templates with homology sequences. The methods provided herein may utilize HDR-mediated repair, NHEJ-mediated repair, prime editing, or a combination thereof.

Prime editing is a versatile and precise genome editing method that directly writes new genetic information into a specified DNA site using a catalytically impaired Cas9 endonuclease fused to an engineered reverse transcriptase, programmed with a prime editing guide RNA (pegRNA) that both specifies the target site and encodes the desired edit. See, e.g., Anzalone et al., Nature, 576:149-157 (2019); WO2021072328; WO2022067130, all of which are incorporated herein by reference in their entirety.

In some embodiments, the methods provided herein for site-directed insertion utilize a site-directed nuclease, including, for example, zinc finger nucleases (ZFNs), transcription activator-like effector nucleases (TALENs), meganucleases, transposases, and clustered regularly interspaced short palindromic repeat (CRISPR)/Cas systems. In some embodiments, the site-directed insertion is through prime editing or mismatch repair-based insertion.

### ZFNs

ZFNs are fusion proteins comprising an array of site-specific DNA binding domains adapted from zinc finger-containing transcription factors attached to the endonuclease domain of the bacterial FokI restriction enzyme. A ZFN may have one or more (e.g., 1, 2, 3, 4, 5, 6, 7, 8, 9, 10, or more) of the DNA binding domains or zinc finger domains. See, e.g., Carroll et al., Genetics Society of America (2011) 188:773-782; Kim et al., Proc. Natl. Acad. Sci. USA (1996) 93:1156-1160. Each zinc finger domain is a small protein structural motif stabilized by one or more zinc ions and usually recognizes a 3- to 4-bp DNA sequence. Tandem domains can thus potentially bind to an extended nucleotide sequence that is unique within a cell's genome.

Various zinc fingers of known specificity can be combined to produce multi-finger polypeptides which recognize about 6, 9, 12, 15, or 18-bp sequences. Various selection and modular assembly techniques are available to generate zinc fingers (and combinations thereof) recognizing specific sequences, including phage display, yeast one-hybrid systems, bacterial one-hybrid and two-hybrid systems, and mammalian cells. Zinc fingers can be engineered to bind a predetermined nucleic acid sequence. Criteria to engineer a zinc finger to bind to a predetermined nucleic acid sequence are known in the art. See, e.g., Sera et al., Biochemistry (2002) 41:7074-7081; Liu et al., Bioinformatics (2008) 24:1850-1857.

ZFNs containing FokI nuclease domains or other dimeric nuclease domains function as a dimer. Thus, a pair of ZFNs are required to target non-palindromic DNA sites. The two individual ZFNs must bind opposite strands of the DNA with their nucleases properly spaced apart. See Bitinaite et al., Proc. Natl. Acad. Sci. USA (1998) 95:10570-10575. To cleave a specific site in the genome, a pair of ZFNs are designed to recognize two sequences flanking the site, one on the forward strand and the other on the reverse strand. Upon binding of the ZFNs on either side of the site, the nuclease domains dimerize and cleave the DNA at the site, generating a DSB with 5′ overhangs. HDR can then be utilized to introduce a specific mutation, with the help of a repair template containing the desired mutation flanked by homology arms. The repair template is usually an exogenous double-stranded DNA vector introduced to the cell. See Miller et al., Nat. Biotechnol. (2011) 29:143-148; Hockemeyer et al., Nat. Biotechnol. (2011) 29:731-734.

### TALENs

TALENs are another example of an artificial nuclease which can be used to edit a target gene. TALENs are derived from DNA binding domains termed TALE repeats, which usually comprise tandem arrays with 10 to 30 repeats that bind and recognize extended DNA sequences. Each repeat is 33 to 35 amino acids in length, with two adjacent amino acids (termed the repeat-variable di-residue, or RVD) conferring specificity for one of the four DNA base pairs. Thus, there is a one-to-one correspondence between the repeats and the base pairs in the target DNA sequences.

TALENs are produced artificially by fusing one or more TALE DNA binding domains (e.g., 1, 2, 3, 4, 5, 6, 7, 8, 9, 10 or more) to a nuclease domain, for example, a FokI endonuclease domain. See Zhang, Nature Biotech. (2011) 29:149-153. Several mutations to FokI have been made for its use in TALENs; these, for example, improve cleavage specificity or activity. See Cermak et al., Nucl. Acids Res. (2011) 39:e82; Miller et al., Nature Biotech. (2011) 29:143-148; Hockemeyer et al., Nature Biotech. (2011) 29:731-734; Wood et al., Science (2011) 333:307; Doyon et al., Nature Methods (2010) 8:74-79; Szczepek et al., Nature Biotech (2007) 25:786-793; Guo et al., J. Mol. Biol. (2010) 200:96. The FokI domain functions as a dimer, requiring two constructs with unique DNA binding domains for sites in the target genome with proper orientation and spacing. Both the number of amino acid residues between the TALE DNA binding domain and the FokI nuclease domain and the number of bases between the two individual TALEN binding sites appear to be important parameters for achieving high levels of activity. Miller et al., Nature Biotech. (2011) 29:143-148.

By combining engineered TALE repeats with a nuclease domain, a site-specific nuclease can be produced specific to any desired DNA sequence. Similar to ZFNs, TALENs can be introduced into a cell to generate DSBs at a desired target site in the genome, and so can be used to knock out genes or knock in mutations in similar, HDR-mediated pathways. See Boch, Nature Biotech. (2011) 29:135-136; Boch et al., Science (2009) 326:1509-1512; Moscou et al., Science (2009) 326:3501.

### Meganucleases

Meganucleases are enzymes in the endonuclease family which are characterized by their capacity to recognize and cut large DNA sequences (from 14 to 40 base pairs). Meganucleases are grouped into families based on their structural motifs which affect nuclease activity and/or DNA recognition. The most widespread and best known meganucleases are the proteins in the LAGLIDADG family, which owe their name to a conserved amino acid sequence. See Chevalier et al., Nucleic Acids Res. (2001) 29(18): 3757-3774. On the other hand, the GIY-YIG family members have a GIY-YIG module, which is 70-100 residues long and includes four or five conserved sequence motifs with four invariant residues, two of which are required for activity. See Van Roey et al., Nature Struct. Biol. (2002) 9:806-811. The His-Cys family meganucleases are characterized by a highly conserved series of histidines and cysteines over a region encompassing several hundred amino acid residues. See Chevalier et al., Nucleic Acids Res. (2001) 29(18):3757-3774. Members of the NHN family are defined by motifs containing two pairs of conserved histidines surrounded by asparagine residues. See Chevalier et al., Nucleic Acids Res. (2001) 29(18):3757-3774.

Because the chance of identifying a natural meganuclease for a particular target DNA sequence is low due to the high specificity requirement, various methods including mutagenesis and high throughput screening methods have been used to create meganuclease variants that recognize unique sequences. Strategies for engineering a meganuclease with altered DNA-binding specificity, e.g., to bind to a predetermined nucleic acid sequence are known in the art. See, e.g., Chevalier et al., Mol. Cell. (2002) 10:895-905; Epinat et al., Nucleic Acids Res (2003) 31:2952-2962; Silva et al., J Mol. Biol. (2006) 361:744-754; Seligman et al., Nucleic Acids Res (2002) 30:3870-3879; Sussman et al., J Mol Biol (2004) 342:31-41; Doyon et al., J Am Chem Soc (2006) 128:2477-2484; Chen et al., Protein Eng Des Sel (2009) 22:249-256; Arnould et al., J Mol Biol. (2006) 355:443-458; Smith et al., Nucleic Acids Res. (2006) 363(2):283-294.

Like ZFNs and TALENs, Meganucleases can create DSBs in the genomic DNA, which can create a frame-shift mutation if improperly repaired, e.g., via NHEJ, leading to a decrease in the expression of a target gene in a cell. Alternatively, foreign DNA can be introduced into the cell along with the meganuclease. Depending on the sequences of the foreign DNA and chromosomal sequence, this process can be used to modify the target gene. See Silva et al., Current Gene Therapy (2011) 11:11-27.

### Transposases

Transposases are enzymes that bind to the end of a transposon and catalyze its movement to another part of the genome by a cut and paste mechanism or a replicative transposition mechanism. By linking transposases to other systems such as the CRISPR/Cas system, new gene editing tools can be developed to enable site specific insertions or manipulations of the genomic DNA. There are two known DNA integration methods using transposons which use a catalytically inactive Cas effector protein and Tn7-like transposons. The transposase-dependent DNA integration does not provoke DSBs in the genome, which may guarantee safer and more specific DNA integration.

### CRISPR/Cas

The CRISPR system was originally discovered in prokaryotic organisms (e.g., bacteria and archaea) as a system involved in defense against invading phages and plasmids that provides a form of acquired immunity. Now it has been adapted and used as a popular gene editing tool in research and clinical applications.

CRISPR/Cas systems generally comprise at least two components: one or more guide RNAs (gRNAs) and a Cas protein. The Cas protein is a nuclease that introduces a DSB into the target site. CRISPR-Cas systems fall into two major classes: class 1 systems use a complex of multiple Cas proteins to degrade nucleic acids; class 2 systems use a single large Cas protein for the same purpose. Class 1 is divided into types I, 111, and IV; class 2 is divided into types II, V, and VI. Different Cas proteins adapted for gene editing applications include, but are not limited to, Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, and Mad7. See, e.g., Jinek et al., Science (2012) 337 (6096):816-821; Dang et al., Genome Biology (2015) 16:280; Ran et al., Nature (2015) 520:186-191; Zetsche et al., Cell (2015) 163:759-771; Strecker et al., Nature Comm. (2019) 10:212; Yan et al., Science (2019) 363:88-91. The most widely used Cas9 is a type II Cas protein and is described herein as illustrative. These Cas proteins may be originated from different source species. For example, Cas9 can be derived from S. pyogenes or S. aureus.

In the original microbial genome, the type II CRISPR system incorporates sequences from invading DNA between CRISPR repeat sequences encoded as arrays within the host genome. Transcripts from the CRISPR repeat arrays are processed into CRISPR RNAs (crRNAs) each harboring a variable sequence transcribed from the invading DNA, known as the “protospacer” sequence, as well as part of the CRISPR repeat. Each crRNA hybridizes with a second transactivating CRISPR RNA (tracrRNA), and these two RNAs form a complex with the Cas9 nuclease. The protospacer-encoded portion of the crRNA directs the Cas9 complex to cleave complementary target DNA sequences, provided that they are adjacent to short sequences known as “protospacer adjacent motifs” (PAMs).

While the foregoing description has focused on Cas9 nuclease, it should be appreciated that other RNA-guided nucleases exist which utilize gRNAs that differ in some ways from those described to this point. For instance, Cpf1 (CRISPR from Prevotella and Franciscella 1; also known as Cas12a) is an RNA-guided nuclease that only requires a crRNA and does not need a tracrRNA to function.

Since its discovery, the CRISPR system has been adapted for inducing sequence specific DSBs and targeted genome editing in a wide range of cells and organisms spanning from bacteria to eukaryotic cells including human cells. In its use in gene editing applications, artificially designed, synthetic gRNAs have replaced the original crRNA:tracrRNA complexes, including in certain embodiments via a single gRNA. For example, the gRNAs can be single guide RNAs (sgRNAs) composed of a crRNA, a tetraloop, and a tracrRNA. The crRNA usually comprises a complementary region (also called a spacer, usually about 20 nucleotides in length) that is user-designed to recognize a target DNA of interest. The tracrRNA sequence comprises a scaffold region for Cas nuclease binding. The crRNA sequence and the tracrRNA sequence are linked by the tetraloop and each have a short repeat sequence for hybridization with each other, thus generating a chimeric sgRNA. One can change the genomic target of the Cas nuclease by simply changing the spacer or complementary region sequence present in the gRNA. The complementary region will direct the Cas nuclease to the target DNA site through standard RNA-DNA complementary base pairing rules.

In order for the Cas nuclease to function, there must be a PAM immediately downstream of the target sequence in the genomic DNA. Recognition of the PAM by the Cas protein is thought to destabilize the adjacent genomic sequence, allowing interrogation of the sequence by the gRNA and resulting in gRNA-DNA pairing when a matching sequence is present. The specific sequence of PAM varies depending on the species of the Cas gene. For example, the most commonly used Cas9 nuclease derived from S. pyogenes recognizes a PAM sequence of 5′-NGG-3′ or, at less efficient rates, 5′-NAG-3′, where “N” can be any nucleotide. Other Cas nuclease variants with alternative PAMs have also been characterized and successfully used for genome editing, which are summarized in Table 17 below.

TABLE 17
Exemplary Cas nuclease variants and their PAM sequences

| CRISPR Nuclease | Source Organism | PAM Sequence (5′→3′) |
|---|---|---|
| SpCas9 | Streptococcus pyogenes | ngg or nag |
| SaCas9 | Staphylococcus aureus | ngrrt or ngrrn |
| NmeCas9 | Neisseria meningitidis | nnnngatt |
| CjCas9 | Campylobacter jejuni | nnnnryac |
| StCas9 | Streptococcus thermophilus | nnagaaw |
| TdCas9 | Treponema denticola | naaaac |
| LbCas12a (Cpf1) | Lachnospiraceae bacterium | tttv |
| AsCas12a (Cpf1) | Acidaminococcus sp. | tttv |
| AacCas12b | Alicyclobacillus acidiphilus | ttn |
| BhCas12b v4 | Bacillus hisashii | attn, tttn, or gttn |

r = a or g;
y = c or t;
w = a ort;
v = a or c or
g; n = any base

In some embodiments, Cas nucleases may comprise one or more mutations to alter their activity, specificity, recognition, and/or other characteristics. For example, the Cas nuclease may have one or more mutations that alter its fidelity to mitigate off-target effects (e.g., eSpCas9, SpCas9-HF1, HypaSpCas9, HeFSpCas9, and evoSpCas9 high-fidelity variants of SpCas9). For another example, the Cas nuclease may have one or more mutations that alter its PAM specificity.

In certain embodiments, the polycistronic vector or fragment thereof may function as a DNA repair template to be integrated into the target site through HDR in associated with a gene editing system (e.g., the CRISPR/Cas system) as described. Generally, the fragment of the polycistronic vector to be inserted would comprise at least the expression cassettes containing the transgene of interest (e.g., the tolerogenic factor, CAR, and/or safety switch expression cassettes) and would optionally also include the promoter. In certain of these embodiments, the fragment containing the expression cassettes and/or promoter to be inserted would be flanked by homologous sequence immediately upstream and downstream of the target, i.e., left homology arm (LHA) and right homology arm (RHA), specifically designed for the target genomic locus to serve as template for HDR. The length of each homology arm is generally dependent on the size of the insert being introduced, with larger insertions requiring longer homology arms.

In some embodiments, the specific genomic locus for site-directed insertion of the polycistronic vector or fragment thereof is selected from the group consisting of a B2M locus, a TAP1 locus, a CIITA locus, a TRAC locus, a TRBC locus, and a safe harbor locus. Non-limiting examples of safe harbor loci include, but are not limited to, an AAVS1 (also known as PPP1 R12C), ABO, CCR5, CLYBL, CXCR4, F3 (also known as CD142), FUT1, HMGB1, KDM5D, LRP1 (also known as CD91), MICA, MICB, RHD, ROSA26, and SHS231 gene locus. The vector or fragment thereof can be inserted in a suitable region of the safe harbor locus, including, for example, an intron, an exon, and/or gene coding region (also known as a CoDing Sequence, or “CDS”). In some embodiments, the safe harbor locus is selected from the group consisting of the AAVS1 locus, the CCR5 locus, and the CLYBL locus. In some embodiments, the insertion occurs in one allele of the specific genomic locus. In some embodiments, the insertion occurs in both alleles of the specific genomic locus. In either of these embodiments, the orientation of the transgene inserted into the target genomic locus can be either the same or the reverse of the direction of the gene in that locus. In some embodiments, insertion of the polycistronic vector or fragment thereof into a genomic locus as described results in knockout (KO) of the endogenous gene.

In some embodiments, provided are host cells or compositions of the same having a genomic locus modified by any of the gene editing systems as described. In some embodiments, the genetic modification is by using a site-directed nuclease selected from the group consisting of Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, Mad7, zinc finger nucleases (ZFNs), transcription activator-like effector nucleases (TALENs), meganucleases, and CRISPR-associated transposases. In certain of these embodiments, the genomic locus modified is a B2M locus, a TAP1 locus, a CIITA locus, a TRAC locus, a TRBC locus, or a safe harbor locus. Non-limiting examples of a safe harbor locus include, but are not limited to, an AAVS1, ABO, CCR5, CLYBL, CXCR4, F3, FUT1, HMGB1, KDM5D, LRP1, MICA, MICB, RHD, ROSA26, and SHS231 gene locus. In some embodiments, the host cells have endogenous gene KO due to modifications (e.g., insertion of a polycistronic vector or a fragment thereof) into a genomic locus as described.

Guide RNAs (gRNAs) for Gene Editing

In some embodiments, provided are gRNAs for use in site-directed insertion of the polycistronic vectors or fragments thereof provided herein, for example, in association with the CRISPR/Cas system. The gRNAs comprise a crRNA sequence, which in turn comprises a complementary region (also called a spacer) that recognizes and binds a complementary target DNA of interest. The length of the spacer or complementary region is generally between 15 and 30 nucleotides, usually about 20 nucleotides in length, although will vary based on the requirements of the specific CRISPR/Cas system. In certain embodiments, the spacer or complementary region is fully complementary to the target DNA sequence. In other embodiments, the spacer is partially complementary to the target DNA sequence, for example at least 80%, 85%, 90%, 95%, 98%, or 99% complementary.

In certain embodiments, the gRNAs provided herein further comprise a tracrRNA sequence, which comprises a scaffold region for binding to a nuclease. The length and/or sequence of the tracrRNA may vary depending on the specific nuclease being used for editing. In certain embodiments, nuclease binding by the gRNA does not require a tracrRNA sequence. In those embodiments where the gRNA comprises a tracrRNA, the crRNA sequence may further comprise a repeat region for hybridization with complementary sequences of the tracrRNA.

In some embodiments, the gRNAs provided herein comprise two or more gRNA molecules, for example, a crRNA and a tracrRNA, as two separate molecules. In other embodiments, the gRNAs are single guide RNAs (sgRNAs), including sgRNAs comprising a crRNA and a tracrRNA on a single RNA molecule. In certain of these embodiments, the crRNA and tracrRNA are linked by an intervening tetraloop.

In some embodiments, one gRNA can be used in association with a site-directed nuclease for targeted editing of a gene locus of interest. In other embodiments, two or more gRNAs targeting the same gene locus of interest can be used in association with a site-directed nuclease.

In some embodiments, exemplary gRNAs (e.g., sgRNAs) for use with various common Cas nucleases that require both a crRNA and tracrRNA, including Cas9 and Cas12b (C2c1), are provided in Table 18. See, e.g., Jinek et al., Science (2012) 337 (6096):816-821; Dang et al., Genome Biology (2015) 16:280; Ran et al., Nature (2015) 520:186-191; Strecker et al., Nature Comm. (2019) 10:212. For each exemplary gRNA, sequences for different portions of the gRNA, including the complementary region or spacer, crRNA repeat region, tetraloop, and tracrRNA, are shown. In some embodiments, the gRNA comprises all or a portion of the nucleotide sequences set forth in SEQ ID NOs:94-97. In some embodiments, the gRNA comprises all or a portion of the nucleotide sequences set forth in SEQ ID NOs:98-101. In some embodiments, the gRNA comprises all or a portion of the nucleotide sequences set forth in SEQ ID NOs:102-105. In some embodiments, the gRNA comprises all or a portion of the nucleotide sequences set forth in SEQ ID NOs:106-109.

In some embodiments, the gRNA comprises a crRNA repeat region comprising, consisting of, or consisting essentially of the nucleotide sequence set forth in SEQ ID NO:95, SEQ ID NO:99, SEQ ID NO:103, or SEQ ID NO:108. In some embodiments, the gRNA comprises a tetraloop comprising, consisting of, or consisting essentially of the nucleotide sequence set forth in SEQ ID NO:96 or SEQ ID NO:107. In some embodiments, the gRNA comprises a tracrRNA comprising, consisting of, or consisting essentially of the nucleotide sequence set forth in SEQ ID NO:97, SEQ ID NO:101, SEQ ID NO:105, or SEQ ID NO:106.

TABLE 18
Exemplary gRNA structure and sequence for CRISPR/Cas

| SEQ ID NO: | Sequence (5′→3′) | Description |
|---|---|---|
| 94 | nnnnnnnnnnnnnnnnnnnn | Exemplary spCas9 1 |
| | | Complementary region |
| | | (spacer) |
| 95 | guuuuagagcua | Exemplary spCas9 1 |
| | | crRNA repeat region |
| 96 | gaaa | Exemplary spCas9 1 |
| | | tetraloop |
| 97 | uagcaaguuaaaauaaggcuaguccguuaucaacuu | Exemplary spCas9 1 |
| | gaaaaaguggcaccgagucggugcuuuuuu | tracrRNA |
| 98 | nnnnnnnnnnnnnnnnnnnn | Exemplary spCas9 2 |
| | | Complementary region |
| | | (spacer) |
| 99 | guuusagagcuaugcug | Exemplary spCas9 2 |
| | | crRNA repeat region |
| 100 | gaaa | Exemplary spCas9 2 |
| | | tetraloop |
| 101 | cagcauagcaaguusaaauaaggcuaguccguuauc | Exemplary spCas9 2 |
| | aacuugaaaaaguggcaccgagucggugcuuuuuu | tracrRNA |
| 102 | nnnnnnnnnnnnnnnnnnnn | Exemplary saCas9 |
| | | Complementary region |
| | | (spacer) |
| 103 | guuuuaguacucug | Exemplary saCas9 |
| | | crRNA repeat region |
| 104 | gaaa | Exemplary saCas9 |
| | | tetraloop |
| 105 | cagaaucuacuaaaacaaggcaaaaugccguguuua | Exemplary saCas9 |
| | ucucgucaacuuguuggcgagauuuuuu | tracrRNA |
| 106 | gucgucuauaggacggcgaggacaacgggaagugcc | Exemplary AkCas12b |
| | aaugugcucuuuccaagagcaaacaccccguuggcuu | tracrRNA |
| | caagaugaccgcucg | |
| 107 | aaaa | Exemplary AkCas12b |
| | | tetraloop |
| 108 | cgagcggucugagaaguggcacu | Exemplary AkCas12b |
| | | crRNA repeat region |
| 109 | nnnnnnnnnnnnnnnnnnnn | Exemplary AkCas12b |
| | | Complementary region |
| | | (spacer) |

S = c or g; n = any base

In some embodiments, the gRNA comprises a complementary region specific to a target gene locus of interest, for example, the B2M locus, the TAP1 locus, the CIITA locus, the TRAC locus, the TRBC locus, or a safe harbor locus selected from the group consisting of an AAVS1, ABO, CCR5, CLYBL, CXCR4, F3, FUT1, HMGB1, KDM5D, LRP1, MICA, MICB, RHD, ROSA26, and SHS231 gene locus. The complementary region may bind a sequence in any region of the target gene locus, including for example, a CDS, an exon, an intron, a sequence spanning a portion of an exon and a portion of an adjacent intron, or a regulatory region (e.g., promoter, enhancer). Where the target sequence is a CDS, exon, intron, or sequence spanning portions of an exon and intron, the CDS, exon, intron, or exon/intron boundary may be defined according to any splice variant of the target gene. In some embodiments, the genomic locus targeted by the gRNA is located within 4000 bp, within 3500 bp, within 3000 bp, within 2500 bp, within 2000 bp, within 1500 bp, within 1000 bp, or within 500 bp of any of the loci or regions thereof as described. Further provided herein are compositions comprising one or more gRNAs provided herein and a Cas protein or a nucleotide sequence encoding a Cas protein. In certain of these embodiments, the one or more gRNAs and a nucleotide sequence encoding a Cas protein are comprised within a vector, for example, a viral vector.

In some embodiments, the gRNAs used herein for site-directed insertion of a transgene comprise a complementary region that recognizes a target sequence in AAVS1. In certain of these embodiments, the target sequence is located in intron 1 of AAVS1. AAVS1 is located at Chromosome 19: 55,090,918-55,117,637 reverse strand, and AAVS1 intron 1 (based on transcript ENSG00000125503) is located at Chromosome 19: 55,117,222-55,112,796 reverse strand. In certain embodiments, the gRNAs target a genomic locus within 4000 bp, within 3500 bp, within 3000 bp, within 2500 bp, within 2000 bp, within 1500 bp, within 1000 bp, or within 500 bp of a site located anywhere at Chromosome 19: 55,117,222-55,112,796. In certain embodiments, the gRNAs target a genomic locus within 4000 bp, within 3500 bp, within 3000 bp, within 2500 bp, within 2000 bp, within 1500 bp, within 1000 bp, or within 500 bp of Chromosome 19: 55,115,674. In certain embodiments, the gRNA is configured to produce a cut site at Chromosome 19: 55,115,674, or at a position within 5, 10, 15, 20, 30, 40, or 50 nucleotides of Chromosome 19: 55,115,674. In certain embodiments, the gRNA is GET000046, also known as “sgAAVS1-1,” described in Li et al., Nat. Methods 16:866-869 (2019). This gRNA comprises a complementary region comprising, consisting of, or consisting essentially of a nucleic acid sequence set forth in SEQ ID NO:110 and targets intron 1 of AAVS1 (also known as PPP1 R12C).

In some embodiments, the gRNAs used herein for site-directed insertion of a transgene comprise a complementary region that recognizes a target sequence in CLYBL. In certain of these embodiments, the target sequence is located in intron 2 of CLYBL. CLYBL is located at Chromosome 13: 99,606,669-99,897,134 forward strand, and CLYBL intron 2 (based on transcript ENST00000376355.7) is located at Chromosome 13: 99,773,011-99,858,860 forward strand. In certain embodiments, the gRNAs target a genomic locus within 4000 bp, within 3500 bp, within 3000 bp, within 2500 bp, within 2000 bp, within 1500 bp, within 1000 bp, or within 500 bp of a site located anywhere at Chromosome 13: 99,773,011-99,858,860. In certain embodiments, the gRNAs target a genomic locus within 4000 bp, within 3500 bp, within 3000 bp, within 2500 bp, within 2000 bp, within 1500 bp, within 1000 bp, or within 500 bp of Chromosome 13: 99,822,980. In certain embodiments, the gRNA is configured to produce a cut site at Chromosome 13: 99,822,980, or at a position within 5, 10, 15, 20, 30, 40, or 50 nucleotides of Chromosome 13: 99,822,980. In certain embodiments, the gRNA is GET000047, which comprises a complementary region comprising, consisting of, or consisting essentially of a nucleic acid sequence set forth in SEQ ID NO:111 and targets intron 2 of CLYBL. The target site is similar to the target site of the TALENs as described in Cerbini et al., PLoS One, 10(1): e0116032 (2015).

In some embodiments, the gRNAs used herein for site-directed insertion of a transgene comprise a complementary region that recognizes a target sequence in CCR5. In certain of these embodiments, the target sequence is located in exon 3 of CCR5. CCR5 is located at Chromosome 3: 46,370,854-46,376,206 forward strand, and CCR5 exon 3 (based on transcript ENST00000292303.4) is located at Chromosome 3: 46,372,892-46,376,206 forward strand. In certain embodiments, the gRNAs target a genomic locus within 4000 bp, within 3500 bp, within 3000 bp, within 2500 bp, within 2000 bp, within 1500 bp, within 1000 bp, or within 500 bp of a site located anywhere at Chromosome 3: 46,372,892-46,376,206. In certain embodiments, the gRNAs target a genomic locus within 4000 bp, within 3500 bp, within 3000 bp, within 2500 bp, within 2000 bp, within 1500 bp, within 1000 bp, or within 500 bp of Chromosome 3: 46,373,180. In certain embodiments, the gRNA is configured to produce a cut site at Chromosome 3: 46,373,180, or at a position within 5, 10, 15, 20, 30, 40, or 50 nucleotides of Chromosome 3: 46,373,180. In certain embodiments the gRNA is GET000048, also known as “crCCR5_D,” described in Mandal et al., Cell Stem Cell 15:643-652 (2014). This gRNA comprises a complementary region comprising, consisting of, or consisting essentially of a nucleic acid sequence set forth in SEQ ID NO:112 and targets exon 3 of CCR5 (alternatively annotated as exon 2 in the Ensembl genome database). See Gomez-Ospina et al., Nat. Comm. 10(1):4045 (2019).

In certain embodiments of the gRNAs used herein, one or more thymines in the complementary region sequences set forth in Table 19 are substituted with uracils.

TABLE 19
Exemplary gRNA complementary region sequences

| SEQ ID NO: | Nucleic Acid Sequence (5′→3′) | Description |
|---|---|---|
| 110 | accccacagtggggccacta | GET000046 guide |
| 111 | tgttggaaggatgaggaaat | GET000047 guide |
| 112 | tcactatgctgccgcccagt | GET000048 guide |

In some embodiments, provided are methods of identifying new loci and/or gRNA sequences for use in the site-directed gene editing approaches as described. For example, for CRISPR/Cas systems, when an existing gRNA for a particular locus (e.g., within a safe harbor locus) is known, an “inch worming” approach can be used to identify additional loci for targeted insertion of transgenes by scanning the flanking regions on either side of the locus for PAM sequences, which usually occurs about every 100 base pairs (bp) across the genome. The PAM sequence will depend on the particular Cas nuclease used because different nucleases usually have different corresponding PAM sequences. The flanking regions on either side of the locus can be between about 500 to 4000 bp long, for example, about 500 bp, about 1000 bp, about 1500 bp, about 2000 bp, about 2500 bp, about 3000 bp, about 3500 bp, or about 4000 bp long. When a PAM sequence is identified within the search range, a new guide can be designed according to the sequence of that locus for use in site-directed insertion of transgenes. Although the CRISPR/Cas system is described as illustrative, any gene editing approaches as described can be used in this method of identifying new loci, including those using ZFNs, TALENs, meganucleases, and transposases.

In some embodiments, the activity, stability, and/or other characteristics of gRNAs can be altered through the incorporation of chemical and/or sequential modifications. As one example, transiently expressed or delivered nucleic acids can be prone to degradation by, e.g., cellular nucleases. Accordingly, the gRNAs described herein can contain one or more modified nucleosides or nucleotides which introduce stability toward nucleases. While not being bound by a particular theory, it is believed that certain modified gRNAs described herein can exhibit a reduced innate immune response when introduced into a population of cells, particularly the cells of the present technology. As used herein, the term “innate immune response” includes a cellular response to exogenous nucleic acids, including single stranded nucleic acids, generally of viral or bacterial origin, which involves the induction of cytokine expression and release, particularly the interferons, and cell death. Other common chemical modifications of gRNAs to improve stabilities, increase nuclease resistance, and/or reduce immune response include 2′-O-methyl modification, 2′-fluoro modification, 2′-O-methyl phosphorothioate linkage modification, and 2′-O-methyl 3′ thioPACE modification.

One common 3′ end modification is the addition of a poly A tract comprising one or more (and typically 5-200) adenine (A) residues. The poly A tract can be contained in the nucleic acid sequence encoding the gRNA or can be added to the gRNA during chemical synthesis, or following in vitro transcription using a polyadenosine polymerase (e.g., E. coli Poly(A)Polymerase). In vivo, poly-A tracts can be added to sequences transcribed from DNA vectors through the use of polyadenylation signals. Examples of such signals are provided in Maeder. Other suitable gRNA modifications include, without limitations, those described in U.S. Patent Application No. US 2017/0073674 A1 and International Publication No. WO 2017/165862 A1, the entire contents of each of which are incorporated by reference herein.

Delivery of Gene Editing Systems into a Host Cell

In some embodiments, provided are compositions comprising one or more components of a gene editing system described herein, including one or more gRNAs, a site-directed nuclease (e.g., a Cas nuclease) or a nucleotide sequence encoding a site-directed nuclease protein, and optionally a transgene for targeted insertion. In some embodiments, the compositions are formulated for delivery into a cell.

In some embodiments, components of a gene editing system provided herein, including one or more gRNAs, a site-directed nuclease (e.g., a Cas nuclease) or a nucleotide sequence encoding a site-directed nuclease protein, and optionally a transgene (e.g., the polycistronic vector of the present technology or a fragment thereof) for targeted insertion, may be delivered into a cell in the form of a delivery vector. The delivery vector can be any type of vector suitable for introduction of nucleotide sequences into a cell, including, for example, plasmids, adenoviral vectors, adeno-associated viral (AAV) vectors, retroviral vectors, lentiviral vectors, phages, and HDR-based donor vectors. The different components may be introduced into a cell together or separately, and may be delivered in a single vector or multiple vectors.

In some embodiments, the delivery vector may be introduced into a cell by any known method in the field, including, for example, viral transformation, calcium phosphate transfection, lipid-mediated transfection, DEAE-dextran, electroporation, microinjection, nucleoporation, liposomes, nanoparticles, or other methods.

In some embodiments, the present technology provides compositions comprising a delivery vector according to various embodiments disclosed herein. In some embodiments, the compositions may further comprise one or more pharmaceutically acceptable carriers, excipients, preservatives, or a combination thereof. A “pharmaceutically acceptable carrier or excipient” refers to a pharmaceutically acceptable material, composition, or vehicle that is involved in carrying or transporting a compound of interest from one tissue, organ, or portion of the body to another tissue, organ, or portion of the body. For example, the carrier or excipient may be a liquid or solid filler, diluent, excipient, solvent, or encapsulating material, or some combination thereof. Each component of the carrier or excipient must be “pharmaceutically acceptable,” in that it must be compatible with the other ingredients of the formulation. It also must be suitable for contact with any tissue, organ, or portion of the body that it may encounter, meaning that it must not carry a risk of toxicity, irritation, allergic response, immunogenicity, or any other complication that excessively outweighs its therapeutic benefits. Suitable excipients include water, saline, dextrose, glycerol, or the like and combinations thereof. In some embodiments, compositions comprising cells as disclosed herein further comprise a suitable infusion media.

In some embodiments, provided are cells or compositions thereof comprising one or more components of a gene editing system described herein, including one or more gRNAs, a site-directed nuclease (e.g., a Cas nuclease) or a nucleotide sequence encoding a site-directed nuclease protein, and optionally a transgene for targeted insertion.

### Methods of Treatment

In some aspects, the present technology provides methods for treating and/or preventing a disease in a subject in need thereof. The method entails administering to the subject a therapeutically effective amount of a population of host cells, or a pharmaceutical composition containing the same, that contain the polycistronic vector according to various embodiments disclosed herein.

In some embodiments, the host cells are T cells. The T cells can be autologous, i.e., obtained from the subject who will receive the T cells after modification. Alternatively, the T cells can be allogeneic, i.e., obtained from someone other than the subject who will receive the T cells after modification. In either of these embodiments, the T cells can be primary T cells obtained from a number of sources, including peripheral blood mononuclear cells, bone marrow, lymph node tissue, cord blood, thymus tissue, tissue from a site of infection, ascites, pleural effusion, spleen tissue, and tumors. In other embodiments, for example, in the case of allogeneic T cells, the T cells can be derived from ESCs or iPSCs.

In some embodiments, the T cells are naïve T cells, helper T cells (CD4+), cytotoxic T cells (CD8+), regulatory T cells (Treg), central memory T cells (T_{CM}), effector memory T cells (T_{EM}), stem cell memory T cells (T_{SCM}), or any combination thereof. More specifically, the T cells can be naïve T cells (not exposed to antigen; increased expression of CD62L, CCR7, CD28, CD3, CD127, and CD45RA, and decreased expression of CD45RO as compared to TcM), memory T cells (antigen-experienced and long-lived), or effector cells (antigen-experienced, cytotoxic). Memory T cells can be further divided into subsets of TcM (increased expression of CD62L, CCR7, CD28, CD127, CD45RO, and CD95, and decreased expression of CD54RA as compared to naïve T cells) and TEM (decreased expression of CD62L, CCR7, CD28, CD45RA, and increased expression of CD127 as compared to naïve T cells or TcM). Effector T cells refer to antigen-experienced CD8+ cytotoxic T cells that has decreased expression of CD62L, CCR7, CD28, and are positive for granzyme and perforin as compared to TcM. Helper T cells are CD4+ cells that influence the activity of other immune cells by releasing cytokines. CD4+ T cells can activate or suppress an adaptive immune response, and which of those two functions is induced will depend on the presence of other cells and signals. T cells can be collected using known techniques, and the various subpopulations or combinations thereof can be enriched or depleted by known techniques, such as by affinity binding to antibodies, flow cytometry, or immunomagnetic selection.

In some embodiments, the host cells are NK cells. NK cells (also defined as large granular lymphocytes) represent a cell lineage differentiated from the common lymphoid progenitor (which also gives rise to B lymphocytes and T lymphocytes). Unlike T-cells, NK cells do not naturally express CD3 at the plasma membrane. Importantly, NK cells do not express a TCR and typically also lack other antigen-specific cell surface receptors. NK cells' cytotoxic activity does not require sensitization but is enhanced by activation with a variety of cytokines including IL-2. NK cells are generally thought to lack appropriate or complete signaling pathways necessary for antigen-receptor-mediated signaling, and thus are not thought to be capable of antigen receptor-dependent signaling, activation and expansion. NK cells are cytotoxic, and they balance activating and inhibitory receptor signaling to modulate their cytotoxic activity. For instance, NK cells expressing CD16 may bind to the Fc domain of antibodies bound to an infected cell, resulting in NK cell activation. By contrast, activity is reduced against cells expressing high levels of MHC class I proteins. On contact with a target cell, NK cells release proteins such as perforin, and enzymes such as proteases (granzymes). Perforin can form pores in the cell membrane of a target cell, inducing apoptosis or cell lysis. In some embodiments, the NK cells are autologous, i.e., obtained from the subject who will receive the NK cells after modification. In some embodiments, the NK cells are allogeneic, i.e., obtained from someone other than the subject who will receive the NK cells after modification. In either of these embodiments, the NK cells can be primary NK cells obtained from a number of sources, including peripheral blood mononuclear cells, bone marrow, lymph node tissue, cord blood, thymus tissue, tissue from a site of infection, ascites, pleural effusion, spleen tissue, and tumors. In some embodiments, the NK cells can be derived or differentiated from ESCs or iPSCs. There are a number of techniques that can be used to generate NK cells from pluripotent stem cells (e.g., iPSCs). See, for example, Zhu et al., Methods Mol Biol. 2019; 2048:107-119; Knorr et al., Stem Cells Transl Med. 2013 2(4):274-83. doi: 10.5966/sctm.2012-0084; Zeng et al., Stem Cell Reports. 2017 Dec. 12; 9(6):1796-1812; Ni et al., Methods Mol Biol. 2013; 1029:33-41; Bernareggi et al., Exp Hematol. 2019 71:13-23; Shankar et al., Stem Cell Res Ther. 2020; 11(1):234, all of which are incorporated herein by reference in their entirety and specifically for the methodologies and reagents for differentiation. Differentiation can be assayed as is known in the art, generally by evaluating the presence of NK cell associated and/or specific markers, including, but not limited to, CD56, KIRs, CD16, NKp44, NKp46, NKG2D, TRAIL, CD122, CD27, CD244, NK1.1, NKG2A/C, NCR1, Ly49, CD49b, CD11b, KLRG1, CD43, CD62L, and/or CD226.

In some embodiments, the host cells are NKT cells. NKT cells are a heterogeneous group of T cells that share properties of both T cells and NK cells. Many of these cells recognize the non-polymorphic CD1d molecule, an antigen-presenting molecule that binds self and foreign lipids and glycolipids. They constitute only approximately 1% of all peripheral blood T cells. In some embodiments, the NKT cells are autologous, i.e., obtained from the subject who will receive the NKT cells after modification. In some embodiments, the NKT cells are allogeneic, i.e., obtained from someone other than the subject who will receive the NKT cells after modification. In either of these embodiments, the NKT cells can be primary NKT cells obtained from a number of sources, including peripheral blood mononuclear cells, bone marrow, lymph node tissue, cord blood, thymus tissue, tissue from a site of infection, ascites, pleural effusion, spleen tissue, and tumors. In some embodiments, the NKT cells can be derived or differentiated from ESCs or iPSCs.

In some embodiments, the host cells are pancreatic islet cells, including, for example, β cells (also referred to as beta cells or β islet cells). Exemplary pancreatic islet cell types include, but are not limited to, pancreatic islet progenitor cells, immature pancreatic islet cells, mature pancreatic islet cells, and the like. In some embodiments, the β islet cells are autologous, i.e., obtained from the subject who will receive the β islet cells after modification. In some embodiments, the β islet cells are allogeneic, i.e., obtained from someone other than the subject who will receive the β islet cells after modification. In either of these embodiments, the β islet cells can be primary β islet cells. In some embodiments, the β islet cells can be derived or differentiated from ESCs or iPSCs.

In some embodiments, the host cells are pluripotent stem cells, for example, ESCs or iPSCs. In some embodiments, the host cells are cells differentiated from ESCs or iPSCs. ESCs and iPSCs have the ability to differentiated into any cell type of the body, including, for example, neurons, astrocytes, oligodendrocytes, retinal epithelial cells, epidermal cells, hair cells, keratinocytes, hepatocytes, pancreatic β islet cells, intestinal epithelial cells, lung alveolar cells, hematopoietic cells, endothelial cells, cardiomyocytes, smooth muscle cells, skeletal muscle cells, renal cells, adipocytes, chondrocytes, and osteocytes. In some embodiments, the host cells are β islet cells or glial progenitor cells (GPCs).

In some embodiments, the disease is cancer, for example, one associated with CD19, CD22, or BCMA expression, i.e., the cancer cell expresses CD19, CD22, or BCMA. In these embodiments, the method comprises contacting the cancer cell with a host cell containing the polycistronic vector of the present technology and expressing the corresponding CAR, such that the CAR is activated in response to the antigen expressed on the cancer cell and subsequently initiates killing of the cancer cell.

In some embodiments, the cancer is a hematologic malignancy. Non-limiting examples of hematologic malignancies include myeloid neoplasm, myelodysplastic syndromes (MDS), myeloproliferative/myelodysplastic syndromes, acute lymphoid leukemia (ALL), chronic lymphocytic leukemia (CLL), acute myeloid leukemia (AML), chronic myelogenous leukemia (CML), B cell acute lymphoid leukemia (B-ALL), T cell acute lymphoid leukemia (T-ALL), T cell lymphoma, and B cell lymphoma. In one embodiment, the hematologic malignancy is a CD19+, B lymphocyte-derived malignancy.

In some embodiments, the disease is an autoimmune disease, including, for example, lupus, systemic lupus erythematosus, rheumatoid arthritis, psoriasis, psoriatic arthritis, multiple sclerosis, Crohn's disease, ulcerative colitis, Addison's disease, Graves' disease, Sjögren's syndrome, Hashimoto's thyroiditis, and celiac disease.

In some embodiments, the disease is diabetes mellitus, including, for example, Type I diabetes, Type II diabetes, prediabetes, and gestational diabetes.

In some embodiments, the disease is a neurological disease, including, for example, catalepsy, epilepsy, encephalitis, meningitis, migraine, Huntington's, Alzheimer's, Parkinson's, Pelizaeus-Merzbacher disease, and multiple sclerosis.

In some embodiments, the population of host cells, or a pharmaceutical composition containing the same, according to the present technology may be administered in a manner appropriate to the disease, condition, or disorder to be treated as determined by persons skilled in the medical art. In any of the above embodiments, The population of host cells, or a pharmaceutical composition containing the same, can be administered intravenously, intraperitoneally, intratumorally, into the bone marrow, into a lymph node, or into the cerebrospinal fluid, so as to encounter the target antigen or cells. An appropriate dose, suitable duration, and frequency of administration will be determined by such factors as a condition of the patient; size, type, and severity of the disease, condition, or disorder; the undesired type or level or activity of the cells, the particular form of the active ingredient; and the method of administration.

In some embodiments, the amount of host cells in a pharmaceutical composition is typically greater than 10² cells, for example, about 1×10², 5×10², 1×10³, 5×10³, 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰ cells, or more.

In some embodiments, the methods comprise administering to the subject a population of host cells, or a pharmaceutical composition containing the same, once a day, twice a day, three times a day, or four times a day for a period of about 3 days, about 5 days, about 7 days, about 10 days, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 1 year, about 1.25 years, about 1.5 years, about 1.75 years, about 2 years, about 2.25 years, about 2.5 years, about 2.75 years, about 3 years, about 3.25 years, about 3.5 years, about 3.75 years, about 4 years, about 4.25 years, about 4.5 years, about 4.75 years, about 5 years, or more than about 5 years. In some embodiments, the population of host cells, or a pharmaceutical composition containing the same, can be administered every day, every other day, every third day, weekly, biweekly (i.e., every other week), every third week, monthly, every other month, or every third month.

In some embodiments, the population of host cells, or a pharmaceutical composition containing the same, may be administered over a pre-determined time period. Alternatively, the population of host cells, or a pharmaceutical composition containing the same, may be administered until a particular therapeutic benchmark is reached. In some embodiments, the methods provided herein include a step of evaluating one or more therapeutic benchmarks in a biological sample, such as, but not limited to, the level of a cancer biomarker, to determine whether to continue administration of the host cells, or the pharmaceutical composition containing the same.

In some embodiments, the method further entails administering one or more other cancer therapies such as surgery, immunotherapy, radiotherapy, and/or chemotherapy to the subject, sequentially or simultaneously.

In some embodiments, the methods further comprise administering the subject a pharmaceutically effective amount of one or more additional therapeutic agents to obtain improved or synergistic therapeutic effects. In some embodiments, the one or more additional therapeutic agents are selected from the group consisting of an immunotherapy agent, a chemotherapy agent, and a biologic agent. In some embodiments, the subject was administered the one or more additional therapeutic agents before administration of the host cells, or the pharmaceutical composition containing the same. In some embodiments, the subject is co-administered the one or more additional therapeutic agents and the host cells, or the pharmaceutical composition containing the same. In some embodiments, the subject was administered the one or more additional therapeutic agents after administration of the host cells, or the pharmaceutical composition containing the same.

As one of ordinary skill in the art would understand, the one or more additional therapeutic agents and the host cells, or the pharmaceutical composition containing the same, can be administered to a subject in need thereof one or more times at the same or different doses, depending on the diagnosis and prognosis of the subject. One skilled in the art would be able to combine one or more of these therapies in different orders to achieve the desired therapeutic results. In some embodiments, the combinational therapy achieves improved or synergistic effects in comparison to any of the treatments administered alone.

### EXAMPLES

### Example 1. Bicistronic Vectors Co-Expressing CD47 and CD19 CAR Provide Sufficient Host-Versus-Graft Protection

In this study, three bicistronic vectors were developed and evaluated for CD47/CD19 CAR expression levels, CD19+ target cell killing, and hypoimmunity in vitro.

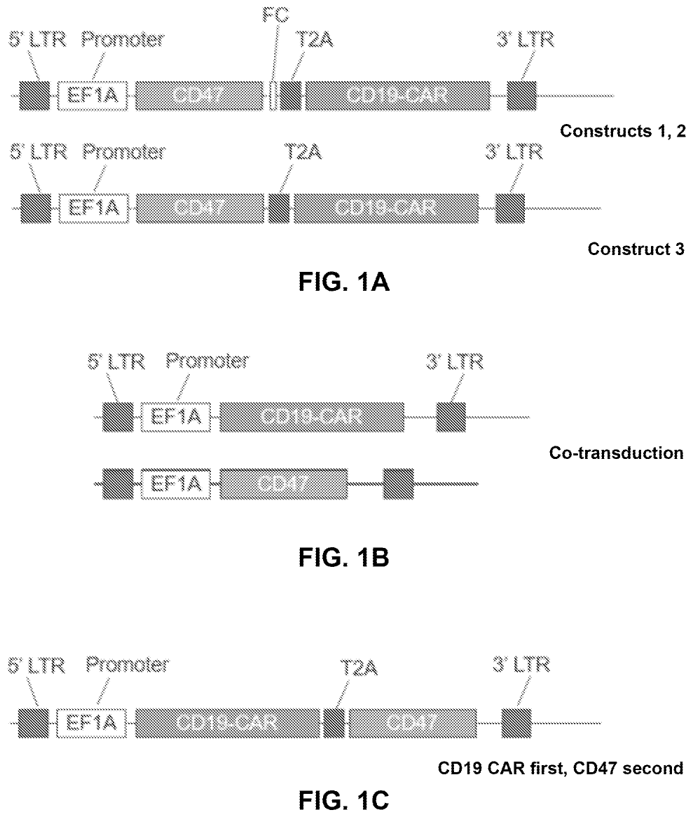

FIG. 1A shows a general design of the bicistronic vector according to certain embodiments of the present technology. As shown, the bicistronic vector has an EF1α promoter, a coding sequence for CD47, a cleavage site, and a coding sequence for CD19 CAR. However, the cleavage site can optionally have a furin site in addition to a 2A site (top) or a 2A site only (bottom).

### CD19 CAR/CD47 Expression

First, the transduction and expression efficiencies of the bicistronic vector design were tested in comparison to transduction of the vectors comprising only a single genetic sequence, as well as the co-transduction design, where two lentiviral vectors respectively encoding CD19 CAR and CD47 were used to co-transduce cells (FIG. 1B). Primary T cells collected from donors were transduced with (1) no construct (control); (2) CD47-encoding lentiviral vector alone; (3) CD19 CAR-encoding lentiviral vector alone; (4) bicistronic vector encoding both CD47 and CD19 CAR; and (5) two lentiviral vectors-one encoding CD19 CAR and one encoding CD47.

Briefly, the T cells were harvested, resuspended in media, counted, and plated for lentiviral transduction. At 7 days post transduction, the expression of CD47 and CD19 CAR was examined by flow cytometry.

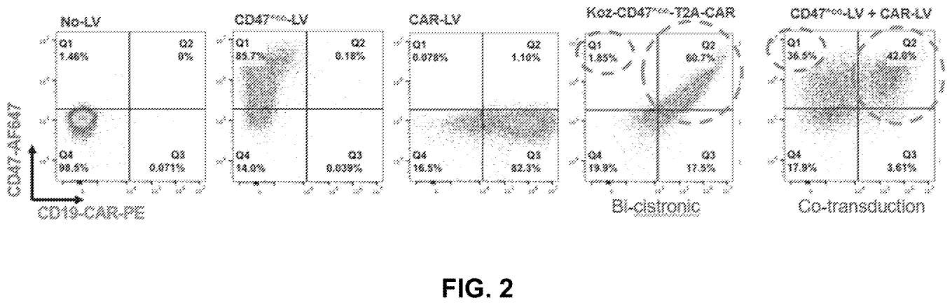

As shown in FIG. 2, bicistronic lentiviral vector treatment resulted in greater CD47/CAR double positive cells than co-transduced primary T cells. In particular, bicistronic lentiviral vector of CD47-T2A-CD19 CAR resulted in 60% CD47+, CD19 CAR+ cells, while co-transduction of CD47 vector and CD19 CAR vector resulted in 42% CD47+, CD19 CAR+ cells. In the co-transduction group, there is a noticeable decrease in total percentage of CD19 CAR+ cells (45%), compared with 82% CD19 CAR+ cells in the single vector group. These data suggest that transduction of T cells with a single bicistronic lentiviral vector produced a greater number of CD47/CD19 CAR double positive cells and more CD19 CAR+ cells than co-transduction of two vectors.

In addition, it was found that CD47-CD19 CAR bicistronic CAR-T cells produced comparable titers and killed target CD19+ Nalm-6 cells in vitro at a level comparable to CD19 CAR-only CAR-T cells (data not shown), suggesting that the CD47-CD19 CAR bicistronic vector is able to generate sufficient levels of functional CD19 CAR as intended.

### CD47 Expression Level and Hypoimmunity

Endogenous CD47 expression on T cells is not sufficient to protect cells from innate immune cell killing in an allogeneic host, which requires high CD47 levels for immune evasion. Therefore, it is important that the bicistronic vector of the present technology will generate sufficient CD47 expression, at least above threshold required to confer hypoimmunity. It was hypothesized that the placement of CD47 ahead of CD19 CAR in the bicistronic vector would help maximize CD47 expression. This hypothesis was tested by comparing to an alternative design of placing CD19 CAR first and CD47 second (FIG. 1C).

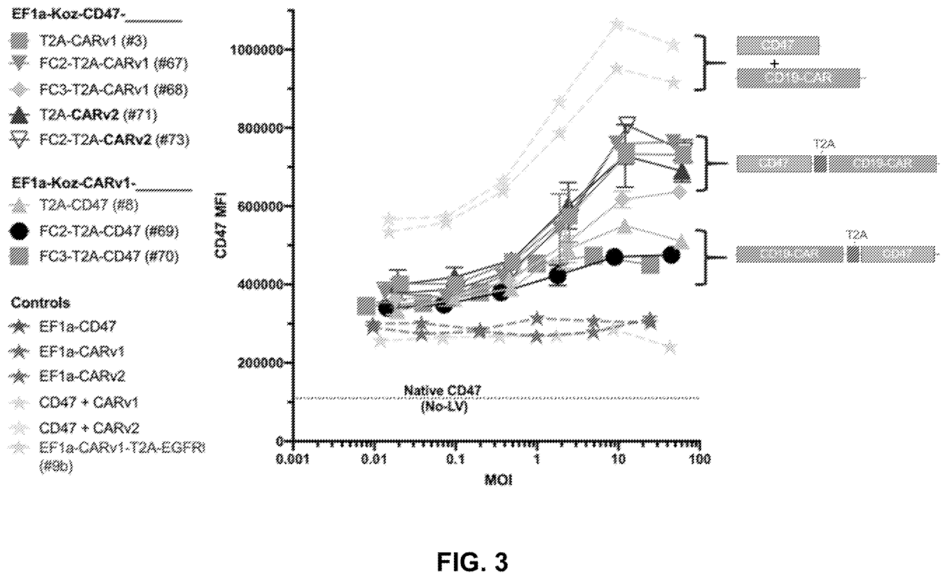

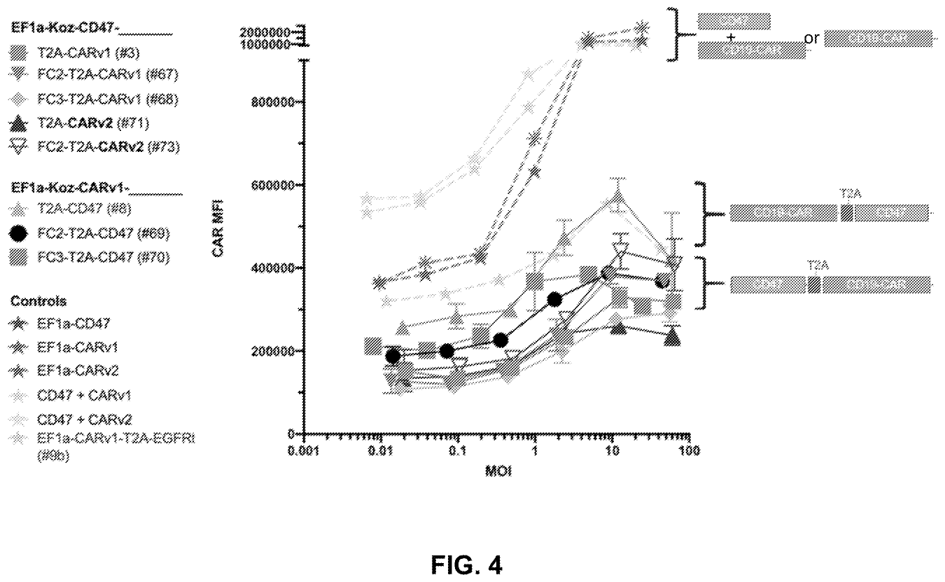

As shown in FIGS. 3 and 4, placing CD47 first in the bicistronic vector produced higher levels of CD47 (6.5× fold increase compared to native level) than placing CD19 CAR first. Conversely, placing CD19 CAR first in the bicistronic vector produced higher levels of CD19 CAR than placing CD47 first (1.7× fold difference).

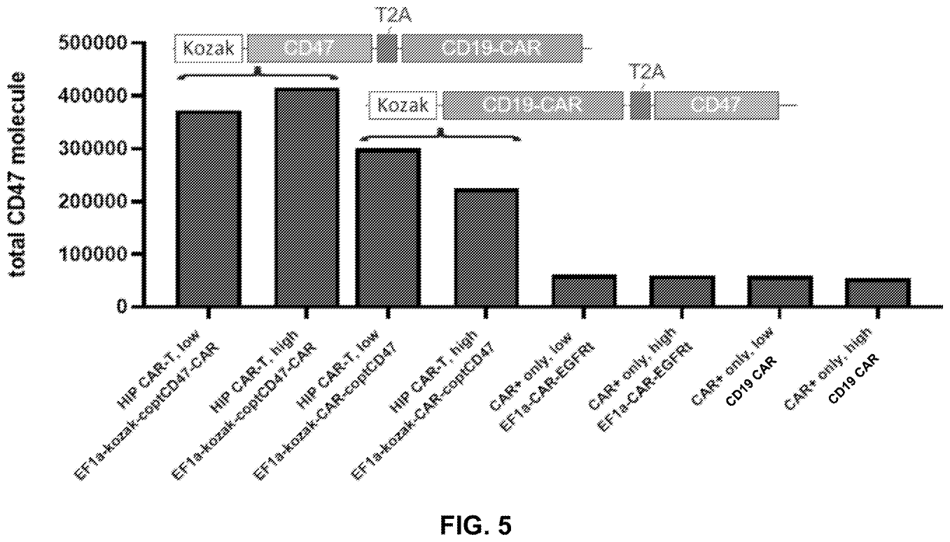

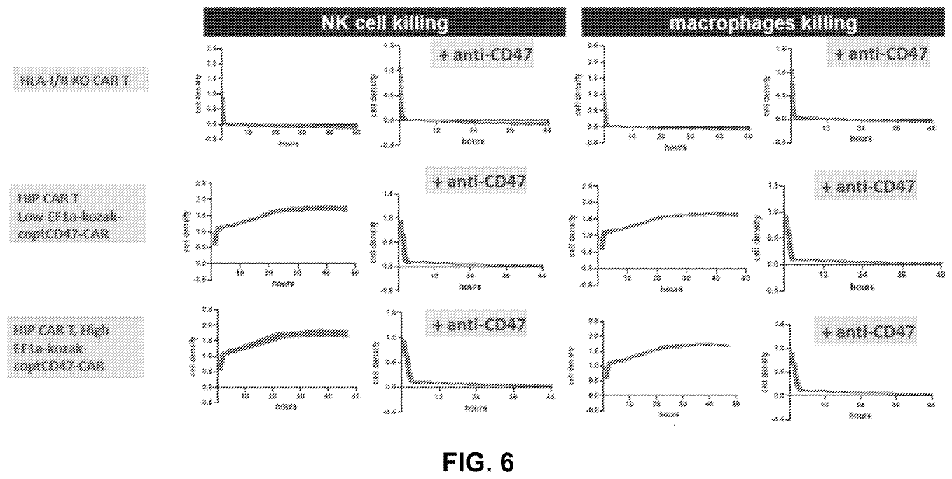

CD47 expression levels were analyzed using Quantibrite™. As shown in FIG. 5, CD47 expression for both the CD47-CD19 CAR and the CD19 CAR-CD47 bicistronic constructs was at or above the level of CD47 expression forma vector comprising a single expression cassette. The hypoimmunity of HIP CAR-T cells (which has HLA I/II knockout (KO) by way of B2M and CIITA deletion) expressing CD47 from the CD47-CD19 CAR bicistronic vector was tested. As shown in FIG. 6, CD47-CD19 CAR-transduced cells exhibited lower NK cell killing and macrophage killing compared with HLA-I/II KO CAR-T cells, and the immune protection was blocked by addition of anti-CD47 antibody. Together, these results show that CAR-T cells expressing CD47 from the CD47-CD19 CAR bicistronic vector are hypoimmune.

### Generation and Testing of Three CD47-CD19 CAR Bicistronic Vectors

Subsequently, three CD47-CD19 CAR bicistronic vectors were identified and characterized for their CD47/CD19 CAR expression levels, CD19+ target cell killing, and hypoimmunity in vitro.

Three top-performing bicistronic vectors: EF1a-Kozak-CD47-FC2-T2A-CD 19 CAR (Construct 1); EF1α-Kozak-CD47-FC3-T2A-CD 19 CAR (Construct 2); and EF1α-Kozak-CD47-T2A-CD 19 CAR (Construct 3), utilizing the Psf2 lentiviral vector backbone, were generated and identified. All three constructs follow the general design as shown in FIG. 1A, in that they all have an EF1α promoter, a coding sequence for CD47, a cleavage site, and are followed by a coding sequence for CD19 CAR. However, the cleavage site of Constructs 1 and 2 has a furin site in addition to a T2A site (Construct 1 has an additional FC3 site; Construct 2 has an additional FC2 site), whereas Construct 3 only has a T2A site (FIG. 1A). Their CD47 and/or CD19 CAR expression levels and CD19+ target cell killing efficiency are summarized in Table 20 below.

TABLE 20
Characterization of bicistronic vector Constructs 1, 2, and 3

| | | | Fold-increase | CD19 CAR | | |
|---|---|---|---|---|---|---|
| | % CD47+/ | | in CD47 MFI | MFI (vs. CD19 | CD19 CAR+ | EC50 |
| Construct | CD19 CAR+ | % CD19 CAR+ | (vs. native) | CAR single vector) | titer (TU/mL) | killing |
| 1 | 77.2 | 84.1 | 7.2 | 0.25 | 2.0E+07 | 0.06 |
| 2 | 77.3 | 84.3 | 6.7 | 0.28 | 1.7E+07 | 0.08 |
| 3 | 75.5 | 82.3 | 7.1 | 0.19 | 2.4E+07 | 0.10 |

Accordingly, Constructs 1, 2, and 3 have high performances in terms of CD47 and/or CD19 CAR expression levels and CD19-targeted immune response, comparable with each other.

### Example 2. Bicistronic Vectors Co-Expressing CD47 and a Safety Switch Enable Controlled Killing of Host Cells

In this study, bicistronic vectors were generated for co-expression of CD47 and a safety switch and tested for controlled killing of host cells with the expression of the safety switch.

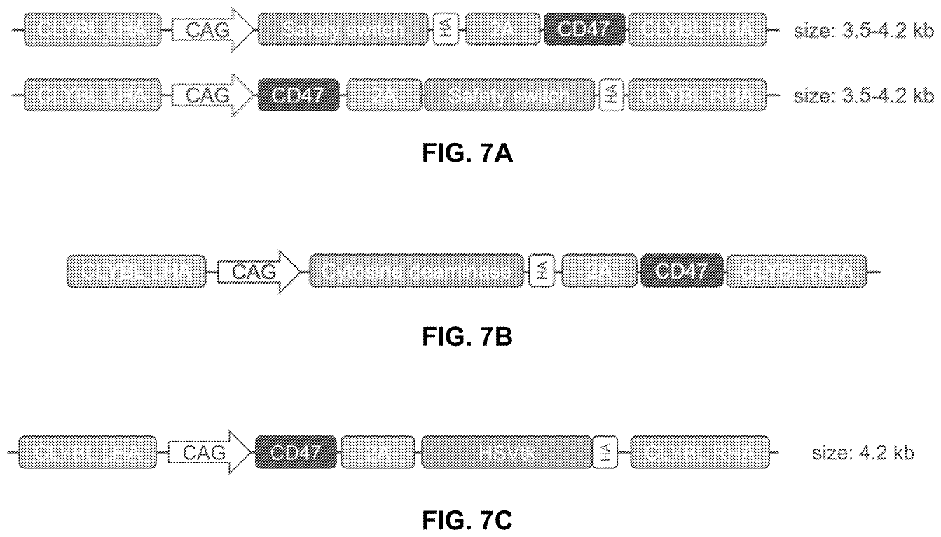

Bicistronic vectors for co-expression of CD47 and a safety switch may include a construct with a general design as shown in FIG. 7A. The construct may include a promoter (e.g., CAG promoter), a coding sequence for CD47, and a coding sequence for a safety switch transgene (e.g., cytosine deaminase (CyD), herpes simplex virus thymidine kinase (HSVtk)) optionally followed by an HA tag. Two versions of the bicistronic vector may be generated, the first one having the coding sequence for the safety switch before the coding sequence for CD47 in the 5′ to 3′ order (FIG. 7A, top), and the second having the coding sequence for CD47 before the coding sequence for the safety switch in the 5′ to 3′ order (FIG. 7A, bottom). A 2A site can be used to separate the safety switch and CD47. The construct can be flanked by left and right homology arms (LHA, RHA) that are specific to a target genomic locus of interest (e.g., the CLYBL safe harbor locus) for directed insertion through homology-directed repair (HDR).

As shown in FIG. 7B, a bicistronic vector that includes a construct having a CAG promoter, a coding sequence for CyD followed by an HA tag, a 2A site, and a coding sequence for CD47 was generated for co-expression of CD47 and CyD. The construct is flanked by CLYBL LHA and RHA for site-directed insertion into the CLYBL safe harbor locus. As described herein, the CyD safety switch could inducibly stop cell growth and/or kill proliferating cells that have the construct in the presence of prodrug 5-fluorocytosine (5-FC).

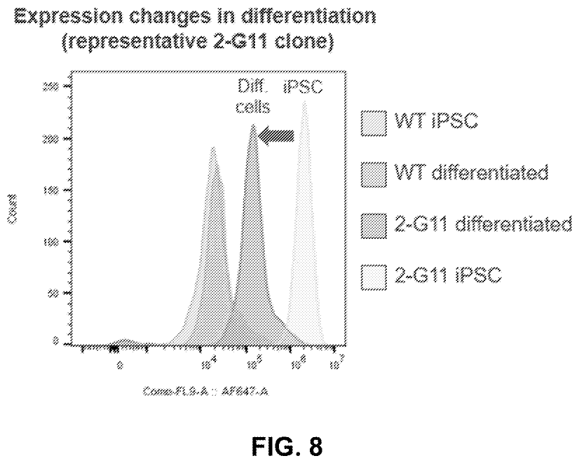

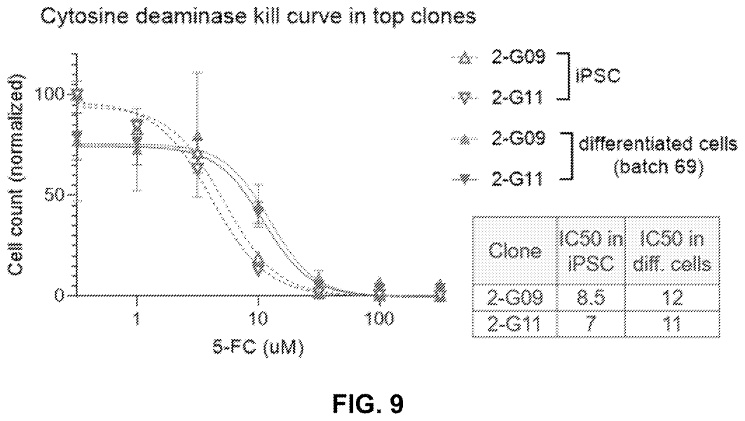

The bicistronic vector was used to transduce induced pluripotent stem cells (iPSCs), which were then allowed to differentiate. Differentiation of CyD-CD47-expressing clones in 2D was not affected by safety switch expression (data not shown here). As shown in FIG. 8, transduced cells had increased CD47 expression compared with wild-type (WT) control, but lower expression of the bicistronic cassette (based on CD47 expression) after insertion into the CLYBL locus was observed post differentiation in transduced cells. Finally, as shown in FIG. 9, based on the results from 2 clones (2-G09 and 2-G11), killing efficiency of CyD in the presence of 5-FC was achieved and maintained (more than 99% killing achieved) in differentiated cells at comparable IC50s relative to iPSCs. These results show effective and controllable killing of cells having the CyD-CD47 bicistronic construct.

Another bicistronic vector for co-expression of CD47 and a different safety switch, HSVtk, was also generated and tested. As shown in FIG. 7C, the bicistronic vector includes a construct having a CAG promoter, a coding sequence for CD47, a 2A site, and a coding sequence for HSVtk followed by an HA tag. HSVtk could convert ganciclovir (GCV) to a toxic metabolite that interferes with DNA synthesis and thereby kills dividing cells. As previously, the construct is flanked by CLYBL LHA and RHA for site-directed insertion into the CLYBL safe harbor locus.

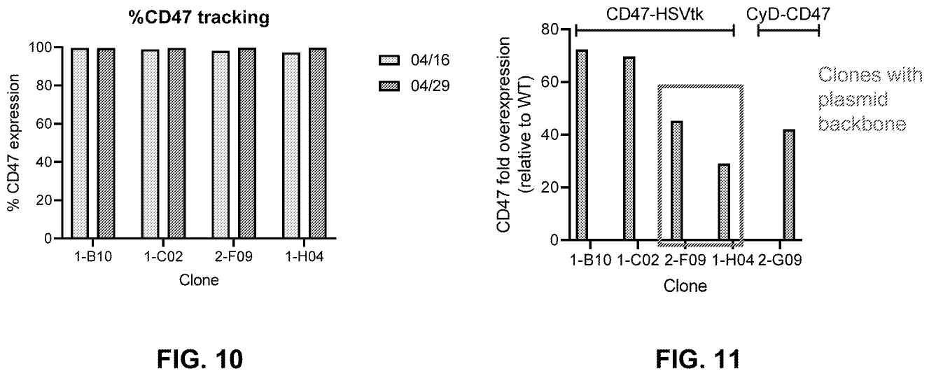

iPSCs were transduced with the CD47-HSVtk bicistronic construct and then allowed to differentiate into clones for testing. As shown in FIG. 10, CD47 expression was tested in several clones (1-B10, 1-C02, 2-F09, and 1-H04) over a period of two weeks, and no silencing of CD47 expression was observed over the two weeks. All clones were at least 98% positive for CD47. As shown in FIG. 11, CD47 overexpression relative to wild-type level was tested in these clones, with the CyD-CK47 clone 2-G09 as a control. About 70-fold overexpression for clones with 1 copy of CD47 was observed, but insertion of plasmid backbone lowered CD47 expression levels.

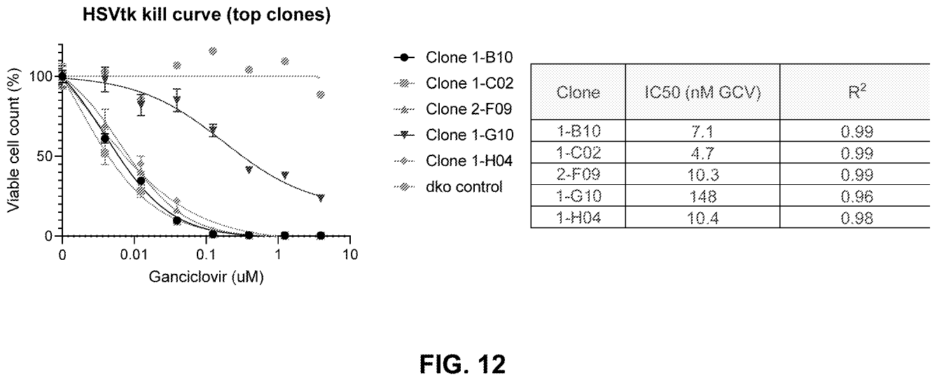

As shown in FIG. 12, killing efficiency and dose response of HSVtk in the presence of various amounts of GCV were tested in the CD47-HSVtk clones after three days by flow cytometry with counting beads. While no GCV toxicity was observed in the control clone in the tested dose range, dose-response HSVtk killing was achieved in the CD47-HSVtk clones with IC50s generally comparable to what is reported in literature (10-100 nM). Together, these results show stable CD47 overexpression and controllable killing of cells having the CD47-safety switch bicistronic construct.

### Example 3. Dual Transduction with CD47-CD19 CAR and CD47-CD22 CAR Bicistronic Vectors Increases Cytotoxicity and Cytokine Response Compared to Single Transduction Controls

In this study, bicistronic vectors for co-expression of CD47 and CD22 CAR were generated, characterized, and tested alongside CD47-CD19 CAR bicistronic vectors for dual transduction of T cells. The single and dual transduced CAR-T cells were then examined for their tumor killing abilities in vitro and in mouse tumor models.

### CD47-CD22 CAR Bicistronic Vector Design and Testing

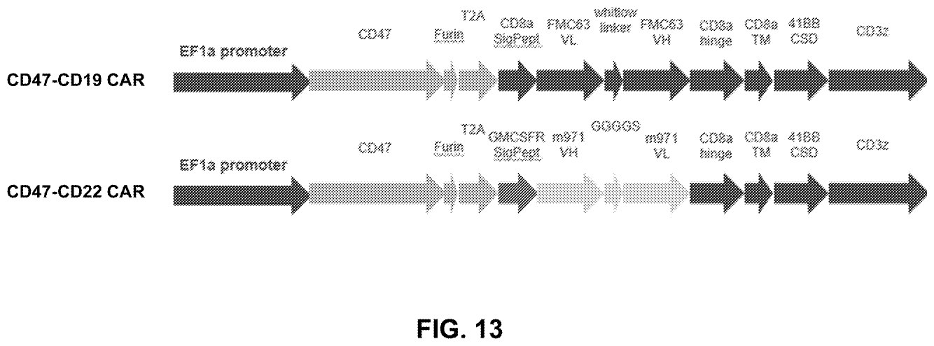

A CD47-CD22 CAR bicistronic vector (PLAS2199) having a design as shown in FIG. 13 was generated. The CD47-CD22 CAR bicistronic vector includes an EF1α promoter, a coding sequence for CD47, a furin site, a T2A site, and a coding sequence for CD22 CAR. The CD22 CAR has the following functional domains: a GMCSFR-α signal peptide, a CD22-specific scFv comprising the V_{H} and V_{L} of m971 connected by a short G₄S linker, a CD8α hinge domain, a CD8α transmembrane domain, a 4-1 BB costimulatory domain, and a CD3ζ signaling domain. The CD22 CAR construct also has a short AAA linker between the V_{L} domain and the CD8α hinge domain.

Next, the physical and functional characteristics of the CD47-CD22 CAR construct was tested and compared with the CD47-CD19 CAR construct as described. A design of the CD47-CD19 CAR is also shown in FIG. 13. The CD19 CAR has the following functional domains: a CD8α signal peptide, a CD19-specific scFv comprising the V_{H} and V_{L} of FMC63 connected by a Whitlow linker, a CD8α hinge domain, a CD8α transmembrane domain, a 4-1BB costimulatory domain, and a CD3ζ signaling domain. The CD19 CAR coding sequence is downstream of a coding sequence for CD47, and the two code sequences are separated by a furin site and a T2A site. Both coding sequences are driven by an EF1α promoter. A CD22 CAR-CD47 bicistronic vector (PLAS2218) was also generated as control by swapping the coding sequence for CD22 CAR and the coding sequence for CD47.

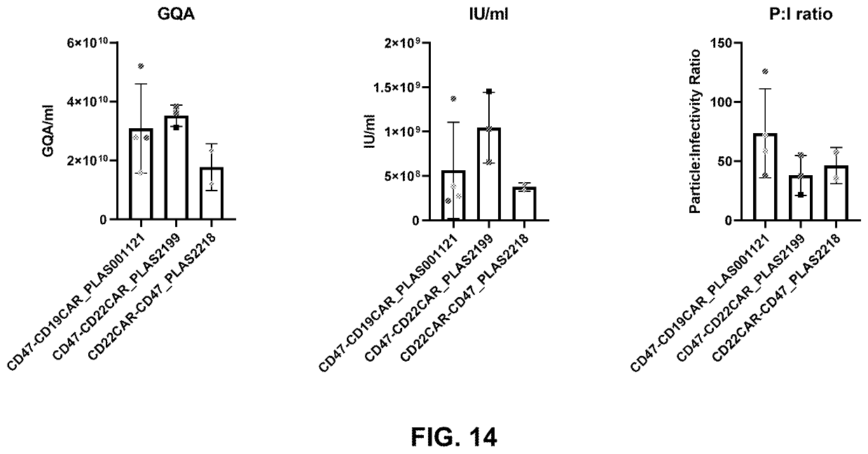

All three bicistronic vectors, CD47-CD19 CAR, CD47-CD22 CAR (PLAS2199), and CD22 CAR-CD47 (PLAS2218), were used to generate lentiviruses, which were then tested in a series of physical and functional assays including genome quantitation assay (GQA), functional titer assay, and particle to infectivity assay. As shown in FIG. 14, the CD47-CD22 CAR lentivirus exhibited similar physical and functional titering profile to the CD47-CD19 CAR lentivirus.

### CD47-CD22 CAR/CD47-CD19 CAR Dual Transduction

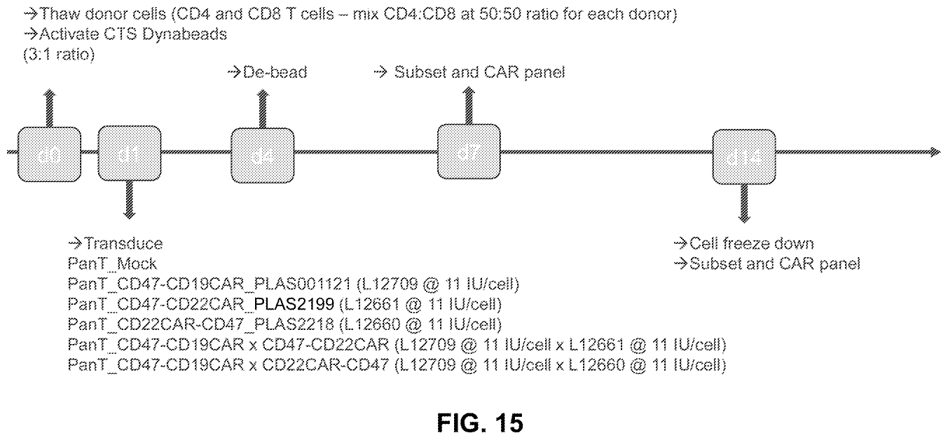

Next, a dual transduction approach using both the CD47-CD22 CAR and CD47-CD19 CAR viruses was tested. A workflow of the dual transduction approach is shown in FIG. 15. Briefly, on Day 0, donor CD4+ and CD8+ T cells were thawed, mixed at 1:1 ratio, and applied to CTS™ Dynabeads™ (Thermo Fisher Scientific) at a 3:1 ratio. On Day 1, the T cells were transduced with lentiviruses. A total of 6 lentivirus treatments were tested: (1) mock; (2) CD47-CD19 CAR lentivirus alone; (3) CD47-CD22 CAR (PLAS2199) lentivirus alone; (4) CD22 CAR-CD47 (PLAS2218) lentivirus alone; (5) CD47-CD19 CAR lentivirus and CD47-CD22 CAR (PLAS2199) lentivirus mixture (CD47-CD19 CAR×CD47-CD22 CAR); and (6) CD47-CD19 CAR lentivirus and CD22 CAR-CD47 (PLAS2218) lentivirus mixture (CD47-CD19 CAR×CD22 CAR-CD47). On Day 4, the cells were separated from the beads, and a subset was subject to subsequent assays on Day 7. On Day 14, another subset of cells was tested again, and the remaining cells were frozen.

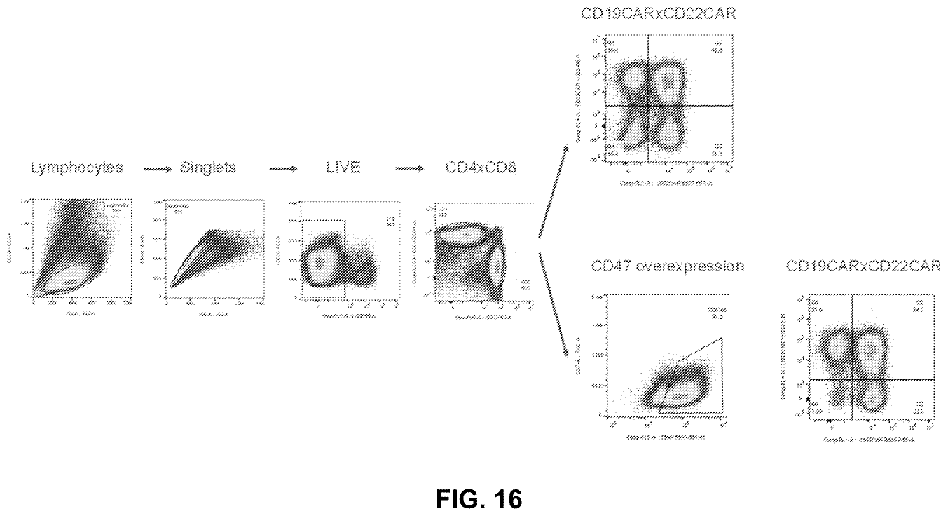

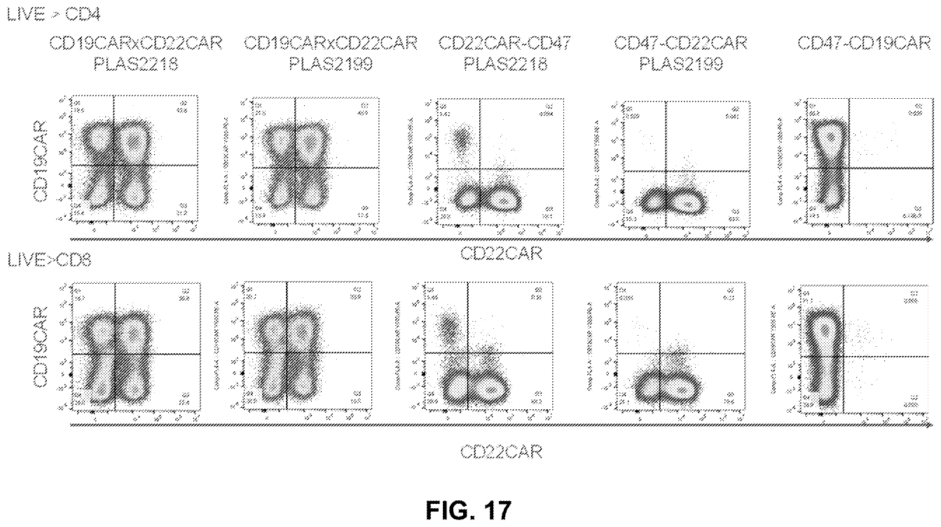

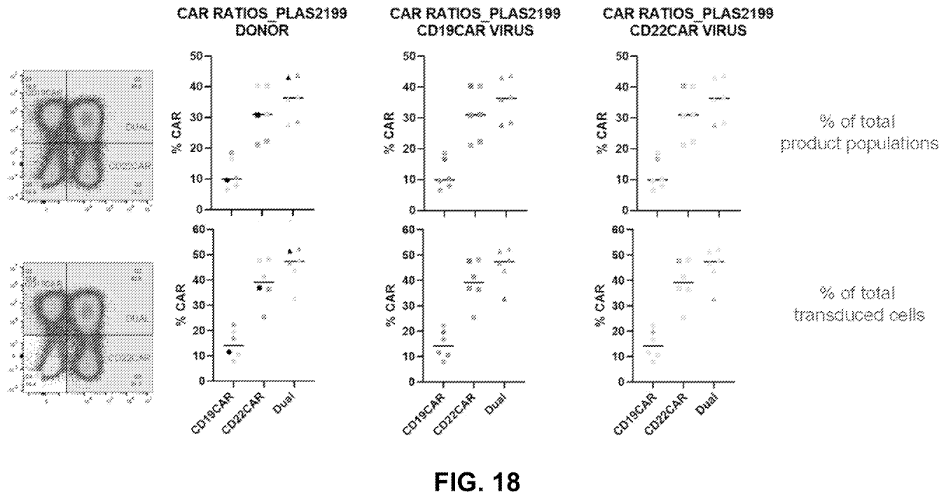

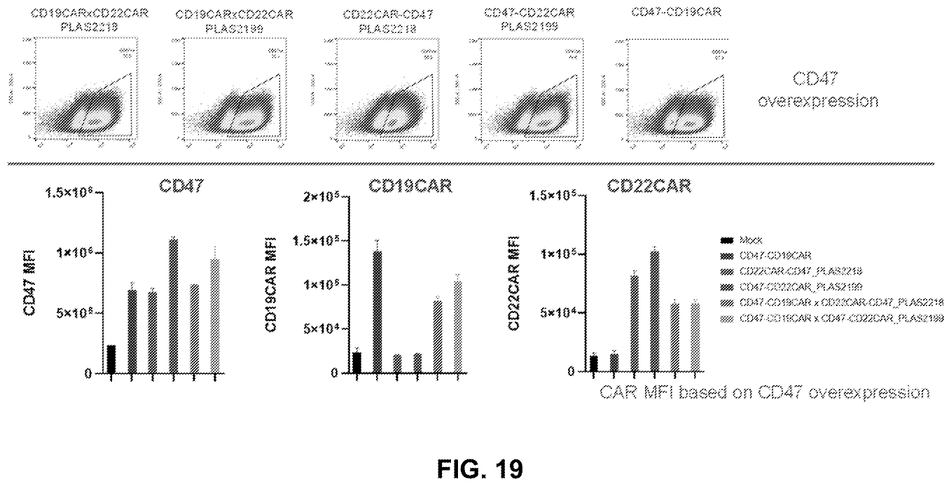

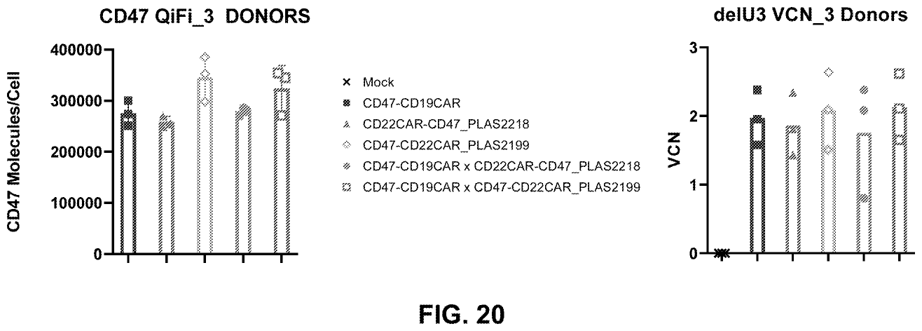

FIG. 16 shows an example gating strategy of flow cytometry analysis of the single or dual transduced T cells to examine CD47, CD19 CAR, and CD22 CAR expression levels. Starting T cells were selected for CD4 and/or CD8 expression, then optionally for CD47 expression, and tested for CD19 and CD22 expression. As shown in FIG. 17, transduction of the CD47-CD19 CAR lentivirus or CD47-CD22 CAR/CD22 CAR-CD47 lentivirus alone led to expression of CD19 CAR and CD22 CAR respectively in both CD4+ and CD8+ T cells as expected, suggesting that the CD47-CD22 CAR/CD22 CAR-CD47 bicistronic lentivirus could effectively transduce pan T cells. Moreover, dual transduction of both CD47-CD19 CAR and CD47-CD22 CAR/CD22 CAR-CD47 lentiviruses resulted in a heterogenous population of T cells exhibiting high levels of CD19 CAR expression, CD22 CAR expression, or both. The dual transduction approach was repeated on different donors, and results show that the dual transduction approach was able to create a consistent product distribution between donors and virus lots (FIG. 18). To note, compared to the CD22 CAR-CD47 design, the CD47-CD22 CAR design was able to afford higher CD47 and CD22 CAR expression levels as measured by median fluorescent intensity (MFI) in flow cytometry in three donors (FIG. 19), consistent with previous results and suggesting that it might be beneficial to place the CAR construct downstream of the CD47 construct. Moreover, the CD47-CD22 CAR construct produced higher CD47 expression level at similar vector copy number (VCN) relative to the CD47-CD19 CAR construct, above the threshold required by hypoimmunity (FIG. 20), suggesting that the T cells produced from the CD47-CD22 CAR construct are hypoimmune. Due to the superior characteristics of the CD47-CD22 CAR construct over the CD22 CAR-CD47 construct, the former was used for subsequent tests.

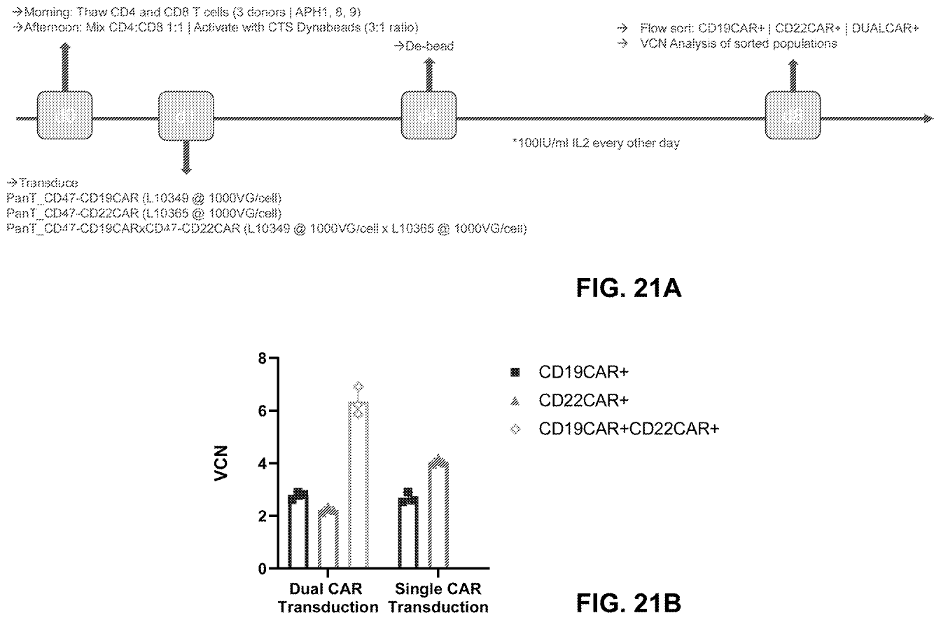

Finally, it was tested whether dual transduced cell populations have higher VCN compared to single transduced cell populations. As shown in FIG. 21A, CD4+ and CD8+ T cells from 3 donors were thawed on Day 0 and mixed at 1:1 ratio before application of CTS™ Dynabeads™ (Thermo Fisher Scientific) at a 3:1 ratio. On Day 1, the T cells were single or dual transduced with lentiviruses: (1) CD47-CD19 CAR lentivirus alone; (2) CD47-CD22 CAR lentivirus alone; and (3) CD47-CD19 CAR lentivirus and CD47-CD22 CAR lentivirus mixture (CD47-CD19 CAR×CD47-CD22 CAR). The cells were separated from the beads on Day 4, and flow cytometry and VCN analysis were conducted on Day 8. As shown in FIG. 21B, dual transduced population had a higher VCN than single CAR transduced cells, and dual transduced product was average of the VCNs of single and dual transduced cells.

### Cytotoxicity of Dual CD47-CD19 CAR/CD47-CD22 CAR Transduced T Cells In Vitro

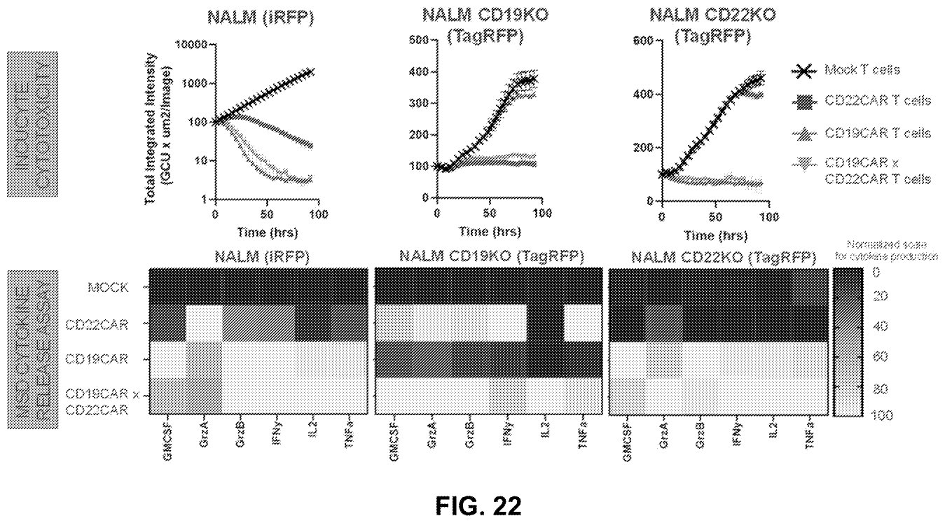

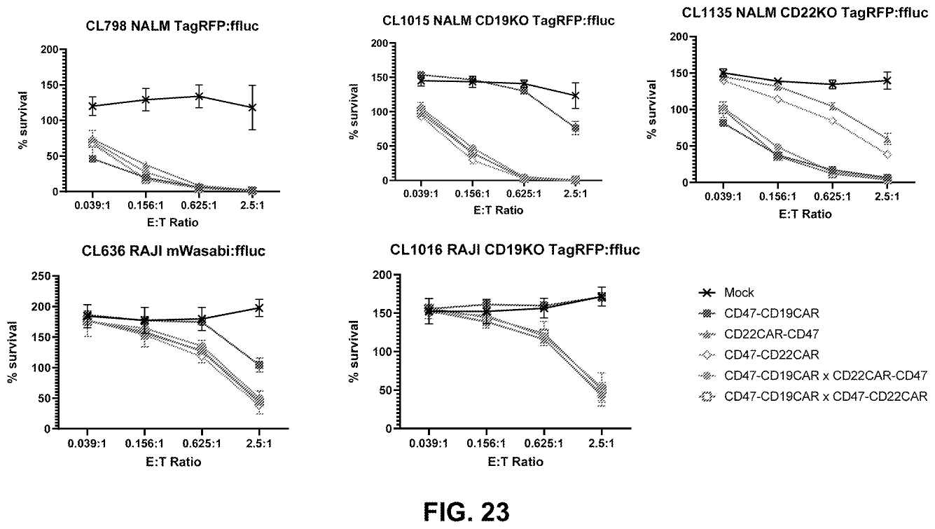

Next, it was tested wither dual CD47-CD19 CAR/CD47-CD22 CAR transduced T cells could inhibit tumor growth in vitro. As shown in FIG. 22, T cells single transduced with CD47-CD19 CAR (CD19 CAR-T cells), single transduced with CD47-CD22 CAR (CD22 CAR-T cells), and dual transduced with CD47-CD19 CAR/CD47-CD22 CAR (CD19 CAR×CD22 CAR-T cells) were tested on RFP-labeled control NALM, CD19 KO NALM, or CD22 KO NALM tumor cells, and cytotoxicity was measured by total integrated intensity of RFP over time. Compared to singled transduced T cells, dual transduced T cells overcame antigen escape and produced cytokine against CD19 or CD22 KO targets in NALM cells. As shown in FIG. 23, CD47-CD22 CAR elicited cytotoxicity against NALM and RAJI tumor cells in an antigen dependent manner.

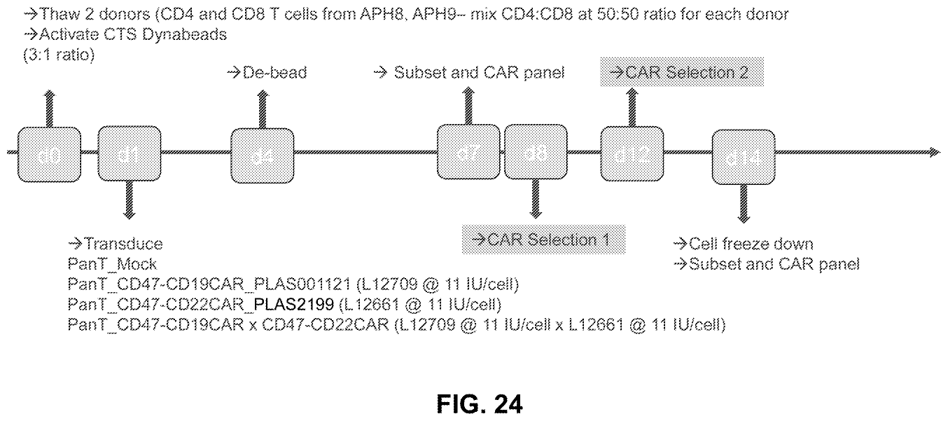

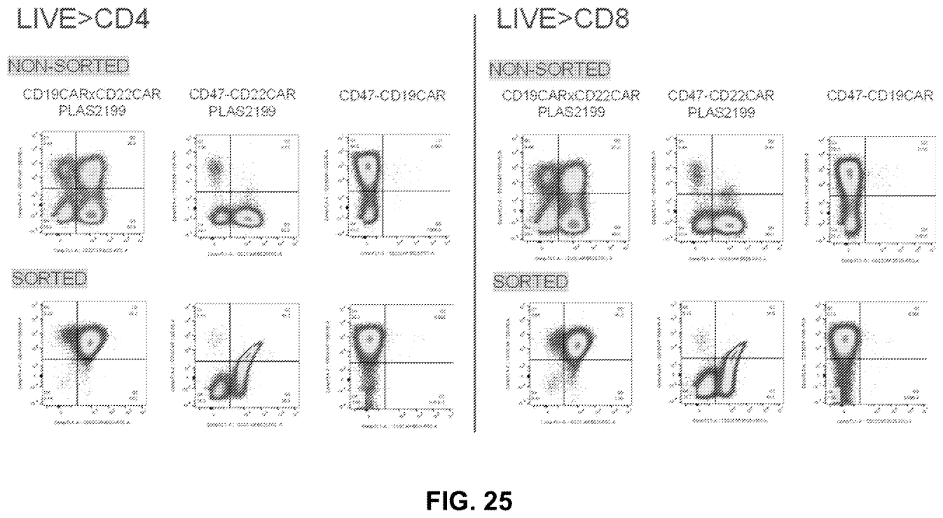

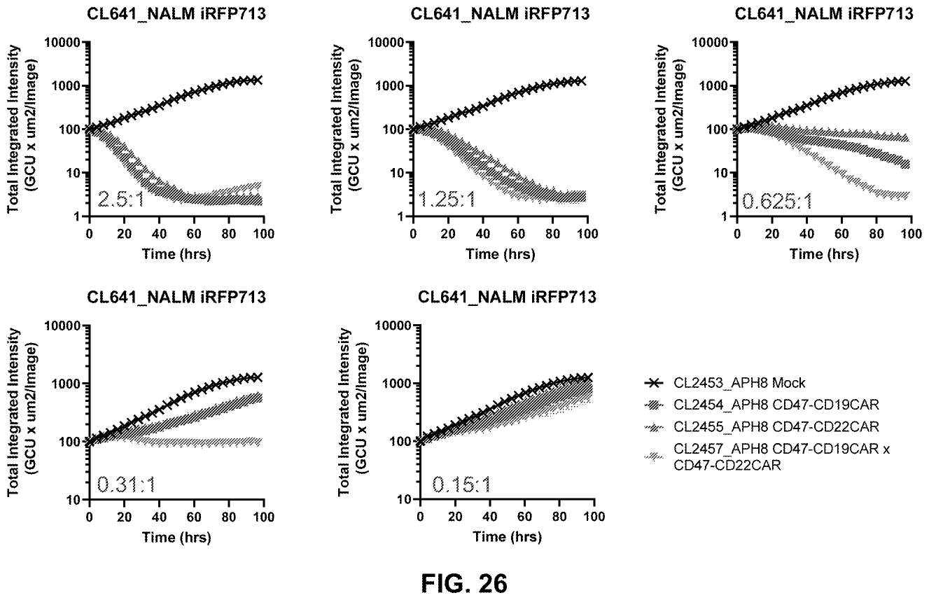

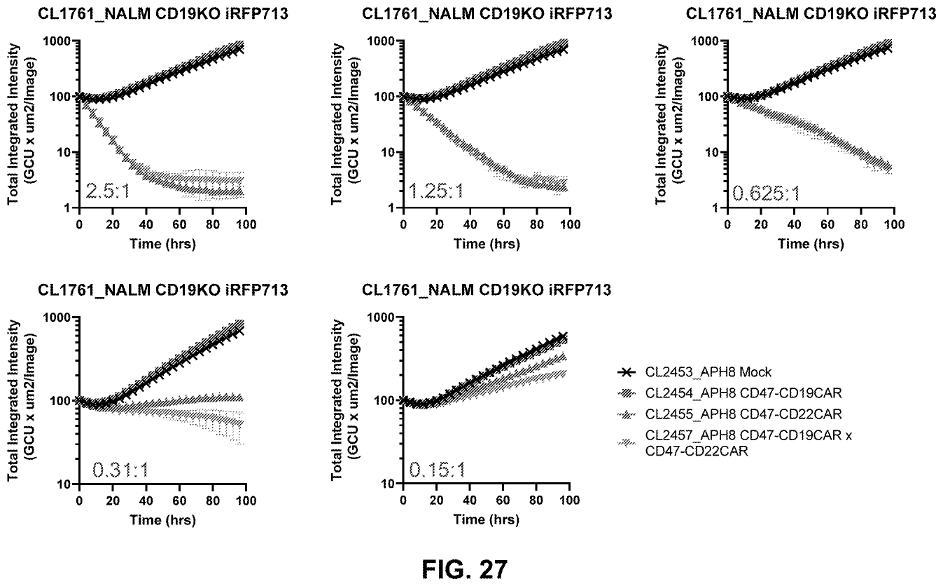

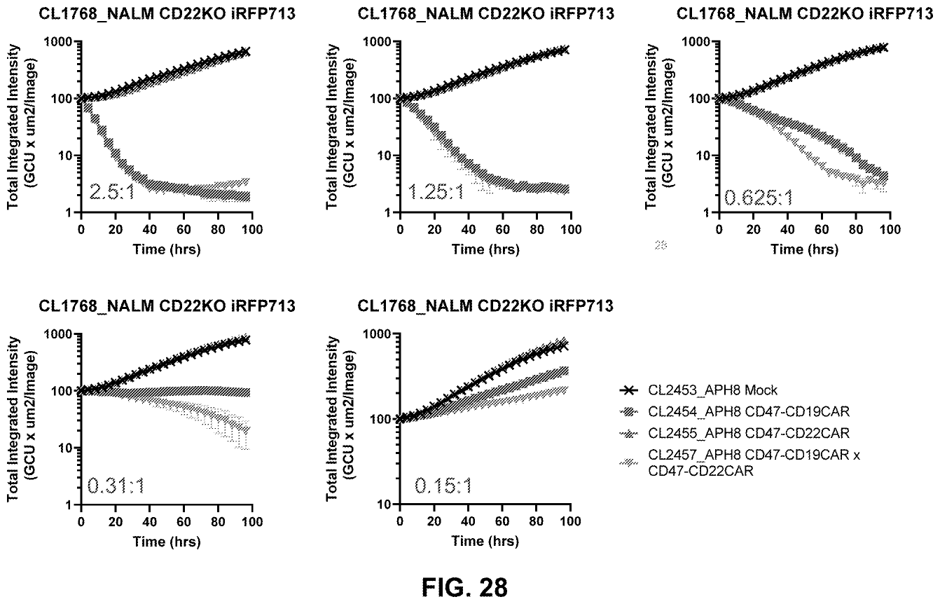

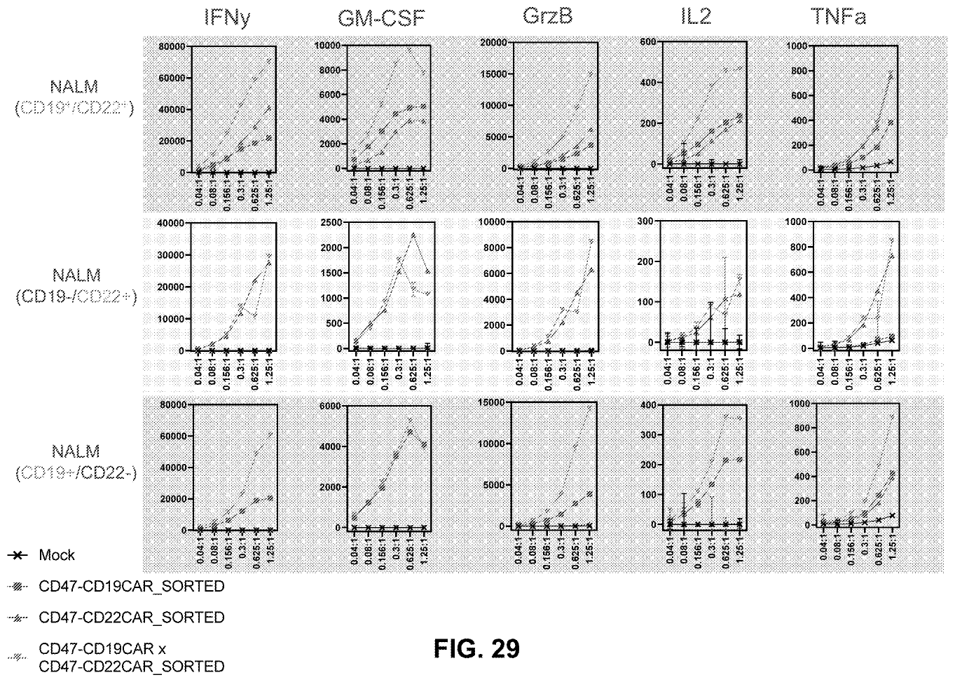

To further examine whether dual transduced CD19 CAR×CD22 CAR-T cells would elicit similar cytotoxicity and cytokine profile relative to single CAR-expressing cells, a study was carried out where single transduced CD19 CAR-T cells, single transduced CD22 CAR-T cells, and dual transduced CD19 CAR×CD22 CAR-T cells were sorted by magnetic selection and then assayed by cytotoxicity (IncuCyte®) and cytokine (Meso Scale Discovery) analyses (see FIG. 24 showing an example workflow). Briefly, CD4+ and CD8+ T cells from 2 donors were thawed on Day 0 and mixed at 1:1 ratio before application of CTS™ Dynabeads™ (Thermo Fisher Scientific) at a 3:1 ratio. On Day 1, T cells were (1) transduced with mock construct; (2) single transduced with CD47-CD19 CAR (CD19 CAR-T cells); (3) single transduced with CD47-CD22 CAR (CD22 CAR-T cells); or (4) dual transduced with CD47-CD19 CAR and CD47-CD22 CAR (CD19 CAR×CD22 CAR-T cells). The cells were separated from the beads on Day 4, and a subset was tested on Day 7. On Day 8, the CD19 CAR-T cells and dual transduced T cells were sorted by anti-idiotype-biotin and anti-biotin microbeads, and the CD22 CAR-T cells were sorted by soluble CD22-biotin. On Day 12, the dual transduced population was sorted again by soluble CD22-biotin for CD22 CAR. The sorting steps were to select a sub-population of the single or dual transduced T cells to enrich the cells that express both CD19 CAR and/or CD22 CAR. As shown in FIG. 25, dual transduced cells were efficiently sorted (purified) to a sub-population where more than 90% of the cells express both CD19 CAR and CD22 CAR. On Day 14, another subset of cells was tested for CAR panel, and the remaining were frozen. As shown in FIG. 26, dual transduced and sorted CAR-T cells more efficiently inhibited tumor cell growth relative to single transduced and sorted CAR-T controls at low effector (E) to target (T) (E:T) ratios. Dual transduced and sorted CAR-T cells also showed more effective control of tumor growth in a CD19 KO (FIG. 27) or CD22 KO (FIG. 28) background. Moreover, dual transduced CAR-T cells elicited higher levels of cytokines relative to single transduced CAR-T cells when tested in control, CD19 KO, and CD22 KO NALM cells (FIG. 29). Taken together, these data suggest dual transduced CAR-T cells exhibit superior cytotoxicity and cytokine responses compared to single transduced CAR-T cells in vitro.

### Cytotoxicity of Dual CD47-CD19 CAR/CD47-CD22 CAR Transduced T Cells In Vivo

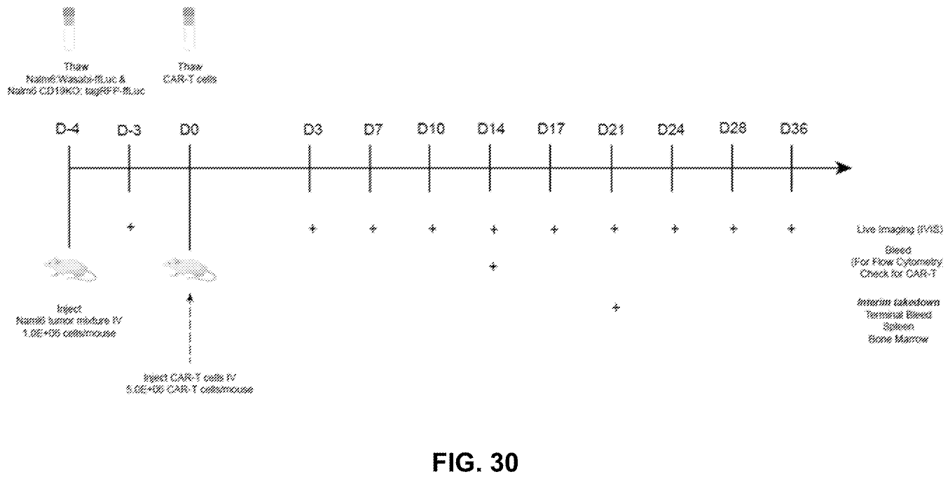

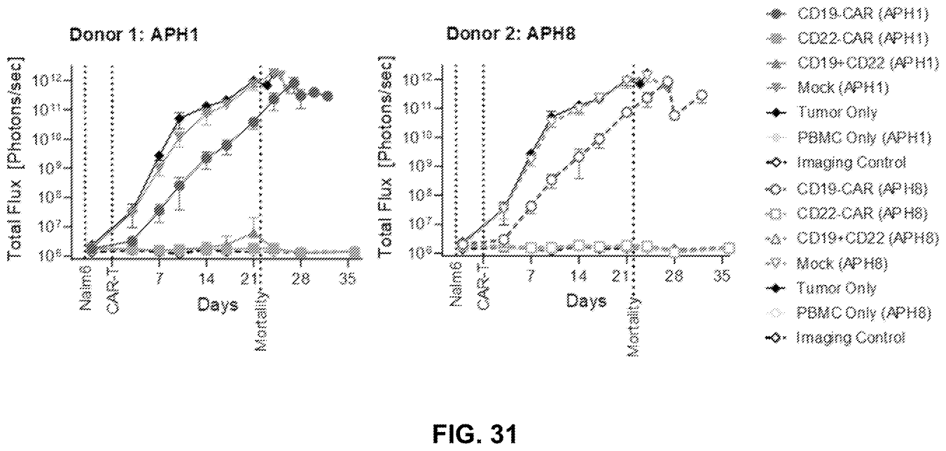

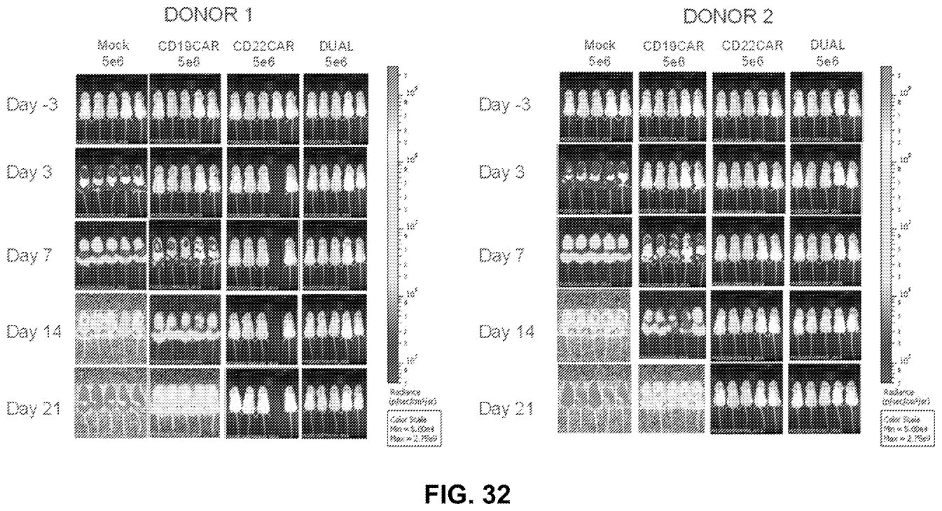

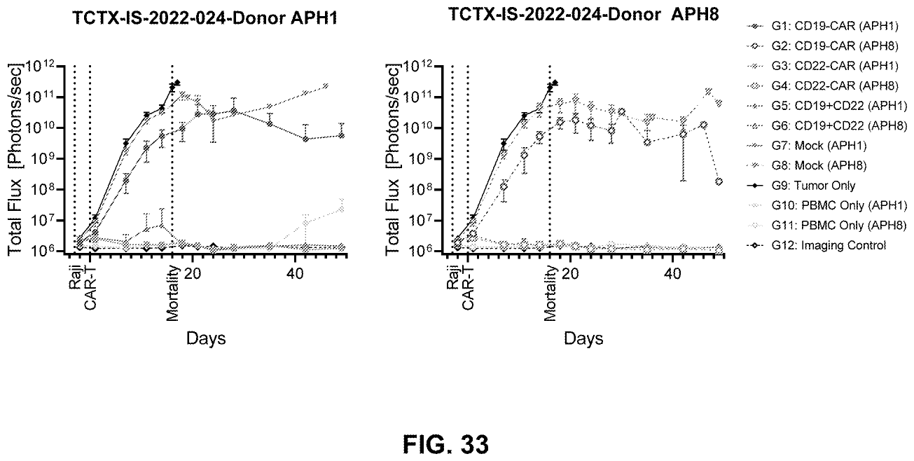

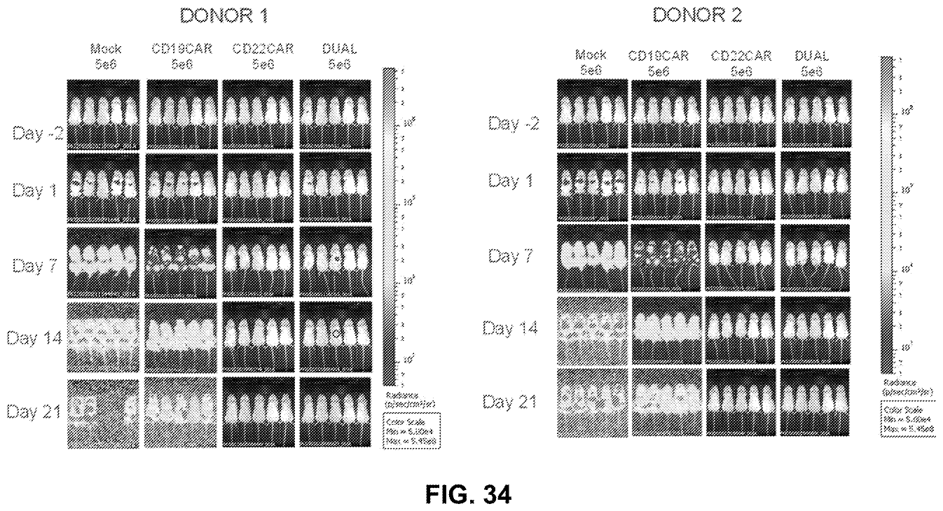

To evaluate the cytotoxicity of dual transduced CD19 CAR×CD22 CAR-T cells in vivo, and to test whether the dual transduced CAR-T cells could overcome CD19 antigen loss, NSG mice were injected intravenously with a mix of CD19-expressing and CD19-negative NALM (1.0×10⁶ cells/mouse) or RAJI (0.5×10⁶ cells/mouse) tumor cells (FIG. 30 showing the NALM model as representative). Three days post tumor cell injection, tumor engrafted mice were treated with intravenous injection of CD19 CAR-T cells, CD22 CAR-T cells, or dual transduced CD19 CAR×CD22 CAR-T cells. Tumor growth was assayed by bioluminescence and Kaplan-Meier survival curve. As shown in FIGS. 31-34, compared with mock and CD19 CAR-T cell treated controls, CD22 CAR-T cell and CD19 CAR×CD22 CAR-T cell treated mice showed controlled tumor burden in CD19 KO NALM (FIGS. 31-32) and CD19 KO RAJI (FIGS. 33-34) mouse models as measured by bioluminescence of tumor cells (FIGS. 31, 33) and whole mouse imaging (FIGS. 32, 34). These data suggest that dual transduced CAR-T cells overcame antigen escape and inhibited NALM and RAJI tumor growth in mouse models, while CD19 CAR-T cells only transiently controlled tumor growth before the mice progressively succumbed to tumor.

### Dual Transduced CD19 CAR×CD22 CAR-T Cells had Increased Cytotoxicity Compared to Mixtures of CD19 CAR- and CD22 CAR-T Cells In Vivo

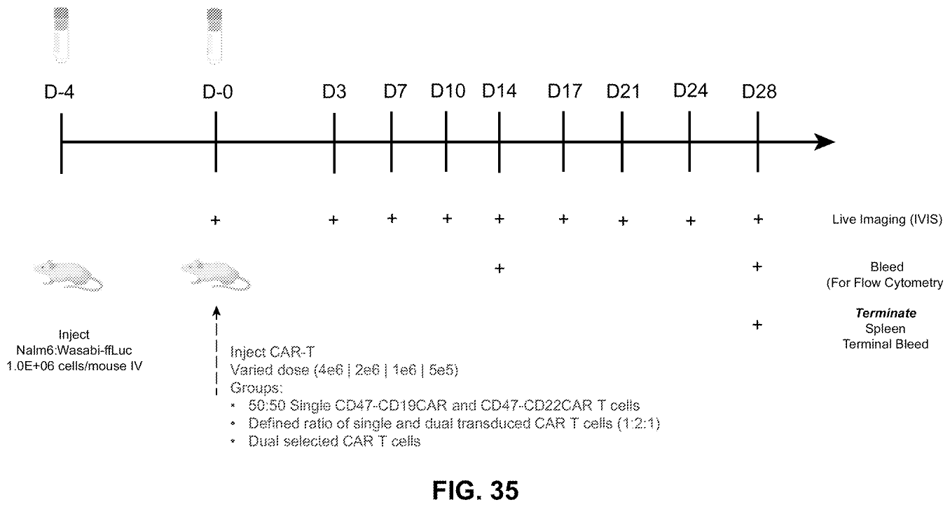

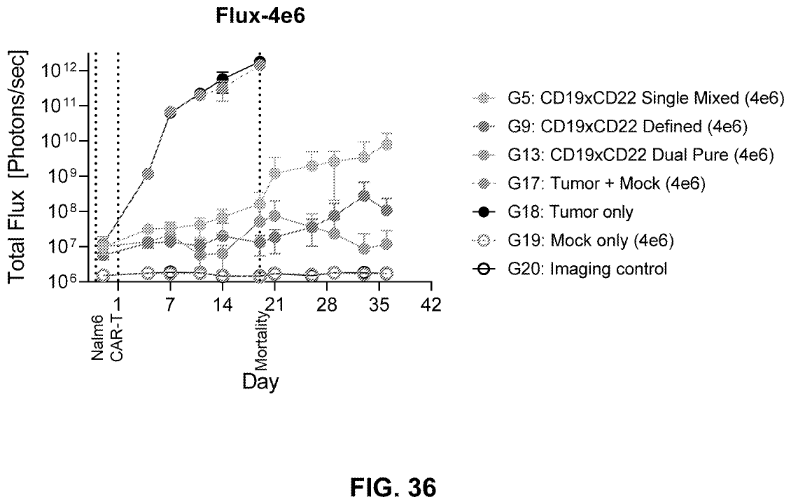

Finally, it was tested whether dual transduced CD19 CAR×CD22 CAR-T cells could more efficiently control tumor growth than a 1:1 mixture of CD19 CAR- and CD22 CAR-T cells. Antitumor activities of dual transduced, or dual transduced and sorted, CD19 CAR×CD22 CAR-T cells were compared to a combined product of CD19 CAR- and CD22 CAR-T cells. As previously, NSG mice were injected intravenously with NALM tumor cells (FIG. 35). Three days post tumor cell injection, tumor engrafted mice were treated with intravenous injection of (1) dual transduced or (2) dual transduced and sorted CD19 CAR×CD22 CAR-T cells or (3) a 50:50 mixture of CD19 CAR- and CD22 CAR-T cells. Tumor growth was assayed by bioluminescence and Kaplan-Meier survival curve. Surprisingly, as shown in FIG. 36, dual transduced, or dual transduced and sorted, CD19×CD22 CAR-T cells exhibited more effective inhibition of tumor growth compared to mixtures of CD19 CAR- and CD22 CAR-T cells, suggesting the tumor killing ability of dual transduced T cells is more than the combined abilities of the single transduced counterparts.

In conclusion, these data demonstrated the high levels of CAR expression, cytotoxicity, hypoimmunity, cytokine response, and tumor killing ability both in vitro and in vivo of CAR-T cells generated by the dual transduction approach. The dual transduced CAR-T cells were able to overcome antigen escape and exhibited better tumor inhibitive performance in tumor engrafted mouse models compared to 1:1 mixtures of single transduced counterparts, and thus may have great potential for use in cell therapy.

### CONCLUSION

The above detailed description of embodiments of the technology are not intended to be exhaustive or to limit the technology to the precise forms disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known components and functions have not been shown or described in detail to avoid unnecessarily obscuring the description of the embodiments of the technology. Where the context permits, singular or plural terms may also include the plural or singular term, respectively. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. A polycistronic vector comprising (a) a first expression cassette comprising a nucleotide sequence encoding a tolerogenic factor, (b) a second expression cassette comprising a nucleotide sequence encoding a chimeric antigen receptor (CAR), and (c) one or more cleavage sites separating the first expression cassette and the second expression cassette, wherein the first expression cassette precedes the second expression cassette in the 5′ to 3′ order.

2. The polycistronic vector of claim 1, wherein the tolerogenic factor is selected from the group consisting of A20/TNFAIP3, CD16, CD16 Fc receptor, CD24, CD35, CD39, CD46, CD47, CD52, CD55, CD59, CD200, CCL22, CTLA4-Ig, C1 inhibitor, CR1, DUX4, FASL, H2-M3, IDO1, IL15-RF, HLA-C, HLA-E, HLA-E heavy chain, HLA-G, IL-10, IL-35, MANF, PD-1, PD-L1, Serpinb9, CCl21, and Mfge8.

3. The polycistronic vector of claim 2, wherein the tolerogenic factor comprises CD47.

4. The polycistronic vector of claim 3, wherein the CD47 is human CD47.

5. The polycistronic vector of claim 4, wherein the human CD47 comprises an amino acid sequence that is at least 80% identical to the amino acid sequence set forth in any one of SEQ ID NOs: 1-5.

6. The polycistronic vector of claim 3, wherein the nucleotide sequence encoding CD47 is at least 80% identical to the nucleotide sequence set forth in any one of SEQ ID NOs: 129-134.

7. The polycistronic vector of claim 6, wherein the nucleotide sequence encoding CD47 is codon-optimized.

8. The polycistronic vector of claim 7, wherein the nucleotide sequence encoding CD47 is at least 80% identical to the nucleotide sequence set forth in SEQ ID NO:135.

9. The polycistronic vector of claim 1, wherein the CAR comprises a CD19 CAR.

10. The polycistronic vector of claim 9, wherein the CD19 CAR comprises a signal peptide, an extracellular binding domain specific to CD19, a hinge domain, a transmembrane domain, an intracellular costimulatory domain, and/or an intracellular signaling domain.

11. The polycistronic vector of claim 10, wherein the signal peptide comprises a CD8α signal peptide, an IgK signal peptide, or a GMCSFR-α signal peptide.

12. The polycistronic vector of claim 10, wherein the extracellular binding domain specific to CD19 comprises an scFv.

13. The polycistronic vector of claim 12, wherein the scFv comprises the light chain variable region (V_{L}) and the heavy chain variable region (V_{H}) of FMC63.

14. The polycistronic vector of claim 12, wherein the scFv comprises one or more complementarity determining regions (CDRs) having amino acid sequences set forth in SEQ ID NOs: 21-23 and 26-28.

15. The polycistronic vector of claim 12, wherein the scFv comprises a light chain with one or more CDRs having amino acid sequences set forth in SEQ ID NOs: 21-23.

16. The polycistronic vector of claim 12, wherein the scFv comprises a heavy chain with one or more CDRs having amino acid sequences set forth in SEQ ID NOs: 26-28.

17. The polycistronic vector of claim 10, wherein the hinge domain comprises a CD8α hinge domain, a CD28 hinge domain, an IgG4 hinge domain, or an IgG4 hinge-CH2-CH3 domain.

18. The polycistronic vector of claim 10, wherein the transmembrane comprises a CD8α transmembrane domain or a CD28 transmembrane domain.

19. The polycistronic vector of claim 10, wherein the intracellular costimulatory domain comprises a 4-1 BB costimulatory domain or a CD28 costimulatory domain.

20. The polycistronic vector of claim 10, wherein the intracellular signaling domain comprises a CD3 zeta (ζ) signaling domain.

21. The polycistronic vector of claim 10, wherein the CD19 CAR comprises an amino acid sequence set forth in SEQ ID NO:117 or is at least 80% identical to the amino acid sequence set forth in SEQ ID NO:117.

22. The polycistronic vector of claim 21, wherein the nucleotide sequence encoding the CD19 CAR comprises a nucleotide sequence set forth in SEQ ID NO:116 or is at least 80% identical to the nucleotide sequence set forth in SEQ ID NO:116.

23. The polycistronic vector of claim 10, wherein the CD19 CAR comprises an amino acid sequence set forth in SEQ ID NO: 32, 34, or 36, or is at least 80% identical to the amino acid sequence set forth in SEQ ID NO: 32, 34, or 36.

24. The polycistronic vector of claim 22, wherein the nucleotide sequence encoding the CD19 CAR comprises a nucleotide sequence set forth in SEQ ID NO: 31, 33, or 35, or is at least 80% identical to the nucleotide sequence set forth in SEQ ID NO: 31, 33, or 35.

25. The polycistronic vector of claim 1, wherein the CAR comprises CD20 CAR.

26. The polycistronic vector of claim 25, wherein the CD20 CAR comprises a signal peptide, an extracellular binding domain specific to CD20, a hinge domain, a transmembrane domain, an intracellular costimulatory domain, and/or an intracellular signaling domain.

27. The polycistronic vector of claim 26, wherein the signal peptide comprises a CD8α signal peptide, an IgK signal peptide, or a GMCSFR-α signal peptide.

28. The polycistronic vector of claim 26, wherein the extracellular binding domain specific to CD20 comprises an scFv.

29. The polycistronic vector of claim 28, wherein the scFv comprises the V_{L} and the V_{H} of Leu16.

30. The polycistronic vector of claim 28, wherein the scFv comprises one or more CDRs having amino acid sequences set forth in SEQ ID NOs: 39-41 and 43-44.

31. The polycistronic vector of claim 28, wherein the scFv comprises a light chain with one or more CDRs having amino acid sequences set forth in SEQ ID NOs: 39-41.

32. The polycistronic vector of claim 28, wherein the scFv comprises a heavy chain with one or more CDRs having amino acid sequences set forth in SEQ ID NOs: 43-44.

33. The polycistronic vector of claim 26, wherein the hinge domain comprises a CD8α hinge domain, a CD28 hinge domain, an IgG4 hinge domain, or an IgG4 hinge-CH2-CH3 domain.

34. The polycistronic vector of claim 26, wherein the transmembrane comprises a CD8α transmembrane domain or a CD28 transmembrane domain.

35. The polycistronic vector of claim 26, wherein the intracellular costimulatory domain comprises a 4-1 BB costimulatory domain or a CD28 costimulatory domain.

36. The polycistronic vector of claim 26, wherein the intracellular signaling domain comprises a CD3 zeta (ζ) signaling domain.

37. The polycistronic vector of claim 1, wherein the CAR comprises CD22 CAR.

38. The polycistronic vector of claim 37, wherein the CD22 CAR comprises a signal peptide, an extracellular binding domain specific to CD22, a hinge domain, a transmembrane domain, an intracellular costimulatory domain, and/or an intracellular signaling domain.

39. The polycistronic vector of claim 38, wherein the signal peptide comprises a CD8α signal peptide, an IgK signal peptide, or a GMCSFR-α signal peptide.

40. The polycistronic vector of claim 38, wherein the extracellular binding domain specific to CD22 comprises an scFv.

41. The polycistronic vector of claim 40, wherein the scFv comprises the V_{H} and the V_{L} of m971.

42. The polycistronic vector of claim 40, wherein the scFv comprises one or more CDRs having amino acid sequences set forth in SEQ ID NOs: 47-49 and 51-53.

43. The polycistronic vector of claim 40, wherein the scFv comprises a heavy chain with one or more CDRs having amino acid sequences set forth in SEQ ID NOs: 47-49.

44. The polycistronic vector of claim 40, wherein the scFv comprises a light chain with one or more CDRs having amino acid sequences set forth in SEQ ID NOs: 51-53.

45. The polycistronic vector of claim 40, wherein the scFv comprises the V_{H} and the V_{L} of m971-L7.

46. The polycistronic vector of claim 40, wherein the scFv comprises one or more CDRs having amino acid sequences set forth in SEQ ID NOs: 56-58 and 60-62.

47. The polycistronic vector of claim 40, wherein the scFv comprises a heavy chain with one or more CDRs having amino acid sequences set forth in SEQ ID NOs: 56-58.

48. The polycistronic vector of claim 40, wherein the scFv comprises a light chain with one or more CDRs having amino acid sequences set forth in SEQ ID NOs: 60-62.

49. The polycistronic vector of claim 38, wherein the hinge domain comprises a CD8α hinge domain, a CD28 hinge domain, an IgG4 hinge domain, or an IgG4 hinge-CH2-CH3 domain.

50. The polycistronic vector of claim 38, wherein the transmembrane comprises a CD8α transmembrane domain or a CD28 transmembrane domain.

51. The polycistronic vector of claim 38, wherein the intracellular costimulatory domain comprises a 4-1 BB costimulatory domain or a CD28 costimulatory domain.

52. The polycistronic vector of claim 38, wherein the intracellular signaling domain comprises a CD3 zeta (ζ) signaling domain.

53. The polycistronic vector of claim 1, wherein the CAR comprises BCMA CAR.

54. The polycistronic vector of claim 53, wherein the BCMA CAR comprises a signal peptide, an extracellular binding domain specific to BCMA, a hinge domain, a transmembrane domain, an intracellular costimulatory domain, and/or an intracellular signaling domain.

55. The polycistronic vector of claim 54, wherein the signal peptide comprises a CD8α signal peptide, an IgK signal peptide, or a GMCSFR-α signal peptide.

56. The polycistronic vector of claim 54, wherein the extracellular binding domain specific to BCMA comprises an scFv.

57. The polycistronic vector of claim 56, wherein the scFv comprises the V_{L} and the V_{H} of C11D5.3.

58. The polycistronic vector of claim 56, wherein the scFv comprises one or more CDRs having amino acid sequences set forth in SEQ ID NOs: 65-67 and 69-71.

59. The polycistronic vector of claim 56, wherein the scFv comprises a light chain with one or more CDRs having amino acid sequences set forth in SEQ ID NOs: 65-67.

60. The polycistronic vector of claim 56, wherein the scFv comprises a heavy chain with one or more CDRs having amino acid sequences set forth in SEQ ID NOs: 69-71.

61. The polycistronic vector of claim 56, wherein the scFv comprises the V_{L} and the V_{H} of C12A3.2.

62. The polycistronic vector of claim 56, wherein the scFv comprises one or more CDRs having amino acid sequences set forth in SEQ ID NOs: 74-76 and 78-80.

63. The polycistronic vector of claim 56, wherein the scFv comprises a light chain with one or more CDRs having amino acid sequences set forth in SEQ ID NOs: 74-76.

64. The polycistronic vector of claim 56, wherein the scFv comprises a heavy chain with one or more CDRs having amino acid sequences set forth in SEQ ID NOs: 78-80.

65. The polycistronic vector of claim 56, wherein the scFv comprises an amino acid sequence set forth in SEQ ID NO:118.

66. The polycistronic vector of claim 56, wherein the scFv comprises one or more CDRs having amino acid sequences set forth in SEQ ID NOs: 120-122 and 124-126.

67. The polycistronic vector of claim 56, wherein the scFv comprises a light chain with one or more CDRs having amino acid sequences set forth in SEQ ID NOs: 120-122.

68. The polycistronic vector of claim 56, wherein the scFv comprises a heavy chain with one or more CDRs having amino acid sequences set forth in SEQ ID NOs: 124-126.

69. The polycistronic vector of claim 54, wherein the extracellular binding domain specific to BCMA comprises a fully human heavy-chain variable domain (FHVH).

70. The polycistronic vector of claim 65, wherein the FHVH comprises FHVH33.

71. The polycistronic vector of claim 65, wherein the FHVH comprises one or more CDRs having amino acid sequences set forth in SEQ ID NOs: 82-84.

72. The polycistronic vector of claim 54, wherein the hinge domain comprises a CD8α hinge domain, a CD28 hinge domain, an IgG4 hinge domain, or an IgG4 hinge-CH2-CH3 domain.

73. The polycistronic vector of claim 54, wherein the transmembrane comprises a CD8α transmembrane domain or a CD28 transmembrane domain.

74. The polycistronic vector of claim 54, wherein the intracellular costimulatory domain comprises a 4-1 BB costimulatory domain or a CD28 costimulatory domain.

75. The polycistronic vector of claim 54, wherein the intracellular signaling domain comprises a CD3 zeta (ζ) signaling domain.

76. The polycistronic vector of any one of claims 1-75, wherein the one or more cleavage sites comprise a self-cleaving site.

77. The polycistronic vector of claim 76, wherein the self-cleaving site comprises a 2A site.

78. The polycistronic vector of claim 77, wherein the 2A site comprises a T2A, P2A, E2A, or F2A site.

79. The polycistronic vector of claim 77, wherein the one or more cleavage sites further comprise a protease site.

80. The polycistronic vector of claim 79, wherein the protease site comprises a furin site.

81. The polycistronic vector of claim 80, wherein the furin site comprises an FC1, FC2, or FC3 site.

82. The polycistronic vector of claim 79, wherein the protease site precedes the 2A site in the 5′ to 3′ order.

83. The polycistronic vector of any one of claims 1-82, further comprising (d) a third expression cassette comprising a nucleotide sequence encoding a safety switch, wherein the third expression cassette is separated from the first expression cassette and/or the second expression cassette by one or more cleavage sites.

84. The polycistronic vector of claim 83, wherein the safety switch is selected from the group consisting of herpes simplex virus thymidine kinase (HSVtk), cytosine deaminase (CyD), nitroreductase (NTR), purine nucleoside phosphorylase (PNP), horseradish peroxidase, inducible caspase 9 (iCasp9), rapamycin-activated caspase 9 (rapaCasp9), CCR4, CD16, CD19, CD20, CD30, EGFR, GD2, HER1, HER2, MUC1, PSMA, RQR8, and a CD47-SIRPα blockade agent.

85. The polycistronic vector of any one of claims 1-84, further comprising a promoter.

86. The polycistronic vector of claim 85, wherein the promoter is a constitutive promoter.

87. The polycistronic vector of claim 86, wherein the constitutive promoter is an EF1α, CMV, SV40, PGK, UBC or CAG promoter.

88. The polycistronic vector of claim 85, wherein the promoter is an inducible promoter.

89. The polycistronic vector of claim 88, wherein the inducible promoter is a Tet-On, Tet-Off, AlcA, LexA, or Cre promoter.

90. The polycistronic vector of any one of claims 1-89, further comprising homology arms flanking the expression cassettes for homology directed repair (HDR)-mediated insertion into a genomic locus.

91. The polycistronic vector of claim 90, wherein the HDR uses a site-directed nuclease selected from the group consisting of Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, Mad7, a zinc finger nuclease (ZFN), a transcription activator-like effector nuclease (TALEN), a meganuclease, and a CRISPR-associated transposase.

92. A polycistronic vector comprising (a) a first expression cassette comprising a nucleotide sequence encoding CD47, (b) a second expression cassette comprising a nucleotide sequence encoding CD19 CAR, and (c) a 2A site separating the first expression cassette and the second expression cassette.

93. A polycistronic vector comprising (a) a first expression cassette comprising a nucleotide sequence encoding CD47, (b) a second expression cassette comprising a nucleotide sequence encoding CD19 CAR, and (c) a furin site and a 2A site separating the first expression cassette and the second expression cassette, wherein the furin site precedes the 2A site in the 5′ to 3′ order.

94. The polycistronic vector of claim 92 or 93, further comprising (d) a third expression cassette comprising a nucleotide sequence encoding a safety switch, wherein the third expression cassette is separated from the first expression cassette and/or the second expression cassette by a 2A site.

95. A polycistronic vector comprising (a) a first expression cassette comprising a nucleotide sequence encoding a tolerogenic factor, (b) a second expression cassette comprising a nucleotide sequence encoding CD19 CAR, and (c) one or more cleavage sites separating the first expression cassette and the second expression cassette, wherein the CD19 CAR comprises a CD8α signal peptide, an FMC63 scFv, a CD8α hinge domain, a CD8α transmembrane domain, a 4-1BB costimulatory domain, and a CD3ζ signaling domain.

96. The polycistronic vector of claim 95, wherein the tolerogenic factor comprises CD47.

97. A polycistronic vector comprising (a) a first expression cassette comprising a nucleotide sequence encoding a tolerogenic factor, (b) a second expression cassette comprising a nucleotide sequence encoding CD19 CAR, and (c) one or more cleavage sites separating the first expression cassette and the second expression cassette, wherein the CD19 CAR comprises a GMCSFR-α signal peptide, an FMC63 scFv, an IgG4 hinge domain, a CD28 transmembrane domain, a 4-1 BB costimulatory domain, and a CD3ζ signaling domain.

98. The polycistronic vector of claim 97, wherein the tolerogenic factor comprises CD47.

99. A polycistronic vector comprising (a) a first expression cassette comprising a nucleotide sequence encoding a tolerogenic factor, (b) a second expression cassette comprising a nucleotide sequence encoding CD19 CAR, and (c) one or more cleavage sites separating the first expression cassette and the second expression cassette, wherein the CD19 CAR comprises a GMCSFR-α signal peptide, an FMC63 scFv, a CD28 hinge domain, a CD28 transmembrane domain, a CD28 costimulatory domain, and a CD3ζ signaling domain.

100. The polycistronic vector of claim 99, wherein the tolerogenic factor comprises CD47.

101. A polycistronic vector comprising (a) a first expression cassette comprising a nucleotide sequence encoding CD47, (b) a second expression cassette comprising a nucleotide sequence encoding CD20 CAR, and (c) a 2A site separating the first expression cassette and the second expression cassette.

102. A polycistronic vector comprising (a) a first expression cassette comprising a nucleotide sequence encoding CD47, (b) a second expression cassette comprising a nucleotide sequence encoding CD20 CAR, and (c) a furin site and a 2A site separating the first expression cassette and the second expression cassette, wherein the furin site precedes the 2A site in the 5′ to 3′ order.

103. The polycistronic vector of claim 101 or 102, further comprising (d) a third expression cassette comprising a nucleotide sequence encoding a safety switch, wherein the third expression cassette is separated from the first expression cassette and/or the second expression cassette by a 2A site.

104. A polycistronic vector comprising (a) a first expression cassette comprising a nucleotide sequence encoding CD47, (b) a second expression cassette comprising a nucleotide sequence encoding CD22 CAR, and (c) a 2A site separating the first expression cassette and the second expression cassette.

105. A polycistronic vector comprising (a) a first expression cassette comprising a nucleotide sequence encoding CD47, (b) a second expression cassette comprising a nucleotide sequence encoding CD22 CAR, and (c) a furin site and a 2A site separating the first expression cassette and the second expression cassette, wherein the furin site precedes the 2A site in the 5′ to 3′ order.

106. The polycistronic vector of claim 104 or 105, further comprising (d) a third expression cassette comprising a nucleotide sequence encoding a safety switch, wherein the third expression cassette is separated from the first expression cassette and/or the second expression cassette by a 2A site.

107. A polycistronic vector comprising (a) a first expression cassette comprising a nucleotide sequence encoding CD47, (b) a second expression cassette comprising a nucleotide sequence encoding CD19 CAR, (c) a third expression cassette comprising a nucleotide sequence encoding CD22 CAR, and (d) a 2A site separating any two neighboring expression cassettes.

108. A polycistronic vector comprising (a) a first expression cassette comprising a nucleotide sequence encoding CD47, (b) a second expression cassette comprising a nucleotide sequence encoding CD19 CAR, (c) a third expression cassette comprising a nucleotide sequence encoding CD22 CAR, and (d) a furin site and a 2A site separating any two neighboring expression cassettes, wherein the furin site precedes the 2A site in the 5′ to 3′ order.

109. The polycistronic vector of claim 107 or 108, further comprising (e) a fourth expression cassette comprising a nucleotide sequence encoding a safety switch, wherein the fourth expression cassette is separated from the first expression cassette, the second expression cassette, and/or the third expression cassette by a 2A site.

110. A polycistronic vector comprising (a) a first expression cassette comprising a nucleotide sequence encoding CD47, (b) a second expression cassette comprising a nucleotide sequence encoding BCMA CAR, and (c) a 2A site separating the first expression cassette and the second expression cassette.

111. A polycistronic vector comprising (a) a first expression cassette comprising a nucleotide sequence encoding CD47, (b) a second expression cassette comprising a nucleotide sequence encoding BCMA CAR, and (c) a furin site and a 2A site separating the first expression cassette and the second expression cassette, wherein the furin site precedes the 2A site in the 5′ to 3′ order.

112. The polycistronic vector of claim 110 or 111, further comprising (d) a third expression cassette comprising a nucleotide sequence encoding a safety switch, wherein the third expression cassette is separated from the first expression cassette and/or the second expression cassette by a 2A site.

113. A polycistronic vector comprising (a) a first expression cassette comprising a nucleotide sequence encoding a tolerogenic factor, (b) a second expression cassette comprising a nucleotide sequence encoding BCMA CAR, and (c) one or more cleavage sites separating the first expression cassette and the second expression cassette, wherein the BCMA CAR comprises a BB2121 binder, a CD8α hinge domain, a CD8α transmembrane domain, a 4-1 BB costimulatory domain, and a CD3ζ signaling domain.

114. A polycistronic vector comprising (a) a first expression cassette comprising a nucleotide sequence encoding a tolerogenic factor, (b) a second expression cassette comprising a nucleotide sequence encoding BCMA CAR, and (c) one or more cleavage sites separating the first expression cassette and the second expression cassette, wherein the BCMA CAR comprises a CD8α signal peptide, an CT103A scFv, a CD8α hinge domain, a CD8α transmembrane domain, a 4-1 BB costimulatory domain, and a CD3ζ signaling domain.

115. The polycistronic vector of claim 114, wherein the BCMA comprises an amino acid sequence set forth in SEQ ID NO:128.

116. The polycistronic vector of any one of claims 113-115, wherein the tolerogenic factor comprises CD47.

117. A polycistronic vector comprising (a) a first expression cassette comprising a nucleotide sequence encoding CD47, (b) a second expression cassette comprising a nucleotide sequence encoding a safety switch, and (c) a 2A site separating the first expression cassette and the second expression cassette.

118. A polycistronic vector comprising (a) a first expression cassette comprising a nucleotide sequence encoding CD47, (b) a second expression cassette comprising a nucleotide sequence encoding a safety switch, and (c) a furin site and a 2A site separating the first expression cassette and the second expression cassette, wherein the furin site precedes the 2A site in the 5′ to 3′ order.

119. A virus containing the polycistronic vector of any one of claims 1 to 118 or a fragment thereof.

120. The virus of claim 119, wherein the virus is an adenovirus, adeno-associated virus, retrovirus, lentivirus, or phage.

121. A host cell containing the polycistronic vector of any one of claims 1 to 118 or a fragment thereof.

122. The host cell of claim 121, wherein the host cell is an autologous cell.

123. The host cell of claim 121, wherein the host cell is an allogeneic cell.

124. The host cell of any one of claims 121-123, wherein the host cell is an embryonic stem cell (ESC) or an induced pluripotent stem cell (iPSC).

125. The host cell of any one of claims 121-123, wherein the host cell is differentiated from an ESC or an iPSC.

126. The host cell of any one of claims 121-123, wherein the host cell is a primary cell.

127. The host cell of claim 125 or 126, wherein the host cell is a T cell, a natural killer (NK) cell, or a natural killer T (NKT) cell.

128. A host cell containing the polycistronic vector of claim 117 or 118 or a fragment thereof.

129. The host cell of claim 128, wherein the host cell is an autologous cell.

130. The host cell of claim 128, wherein the host cell is an allogeneic cell.

131. The host cell of any one of claims 128-130, wherein the host cell is an ESC or an iPSC.

132. The host cell of any one of claims 128-130, wherein the host cell is differentiated from an ESC or an iPSC.

133. The host cell of any one of claims 128-130, wherein the host cell is a primary cell.

134. The host cell of claim 132 or 133, wherein the host cell is a β islet cell.

135. The host cell of claim 132 or 133, wherein the host cell is a glial progenitor cell (GPC).

136. The host cell of any one of claims 121-135, wherein the polycistronic vector or fragment thereof is inserted into a specific genomic locus of the host cell selected from the group consisting of a B2M locus, a TAP1 locus, a CIITA locus, a TRAC locus, a TRBC locus, a MIC-A locus, a MIC-B locus, and a safe harbor locus.

137. The host cell of claim 136, wherein the safe harbor locus is selected from the group consisting of an AAVS1, ABO, CCR5, CLYBL, CXCR4, F3, FUT1, HMGB1, KDM5D, LRP1, MICA, MICB, RHD, ROSA26, and SHS231 locus.

138. The host cell of claim 136 or 137, wherein the insertion is by HDR.

139. The host cell of claim 138, wherein the HDR uses a site-directed nuclease selected from the group consisting of Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, Mad7, a zinc finger nuclease (ZFN), a transcription activator-like effector nuclease (TALEN), a meganuclease, and a CRISPR-associated transposase.

140. The host cell of claim any one of claims 121-139, wherein the host cell is modified to have reduced expression of one or more MHC I molecules and/or one or more MHC II molecules, optionally, wherein the one or more MHC I molecules are selected from the group consisting of HLA-A, HLA-B, HLA-C, and optionally, wherein the one or more MHC II molecules are selected from the group consisting of HLA-DR, HLA-DQ, HLA-DP, HLA-DM, and HLA-DO.

141. The host cell of claim 140, wherein the host cell has reduced expression of B2M, TAP1, and/or CIITA.

142. The host cell of claim 141, wherein host cell has B2M, TAP1, and/or CIITA knockout.

143. The host cell of claim 142, wherein the B2M, TAP1, and/or CIITA knockout occur in both alleles.

144. The host cell of claim 140, wherein the host cell has reduced expression of MIC-A and/or MIC-B.

145. The host cell of claim 144, wherein host cell has MIC-A and/or MIC-B knockout.

146. The host cell of claim 145, wherein the MIC-A and/or MIC-B knockout occur in both alleles.

147. A T cell containing a polycistronic vector or fragments thereof, wherein the polycistronic vector comprising a first expression cassette comprising a nucleotide sequence encoding CD47 a second expression cassette comprising a nucleotide sequence encoding a CAR.

148. A T cell containing a polycistronic vector or a fragment thereof, wherein the polycistronic vector comprising a first expression cassette comprising a nucleotide sequence encoding CD47 a second expression cassette comprising a nucleotide sequence encoding a CAR, and wherein the T cell has B2M, TAP1, and/or CIITA knockout.

149. The T cell of claim 148, wherein the B2M, TAP1, and/or CIITA knockout occur in both alleles.

150. The T cell of any one of claims 147-149, wherein the polycistronic vector or fragment thereof is inserted into a specific genomic locus of the T cell selected from the group consisting of a B2M locus, a TAP1 locus, a CIITA locus, a TRAC locus, a TRBC locus, a MIC-A locus, a MIC-B locus, and a safe harbor locus.

151. The T cell of claim 150, wherein the safe harbor locus is selected from the group consisting of an AAVS1, ABO, CCR5, CLYBL, CXCR4, F3, FULT1, HMGB1, KDM5D, LRP1, MICA, MICB, RHD, ROSA26, and SHS231 locus

152. The T cell of any one of claims 147-151, wherein the CAR comprises CD19 CAR, CD20 CAR, CD22 CAR, or BCMA CAR.

153. The T cell of claim 152, wherein the CAR is CD19 CAR comprising a CD8α signal peptide, an FMC63 scFv, a CD8α hinge domain, a CD8α transmembrane domain, a 4-1 BB costimulatory domain, and a CD3ζ signaling domain.

154. The T cell of claim 152, wherein the CAR is CD19 CAR comprising a GMCSFR-α signal peptide, an FMC63 scFv, an IgG4 hinge domain, a CD28 transmembrane domain, a 4-1 BB costimulatory domain, and a CD3ζ signaling domain.

155. The T cell of claim 152, wherein the CAR is CD19 CAR comprising a GMCSFR-α signal peptide, an FMC63 scFv, a CD28 hinge domain, a CD28 transmembrane domain, a CD28 costimulatory domain, and a CD3ζ signaling domain.

156. The T cell of claim 152, wherein the CAR is CD19 CAR comprising an amino acid sequence set forth in SEQ ID NO:117 or is at least 80% identical to the amino acid sequence set forth in SEQ ID NO:117.

157. The T cell of claim 152, wherein the CAR is CD19 CAR comprising an amino acid sequence set forth in SEQ ID NO: 32, 34, or 36, or an amino acid sequence that is at least 80% identical to the amino acid sequence set forth in SEQ ID NO: 32, 34, or 36.

158. The T cell of claim 152, wherein the CAR is BCMA CAR comprising a CD8α signal peptide, a CT103A scFv, a CD8α hinge domain, a CD8α transmembrane domain, a 4-1 BB costimulatory domain, and a CD3ζ signaling domain.

159. The T cell of claim 152, wherein the CAR is BCMA CAR comprising an amino acid sequence set forth in SEQ ID NO:128 or is at least 80% identical to the amino acid sequence set forth in SEQ ID NO:128.

160. A cell having a genomic locus modified by HDR, wherein the genomic locus is selected from the group consisting of a B2M locus, a TAP1 locus, a CIITA locus, a TRAC locus, a TRBC locus, a MIC-A locus, a MIC-B locus, and a safe harbor locus.

161. The cell of claim 160, wherein the safe harbor locus is selected from the group consisting of an AAVS1, ABO, CCR5, CLYBL, CXCR4, F3, FULT1, HMGB1, KDM5D, LRP1, MICA, MICB, RHD, ROSA26, and SHS231 locus.

162. The cell of claim 160 or 161, wherein the HDR uses a site-directed nuclease selected from the group consisting of Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, Mad7, a zinc finger nuclease (ZFN), a transcription activator-like effector nuclease (TALEN), a meganuclease, and a CRISPR-associated transposase.

163. An iPSC-derived β islet cell having (1) reduced expression of MHC I and/or MHC II; and (2) a transgene comprising CD47 and a safety switch inserted at a safe harbor locus, wherein the safe harbor locus is selected from the group consisting of an AAVS1, ABO, CCR5, CLYBL, CXCR4, F3, FULT1, HMGB1, KDM5D, LRP1, MICA, MICB, RHD, ROSA26, and SHS231 locus.

164. An iPSC-derived β islet cell having (1) reduced expression of MHC I and/or MHC II; and (2) a transgene comprising CD47 and HSVtk flanked by CLYBL homology arms, wherein the transgene is inserted at the CLYBL locus.

165. The iPSC-derived β islet cell of claim 163 or 164, wherein the cell has B2M, TAP1, and/or CIITA knockout.

166. The iPSC-derived β islet cell of claim 165, wherein the B2M, TAP1, and/or CIITA knockout occur in both alleles.

167. An ESC-derived GPC having (1) reduced expression of MHC I and/or MHC II; and (2) a transgene comprising CD47 and a safety switch inserted at a safe harbor locus, wherein the safe harbor locus is selected from the group consisting of an AAVS1, ABO, CCR5, CLYBL, CXCR4, F3, FULT1, HMGB1, KDM5D, LRP1, MICA, MICB, RHD, ROSA26, and SHS231 locus.

168. An ESC-derived GPC having (1) reduced expression of MHC I and/or MHC II; and (2) a transgene comprising CD47 and HSVtk flanked by CLYBL homology arms, wherein the transgene is inserted at the CLYBL locus.

169. The ESC-derived GPC of claim 167 or 168, wherein the cell has B2M, TAP1, and/or CIITA knockout.

170. The ESC-derived GPC of claim 169, wherein the B2M, TAP1, and/or CIITA knockout occur in both alleles.

171. A composition comprising the polycistronic vector of any one of claims 1 to 118.

172. A composition comprising the virus of claim 119 or 120.

173. A pharmaceutical composition comprising the host cell or cell of any one of claims 121-170.

174. A guide RNA (gRNA) for use in HDR-mediated insertion of a transgene into a genomic locus selected from the group consisting of a B2M locus, a TAP1 locus, a CIITA locus, a TRAC locus, a TRBC locus, a MIC-A locus, a MIC-B locus, and a safe harbor locus.

175. The gRNA of claim 174, wherein the safe harbor locus is selected from the group consisting of an AAVS1, ABO, CCR5, CLYBL, CXCR4, F3, FUT1, HMGB1, KDM5D, LRP1, MICA, MICB, RHD, ROSA26, and SHS231 locus.

176. The gRNA of claim 174 or 175, wherein the gRNA comprises a crRNA and optionally a tracrRNA.

177. The gRNA of claim 176, wherein the gRNA comprises a crRNA and a tracrRNA as two separate molecules.

178. The gRNA of claim 176, wherein the gRNA comprises a crRNA and a tracrRNA as a single guide RNA (sgRNA).

179. The gRNA of claim 178, wherein the sgRNA comprises a complementary region, a crRNA repeat region, a tetraloop, and a tracrRNA.

180. The gRNA of claim 179, wherein the crRNA repeat region comprises, consists of, or consists essentially of a nucleotide sequence set forth in in SEQ ID NO:95, SEQ ID NO:99, SEQ ID NO:103, or SEQ ID NO:108.

181. The gRNA of claim 179 or 180, wherein the tetraloop comprises, consists of, or consists essentially of a nucleotide sequence set forth in SEQ ID NO:96 or SEQ ID NO:107.

182. The gRNA of any one of claims 179-181, wherein the tracrRNA comprises, consists of, or consists essentially of a nucleotide sequence set forth in SEQ ID NO:97, SEQ ID NO:101, SEQ ID NO:105, or SEQ ID NO:106.

183. The gRNA of any one of claims 176-182, wherein the crRNA comprises a complementary region specific to a region of the AAVS1, CLYBL, or CCR5 locus.

184. The gRNA of claim 183, wherein the region is a coding sequence (CDS), an exon, an intron, a sequence spanning a portion of an exon and a portion of an adjacent intron, or a regulatory region.

185. The gRNA of claim 183 or 184, wherein the complementary region comprises, consists of, or consists essentially of a nucleotide sequence set forth in SEQ ID NO:110, SEQ ID NO:111, or SEQ ID NO:112.

186. A gRNA for use in HDR-mediated insertion of a transgene into a genomic locus, wherein the genomic locus is located within 4000 bp of a locus at Chromosome 19: 55,117,222-55,112,796.

187. The gRNA of claim 186, wherein the genomic locus is located at Chromosome 19: 55,115,674 or at a position within 5, 10, 15, 20, 30, 40, or 50 nucleotides of Chromosome 19: 55,115,674.

188. A gRNA for use in HDR-mediated insertion of a transgene into a genomic locus, wherein the genomic locus is located within 4000 bp of a locus at Chromosome 13: 99,773,011-99,858,860.

189. The gRNA of claim 188, wherein the genomic locus is located at Chromosome 13: 99,822,980, or at a position within 5, 10, 15, 20, 30, 40, or 50 nucleotides of Chromosome 13: 99,822,980.

190. A gRNA for use in HDR-mediated insertion of a transgene into a genomic locus, wherein the genomic locus is located within 4000 bp of a locus at Chromosome 3: 46,372,892-46,376,206.

191. The gRNA of claim 190, wherein the genomic locus is located at Chromosome 3: 46,373,180, or at a position within 5, 10, 15, 20, 30, 40, or 50 nucleotides of Chromosome 3: 46,373,180.

192. A composition comprising the gRNA of any one of claims 174-191.

193. The composition of claim 192, further comprising a site-directed nuclease or a nucleotide sequence encoding a site-directed nuclease protein.

194. The composition of claim 193, wherein the site-directed nuclease is selected from the group consisting of Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, Mad7, a zinc finger nuclease (ZFN), a transcription activator-like effector nuclease (TALEN), a meganuclease, and a CRISPR-associated transposase.

195. The composition of any one of claims 192-194, wherein the composition is formulated for delivery into a cell.

196. A cell comprising the gRNA of any one of claims 174-191.

197. The cell of claim 196, further comprising a site-directed nuclease or a nucleotide sequence encoding a site-directed nuclease protein.

198. The composition of claim 197, wherein the site-directed nuclease is selected from the group consisting of Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, Mad7, a zinc finger nuclease (ZFN), a transcription activator-like effector nuclease (TALEN), a meganuclease, and a CRISPR-associated transposase.

199. A method of HDR-mediated insertion of a transgene into a genomic locus, comprising introducing a gRNA, a site-directed nuclease or a nucleotide sequence encoding a site-directed nuclease, and a transgene flanked by homology arms into a host cell, wherein the sited-directed nuclease is selected from the group consisting of Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, Mad7, a zinc finger nuclease (ZFN), a transcription activator-like effector nuclease (TALEN), a meganuclease, and a CRISPR-associated transposase.

200. The method of claim 199, wherein the genomic locus is selected from the group consisting of a B2M locus, a TAP1 locus, a CIITA locus, a TRAC locus, a TRBC locus, a MIC-A locus, a MIC-B locus, and a safe harbor locus.

201. The method of claim 200, wherein the safe harbor locus is selected from the group consisting of an AAVS1, ABO, CCR5, CLYBL, CXCR4, F3, FULT1, HMGB1, KDM5D, LRP1, MICA, MICB, RHD, ROSA26, and SHS231 locus.

202. The method of any one of claims 199-201, wherein the gRNA comprises a crRNA and optionally a tracrRNA.

203. The method of claim 202, wherein the gRNA comprises a crRNA and a tracrRNA as two separate molecules.

204. The method of claim 202, wherein the gRNA comprises a crRNA and a tracrRNA as a single guide RNA (sgRNA).

205. The method of claim 204, wherein the sgRNA comprises a complementary region, a crRNA repeat region, a tetraloop, and a tracrRNA.

206. The method of claim 205, wherein the crRNA repeat region comprises, consists of, or consists essentially of a nucleotide sequence set forth in in SEQ ID NO:95, SEQ ID NO:99, SEQ ID NO:103, or SEQ ID NO:108.

207. The method of claim 205 or 206, wherein the tetraloop comprises, consists of, or consists essentially of a nucleotide sequence set forth in SEQ ID NO:96 or SEQ ID NO:107.

208. The method of any one of claims 205-207, wherein the tracrRNA comprises, consists of, or consists essentially of a nucleotide sequence set forth in SEQ ID NO:97, SEQ ID NO:101, SEQ ID NO:105, or SEQ ID NO:106.

209. The method of any one of claims 202-208, wherein the crRNA comprises a complementary region specific to a region of the AAVS1, CLYBL, or CCR5 locus.

210. The method of claim 209, wherein the region is a coding sequence (CDS), an exon, an intron, a sequence spanning a portion of an exon and a portion of an adjacent intron, or a regulatory region.

211. The method of claim 209 or 210, wherein the complementary region comprises, consists of, or consists essentially of a nucleotide sequence set forth in SEQ ID NO:110, SEQ ID NO:111, or SEQ ID NO:112.

212. The method of any one of claims 199-211, wherein the genomic locus is located within 4000 bp of a locus at Chromosome 19: 55,117,222-55,112,796.

213. The method of claim 212, wherein the genomic locus is located at Chromosome 19: 55,115,674 or at a position within 5, 10, 15, 20, 30, 40, or 50 nucleotides of Chromosome 19: 55,115,674.

214. The method of any one of claims 199-211, wherein the genomic locus is located within 4000 bp of a locus at Chromosome 13: 99,773,011-99,858,860.

215. The method of claim 214, wherein the genomic locus is located at Chromosome 13: 99,822,980, or at a position within 5, 10, 15, 20, 30, 40, or 50 nucleotides of Chromosome 13: 99,822,980.

216. The method of any one of claims 199-211, wherein the genomic locus is located within 4000 bp of a locus at Chromosome 3: 46,372,892-46,376,206.

217. The method of claim 216, wherein the genomic locus is located at Chromosome 3: 46,373,180, or at a position within 5, 10, 15, 20, 30, 40, or 50 nucleotides of Chromosome 3: 46,373,180.

218. The method of any one of claims 199-217, wherein the transgene comprises (a) a first expression cassette comprising a nucleotide sequence encoding a tolerogenic factor, (b) a second expression cassette comprising a nucleotide sequence encoding a chimeric antigen receptor (CAR), (c) a third expression cassette comprising a nucleotide sequence encoding a safety switch, and/or (d) one or more cleavage sites separating the first expression cassette, the second expression cassette, and/or the third expression cassette.

219. A method of identifying new genomic loci for HDR-mediated insertion of a transgene, comprising (a) locating a genomic locus based on a known gRNA; and (b) scanning a region of about 500 to 4000 bp on either side of the genomic locus for a PAM sequence.

220. The method of claim 219, wherein the genomic locus is located within 4000 bp of a locus at Chromosome 19: 55,117,222-55,112,796, Chromosome 13: 99,773,011-99,858,860, or Chromosome 3: 46,372,892-46,376,206.

221. The method of claim 220, wherein the genomic locus is located at a position selected from the group consisting of Chromosome 19: 55,115,674 or a position within 5, 10, 15, 20, 30, 40, or 50 nucleotides of Chromosome 19: 55,115,674; Chromosome 13: 99,822,980, or a position within 5, 10, 15, 20, 30, 40, or 50 nucleotides of Chromosome 13: 99,822,980; and Chromosome 3: 46,373,180, or a position within 5, 10, 15, 20, 30, 40, or 50 nucleotides of Chromosome 3: 46,373,180.

222. A method of treating a disease in a subject in need thereof, comprising administering the subject the host cell or cell of any one of claims 121-168, or the pharmaceutical composition of claim 173.

223. The method of claim 222, wherein the disease is cancer.

224. The method of claim 223, wherein the cancer is associated with CD19, CD20, CD22, and/or BCMA expression.

225. The method of claim 223, wherein the cancer is a hematologic malignancy.

226. The method of claim 225, wherein the hematologic malignancy is selected from the group consisting of myeloid neoplasm, myelodysplastic syndromes (MDS), myeloproliferative/myelodysplastic syndromes, acute lymphoid leukemia (ALL), chronic lymphocytic leukemia (CLL), acute myeloid leukemia (AML), chronic myelogenous leukemia (CML), B cell acute lymphoid leukemia (B-ALL), T cell acute lymphoid leukemia (T-ALL), T cell lymphoma, and B cell lymphoma.

227. The method of claim 222, wherein the disease is an autoimmune disease.

228. The method of claim 227, wherein the autoimmune disease is selected from the group consisting of lupus, systemic lupus erythematosus, rheumatoid arthritis, psoriasis, psoriatic arthritis, multiple sclerosis, Crohn's disease, ulcerative colitis, Addison's disease, Graves' disease, Sjögren's syndrome, Hashimoto's thyroiditis, and celiac disease.

229. The method of claim 222, wherein the disease is diabetes mellitus.

230. The method of claim 229, wherein the diabetes is selected from the group consisting of Type I diabetes, Type II diabetes, prediabetes, and gestational diabetes.

231. The method of claim 222, wherein the disease is a neurological disease.

232. The method of claim 231, wherein the neurological disease is selected from the group consisting of catalepsy, epilepsy, encephalitis, meningitis, migraine, Huntington's, Alzheimer's, Parkinson's, Pelizaeus-Merzbacher disease, and multiple sclerosis.

233. A composition comprising a first polycistronic vector and a second polycistronic vector, wherein:
the first polycistronic vector comprises a nucleotide sequence encoding a first tolerogenic factor and a nucleotide sequence encoding a first CAR separated by one or more cleavage sites; and
the second polycistronic vector comprises a nucleotide sequence encoding a second tolerogenic factor and a nucleotide sequence encoding a second CAR separated by one or more cleavage sites.

234. The composition of claim 233, wherein the first tolerogenic factor and the second tolerogenic factor are independently selected from the group consisting of A20/TNFAIP3, CD16, CD16 Fc receptor, CD24, CD35, CD39, CD46, CD47, CD52, CD55, CD59, CD200, CCL22, CTLA4-Ig, C1 inhibitor, CR1, DUX4, FASL, H2-M3, IDO1, IL15-RF, HLA-C, HLA-E, HLA-E heavy chain, HLA-G, IL-10, IL-35, MANF, PD-1, PD-L1, Serpinb9, CC121, and Mfge8.

235. The composition of claim 234, wherein the first tolerogenic factor and the second tolerogenic factor comprise CD47.

236. The composition of claim 235, wherein the CD47 comprises an amino acid sequence that is at least 80% identical to the amino acid sequence set forth in any one of SEQ ID NOs: 1-5.

237. The composition of any one of claims 233-236, wherein the first CAR and the second CAR and different and independently selected from the group consisting of CD19 CAR, CD20 CAR, CD22 CAR, and BCMA CAR.

238. The composition of any one of claims 233-236, wherein the first CAR and/or the second CAR comprises an amino acid sequence set forth in any one of SEQ ID NOs: 32, 34, 36, 117, 128, and 136, or is at least 80% identical to the amino acid sequence set forth in any one of SEQ ID NOs: 32, 34, 36, 117, 128, and 136.

239. The composition of any one of claims 233-238, wherein the first CAR is CD19 CAR and the second CAR is CD22 CAR.

240. The composition of any one of claims 233-239, wherein the one or more cleavage sites comprise a self-cleaving site.

241. The composition of claim 240, wherein the self-cleaving site comprises a 2A site.

242. The composition of claim 241, wherein the 2A site comprises a T2A, P2A, E2A, or F2A site.

243. The composition of claim 241, wherein the one or more cleavage sites further comprise a protease site.

244. The composition of claim 243, wherein the protease site comprises a furin site.

245. The composition of claim 244, wherein the furin site comprises an FC1, FC2, or FC3 site.

246. The composition of claim 243, wherein the protease site precedes the 2A site in the 5′ to 3′ order.

247. A composition comprising a first virus comprising a first polycistronic vector and a second virus comprising a second polycistronic vector, wherein:
the first polycistronic vector comprises a nucleotide sequence encoding a first tolerogenic factor and a nucleotide sequence encoding a first CAR separated by one or more cleavage sites; and
the second polycistronic vector comprises a nucleotide sequence encoding a second tolerogenic factor and a nucleotide sequence encoding a second CAR separated by one or more cleavage sites.

248. The composition of claim 247, wherein the first virus and/or the second virus is an adenovirus, adeno-associated virus, retrovirus, lentivirus, or phage.

249. The composition of claim 247 or 248, wherein the first tolerogenic factor and the second tolerogenic factor are independently selected from the group consisting of A20/TNFAIP3, CD16, CD16 Fc receptor, CD24, CD35, CD39, CD46, CD47, CD52, CD55, CD59, CD200, CCL22, CTLA4-Ig, C1 inhibitor, CR1, DUX4, FASL, H2-M3, IDO1, IL15-RF, HLA-C, HLA-E, HLA-E heavy chain, HLA-G, IL-10, IL-35, MANF, PD-1, PD-L1, Serpinb9, CCl21, and Mfge8.

250. The composition of claim 249, wherein the first tolerogenic factor and the second tolerogenic factor comprise CD47.

251. The composition of claim 250, wherein the CD47 comprises an amino acid sequence that is at least 80% identical to the amino acid sequence set forth in any one of SEQ ID NOs: 1-5.

252. The composition of any one of claims 247-251, wherein the first CAR and the second CAR and different and independently selected from the group consisting of CD19 CAR, CD20 CAR, CD22 CAR, and BCMA CAR.

253. The composition of any one of claims 247-251, wherein the first CAR and/or the second CAR comprises an amino acid sequence set forth in any one of SEQ ID NOs: 32, 34, 36, 117, 128, and 136, or is at least 80% identical to the amino acid sequence set forth in any one of SEQ ID NOs: 32, 34, 36, 117, 128, and 136.

254. The composition of any one of claims 247-253, wherein the first CAR is CD19 CAR and the second CAR is CD22 CAR.

255. The composition of any one of claims 247-254, wherein the one or more cleavage sites comprise a self-cleaving site.

256. The composition of claim 255, wherein the self-cleaving site comprises a 2A site.

257. The composition of claim 256, wherein the 2A site comprises a T2A, P2A, E2A, or F2A site.

258. The composition of claim 256, wherein the one or more cleavage sites further comprise a protease site.

259. The composition of claim 258, wherein the protease site comprises a furin site.

260. The composition of claim 259, wherein the furin site comprises an FC1, FC2, or FC3 site.

261. The composition of claim 258, wherein the protease site precedes the 2A site in the 5′ to 3′ order.

262. A method of generating a heterogenous population of host cells, comprising introducing the composition of any one of claims 233-261 to the population of host cells.

263. A heterogenous population of host cells generated by the method of claim 262.

264. The population of host cells of claim 263, wherein the host cells are autologous cells.

265. The population of host cells of claim 263, wherein the host cells are allogeneic cells.

266. The population of host cells of any one of claims 263-265, wherein the host cells are ESCs or iPSCs.

267. The population of host cells of any one of claims 263-265, wherein the host cells are differentiated from an ESC or an iPSC.

268. The population of host cells of any one of claims 263-265, wherein the host cells are primary cells.

269. The population of host cells of claim 267 or 268, wherein the host cells are T cells, NK cells, or NKT cells.

270. A pharmaceutical composition comprising the population of host cells of any one of claims 263-269.

271. A method of treating a disease in a subject in need thereof, comprising administering the subject the population of host cells of any one of claims 263-269, or the pharmaceutical composition of claim 270.

272. The method of claim 271, wherein the disease is cancer.

273. The method of claim 272, wherein the cancer is associated with CD19, CD20, CD22, and/or BCMA expression.

274. The method of claim 272, wherein the cancer is a hematologic malignancy.

275. The method of claim 274, wherein the hematologic malignancy is selected from the group consisting of myeloid neoplasm, myelodysplastic syndromes (MDS), myeloproliferative/myelodysplastic syndromes, acute lymphoid leukemia (ALL), chronic lymphocytic leukemia (CLL), acute myeloid leukemia (AML), chronic myelogenous leukemia (CML), B cell acute lymphoid leukemia (B-ALL), T cell acute lymphoid leukemia (T-ALL), T cell lymphoma, and B cell lymphoma.

276. The method of claim 271, wherein the disease is an autoimmune disease.

277. The method of claim 276, wherein the autoimmune disease is selected from the group consisting of lupus, systemic lupus erythematosus, rheumatoid arthritis, psoriasis, psoriatic arthritis, multiple sclerosis, Crohn's disease, ulcerative colitis, Addison's disease, Graves' disease, Sjögren's syndrome, Hashimoto's thyroiditis, and celiac disease.

278. The method of claim 271, wherein the disease is diabetes mellitus.

279. The method of claim 278, wherein the diabetes is selected from the group consisting of Type I diabetes, Type II diabetes, prediabetes, and gestational diabetes.

280. The method of claim 271, wherein the disease is a neurological disease.

281. The method of claim 280, wherein the neurological disease is selected from the group consisting of catalepsy, epilepsy, encephalitis, meningitis, migraine, Huntington's, Alzheimer's, Parkinson's, Pelizaeus-Merzbacher disease, and multiple sclerosis.

282. A host cell having reduced expression of one or more MHC class I molecules, one or more MHC class II molecules, MIC-A, and/or MIC-B and increased expression of one or more tolerogenic factors selected from the group consisting of HLA-E, CD24, CD47, PD-L1, CD46, CD55, CD59, and C1 inhibitor, wherein the one or more tolerogenic factors are carried by the polycistronic vector of any one of claims 1 to 118 or a fragment thereof.

283. The host cell of claim 282, wherein the reduced expression of MHC class I molecules is by reduced expression of B2M.

284. The host cell of claim 282, wherein the reduced expression of MHC class II molecules is by reduced expression of CIITA.

285. The host cell of claim 282, wherein the reduced expression of MHC class I molecules and/or MHC class II molecules is by reduced expression of MIC-A and/or MIC-B.

286. The host cell of claim 282, wherein the host cell has knockout of one or more MHC class I molecules, one or more MHC class II molecules, MIC-A, and/or MIC-B.

287. The host cell of claim 282, wherein the host cell has knockout of one or more MHC class I molecules, one or more MHC class II molecules, MIC-A, and MIC-B.

288. The host cell of claim 282, wherein the host cell has knockout of MHC class I molecules, MHC class II molecules, MIC-A, and MIC-B.

289. The host cell of any one of claims 286-288, wherein the knockout is by use of a site-directed nuclease selected from the group consisting of Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, Mad7, a zinc finger nuclease (ZFN), a transcription activator-like effector nuclease (TALEN), a meganuclease, and a CRISPR-associated transposase.

290. The host cell of any one of claims 282-289, wherein the one or more tolerogenic factors comprise HLA-E.

291. The host cell of any one of claims 282-289, wherein the one or more tolerogenic factors is one or more tolerogenic factors comprise CD24.

292. The host cell of any one of claims 282-289, wherein the one or more tolerogenic factors comprise CD47.

293. The host cell of any one of claims 282-289, wherein the one or more tolerogenic factors comprise PD-L1.

294. The host cell of any one of claims 282-289, wherein the one or more tolerogenic factors comprise CD24, CD47, and PD-L1.

295. The host cell of any one of claims 282-289, wherein the one or more tolerogenic factors comprise CD46.

296. The host cell of any one of claims 282-289, wherein the one or more tolerogenic factors comprise CD55.

297. The host cell of any one of claims 282-289, wherein the one or more tolerogenic factors comprise CD59.

298. The host cell of any one of claims 282-289, wherein the one or more tolerogenic factors comprise C1 inhibitor.

299. The host cell of any one of claims 282-289, wherein the one or more tolerogenic factors comprise CD46, CD55, CD59, and C1 inhibitor.

300. The host cell of any one of claims 282-289, wherein the one or more tolerogenic factors comprise HLA-E, CD24, CD47, PD-L1, CD46, CD55, CD59, and C1 inhibitor.

301. The host cell of any one of claims 282-300, wherein the host cell is a pluripotent stem cell (PSC).

302. The host cell of claim 301, wherein the PSC is an ESC or an iPSC.

303. The host cell of any one of claims 282-300, wherein the host cell is differentiated from an ESC or an iPSC.

304. The host cell of any one of claims 282-300, wherein the host cell is a primary cell.

305. The host cell of claim 303 or 304, wherein the host cell is a T cell, a NK cell, a NKT cell, a β islet cell, or a GPC.

306. The host cell of any one of claims 282-305, wherein the polycistronic vector further comprises a nucleotide sequence encoding an additional exogenous component.

307. The host cell of claim 306, wherein the additional exogenous component is a safety switch selected from the group consisting of herpes simplex virus thymidine kinase (HSVtk), cytosine deaminase (CyD), nitroreductase (NTR), purine nucleoside phosphorylase (PNP), horseradish peroxidase, inducible caspase 9 (iCasp9), and rapamycin-activated caspase 9 (rapaCasp9).

308. A T cell having reduced expression of one or more MHC class I molecules, one or more MHC class II molecules, MIC-A, and/or MIC-B and increased expression of one or more tolerogenic factors selected from the group consisting of HLA-E, CD24, CD47, PD-L1, CD46, CD55, CD59, and C1 inhibitor, wherein the one or more tolerogenic factors are carried by the polycistronic vector of any one of claims 1 to 118 or a fragment thereof.

309. An NK cell having reduced expression of one or more MHC class I molecules, one or more MHC class II molecules, MIC-A, and/or MIC-B and increased expression of one or more tolerogenic factors selected from the group consisting of HLA-E, CD24, CD47, PD-L1, CD46, CD55, CD59, and C1 inhibitor, wherein the one or more tolerogenic factors are carried by the polycistronic vector of any one of claims 1 to 118 or a fragment thereof.

310. An islet cell having reduced expression of one or more MHC class I molecules, one or more MHC class II molecules, MIC-A, and/or MIC-B and increased expression of one or more tolerogenic factors selected from the group consisting of HLA-E, CD24, CD47, PD-L1, CD46, CD55, CD59, and C1 inhibitor, wherein the one or more tolerogenic factors are carried by the polycistronic vector of any one of claims 1 to 118 or a fragment thereof.

311. The cell of any one of claims 308-310, wherein the reduced expression of MHC class I molecules is by reduced expression of B2M.

312. The cell of any one of claims 308-310, wherein the reduced expression of MHC class II molecules is by reduced expression of CIITA.

313. The cell of any one of claims 308-310, wherein the reduced expression of MHC class I molecules and/or MHC class II molecules is by reduced expression of MIC-A and/or MIC-B.

314. The cell of any one of claims 308-310, wherein the cell has knockout of one or more MHC class I molecules, one or more MHC class II molecules, MIC-A, and/or MIC-B.

315. The cell of any one of claims 308-310, wherein the cell has knockout of one or more MHC class I molecules, one or more MHC class II molecules, MIC-A, and MIC-B.

316. The cell of any one of claims 308-310, wherein the cell has knockout of MHC class I molecules, MHC class II molecules, MIC-A, and MIC-B.

317. The cell of any one of claims 314-316, wherein the knockout is by use of a site-directed nuclease selected from the group consisting of Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, Mad7, a zinc finger nuclease (ZFN), a transcription activator-like effector nuclease (TALEN), a meganuclease, and a CRISPR-associated transposase.

318. The cell of any one of claims 308-317, wherein the one or more tolerogenic factors comprise HLA-E.

319. The cell of any one of claims 308-317, wherein the one or more tolerogenic factors is one or more tolerogenic factors comprise CD24.

320. The cell of any one of claims 308-317, wherein the one or more tolerogenic factors comprise CD47.

321. The cell of any one of claims 308-317, wherein the one or more tolerogenic factors comprise PD-L1.

322. The cell of any one of claims 308-317, wherein the one or more tolerogenic factors comprise CD24, CD47, and PD-L1.

323. The cell of any one of claims 308-317, wherein the one or more tolerogenic factors comprise CD46.

324. The cell of any one of claims 308-317, wherein the one or more tolerogenic factors comprise CD55.

325. The cell of any one of claims 308-317, wherein the one or more tolerogenic factors comprise CD59.

326. The cell of any one of claims 308-317, wherein the one or more tolerogenic factors comprise C1 inhibitor.

327. The cell of any one of claims 308-317, wherein the one or more tolerogenic factors comprise CD46, CD55, CD59, and C1 inhibitor.

328. The cell of any one of claims 308-317, wherein the one or more tolerogenic factors comprise HLA-E, CD24, CD47, PD-L1, CD46, CD55, CD59, and C1 inhibitor.

329. The cell of any one of claims 308-328, wherein the cell is differentiated from an ESC or an iPSC.

330. The cell of any one of claims 308-328, wherein the cell is a primary cell.

331. The cell of any one of claims 308-330, wherein the polycistronic vector further comprises a nucleotide sequence encoding an additional exogenous component.

332. The cell of claim 331, wherein the additional exogenous component is a safety switch selected from the group consisting of herpes simplex virus thymidine kinase (HSVtk), cytosine deaminase (CyD), nitroreductase (NTR), purine nucleoside phosphorylase (PNP), horseradish peroxidase, inducible caspase 9 (iCasp9), and rapamycin-activated caspase 9 (rapaCasp9).


SEQUENCE LISTING (138 sequences)

SEQ ID NO: 1 - protein, 305 aa, synthetic construct
  REGION 1..305: note = Synthetic: CD47-201 amino acid sequence
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRNN

SEQ ID NO: 2 - protein, 323 aa, synthetic construct
  REGION 1..323: note = Synthetic: CD47-202 amino acid sequence
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 3 - protein, 86 aa, synthetic construct
  REGION 1..86: note = Synthetic: CD47-203 amino acid sequence
  VARIANT 1: note = Xaa can be any naturally occurring amino acid
XWTESLYCGVYTNAWPSSDFRFEYLSSSTITWTSLYEICGVQRDTYSQLKGKKRQASNQKTIQPPRKAVEEPLNAFKESKGMMNDE

SEQ ID NO: 4 - protein, 109 aa, synthetic construct
  REGION 1..109: note = Synthetic: CD47-205 amino acid sequence
  VARIANT 1: note = Xaa can be any naturally occurring amino acid
XKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNE

SEQ ID NO: 5 - protein, 183 aa, synthetic construct
  REGION 1..183: note = Synthetic: CD47-206 amino acid sequence
MEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVF

SEQ ID NO: 6 - protein, 21 aa, synthetic construct
  REGION 1..21: note = Synthetic: CD8-alpha signal peptide
MALPVTALLLPLALLLHAARP

SEQ ID NO: 7 - protein, 20 aa, synthetic construct
  REGION 1..20: note = Synthetic: IgK signal peptide
METDTLLLWVLLLWVPGSTG

SEQ ID NO: 8 - protein, 22 aa, synthetic construct
  REGION 1..22: note = Synthetic: GMCSFR--alpha (CSF2RA) signal peptide
MLLLVTSLLLCELPHPAFLLIP

SEQ ID NO: 9 - protein, 45 aa, synthetic construct
  REGION 1..45: note = Synthetic: CD8-alpha hinge domain
TTTPAPRPPTPAPTIASQPLSLRPEACRPAAGGAVHTRGLDFACD

SEQ ID NO: 10 - protein, 39 aa, synthetic construct
  REGION 1..39: note = Synthetic: CD28 hinge domain
IEVMYPPPYLDNEKSNGTIIHVKGKHLCPSPLFPGPSKP

SEQ ID NO: 11 - protein, 12 aa, synthetic construct
  REGION 1..12: note = Synthetic: IgG4 hinge domain
ESKYGPPCPPCP

SEQ ID NO: 12 - protein, 12 aa, synthetic construct
  REGION 1..12: note = Synthetic: IgG4 hinge domain
ESKYGPPCPSCP

SEQ ID NO: 13 - protein, 229 aa, synthetic construct
  REGION 1..229: note = Synthetic: IgG4 hinge-CH2-CH3 domain
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 14 - protein, 24 aa, synthetic construct
  REGION 1..24: note = Synthetic: CD8-alpha transmembrane domain
IYIWAPLAGTCGVLLLSLVITLYC

SEQ ID NO: 15 - protein, 27 aa, synthetic construct
  REGION 1..27: note = Synthetic: CD28 transmembrane domain
FWVLVVVGGVLACYSLLVTVAFIIFWV

SEQ ID NO: 16 - protein, 42 aa, synthetic construct
  REGION 1..42: note = Synthetic: 4-1BB costimulatory domain
KRGRKKLLYIFKQPFMRPVQTTQEEDGCSCRFPEEEEGGCEL

SEQ ID NO: 17 - protein, 41 aa, synthetic construct
  REGION 1..41: note = Synthetic: CD28 costimulatory domain
RSKRSRLLHSDYMNMTPRRPGPTRKHYQPYAPPRDFAAYRS

SEQ ID NO: 18 - protein, 112 aa, synthetic construct
  REGION 1..112: note = Synthetic: CD3-zeta signaling domain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 19 - protein, 245 aa, synthetic construct
  REGION 1..245: note = Synthetic: Anti-CD19 FMC63 scFv entire sequence, with Whitlow linker
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 20 - protein, 107 aa, synthetic construct
  REGION 1..107: note = Synthetic: Anti-CD19 FMC63 scFv light chain variable region
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 21 - protein, 6 aa, synthetic construct
  REGION 1..6: note = Synthetic: Anti-CD19 FMC63 scFv light chain CDR1
QDISKY

SEQ ID NO: 22 - skipped

SEQ ID NO: 23 - protein, 9 aa, synthetic construct
  REGION 1..9: note = Synthetic: Anti-CD19 FMC63 scFv light chain CDR3
QQGNTLPYT

SEQ ID NO: 24 - protein, 18 aa, synthetic construct
  REGION 1..18: note = Synthetic: Whitlow linker
GSTSGSGKPGSGEGSTKG

SEQ ID NO: 25 - protein, 120 aa, synthetic construct
  REGION 1..120: note = Synthetic: Anti-CD19 FMC63 scFv heavy chain variable region
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 26 - protein, 8 aa, synthetic construct
  REGION 1..8: note = Synthetic: Anti-CD19 FMC63 scFv heavy chain CDR1
GVSLPDYG

SEQ ID NO: 27 - protein, 7 aa, synthetic construct
  REGION 1..7: note = Synthetic: Anti-CD19 FMC63 scFv heavy chain CDR2
IWGSETT

SEQ ID NO: 28 - protein, 14 aa, synthetic construct
  REGION 1..14: note = Synthetic: Anti-CD19 FMC63 scFv heavy chain CDR3
AKHYYYGGSYAMDY

SEQ ID NO: 29 - protein, 242 aa, synthetic construct
  REGION 1..242: note = Synthetic: Anti-CD19 FMC63 scFv entire sequence, with 3xG4S linker
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 30 - protein, 15 aa, synthetic construct
  REGION 1..15: note = Synthetic: 3xG4S linker
GGGGSGGGGSGGGGS

SEQ ID NO: 31 - DNA, 1458 bp, synthetic construct
  misc_feature 1..1458: note = Synthetic: Tisagenlecleucel CD19 CAR nucleotide sequence
  source 1..1458: mol_type = other DNA
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 32 - protein, 486 aa, synthetic construct
  REGION 1..486: note = Synthetic: Tisagenlecleucel CD19 CAR amino acid sequence
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 33 - DNA, 1383 bp, synthetic construct
  misc_feature 1..1383: note = Synthetic: Lisocabtagene maraleucel CD19 CAR nucleotide sequence
  source 1..1383: mol_type = other DNA
atgctgctgctggtgaccagcctgctgctgtgcgagctgccccaccccgcctttctgctgatccccgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcgaatctaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaagg

SEQ ID NO: 34 - protein, 461 aa, synthetic construct
  REGION 1..461: note = Synthetic: Lisocabtagene maraleucel CD19 CAR amino acid sequence
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 35 - DNA, 1467 bp, synthetic construct
  misc_feature 1..1467: note = Synthetic: Axicabtagene ciloleucel CD19 CAR nucleotide sequence
  source 1..1467: mol_type = other DNA
atgcttctcctggtgacaagccttctgctctgtgagttaccacacccagcattcctcctgatcccagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 36 - protein, 489 aa, synthetic construct
  REGION 1..489: note = Synthetic: Axicabtagene ciloleucel CD19 CAR amino acid sequence
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 37 - protein, 246 aa, synthetic construct
  REGION 1..246: note = Synthetic: Anti-CD20 Leu16 scFv entire sequence, with Whitlow linker
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGSGKPGSGEGSTKGEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSS

SEQ ID NO: 38 - protein, 106 aa, synthetic construct
  REGION 1..106: note = Synthetic: Anti-CD20 Leu16 scFv light chain variable region
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIK

SEQ ID NO: 39 - protein, 10 aa, synthetic construct
  REGION 1..10: note = Synthetic: Anti-CD20 Leu16 scFv light chain CDR1
RASSSVNYMD

SEQ ID NO: 40 - protein, 7 aa, synthetic construct
  REGION 1..7: note = Synthetic: Anti-CD20 Leu16 scFv light chain CDR2
ATSNLAS

SEQ ID NO: 41 - protein, 9 aa, synthetic construct
  REGION 1..9: note = Synthetic: Anti-CD20 Leu16 scFv light chain CDR3
QQWSFNPPT

SEQ ID NO: 42 - protein, 122 aa, synthetic construct
  REGION 1..122: note = Synthetic: Anti-CD20 Leu16 scFv heavy chain
EVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSS

SEQ ID NO: 43 - protein, 5 aa, synthetic construct
  REGION 1..5: note = Synthetic: Anti-CD20 Leu16 scFv heavy chain CDR1
SYNMH

SEQ ID NO: 44 - protein, 17 aa, synthetic construct
  REGION 1..17: note = Synthetic: Anti-CD20 Leu16 scFv heavy chain CDR2
AIYPGNGDTSYNQKFKG

SEQ ID NO: 45 - protein, 246 aa, synthetic construct
  REGION 1..246: note = Synthetic: Anti-CD22 m971 scFv entire sequence, with 3xG4S linker
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSGGGGSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIK

SEQ ID NO: 46 - protein, 124 aa, synthetic construct
  REGION 1..124: note = Synthetic: Anti-CD22 m971 scFv heavy chain variable region
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSS

SEQ ID NO: 47 - protein, 10 aa, synthetic construct
  REGION 1..10: note = Synthetic: Anti-CD22 m971 scFv heavy chain CDR1
GDSVSSNSAA

SEQ ID NO: 48 - protein, 9 aa, synthetic construct
  REGION 1..9: note = Synthetic: Anti-CD22 m971 scFv heavy chain CDR2
TYYRSKWYN

SEQ ID NO: 49 - protein, 14 aa, synthetic construct
  REGION 1..14: note = Synthetic: Anti-CD22 m971 scFv heavy chain CDR3
AREVTGDLEDAFDI

SEQ ID NO: 50 - protein, 107 aa, synthetic construct
  REGION 1..107: note = Synthetic: Anti-CD22 m971 scFv light chain
DIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIK

SEQ ID NO: 51 - protein, 6 aa, synthetic construct
  REGION 1..6: note = Synthetic: Anti-CD22 m971 scFv light chain CDR1
QTIWSY

SEQ ID NO: 52 - skipped

SEQ ID NO: 53 - protein, 9 aa, synthetic construct
  REGION 1..9: note = Synthetic: Anti-CD22 m971 scFv light chain CDR3
QQSYSIPQT

SEQ ID NO: 54 - protein, 246 aa, synthetic construct
  REGION 1..246: note = Synthetic: Anti-CD22 m971-L7 scFv entire sequence, with 3xG4S linker
QVQLQQSGPGMVKPSQTLSLTCAISGDSVSSNSVAWNWIRQSPSRGLEWLGRTYYRSTWYNDYAVSMKSRITINPDTNKNQFSLQLNSVTPEDTAVYYCAREVTGDLEDAFDIWGQGTMVTVSSGGGGSGGGGSGGGGSDIQMIQSPSSLSASVGDRVTITCRASQTIWSYLNWYRQRPGEAPNLLIYAASSLQSGVPSRFSGRGSGTDFTLTISSLQAEDFATYYCQQSYSIPQTFGQGTKLEIK

SEQ ID NO: 55 - protein, 124 aa, synthetic construct
  REGION 1..124: note = Synthetic: Anti-CD22 m971-L7 scFv heavy chain variable region
QVQLQQSGPGMVKPSQTLSLTCAISGDSVSSNSVAWNWIRQSPSRGLEWLGRTYYRSTWYNDYAVSMKSRITINPDTNKNQFSLQLNSVTPEDTAVYYCAREVTGDLEDAFDIWGQGTMVTVSS

SEQ ID NO: 56 - protein, 10 aa, synthetic construct
  REGION 1..10: note = Synthetic: Anti-CD22 m971-L7 scFv heavy chain CDR1
GDSVSSNSVA

SEQ ID NO: 57 - protein, 9 aa, synthetic construct
  REGION 1..9: note = Synthetic: Anti-CD22 m971-L7 scFv heavy chain CDR2
TYYRSTWYN

SEQ ID NO: 58 - protein, 14 aa, synthetic construct
  REGION 1..14: note = Synthetic: Anti-CD22 m971-L7 scFv heavy chain CDR3
AREVTGDLEDAFDI

SEQ ID NO: 59 - protein, 107 aa, synthetic construct
  REGION 1..107: note = Synthetic: Anti-CD22 m971-L7 scFv light chain variable region
DIQMIQSPSSLSASVGDRVTITCRASQTIWSYLNWYRQRPGEAPNLLIYAASSLQSGVPSRFSGRGSGTDFTLTISSLQAEDFATYYCQQSYSIPQTFGQGTKLEIK

SEQ ID NO: 60 - protein, 6 aa, synthetic construct
  REGION 1..6: note = Synthetic: Anti-CD22 m971-L7 scFv light chain CDR1
QTIWSY

SEQ ID NO: 61 - skipped

SEQ ID NO: 62 - protein, 9 aa, synthetic construct
  REGION 1..9: note = Synthetic: Anti-CD22 m971-L7 scFv light chain CDR3
QQSYSIPQT

SEQ ID NO: 63 - protein, 246 aa, synthetic construct
  REGION 1..246: note = Synthetic: Anti-BCMA C11D5.3 scFv entire sequence, with Whitlow linker
DIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 64 - protein, 111 aa, synthetic construct
  REGION 1..111: note = Synthetic: Anti-BCMA C11D5.3 scFv light chain variable region
DIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIK

SEQ ID NO: 65 - protein, 15 aa, synthetic construct
  REGION 1..15: note = Synthetic: Anti-BCMA C11D5.3 scFv light chain CDR1
RASESVSVIGAHLIH

SEQ ID NO: 66 - protein, 7 aa, synthetic construct
  REGION 1..7: note = Synthetic: Anti-BCMA C11D5.3 scFv light chain CDR2
LASNLET

SEQ ID NO: 67 - protein, 9 aa, synthetic construct
  REGION 1..9: note = Synthetic: Anti-BCMA C11D5.3 scFv light chain CDR3
LQSRIFPRT

SEQ ID NO: 68 - protein, 117 aa, synthetic construct
  REGION 1..117: note = Synthetic: Anti-BCMA C11D5.3 scFv heavy chain variable region
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 69 - protein, 5 aa, synthetic construct
  REGION 1..5: note = Synthetic: Anti-BCMA C11D5.3 scFv heavy chain CDR1
DYSIN

SEQ ID NO: 70 - protein, 17 aa, synthetic construct
  REGION 1..17: note = Synthetic: Anti-BCMA C11D5.3 scFv heavy chain CDR2
WINTETREPAYAYDFRG

SEQ ID NO: 71 - protein, 8 aa, synthetic construct
  REGION 1..8: note = Synthetic: Anti-BCMA C11D5.3 scFv heavy chain CDR3
DYSYAMDY

SEQ ID NO: 72 - protein, 246 aa, synthetic construct
  REGION 1..246: note = Synthetic: Anti-BCMA C12A3.2 scFv entire sequence, with Whitlow linker
DIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSS

SEQ ID NO: 73 - protein, 111 aa, synthetic construct
  REGION 1..111: note = Synthetic: Anti-BCMA C12A3.2 scFv light chain variable region
DIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIK

SEQ ID NO: 74 - protein, 15 aa, synthetic construct
  REGION 1..15: note = Synthetic: Anti-BCMA C12A3.2 scFv light chain CDR1
RASESVTILGSHLIY

SEQ ID NO: 75 - protein, 7 aa, synthetic construct
  REGION 1..7: note = Synthetic: Anti-BCMA C12A3.2 scFv light chain CDR2
LASNVQT

SEQ ID NO: 76 - protein, 9 aa, synthetic construct
  REGION 1..9: note = Synthetic: Anti-BCMA C12A3.2 scFv light chain CDR3
LQSRTIPRT

SEQ ID NO: 77 - protein, 117 aa, synthetic construct
  REGION 1..117: note = Synthetic: Anti-BCMA C12A3.2 scFv heavy chain variable region
QIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSS

SEQ ID NO: 78 - protein, 5 aa, synthetic construct
  REGION 1..5: note = Synthetic: Anti-BCMA C12A3.2 scFv heavy chain CDR1
HYSMN

SEQ ID NO: 79 - protein, 17 aa, synthetic construct
  REGION 1..17: note = Synthetic: Anti-BCMA C12A3.2 scFv heavy chain CDR2
RINTESGVPIYADDFKG

SEQ ID NO: 80 - protein, 8 aa, synthetic construct
  REGION 1..8: note = Synthetic: Anti-BCMA C12A3.2 scFv heavy chain CDR3
DYLYSLDF

SEQ ID NO: 81 - protein, 121 aa, synthetic construct
  REGION 1..121: note = Synthetic: Anti-BCMA FHVH33 entire sequence
EVQLLESGGGLVQPGGSLRLSCAASGFTFSSYAMSWVRQAPGKGLEWVSSISGSGDYIYYADSVKGRFTISRDISKNTLYLQMNSLRAEDTAVYYCAKEGTGANSSLADYRGQGTLVTVSS

SEQ ID NO: 82 - protein, 8 aa, synthetic construct
  REGION 1..8: note = Synthetic: Anti-BCMA FHVH33 CDR1
GFTFSSYA

SEQ ID NO: 83 - protein, 8 aa, synthetic construct
  REGION 1..8: note = Synthetic: Anti-BCMA FHVH33 CDR2
ISGSGDYI

SEQ ID NO: 84 - protein, 14 aa, synthetic construct
  REGION 1..14: note = Synthetic: Anti-BCMA FHVH33 CDR3
AKEGTGANSSLADY

SEQ ID NO: 85 - protein, 21 aa, synthetic construct
  REGION 1..21: note = Synthetic: T2A
  VARIANT 1..3: note = "GSG" is optional
GSGEGRGSLLTCGDVEENPGP

SEQ ID NO: 86 - protein, 22 aa, synthetic construct
  REGION 1..22: note = Synthetic: P2A
  VARIANT 1..3: note = "GSG" is optional
GSGATNFSLLKQAGDVEENPGP

SEQ ID NO: 87 - protein, 23 aa, synthetic construct
  REGION 1..23: note = Synthetic: E2A
  VARIANT 1..3: note = "GSG" is optional
GSGQCTNYALLKLAGDVESNPGP

SEQ ID NO: 88 - protein, 25 aa, synthetic construct
  REGION 1..25: note = Synthetic: F2A
  VARIANT 1..3: note = "GSG" is optional
GSGVKQTLNFDLLKLAGDVESNPGP

SEQ ID NO: 89 - protein, 7 aa, synthetic construct
  REGION 1..7: note = Synthetic: FC1
  VARIANT 1..3: note = "GSG" is optional
RRRRGSG

SEQ ID NO: 90 - protein, 7 aa, synthetic construct
  REGION 1..7: note = Synthetic: FC2
  VARIANT 1..3: note = "GSG" is optional
RKRRGSG

SEQ ID NO: 91 - protein, 15 aa, synthetic construct
  REGION 1..15: note = Synthetic: FC3
  VARIANT 1..3: note = "GSG" is optional
RKRRGSGTPDPWGSG

SEQ ID NO: 92 - DNA, 13 bp, synthetic construct
  misc_feature 1..13: note = Synthetic
  misc_difference 1..3: note = "gcc" is optional
  source 1..13: mol_type = other DNA
gccgccrccatgg

SEQ ID NO: 93 - DNA, 589 bp, synthetic construct
  misc_feature 1..589: note = Synthetic
  source 1..589: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 94 - skipped

SEQ ID NO: 95 - RNA, 12 nt, synthetic construct
  misc_feature 1..12: note = Synthetic: Exemplary spCas9 1 crRNA repeat region
  source 1..12: mol_type = other RNA
gttttagagcta

SEQ ID NO: 96 - skipped

SEQ ID NO: 97 - RNA, 66 nt, synthetic construct
  misc_feature 1..66: note = Synthetic: Exemplary spCas9 1 tracrRNA
  source 1..66: mol_type = other RNA
tagcaagttaaaataaggctagtccgttatcaacttgaaaaagtggcaccgagtcggtgctttttt

SEQ ID NO: 98 - skipped

SEQ ID NO: 99 - RNA, 17 nt, synthetic construct
  misc_feature 1..17: note = Synthetic: Exemplary spCas9 2 crRNA repeat region
  source 1..17: mol_type = other RNA
gtttsagagctatgctg

SEQ ID NO: 100 - skipped

SEQ ID NO: 101 - RNA, 71 nt, synthetic construct
  misc_feature 1..71: note = Synthetic: Exemplary spCas9 2 tracrRNA
  source 1..71: mol_type = other RNA
cagcatagcaagttsaaataaggctagtccgttatcaacttgaaaaagtggcaccgagtcggtgctttttt

SEQ ID NO: 102 - skipped

SEQ ID NO: 103 - RNA, 14 nt, synthetic construct
  misc_feature 1..14: note = Synthetic: Exemplary saCas9 crRNA repeat region
  source 1..14: mol_type = other RNA
gttttagtactctg

SEQ ID NO: 104 - skipped

SEQ ID NO: 105 - RNA, 64 nt, synthetic construct
  misc_feature 1..64: note = Synthetic: Exemplary saCas9 tracrRNA
  source 1..64: mol_type = other RNA
cagaatctactaaaacaaggcaaaatgccgtgtttatctcgtcaacttgttggcgagatttttt

SEQ ID NO: 106 - RNA, 88 nt, synthetic construct
  misc_feature 1..88: note = Synthetic: Exemplary AkCas12b tracrRNA
  source 1..88: mol_type = other RNA
gtcgtctataggacggcgaggacaacgggaagtgccaatgtgctctttccaagagcaaacaccccgttggcttcaagatgaccgctcg

SEQ ID NO: 107 - skipped

SEQ ID NO: 108 - RNA, 23 nt, synthetic construct
  misc_feature 1..23: note = Synthetic: Exemplary AkCas12b crRNA repeat region
  source 1..23: mol_type = other RNA
cgagcggtctgagaagtggcact

SEQ ID NO: 109 - skipped

SEQ ID NO: 110 - DNA, 20 bp, synthetic construct
  misc_feature 1..20: note = Synthetic: GET000046 guide
  source 1..20: mol_type = other DNA
accccacagtggggccacta

SEQ ID NO: 111 - DNA, 20 bp, synthetic construct
  misc_feature 1..20: note = Synthetic: GET000047 guide
  source 1..20: mol_type = other DNA
tgttggaaggatgaggaaat

SEQ ID NO: 112 - DNA, 20 bp, synthetic construct
  misc_feature 1..20: note = Synthetic: GET000048 guide
  source 1..20: mol_type = other DNA
tcactatgctgccgcccagt

SEQ ID NO: 113 - protein, 42 aa, synthetic construct
  REGION 1..42: note = Synthetic: CD28 hinge domain
AAAIEVMYPPPYLDNEKSNGTIIHVKGKHLCPSPLFPGPSKP

SEQ ID NO: 114 - protein, 28 aa, synthetic construct
  REGION 1..28: note = Synthetic: CD28 transmembrane domain
MFWVLVVVGGVLACYSLLVTVAFIIFWV

SEQ ID NO: 115 - protein, 112 aa, synthetic construct
  REGION 1..112: note = Synthetic: CD3-zeta signaling domain (with Q to K mutation at position 14)
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 116 - DNA, 1467 bp, synthetic construct
  misc_feature 1..1467: note = Synthetic: Exemplary CD19 CAR nucleotide sequence
  source 1..1467: mol_type = other DNA
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 117 - protein, 489 aa, synthetic construct
  REGION 1..489: note = Synthetic: Exemplary CD19 CAR amino acid sequence
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 118 - protein, 243 aa, synthetic construct
  REGION 1..243: note = Synthetic: Anti-BCMA CT103A scFv entire sequence, with Whitlow linker
DIQMTQSPSSLSASVGDRVTITCRASQSISSYLNWYQQKPGKAPKLLIYAASSLQSGVPSRFSGSGSGTDFTLTISSLQPEDFATYYCQQKYDLLTFGGGTKVEIKGSTSGSGKPGSGEGSTKGQLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARDRGDTILDVWGQGTMVTVSS

SEQ ID NO: 119 - protein, 106 aa, synthetic construct
  REGION 1..106: note = Synthetic: Anti-BCMA CT103A scFv light chain variable region
DIQMTQSPSSLSASVGDRVTITCRASQSISSYLNWYQQKPGKAPKLLIYAASSLQSGVPSRFSGSGSGTDFTLTISSLQPEDFATYYCQQKYDLLTFGGGTKVEIK

SEQ ID NO: 120 - protein, 6 aa, synthetic construct
  REGION 1..6: note = Synthetic: Anti-BCMA CT103A scFv light chain CDR1
QSISSY

SEQ ID NO: 121 - skipped

SEQ ID NO: 122 - protein, 8 aa, synthetic construct
  REGION 1..8: note = Synthetic: Anti-BCMA CT103A scFv light chain CDR3
QQKYDLLT

SEQ ID NO: 123 - protein, 119 aa, synthetic construct
  REGION 1..119: note = Synthetic: Anti-BCMA CT103A scFv heavy chain variable region
QLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARDRGDTILDVWGQGTMVTVSS

SEQ ID NO: 124 - protein, 10 aa, synthetic construct
  REGION 1..10: note = Synthetic: Anti-BCMA CT103A scFv heavy chain CDR1
GGSISSSSYY

SEQ ID NO: 125 - protein, 7 aa, synthetic construct
  REGION 1..7: note = Synthetic: Anti-BCMA CT103A scFv heavy chain CDR2
ISYSGST

SEQ ID NO: 126 - protein, 11 aa, synthetic construct
  REGION 1..11: note = Synthetic: Anti-BCMA CT103A scFv heavy chain CDR3
ARDRGDTILDV

SEQ ID NO: 127 - DNA, 1503 bp, synthetic construct
  misc_feature 1..1503: note = Synthetic: Exemplary BCMA CAR nucleotide sequence
  source 1..1503: mol_type = other DNA
atggccttaccagtgaccgccttgctcctgccgctggccttgctgctccacgccgccaggccggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcagcaaaaatacgacctcctcacttttggcggagggaccaaggttgagatcaaaggcagcaccagcggctccggcaagcctggctctggcgagggcagcacaaagggacagctgcagctgcaggagtcgggcccaggactggtgaagccttcggagaccctgtccctcacctgcactgtctctggtggctccatcagcagtagtagttactactggggctggatccgccagcccccagggaaggggctggagtggattgggagtatctcctatagtgggagcacctactacaacccgtccctcaagagtcgagtcaccatatccgtagacacgtccaagaaccagttctccctgaagctgagttctgtgaccgccgcagacacggcggtgtactactgcgccagagatcgtggagacaccatactagacgtatggggtcagggtacaatggtcaccgtcagctcattcgtgcccgtgttcctgcccgccaaacctaccaccacccctgcccctagacctcccaccccagccccaacaatcgccagccagcctctgtctctgcggcccgaagcctgtagacctgctgccggcggagccgtgcacaccagaggcctggacttcgcctgcgacatctacatctgggcccctctggccggcacctgtggcgtgctgctgctgagcctggtgatcaccctgtactgcaaccaccggaacaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcagatccgccgacgcccctgcctaccagcagggacagaaccagctgtacaacgagctgaacctgggcagacgggaagagtacgacgtgctggacaagcggagaggccgggaccccgagatgggcggaaagcccagacggaagaacccccaggaaggcctgtataacgaactgcagaaagacaagatggccgaggcctacagcgagatcggcatgaagggcgagcggaggcgcggcaagggccacgatggcctgtaccagggcctgagcaccgccaccaaggacacctacgacgccctgcacatgcaggccctgccccccaga

SEQ ID NO: 128 - protein, 501 aa, synthetic construct
  REGION 1..501: note = Synthetic: Exemplary BCMA CAR amino acid sequence
MALPVTALLLPLALLLHAARPDIQMTQSPSSLSASVGDRVTITCRASQSISSYLNWYQQKPGKAPKLLIYAASSLQSGVPSRFSGSGSGTDFTLTISSLQPEDFATYYCQQKYDLLTFGGGTKVEIKGSTSGSGKPGSGEGSTKGQLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARDRGDTILDVWGQGTMVTVSSFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 129 - DNA, 918 bp, synthetic construct
  misc_feature 1..918: note = Synthetic: CD47-201 CDS nucleotide sequence
  source 1..918: mol_type = other DNA
atgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggcttccaatcagaagactatacaacctcctaggaataactga

SEQ ID NO: 130 - DNA, 972 bp, synthetic construct
  misc_feature 1..972: note = Synthetic: CD47-202 CDS nucleotide sequence
  source 1..972: mol_type = other DNA
atgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggcttccaatcagaagactatacaacctcctaggaaagctgtagaggaaccccttaatgcattcaaagaatcaaaaggaatgatgaatgatgaataa

SEQ ID NO: 131 - DNA, 258 bp, synthetic construct
  misc_feature 1..258: note = Synthetic: CD47-203 CDS nucleotide sequence
  source 1..258: mol_type = other DNA
tggactgagtctctgtattgcggcgtgtataccaatgcatggccctcttctgatttcaggtttgagtatcttagctctagcacaattacttggactagtttatatgaaatttgtggggttcagagggacacctacagtcagttgaaaggcaagaagagacaagcttccaatcagaagactatacaacctcctaggaaagctgtagaggaaccccttaatgcattcaaagaatcaaaaggaatgatgaatgatgaataa

SEQ ID NO: 132 - DNA, 6796 bp, synthetic construct
  misc_feature 1..6796: note = Synthetic: CD47-204 CDS nucleotide sequence
  source 1..6796: mol_type = other DNA
agatattttcttgttcaatttaaggagaggtaaatttggtatcaatagaaaaaatgtttctgaaaaatttaaaccctggaaatgtatttatggcatggagtcagatgtttcagggagagaagaacaaatcaagaagcattgcaagtatgctcatatggaatgcttaaggcttgtggttaaaaaatatatatatatggctgtcaatgtcttaggctcatggtagcagcagaaatcgtaataattcttttgtcacatgggttatatccatattggagagaattaactcaggtgaaattaacttgtacactgtttggttttataatatttagagggatcacaactgactgatgtccctttgaagtaccattcttcataaatctttttttttcagaatgggccagccaactgtgacatcccttggatcggagatttagaactagaaagtattctttctacattattagggaagaaaaggagttacttggcggttagcaatattctattttgttttgttttgtttttagagacagggtctcattatgttgaccaggctggcctcgagctcctgggctcaagcaatgctcccacctcagcctcccaagtagctgggactacaggcatgtgccactacacctggcagtgtttattctgataaatacatttatgagctcaaaaatgtaactctaaaaccttatctctgaacttccatattaccatcagaaatttagatagttgtttagttctctttttctttgtagaacatagatataaggcatggtttcattgaagtcagttgtatatacatgtaactatcctgatgttcccaaataaagctctgtatttctgcttagtttattggggaggctgctaaatgtagtgcatcccaacccattttaccctgttctactttaaaaagaggttggcttcttgtttggatacaaggaccaagtcactcccccaggttcctccacagtaagggaggcctatttaaagccgcccatggcactaacagaaactggactcctatgagctcagatacataactgggcctcacaggggtgggacagtatgtagtctaggaattggaaggatccattccatatcaaagaactgaagcatcgtgttgccctctcagcagcaagagtaaggtgatgcccctgtcagttatagttcctgagttcctctgtctttgattctttgcctattagccagctagctcaccctcttgtttatgccactgttttttatcctattcatgccttctcacagacaacttttcttacctacagctttggactcatccttgtctcctttctgtttctttttcactttcccttcccatcaccaactttctgggtttttttctgtttcttcttagagtccagtggcagggagaaacttgtcagtccagtctgttgccatttttcctgtttgagaaagactcaccagcttttggctggctcacagattggctttccttgggtcaggacccacccttttccctgccagctttggaagcttgacagaattcgagtgtgcagtggtggtaaataaatagtaaggaacacagagcagtcctggaggcgtgcctccatctgctgatgagaaaatccagtgctgtcatccagcccaggtcccagcggaatgggcctctctgttcagtaggatccccctcctgctgagtggttcatggcatgtttctgttcaacgcttttccatctgtaggattcttattctgtatttatttgtttttttgggtttttttattttttgagatggagtctcgctctgtcgcccaggctggagtgcagtggcacgaccccagctcgctgcagcctctgcctcccaggacgagggagatcctcccacctcagccttccacgtagctgggactacaggcatgcaccacaggcatgcaccaccacgccagctaatttttgtatttttggtagagacagggttgcatcatgttgcccaggctggtcttgaatgcctgagctcaagcaatctatttgccttggcctcccaaagtgctgggattacaggcatgagccaccacggccagccttctcatttgttttttttataaggaagctatctcttcttccctccccaactagggtattctttttccctttcgtcactttgctcatgtactgtattccttcaacttcattaatgaatccatttggaagcagtgaaaaaggcaactcagaaagctaagaagaaatagatagaggaatactcagagctatctgagtattttctttagtttgttagctctttggagctttgaaactggaaagacccagggagtgatgtggagaaagagactgagcttgtaagacacaggagcagtgagctaagggagatggagtagtggggacaaattctggcacattctgtctacactctgggtagatagaggagggaggatggagcacccatggtgggggtatgttggtgacagcattttcccaccagccagtgtaacaagtggctgatttgggggaaagatggcataaacaaatgagagaatgtgtttactatttgatgtagatgggttatttgcttcatttttcaaatcagtgtatataatcaagaatattcagcatgtttgaatagactgtcagagctggaactctttcattaacatctctggcacctttagttttagccctgaacattttatcttaaaattaaacattaccaaatgccttagtttatttcatttattaaatttatattcttatttgttatttatatcagcttccaatcagaagactatacaacctcctaggaataactgaagtgaagtgatggactccgatttggagagtagtaagacgtgaaaggaatacacttgtgtttaagcaccatggccttgatgattcactgttggggagaagaaacaagaaaagtaactggttgtcacctatgagacccttacgtgattgttagttaagtttttattcaaagcagctgtaatttagttaataaaataattatgatctatgttgtttgcccaattgagatccagttttttgttgttatttttaatcaattaggggcaatagtagaatggacaatttccaagaatgatgcctttcaggtcctagggcctctggcctctaggtaaccagtttaaattggttcagggtgataactacttagcactgccctggtgattacccagagatatctatgaaaaccagtggcttccatcaaacctttgccaactcaggttcacagcagctttgggcagttatggcagtatggcattagctgagaggtgtctgccacttctgggtcaatggaataataaattaagtacaggcaggaatttggttgggagcatcttgtatgatctccgtatgatgtgatattgatggagatagtggtcctcattcttgggggttgccattcccacattcccccttcaacaaacagtgtaacaggtccttcccagatttagggtacttttattgatggatatgttttccttttattcacataaccccttgaaaccctgtcttgtcctcctgttacttgcttctgctgtacaagatgtagcaccttttctcctctttgaacatggtctagtgacacggtagcaccagttgcaggaaggagccagacttgttctcagagcactgtgttcacacttttcagcaaaaatagctatggttgtaacatatgtattcccttcctctgatttgaaggcaaaaatctacagtgtttcttcacttcttttctgatctggggcatgaaaaaagcaagattgaaatttgaactatgagtctcctgcatggcaacaaaatgtgtgtcaccatcaggccaacaggccagcccttgaatggggatttattactgttgtatctatgttgcatgataaacattcatcaccttcctcctgtagtcctgcctcgtactccccttcccctatgattgaaaagtaaacaaaacccacatttcctatcctggttagaagaaaattaatgttctgacagttgtgatcgcctggagtacttttagacttttagcattcgttttttacctgtttgtggatgtgtgtttgtatgtgcatacgtatgagataggcacatgcatcttctgtatggacaaaggtggggtacctacaggagagcaaaggttaattttgtgcttttagtaaaaacatttaaatacaaagttctttattgggtggaattatatttgatgcaaatatttgatcacttaaaacttttaaaacttctaggtaatttgccacgctttttgactgctcaccaataccctgtaaaaatacgtaattcttcctgtttgtgtaataagatattcatatttgtagttgcattaataatagttatttcttagtccatcagatgttcccgtgtgcctcttttatgccaaattgattgtcatatttcatgttgggaccaagtagtttgcccatggcaaacctaaatttatgacctgctgaggcctctcagaaaactgagcatactagcaagacagctcttcttgaaaaaaaaaatatgtatacacaaatatatacgtatatctatatatacgtatgtatatacacacatgtatattcttccttgattgtgtagctgtccaaaataataacatatatagagggagctgtattcctttatacaaatctgatggctcctgcagcactttttccttctgaaaatatttacattttgctaacctagtttgttactttaaaaatcagttttgatgaaaggagggaaaagcagatggacttgaaaaagatccaagctcctattagaaaaggtatgaaaatctttatagtaaaattttttataaactaaagttgtaccttttaatatgtagtaaactctcatttatttggggttcgctcttggatctcatccatccattgtgttctctttaatgctgcctgccttttgaggcattcactgccctagacaatgccaccagagatagtgggggaaatgccagatgaaaccaactcttgctctcactagttgtcagcttctctggataagtgaccacagaagcaggagtcctcctgcttgggcatcattgggccagttccttctctttaaatcagatttgtaatggctcccaaattccatcacatcacatttaaattgcagacagtgttttgcacatcatgtatctgttttgtcccataatatgctttttactccctgatcccagtttctgctgttgactcttccattcagttttatttattgtgtgttctcacagtgacaccatttgtccttttctgcaacaacctttccagctacttttgccaaattctatttgtcttctccttcaaaacattctcctttgcagttcctcttcatctgtgtagctgctcttttgtctcttaacttaccattcctatagtactttatgcatctctgcttagttctattagttttttggccttgctcttctccttgattttaaaattccttctatagctagagcttttctttctttcattctctcttcctgcagtgttttgcatacatcagaagctaggtacataagttaaatgattgagagttggctgtatttagatttatcactttttaatagggtgagcttgagagttttctttctttctgttttttttttttgttttttttttttttttttttttttttttttttgactaatttcacatgctctaaaaaccttcaaaggtgattatttttctcctggaaactccaggtccattctgtttaaatccctaagaatgtcagaattaaaataacagggctatcccgtaattggaaatatttcttttttcaggatgctatagtcaatttagtaagtgaccaccaaattgttatttgcactaacaaagctcaaaacacgataagtttactcctccatctcagtaataaaaattaagctgtaatcaaccttctaggtttctcttgtcttaaaatgggtattcaaaaatggggatctgtggtgtatgtatggaaacacatactccttaatttacctgttgttggaaactggagaaatgattgtcgggcaaccgtttattttttattgtattttatttggttgagggatttttttataaacagttttacttgtgtcatattttaaaattactaactgccatcacctgctggggtcctttgttaggtcattttcagtgactaatagggataatccaggtaactttgaagagatgagcagtgagtgaccaggcagtttttctgcctttagctttgacagttcttaattaagatcattgaagaccagctttctcataaatttctctttttgaaaaaaagaaagcatttgtactaagctcctctgtaagacaacatcttaaatcttaaaagtgttgttatcatgactggtgagagaagaaaacattttgtttttattaaatggagcattatttacaaaaagccattgttgagaattagatcccacatcgtataaatatctattaaccattctaaataaagagaactccagtgttgctatgtgcaagatcctctcttggagcttttttgcatagcaattaaaggtgtgctatttgtcagtagccatttttttgcagtgatttgaagaccaaagttgttttacagctgtgttaccgttaaaggtttttttttttatatgtattaaatcaatttatcactgtttaaagctttgaatatctgcaatctttgccaaggtacttttttatttaaaaaaaaacataactttgtaaatattaccctgtaatattatatatacttaataaaacattttaagctat

SEQ ID NO: 133 - DNA, 327 bp, synthetic construct
  misc_feature 1..327: note = Synthetic: CD47-205 CDS nucleotide sequence
  source 1..327: mol_type = other DNA
aagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggcttccaatcagaagactatacaacctcctaggaaagctgtagaggaaccccttaatgaataa

SEQ ID NO: 134 - DNA, 549 bp, synthetic construct
  misc_feature 1..549: note = Synthetic: CD47-206 CDS nucleotide sequence
  source 1..549: mol_type = other DNA
atggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgttt

SEQ ID NO: 135 - DNA, 969 bp, synthetic construct
  misc_feature 1..969: note = Synthetic: Codon-optimized nucleotide sequence encoding CD47
  source 1..969: mol_type = other DNA
atgtggcccctggtcgccgccctgttgctgggctcggcatgctgcggatcagctcagctactgtttaataaaacaaaatctgtagaattcacgttttgtaacgacactgtcgtgatcccatgctttgttactaatatggaggcacaaaacaccactgaagtgtacgtgaagtggaaattcaaaggcagagacatttacacctttgacggcgccctcaacaagtccaccgtgcccactgactttagtagcgcaaaaattgaggtcagccaattactaaaaggagatgcctctttgaagatggacaagagcgatgctgtcagccacacagggaactacacttgtgaagtaacagagttaacccgcgaaggtgaaacgatcatcgagctgaagtatcgagtggtgtcctggttttctccgaacgagaatatccttatcgtaattttcccaattttcgctatcctcctgttctggggccagtttggtatcaagacactcaaatatcggtccggtgggatggatgagaagacaattgccctgcttgttgctggactcgtgatcaccgtcatcgtgattgttggggccatccttttcgtcccaggggagtacagcctgaagaatgctacgggcctgggattaattgtgacctctacagggatactcatcctgcttcactactatgtgttcagtaccgcgattggactgacctccttcgtcattgccatattggtgattcaggtgatagcctacatcctcgccgtggttggcctgagtctctgtatcgcggcgtgcatacccatgcatggccctcttctgatttcagggttgagtatcctcgcactagcacagttgctgggactggtttatatgaaatttgtggcctccaaccagaagactatacagcctcctaggaaggctgtagaggagcccctgaatgcattcaaggaatcaaaaggcatgatgaatgatgaa

SEQ ID NO: 136 - protein, 484 aa, synthetic construct
  REGION 1..484: note = Synthetic: Exemplary CD22 CAR amino acid sequence
MLLLVTSLLLCELPHPAFLLIPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 137 - DNA, 20 bp, synthetic construct
  misc_feature 1..20: note = Synthetic
  source 1..20: mol_type = other DNA
gatgaccctggctcatatca

SEQ ID NO: 138 - DNA, 20 bp, synthetic construct
  misc_feature 1..20: note = Synthetic
  source 1..20: mol_type = other DNA
gtttctgcctgtcatagcgc